SEQUENCE LISTING (69 sequences)

SEQ ID NO: 1 - protein, 4 aa, Artificial Sequence
  Synthetic peptide
GFLG

SEQ ID NO: 2 - protein, 4 aa, Artificial Sequence
  Synthetic peptide
KPLR

SEQ ID NO: 3 - protein, 4 aa, Artificial Sequence
  Synthetic peptide
YLLL

SEQ ID NO: 4 - protein, 12 aa, Artificial Sequence
  Synthetic peptide
DDDDDDDDDDDD

SEQ ID NO: 5 - protein, 11 aa, Artificial Sequence
  Synthetic peptide
DDDDDDDDDDD

SEQ ID NO: 6 - protein, 10 aa, Artificial Sequence
  Synthetic peptide
DDDDDDDDDD

SEQ ID NO: 7 - protein, 9 aa, Artificial Sequence
  Synthetic peptide
DDDDDDDDD

SEQ ID NO: 8 - protein, 8 aa, Artificial Sequence
  Synthetic peptide
DDDDDDDD

SEQ ID NO: 9 - protein, 7 aa, Artificial Sequence
  Synthetic peptide
DDDDDDD

SEQ ID NO: 10 - protein, 6 aa, Artificial Sequence
  Synthetic peptide
DDDDDD

SEQ ID NO: 11 - protein, 5 aa, Artificial Sequence
  Synthetic peptide
DDDDD

SEQ ID NO: 12 - protein, 4 aa, Artificial Sequence
  Synthetic peptide
DDDD

SEQ ID NO: 13 - protein, 3 aa, Artificial Sequence
  Synthetic peptide
  misc_feature (1)..(1): Xaa is Beta-alanine
  misc_feature (3)..(3): Xaa is a suitable counterion
XVX

SEQ ID NO: 14 - protein, 5 aa, Artificial Sequence
  Synthetic peptide
  misc_feature (1)..(1): Xaa is beta-alanine
  misc_feature (5)..(5): Xaa is a suitable counterion
XSPVX

SEQ ID NO: 15 - protein, 5 aa, Artificial Sequence
  Synthetic peptide
  misc_feature (1)..(1): Xaa is beta-alanine
  misc_feature (5)..(5): Xaa is a suitable counterion
XSKVX

SEQ ID NO: 16 - protein, 5 aa, Artificial Sequence
  Synthetic peptide
  misc_feature (1)..(1): Xaa is beta-alanine
  misc_feature (5)..(5): Xaa is a suitable counterion
XSKVX

SEQ ID NO: 17 - protein, 3 aa, Artificial Sequence
  Synthetic peptide
  misc_feature (1)..(1): Xaa is beta-alanine
XVK

SEQ ID NO: 18 - protein, 3 aa, Artificial Sequence
  Synthetic peptide
  misc_feature (1)..(1): Xaa is beta-alanine
XVK

SEQ ID NO: 19 - protein, 3 aa, Artificial Sequence
  Synthetic peptide
  misc_feature (1)..(1): Xaa is beta-alanine
  misc_feature (3)..(3): Xaa is alpha-aminobutyric acid
XVX

SEQ ID NO: 20 - protein, 5 aa, Artificial Sequence
  Synthetic peptide
  misc_feature (1)..(1): Xaa is beta-alanine
  misc_feature (5)..(5): Xaa is alpha-aminobutyric acid
XSPVX

SEQ ID NO: 21 - protein, 5 aa, Artificial Sequence
  Synthetic peptide
  misc_feature (1)..(1): Xaa is beta-alanine
  misc_feature (5)..(5): Xaa is alpha-aminobutyric acid
XSKVX

SEQ ID NO: 22 - protein, 5 aa, Artificial Sequence
  Synthetic peptide
  misc_feature (1)..(1): Xaa is beta-alanine
  misc_feature (5)..(5): Xaa is alpha-aminobutyric acid
XSKVX

SEQ ID NO: 23 - protein, 5 aa, Artificial Sequence
  Synthetic peptide
  misc_feature (1)..(1): Xaa is beta-alanine
  misc_feature (5)..(5): Xaa is alpha-aminobutyric acid
XSKSX

SEQ ID NO: 24 - protein, 5 aa, Artificial Sequence
  Synthetic peptide
  misc_feature (1)..(1): Xaa is beta-alanine
  misc_feature (5)..(5): Xaa is alpha-aminobutyric acid
XSKSX

SEQ ID NO: 25 - protein, 3 aa, Artificial Sequence
  Synthetic peptide
  misc_feature (1)..(1): Xaa is beta-alanine
  misc_feature (3)..(3): Xaa is norleucine
XVX

SEQ ID NO: 26 - protein, 5 aa, Artificial Sequence
  Synthetic peptide
  misc_feature (1)..(1): Xaa is beta-alanine
  misc_feature (5)..(5): Xaa is norleucine
XSPVX

SEQ ID NO: 27 - protein, 5 aa, Artificial Sequence
  Synthetic peptide
  misc_feature (1)..(1): Xaa is beta-alanine
  misc_feature (5)..(5): Xaa is norleucine
XSKVX

SEQ ID NO: 28 - protein, 5 aa, Artificial Sequence
  Synthetic peptide
  misc_feature (1)..(1): Xaa is beta-alanine
  misc_feature (5)..(5): Xaa is norleucine
XSKVX

SEQ ID NO: 29 - protein, 5 aa, Artificial Sequence
  Synthetic peptide
  misc_feature (1)..(1): Xaa is beta-alanine
  misc_feature (5)..(5): Xaa is norleucine
XSKSX

SEQ ID NO: 30 - protein, 5 aa, Artificial Sequence
  Synthetic peptide
  misc_feature (1)..(1): Xaa is beta-alanine
  misc_feature (5)..(5): Xaa is norleucine
XSKSX

SEQ ID NO: 31 - protein, 5 aa, Artificial Sequence
  Synthetic peptide
  misc_feature (1)..(1): Xaa is beta-alanine
  misc_feature (5)..(5): Xaa is norleucine
XSPVX

SEQ ID NO: 32 - protein, 5 aa, Artificial Sequence
  Synthetic peptide
  misc_feature (1)..(1): Xaa is beta-alanine
  misc_feature (5)..(5): Xaa is norleucine
XSKSX

SEQ ID NO: 33 - protein, 5 aa, Artificial Sequence
  Synthetic peptide
FFFFF

SEQ ID NO: 34 - protein, 5 aa, Artificial Sequence
  Synthetic peptide
FFFFF

SEQ ID NO: 35 - protein, 5 aa, Artificial Sequence
  Synthetic peptide
WWWWW

SEQ ID NO: 36 - protein, 5 aa, Artificial Sequence
  Synthetic peptide
WWWWW

SEQ ID NO: 37 - protein, 5 aa, Artificial Sequence
  Synthetic peptide
  misc_feature (1)..(5): Xaa is paraphenylalanine
XXXXX

SEQ ID NO: 38 - protein, 5 aa, Artificial Sequence
  Synthetic peptide
  misc_feature (1)..(5): Xaa is paraphenylalanine
XXXXX

SEQ ID NO: 39 - protein, 10 aa, Artificial Sequence
  Synthetic peptide
  misc_feature (1)..(10): Xaa is paraphenylalanine
XXXXXXXXXX

SEQ ID NO: 40 - protein, 10 aa, Artificial Sequence
  Synthetic peptide
  misc_feature (1)..(10): Xaa is paraphenylalanine
XXXXXXXXXX

SEQ ID NO: 41 - protein, 20 aa, Artificial Sequence
  Synthetic peptide
  misc_feature (1)..(20): Xaa is paraphenylalanine
XXXXXXXXXXXXXXXXXXXX

SEQ ID NO: 42 - protein, 20 aa, Artificial Sequence
  Synthetic peptide
  misc_feature (1)..(20): Xaa is paraphenylalanine
XXXXXXXXXXXXXXXXXXXX

SEQ ID NO: 43 - protein, 3 aa, Artificial Sequence
  Synthetic peptide
EEE

SEQ ID NO: 44 - protein, 5 aa, Artificial Sequence
  Synthetic peptide
EEEEE

SEQ ID NO: 45 - protein, 5 aa, Artificial Sequence
  Synthetic peptide
EEEEE

SEQ ID NO: 46 - protein, 5 aa, Artificial Sequence
  Synthetic peptide
EWEWE

SEQ ID NO: 47 - protein, 5 aa, Artificial Sequence
  Synthetic peptide
WEWEW

SEQ ID NO: 48 - protein, 10 aa, Artificial Sequence
  Synthetic peptide
WWEWWWWEWW

SEQ ID NO: 49 - protein, 5 aa, Artificial Sequence
  Synthetic peptide
WWEWW

SEQ ID NO: 50 - protein, 5 aa, Artificial Sequence
  Synthetic peptide
EWEWE

SEQ ID NO: 51 - protein, 10 aa, Artificial Sequence
  Synthetic peptide
EWEWEEWEWE

SEQ ID NO: 52 - protein, 10 aa, Artificial Sequence
  Synthetic peptide
WEWEWWEWEW

SEQ ID NO: 53 - protein, 15 aa, Artificial Sequence
  Synthetic peptide
GGEGGEGGEGGEGGE

SEQ ID NO: 54 - protein, 15 aa, Artificial Sequence
  Synthetic peptide
GGEGGWGGEGGWGGE

SEQ ID NO: 55 - protein, 15 aa, Artificial Sequence
  Synthetic peptide
GGEGGWGGEGGWGGE

SEQ ID NO: 56 - protein, 15 aa, Artificial Sequence
  Synthetic peptide
GGEGGWGGEGGWGGE

SEQ ID NO: 57 - protein, 30 aa, Artificial Sequence
  Synthetic peptide
KGWGWGKGWGWGKGWGWGKGWGWGKGWGWG

SEQ ID NO: 58 - protein, 30 aa, Artificial Sequence; identical to SEQ ID NO: 57
  Synthetic peptide

SEQ ID NO: 59 - protein, 11 aa, Artificial Sequence
  Synthetic peptide
  misc_feature (1)..(11): Xaa is paraphenylalanine
XXXXXXXXXXX

SEQ ID NO: 60 - protein, 21 aa, Artificial Sequence
  Synthetic peptide
  misc_feature (1)..(21): Xaa is paraphenylalanine
XXXXXXXXXXXXXXXXXXXXX

SEQ ID NO: 61 - protein, 21 aa, Artificial Sequence
  Synthetic peptide
  misc_feature (1)..(21): Xaa is paraphenylalanine
XXXXXXXXXXXXXXXXXXXXX

SEQ ID NO: 62 - protein, 6 aa, Artificial Sequence
  Synthetic peptide
  MOD_RES (1)..(1): Xaa is aminohexanoic acid
XWWWWW

SEQ ID NO: 63 - protein, 11 aa, Artificial Sequence
  Synthetic peptide
  misc_feature (1)..(11): Xaa is paraphenylalanine
XXXXXXXXXXX

SEQ ID NO: 64 - protein, 6 aa, Artificial Sequence
  Synthetic peptide
  MOD_RES (1)..(1): Xaa is aminohexanoic acid
XEWEWE

SEQ ID NO: 65 - protein, 5 aa, Artificial Sequence
  Synthetic peptide
EWWWE

SEQ ID NO: 66 - protein, 6 aa, Artificial Sequence
  Synthetic peptide
  MOD_RES (1)..(1): Xaa is aminohexanoic
XWWWWW

SEQ ID NO: 67 - protein, 8 aa, Artificial Sequence
  Synthetic peptide
  MOD_RES (1)..(1): Xaa is beta alanine
  misc_feature (3)..(3): Xaa is aminohexanoic acid
XVXWWWWW

SEQ ID NO: 68 - protein, 6 aa, Artificial Sequence
  Synthetic peptide
  misc_feature (1)..(1): Xaa is aminohexanoic acid
XWWWWW

SEQ ID NO: 69 - protein, 6 aa, Artificial Sequence
  Synthetic peptide
  misc_feature (1)..(1): Xaa is aminohexanoic acid
XWWWWW

(19) United States
(12) Patent Application Publication
(10) Pub. No.: US 2024/0382614 A1
(43) Pub. Date: Nov. 21, 2024

(54) Self-Assembling Nanoparticles Based On Amphiphilic Peptides for Drug Delivery Applications

(71) Applicants: Barinthus Biotherapeutics North America, Inc., Germantown, MD (US); THE UNITED STATES OF AMERICA, AS REPRESENTED BY THE SECRETARY, DEPARTMENT OF HEALTH AND HUMAN SERVIC, Bethesda, MD (US)

(72) Inventors: Geoffrey M. Lynn, McLean, VA (US); Yaling Zhu, Gaithersburg, MD (US); Sarah R. Nichols, Mount Airy, MD (US); Qiuyin Ren, Silver Spring, MD (US); Andrew S. Ishizuka, Brookline, MA (US)

(73) Assignees: Barinthus Biotherapeutics North America, Inc., Germantown, MD (US); The United States of America, as Represented by the Secretary, Department of Health and Human Servic, Bethesda, MD (US)

(21) Appl. No.: 18/570,579

(22) PCT Filed: Jun. 16, 2022
(86) PCT No.: PCT/US2022/033819

§ 371 Date: Dec. 14, 2023

Related U.S. Application Data
(60) Provisional application No. 63/284,375, filed on Nov. 30, 2021.
(60) Provisional application No. 63/211,336, filed on Jun. 16, 2021.

(51) Int. Cl. A61K 47/69; A61K 45/06; A61K 47/60; A61K 47/61; A61P 37/06

(52) U.S. Cl. CPC A61K 47/6907; A61K 45/06; A61K 47/60; A61K 47/61; A61P 37/06

(57) ABSTRACT

The present disclosure relates to a composition comprising a first amphiphile and optionally a second amphiphile each having the formula S-[B]-[U]-H-[D] and at least one drug molecule is noncovalently associated with or covalently bonded directly or via a suitable linker X1 to the first amphiphile and/or to the optional second amphiphile. The composition is useful in treating a cancer, an infectious disease or an inflammatory disease.

### CROSS-REFERENCE TO RELATED APPLICATIONS

This application is a national phase entry pursuant to 35 U.S.C. § 371 of International Application No. PCT/US2022/033819, filed on Jun. 16, 2022, which claims the benefit of, and priority to, U.S. Provisional Patent Application Nos. 63/284,375, filed Nov. 30, 2021, and 63/211,336, filed Jun. 16, 2021, the contents of each of which are hereby incorporated by reference in their entirety.

### GOVERNMENT RIGHTS

This invention was created in the performance of a Cooperative Research and Development Agreement with the National Institutes of Health, an Agency of the Department of Health and Human Services. The Government of the United States has certain rights in this invention.

### SEQUENCE LISTING

The instant application contains a Sequence Listing which has been submitted electronically in ASCII format and is hereby incorporated by reference in its entirety. Said ASCII copy, created on Jul. 17, 2024, is named BARI_011_SL.txt, and is 17,494 bytes in size.

### TECHNICAL FIELD

The present disclosure relates to novel amphiphile compositions, particularly polymer-based amphiphile compositions, that can be used to form nanoparticles, including micelle structures or polymersomes, methods of manufacturing the amphiphile compositions, processes for formulating drug molecules with the amphiphile compositions that form nanoparticles, and therapeutic uses of the nanoparticles for drug delivery.

### BACKGROUND

Most promising cancer therapies are limited by off-target toxicity due to insufficient selectively of the treatment. Moreover, many immunomodulators for treating inflammatory diseases often result in serious adverse events due to broad immunosuppression or other off-target toxicities. Even localized infections often require systemic treatments that are accompanied by systemic side effects. Therefore, based on these challenges, there remains a need for drug delivery platforms that improve selectivity of drug molecule targeting to specific tissues, particularly for treating cancer, infectious diseases and inflammatory diseases.

Current drug delivery platforms are limited by use of empirical, trial-and-error formulation processes that often lead to inadequate and/or variable drug molecule loading; high uptake by cells of the reticuloendothelial system localized in liver and spleen leading to poor drug molecule accumulation in target tissues; and/or dependence on antigens that restricts the potential reach of a given treatment.

Discussed herein are improved compositions and methods of manufacturing nanosized carriers of drugs (referred to as “nanomedicines”) that address contemporary challenges.

### SUMMARY

The present disclosure provides a composition comprising a first amphiphile and optionally a second amphiphile each having the formula S-[B]-[U]-H, wherein S, independently for each occurrence, is a solubilizing block;
B, independently for each occurrence, is a spacer;
H, independently for each occurrence, is a hydrophobic block;
U, independently for each occurrence, is a linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof; and
at least one drug molecule D is noncovalently associated with or covalently bonded directly or via a suitable linker X1 to the first amphiphile and/or the optional second amphiphile.

The present disclosure also provides a method of preventing or inhibiting an immune response in a subject against a second drug molecule (D2) or an expression system (D2e), wherein the method comprises administering to the subject a composition comprising:
at least one amphiphile; and
a first drug molecule (D1) selected from inhibitors of mTORC1 and/or mTORC2,
wherein the composition is administered to the subject at a time 1 (T1) and and the second drug molecule (D2) or the expression system (D2e) is administered to the subject at a time (T2).

### BRIEF DESCRIPTION OF FIGURES

FIG. 1 shows a cartoon schematic of a non-limiting example of an amphiphile of formula S-B-[U]-H-D, wherein the solubilizing block (S) comprise a dendron amplifier, and a particle comprising amphiphiles of formula S-B-[U]-H-D, wherein the solubilizing block comprises a dendron amplifier.

FIG. 2 shows a cartoon schematic of a non-limiting example of a particle assembled from a first amphiphile and a second amphiphile, wherein the first amphiphile has the formula S-B-[U]-H-D and the solubilizing block (S) comprises a dendron amplifier, and the second amphiphile has the formula S-[B]-[U]-H-D, wherein the solubilizing block comprises a linear polymer.

FIG. 3A-3E shows cartoon schematics of non-limiting examples of amphiphiles of formula S-[B]-[U]-H-D. (A) and (B) show cartoon schematics of an amphiphile with the formula S-B-[U]-H-D, wherein S comprises a dendron amplifier, with varying lengths of the spacer (B). (C) and (D) show cartoon schematics of an amphiphile with the formula S-[B]-[U]-H-D, wherein S comprises a linear polymer, with varying lengths of the spacer (B). (E) shows a cartoon schematic of an amphiphile with the formula S-B-[U]-H-D, wherein S and B are linked via the linker X6.

FIG. 4A-4B shows the pH-responsiveness of exemplary amphiphiles with S blocks comprising carboxylic acid solubilizing groups. (A) Structures of the amphiphiles: Compounds 107, 122 and 121 comprise a PEG-based dendron amplifier with R²⁵ selected from beta-alanine, glycine and —OH, respectively. Compound 124 comprises a peptide-based dendron amplifier with R²⁶ selected from glutaric acid and Compounds 125 and 126 both comprise a peptide-based dendron amplifier with R²⁶ selected from succinic acid, but for compound 125 the spacer B is absent. U comprises a triazole and the H block is Ahx-2B3W2 (Compound 56). (B) Absorbance at 490 nm (turbidity) was assessed for each of the different amphiphiles at 0.1 mM in PBS buffer at the specified pH. Absorbance values greater than about 0.04 in this study indicate that aggregation is occurring.

FIG. 5 shows the pH-dependent change in cell uptake for different compositions of amphiphiles. Particles comprising a fluorescent dye molecule (Cyanine 5) and an amphiphile comprising either an amine solubilizing groups (+8 net charge peptide antigen conjugate (PAC) KKKKKKKKVRKARDETAALLNSAVLGAAPLFVPPADSPVZX(DBCO-Ahx-2B3W2) and Compound 100), mannose solubilizing groups (Compound 97), or carboxylic acid solubilizing groups (Compounds 121, 124, 126 and 107) were added at 20 uM concentration to murine (C57BL/6)-derived splenocytes in HEPES buffer at different pH values (7.4, 7.0 and 6.5) and then incubated for 2 hours at 37° C. Following 2 hours of incubation, cells were washed, stained with a viability-dye and then fixed, prior to analyzing by flow cytometry for the percent of cells that were positive for Cyanine 5 (Cy5) dye uptake as well as the geometric mean Cy5 fluorescence intensity. The data show the fold change in uptake at a given pH relative to uptake for that same group at pH 7.4.

FIG. 6A-6B shows how the composition of particles comprising amphiphiles impacts tumor selectivity. Particles comprising a fluorophore (AlexaFluor647) and either a first amphiphile (groups 1 to 4), a first amphiphile and a second amphiphile (groups 5-8) or a positively charged peptide antigen conjugate (group 9), based on the compositions summarized in Table 6, were injected intravenously to albino BL/6 mice at time 0 and then organ fluorescence was at several timepoints thereafter to assess particle distribution and kinetics in different tissues. The data show (A) the fluorescence intensity in tumor and (B) the ratio of liver to tumor uptake.

FIG. 7 shows the kinetics of enzyme (cathepsin)-mediated hydrolysis for different peptide-AMC substrates. The different peptide-AMC substrates (Compounds 8-25) summarized in Table leach at 0.3 mM in 25 mM MES buffer at pH 5.0 were incubated with 2 ng/mL of Cathepsin B (Sino Biological) at 37° C. At various timepoints after addition of Cathepsin B, aliquots of the reaction mixtures were taken added to an equal volume of cold acetonitrile to quench the reaction, and then the resulting solution was injected onto an HPLC to analyze conversion of the peptide-AMC substrate to the products of the cleavage, i.e., peptide and AMC. The data show the percent of peptide-AMC that is cleaved at various timepoints and indicate that norleucine (nL) and alpha-amino butyric (B or aBut) are tolerated at the P1 position.

FIG. 8 shows the magnitude of CD8 T cell responses induced against an antigen, E7, following administration of different treatments (Table 7) to C57BL/6 mice (n=3/group).

FIG. 9 shows the magnitude of CD4 T cells induced in vitro following incubation of different treatments (Table 8) with a coculture of antigen-presenting cells and OT-II CD4 T cell clones.

FIG. 10 shows the magnitude of CD4 T cells induced in vitro following incubation of different treatments (Table 9) with a coculture of antigen-presenting cells and OT-II CD4 T cell clones.

FIG. 11 shows the magnitude of IFNγ and IL-17 induced in vitro following incubation of different treatments (Table 10) with a coculture of antigen-presenting cells and OT-II CD4 T cell clones.

FIG. 12 shows the % change in body weight of C57BL/6 mice following different treatments (Table 11).

FIG. 13 shows the % change in body weight of C57BL/6 mice following different treatments (Table 12).

FIG. 14A-14C shows the impact that varying composition of amphiphile and drug molecule of Formula IV have on innate immune activation and tolerability/morbidity in vivo following IV administration. (A) C57BL/6 mice were treated days 0, 7 and 14 with either DMSO/PBS (vehicle control), small molecule TLR-7/8a (R848) or a composition comprising an amphiphile of formula S-B-U-H-D (See table 15), wherein D is an imidazoquinoline of Formula IV, either 2B or 2BXy, according to the study schedule shown. (B) IP-10 levels were assessed from serum of animals by ELISA 10 hours after treatment and (C) percent body (expressed as the percent of body weight immediately prior to treatment) was assessed 24 hours after the 3^{rd} treatment.

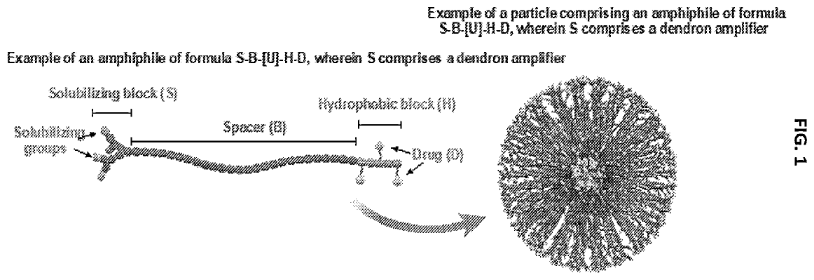

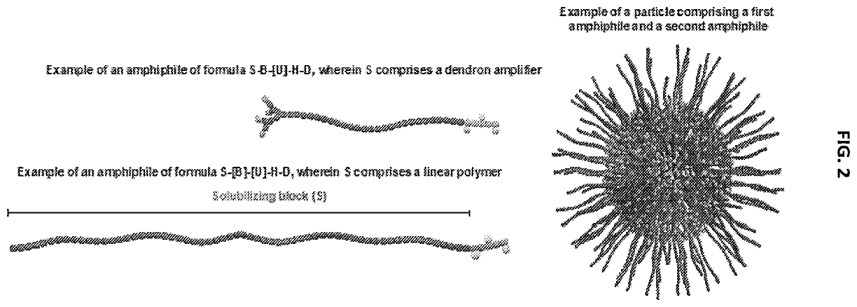

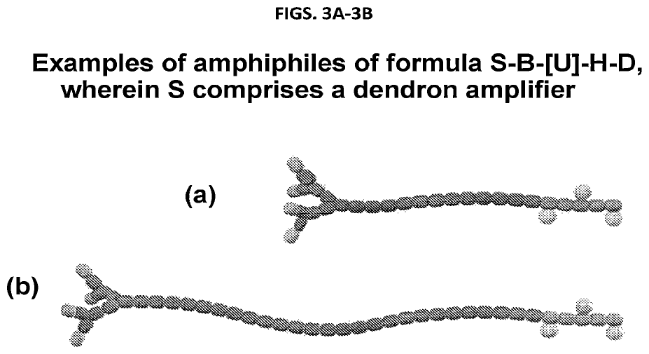

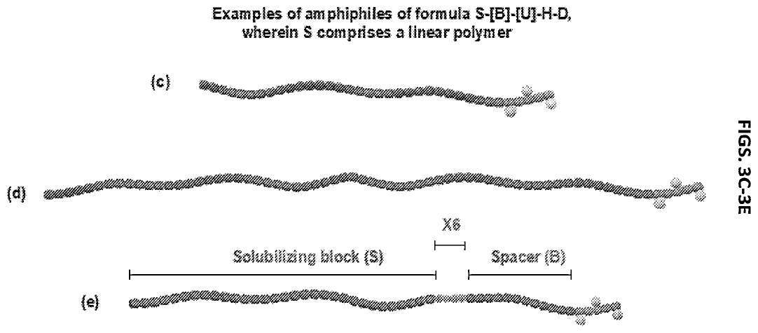

### DEFINITIONS

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods. For example, “about 10” refers to 9.5 to 10.5. A ratio of “about 5:1” refers to a ratio from 4.75:1 to 5.25:1.

Administration: To provide or give to a subject an agent, for example, a nanomedicine composition comprising amphiphile(s) and drug(s) as described herein, by any effective route. Exemplary routes of administration include, but are not limited to, oral, injection (such as subcutaneous, intramuscular, intradermal, intraperitoneal, and intravenous), transdermal, topical, intranasal, vaginal, and inhalation routes.

“Administration or” and “administering a” compound should be understood to mean providing a compound, a prodrug of a compound, or a pharmaceutical composition as described herein. The compound or composition can be administered by another person to the subject or it can be self-administered by the subject.

Antigen: Any molecule that contains an epitope that binds to a T cell or B cell receptor and can stimulate an immune response, in particular, a B cell response and/or a T cell response in a subject. The epitopes may comprise peptides, glycopeptides, lipids or any suitable molecules that contain an epitope that can interact with components of specific B cell or T cell receptors. Such interactions may generate a response by the immune cell. “Epitope” refers to the region of a peptide antigen to which B-cell receptors and T-cell receptors interact.

Antigen-presenting cell (APC): Any cell that presents antigen bound to MHC class I or class II molecules to T cells, including but not limited to monocytes, macrophages, dendritic cells, B cells, T cells and Langerhans cells.

Amphiphilic: The term “amphiphilic” is used herein to mean a substance containing both hydrophilic or polar and hydrophobic groups.

CD4: Cluster of differentiation 4, a surface glycoprotein that interacts with MHC Class II molecules present on the surface of other cells. A subset of T cells express CD4 and these cells are commonly referred to as helper T cells or CD4 T cells.

CD8: Cluster of differentiation 8, a surface glycoprotein that interacts with MHC Class I molecules present on the surface of other cells. A subset of T cells express CD8 and these cells are commonly referred to as cytotoxic T cells (CTLs), killer T cells or CD8 T cells.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units. The charge of a molecule can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom in a molecule may be considered to have a full integer value of charge when the number of electrons assigned to that atom is less than or equal to the atomic number of the atom. The charge of the molecule is determined by summing the charge of each atom comprising the molecule. Those skilled in the art are familiar with the process of estimating charge of a molecule by summing the formal charge of each atom in a molecule. “Charged functional groups refer to functional groups that may be permanently charged or have charge depending on the pH. Charged functional groups may be partial or full integer values of charge, which may be positive or negative, are referred to as positively charged functional groups or negatively charged functional groups, respectively. The portion of a molecule that comprises one or more charged functional groups, which may be positive or negative, is referred to as a “charged group,” e.g., positively charged group or negatively charged group. Charged groups may comprise positive functional groups, negative functional groups or both positive and negative functional groups. The net charge of the charged group may be positive, negative or neutral. Charged monomers refer to monomers that comprise charged groups. Charged amino acids are a type of charged monomer. Note: the net charge of a particle comprising amphiphiles further comprising charged groups, e.g., charged monomers, such as charged amino acids, can be estimated by summing the charge of each functional group within the amphiphile.

Chemotherapeutic: is a type of drug molecule (D) defined broadly as any pharmaceutically active molecule useful in the treatment of cancer and includes growth inhibitory agents or cytotoxic agents, including alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylmelamines including altretamine, triethylenemelamine, triethylenephosphoramide, triethylenethiophosphoramide and trimethylolomelamine; nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL®), docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™) vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in Wiemann et al., 1985, in Medical Oncology (Calabresi et al, eds.), Chapter 10, McMillan Publishing, are also suitable chemotherapeutic agents.

Chemotherapeutics (also referred to as chemotherapeutic agents) are pharmaceutically active compounds and may therefore be referred to herein generally as drugs or drug molecules, or “D” in formulae. For clarity, the terms chemotherapeutic(s) and chemotherapeutic agent(s) are used herein to describe any synthetic or naturally occurring molecules useful for cancer treatment, though, certain classes of drug molecules may alternatively be described by their mechanism of action, e.g., angiogenesis inhibitors are chemotherapeutics that inhibit angiogenesis. While certain immunomodulators, e.g., immunostimulants, may be useful for cancer treatment, immunomodulators, inclusive of immunostimulants and immunosuppressants are not referred to as chemotherapeutics in this specification.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group with an alkyne to form a triazole linker through strain-promoted [3+2]azide-alkyne cyclo-addition.

Copolymer: A polymer derived from two (or more) different monomers, as opposed to a homopolymer where only one monomer is used. Since a copolymer includes at least two types of constituent units (also structural units), copolymers may be classified based on how these units are arranged along the chain. A copolymer may be a statistical (or random) copolymer wherein the two or monomer units are distributed randomly; the copolymer may be an alternating copolymer wherein the two or more monomer units are distributed in an alternating sequence; or, e.g., the copolymer, e.g., a poly(amino acid) may be produced by solid-phase peptide synthesis (SPPS) and have a specific order of monomer units. The term “block copolymer” refers generically to a polymer composed of two or more contiguous blocks of different constituent monomers or comonomers (if a block comprises two or more different monomers). Block copolymer may be used herein to refer to a copolymer that comprises two or more homopolymer subunits, two or more copolymer subunits or one or more homopolymer subunits and one or more copolymer subunits, wherein the subunits may be linked directly by covalent bonds or the subunits may be linked indirectly via an intermediate non-repeating subunit, such as a junction block or linker. Blocks may be based on linear and/or brush architectures. Block copolymers with two or three distinct blocks are referred to herein as “diblock copolymers” and “triblock copolymers,” respectively. Copolymers may be referred to generically as polymers, e.g., a statistical copolymer may be referred to as a polymer or copolymer. Similarly, a block copolymer may be referred to generically as a polymer. While a copolymer used in herein means a polymer comprising two or more types of monomers, terpolymer is a copolymer with three monomer units.

Critical micelle concentration (CMC): Refers to the concentration of a material above which micelles spontaneously form to satisfy thermodynamic equilibrium.

Drug: refers to any pharmaceutically active molecule—including, without limitation, proteins, peptides, sugars, saccharides, nucleosides, inorganic compounds, lipids, nucleic acids, small synthetic chemical compounds, macrocycles, etc.—that has a physiological effect when ingested or otherwise introduced into the body. Pharmaceutically active compounds can be selected from a variety of known classes of compounds, including, for example, analgesics, anesthetics, anti-inflammatory agents, anthelmintics, anti-arrhythmic agents, antiasthma agents, antibiotics (including penicillins), anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin, biphosphonates, prostaglandins, radio-pharmaceuticals, hormones, sex hormones (including steroids), anti-allergic agents, stimulants and anoretics, steroids, sympathomimetics, thyroid agents, vaccines, vasodilators, and xanthines. Drugs may also be referred to as pharmaceutically active agents, pharmaceutically active substances or biologically active compounds or bioactive molecules. Any drug molecules in the formulae described herein are abbreviated “D.”

Drug delivery: A method or process of administering a pharmaceutical compound to achieve a therapeutic effect in humans or animals.

Effective amount: The amount of a compound, material, or composition effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art.

Expression system: are any form of one or more strands of nucleic acid (DNA or RNA) that are designed to produce a protein in a subject. This includes but is not limited to any length (e.g., number of base pairs) of single stranded DNA, double stranded DNA, single stranded RNA and double stranded RNA that encodes one or more proteins. The expression system may be natural or fully synthetic or may comprise both natural and synthetic elements, e.g., non-natural modifications that stabilize the DNA or RNA. The strands of nucleic acid may be linear or circular. Expression systems comprising nucleic acids can exist as a solid or in solution and may occur as any possible salt form. The expression system may be naked, i.e., uncomplexed DNA or RNA or may be complexes with any complexing agent, such as any possible polymer or lipid useful for complexing nucleic acids, which are well known in the art. The expression system may also be a naturally occurring or engineered virus. Engineered viruses are those that have been modified using recombinant techniques.

Graft copolymer: A polymer having a main polymer chain (e.g., polymer A) with one or more sidechains of a second polymer (e.g., polymer B). The first polymer A is linked through its monomers and sidechains to the second polymer B, which is bonded to individual monomers of polymer A thereby branching off from the chain of polymer A. A first polymer linked through an end group to a second polymer may be described as a block polymer (e.g., A-B type di-block) or an end-grafted polymer.

Hydropathy index/GRAVY value: Is a number representing the hydrophobic or hydrophilic characteristics of an amino acid or sequence of amino acids. There are a variety of scales that can be used to describe the relative hydrophobic and hydrophilic characteristics of amino acids comprising peptides. In the present disclosure, the Hydropathy scale of Kyte and Doolittle (Kyte J, Doolittle R F, J. Mol. Biol 157: 105-32, 1983) is used to calculate the grand average of hydropathy (GRAVY) value, sometimes referred to as the GRAVY score. The GRAVY value of a peptide is the sum of the Hydropathy values of all amino acids comprising the peptide divided by the length (i.e., number of amino acids) of the peptide. The GRAVY value is a relative value. The larger the GRAVY value, the more hydrophobic a peptide sequence is considered, whereas the lower the GRAVY value, the more hydrophilic a peptide sequence is considered.

Hydrophilic: Refers to the tendency of a material to disperse freely or be solubilized in aqueous solutions (sometimes referred to as aqueous media). A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble at concentrations of at least 1.0 mg/mL or more. Hydrophilic group refers to the portion of a molecule that is polar and/or charged and has good water solubility.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble in water, or are soluble in water only at concentrations of 1 mg/mL or less, typically 0.1 mg/mL or less or more preferably 0.01 mg/mL or less. Hydrophobic monomers are monomers, e.g., hydrophobic amino acids, that comprise hydrophobic groups and form polymers that are insoluble in water or insoluble in water at certain temperatures, pH and salt concentration. Hydrophobic group refers to a portion of a molecule that is hydrophobic. For example, a styrene monomer may be referred to as a hydrophobic monomer because poly(styrene) is a water insoluble polymer. Hydrophobic drugs refer to drug molecules that are insoluble or soluble only at concentrations of about 1.0 mg/mL or less in aqueous solutions at pH of about pH 7.4. Amphiphilic drugs are drug molecules that have the tendency to assemble into supramolecular structures, e.g., micelles, in aqueous solutions and/or have limited solubility in aqueous solutions at pH of about pH 7.4.

Immune response: A change in the activity of a cell of the immune system, such as a B cell, T cell, or monocyte, as a result of a stimulus, either directly or indirectly, such as through a cellular or cytokine intermediary. In certain embodiments, the response is specific for a particular antigen (an “antigen-specific response”). An immune response may comprise a T cell response, such as a CD4 T cell response or a CD8 T cell response. Such an immune response may result in the production of additional T cell progeny and/or in the movement of T cells. In other embodiments, the response is a B cell response, and results in the production of specific antibodies or the production of additional B cell progeny. In yet other embodiments, the response is an antigen-presenting cell response. An antigen may be used to stimulate an immune response leading to the activation of cytotoxic T cells that kills virally infected cells or cancerous cells. In other embodiments, an antigen may be used to induce tolerance or immune suppression. A tolerogenic response may result from the unresponsiveness of a T cell or B cell to an antigen. A suppressive immune response may result from the priming and/or activation of regulatory cells, such as regulatory T cells, or the trans-differentiation of effectors cells to regulatory cells that downregulate the immune response, i.e., dampen the immune response.

Immunogenic composition: A formulation of materials comprising an antigen and/or immunomodulator that induces a measurable immune response.

Immunomodulators: refers to a type of drug that modulates the activity of cells of the immune system, which includes immunostimulants and immunosuppressants.

Immunostimulants: refers to any synthetic or naturally occurring drugs that promote pro-inflammatory and/or cytotoxic activity by immune cells. Exemplary immunostimulants include pattern recognition receptor (PRR) agonists, such as synthetic or naturally occurring agonists of Toll-like receptors (TLRs), stimulator of interferon gene agonists (STINGa), nucleotide-binding oligomerization domain-like receptor (NLR) agonists, retinoic acid-inducible gene-I-like receptors (RLR) agonists and certain C-type lectin receptor (CLR), as well as certain cytokines (e.g., certain interleukins), such as IL-2; certain chemokines or small molecules that bind chemokine receptors; certain antibodies, antibody fragments or synthetic peptides that activate immune cells, e.g., through binding to stimulatory receptors, e.g., anti-CD40, or, e.g., by blocking inhibitory receptors, e.g., anti-CTLA4, anti-PD1, etc. Various immunostimulants suitable for the practice of the present disclosure are described throughout the specification. For clarity, certain pharmaceutically active compounds that stimulate the immune system may be referred to as immunostimulants or more generally as drug molecules (abbreviated “D” in formulae).

Immunosuppressants: refers to any synthetic or naturally occurring drugs that suppress pro-inflammatory and/or cytotoxic activity by immune cells or the humoral immune system, e.g., antibodies and complement proteins. Immunosuppressants may mediate effects through one or more of the following mechanisms of action: by priming suppressor cells, e.g., regulatory T cells; killing, inhibiting or deactivating proinflammatory cells, cytotoxic cells and/or B cells; trans-differentiating proinflammatory and/or cytotoxic T cells to suppressor cells; and/or sequestering and/or limiting the mobility of proinflammatory cells, cytotoxic cells and/or B cells. Exemplary immunosuppressants include synthetic or naturally occurring agonists of the aryl hydrocarbon receptor (AHR); certain steroids, including glucocorticoids; certain histone deacetylase inhibitors (HDACS), such as inhibitors of HDAC9; retinoic acid receptor agonists; mammalian target of rapamycin (mTOR) inhibitors, such as rapamycin; certain cyclin dependent kinase (CDK) inhibitors; certain adenosine receptor agonists; agonists of PD1; and other molecules that suppress proinflammatory or cytotoxic activity by immune cells or antibodies. Various immunosuppressants suitable for the practice of the present disclosure are described throughout the specification and include Treg promoting immunomodulators. For clarity, immunosuppressants may be referred to more generally as drug molecules (abbreviated “D” in formulae).

In vivo delivery: Administration of a composition, such as a composition comprising amphiphilic block copolymers and drug(s), by topical, transdermal, suppository (rectal, vaginal), pessary (vaginal), intravenous, oral, subcutaneous, intraperitoneal, intrathecal, intramuscular, intracranial, inhalational, oral, or any other suitable route to a subject.

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker (sometimes referred to as linker molecule). The molecules may be linked indirectly by interposition of a component that binds non-covalently to both molecules independently. The term “Linker,” sometimes abbreviated “X,” used in chemical formulae herein means any suitable linker molecule. Specific, preferred linkers may be indicated by other symbols, such as X1, X2, X3, X4, X5 and U. Various linkers are described throughout the specification.

A “bilayer membrane” or “bilayer(s)” is a self-assembled membrane of amphiphiles or super-amphiphiles in aqueous solutions.

Micelles: Spherical receptacles having a single monolayer defining a closed compartment. Generally, amphiphilic molecules spontaneously form micellar structures in polar solvents. In contrast to bilayers, e.g., liposomal bilayers, micelles are “sided” in that they project a hydrophilic, polar outer surface and display a hydrophobic interior surface.

Mol %: Refers to the percentage of a particular type of monomeric unit (or “monomer”) that is present in a polymer. For example, a polymer having 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” or “monomer unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) comprise amino acid monomeric units. Monomeric units may also be referred to as monomers or monomer units or the like.

Nanomedicine: The term nanomedicine may be used to describe nanosized carriers of one or more drug molecules that can be used as a medicament. For instance, a nanoparticle micelle alone may be referred to as a nanocarrier, whereas a nanoparticle micelle further comprising a drug molecule, may be referred to as a nanomedicine comprising a nanocarrier and a drug molecule.

Net charge: The sum of electrostatic charges carried by a molecule or, if specified, a portion or section of a molecule.

Particle: A nano- or micro-sized supramolecular structure composed of an assembly of molecules. For example, amphiphiles of the present disclosure form particles in aqueous solution. In some embodiments, particle formation by the amphiphiles is dependent on pH or temperature. In some embodiments, the nanoparticles composed of amphiphiles have an average diameter between 5 nanometers (nm) to 500 nm. In some embodiments, the nanoparticles composed of amphiphiles form micelles and have an average diameter between 5 nanometers (nm) to 50 nm, such as between 10 and 30 nm. In some embodiments, the nanoparticles composed of amphiphiles may be larger than 100 nm.

Pattern recognition receptors (PRRs): Receptors expressed by various cell populations, particularly innate immune cells that bind to a diverse group of synthetic and naturally occurring molecules. There are several classes of PRRs. Non-limiting examples of PRRs include Toll-like receptors (TLRs), RIG-I-like receptors (RLRs), NOD-like receptors (NLRs), Stimulator of Interferon Genes receptor (STING), and C-type lectin receptors (CLRs). Agonists of such PRRs are referred to as immunostimulant drugs and can be used to enhance and/or modify an immune response to an antigen. For more information on pattern recognition receptors, see Wales et al., Biochem Soc Trans., 35:1501-1503, 2007.

Peptide or polypeptide: Two or more natural or non-natural amino acid residues that are joined together in a series through one or more amide bonds. The amino acid residues may contain post-translational modification(s) (e.g., glycosylation, citrullination, homocitrullination, oxidation and/or phosphorylation). Such modifications may mimic post-translational modifications that occur naturally in vivo or may be non-natural. Any one or more of the components of the amphiphiles may comprise peptides.

Peptide Modifications: Peptides may be altered or otherwise synthesized with one or more of several modifications as set forth below. In addition, analogs (non-peptide organic molecules), derivatives (chemically functionalized peptide molecules obtained starting from a peptide) and variants (homologs) of these peptides can be utilized in the methods described herein. The peptides described herein comprise a sequence of amino acids, analogs, derivatives, and variants, which may be either L- and/or D-versions. Unless otherwise specified, any peptide sequences referenced herein comprise L amino acids, preferably exclusively L amino acids. Such peptides may contain peptides, analogs, derivatives, and variants that are naturally occurring and otherwise.

Peptides can be modified through any of a variety of chemical techniques to produce derivatives having similar activity as the unmodified peptides, and optionally having other desirable properties. For example, carboxylic acid groups of the peptide, whether at the carboxyl terminus or at a side chain, can be provided in the form of a salt of a pharmaceutically-acceptable cation or esterified to form a CC₁-CC₁₆ ester, wherein CC refers to a carbon chain (and thus, CC1 refers to a single carbon and CC16 refers to 16 carbons), or converted to an amide. Amino groups of the peptide, whether at the amino terminus or at a side chain, can be in the form of a pharmaceutically-acceptable acid addition salt, such as the HCl, HBr, acetic, trifluoroacetic, formic, benzoic, toluene sulfonic, maleic, tartaric and other organic salts, or can be modified or converted to an amide, e.g., by acetylation.

Peptides may be modified to contain substituent groups that contain a positive or negative charge or both. The positive and/or negative charge may be affected by the pH at which the peptide is present.

Hydroxyl groups of the peptide side chains may be converted to C₁-C₁₆ alkoxy or to a C₁-C₁₆ ester using well-recognized techniques, or the hydroxyl groups may be converted (e.g., sulfated or phosphorylated) to introduce negative charge. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be used to form disulfide bonds or thioethers, for example through reaction with a maleimide. Thiols may be protected with any of a number of well-recognized protecting groups, such as acetamide groups. Those skilled in the art will also recognize methods for introducing cyclic structures into the peptides of this invention to select and provide conformational constraints to the structure that result in enhanced stability. Reference may be made to Greene et al., “Greene's Protective Groups in Organic Synthesis” Fourth Edition, John Wiley & Sons, Inc. 2006 for details of additional modifications that can be made to functional groups.

Pharmaceutically acceptable vehicles: The pharmaceutically acceptable vehicles (or carriers) useful in this disclosure include conventional carriers, excipients, and diluents. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, such as one or more therapeutic cancer vaccines, and additional pharmaceutical agents.

Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as an ointment or cream.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

Polar: A description of the properties of matter. Polar is a relative term and may describe a molecule or a portion of a molecule that has partial charge that arises from differences in electronegativity between atoms bonded together in a molecule, such as the bond between nitrogen and hydrogen. Polar molecules prefer interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may prefer interacting with a polar solvent such as water. In specific, non-limiting cases, introduction of additional polar groups may increase the solubility of a portion of a molecule.

Polymer: A molecule containing repeating structural units (monomers). As described in greater detail throughout the disclosure, polymers may be used for any number of components of amphiphiles and drug molecule conjugates and may be natural or synthetic. Various compositions of polymers useful for the practice of the invention are discussed in greater detail elsewhere. Note: polymer is used throughout the specification to broadly encompass molecules with as few as three or more monomers, which may sometimes be referred to as oligomers.

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomer units resulting in the splitting out of a water molecule. In other forms of polymerization, a monomer is added one at a time to a growing chain through the staged introduction of activated monomers, such as during solid phase peptide synthesis (SPPS).

Polymersome: Vesicle, which is assembled from synthetic multi-block polymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self-assembly,” a spontaneous, entropy-driven process of preparing a closed semi-permeable membrane.

Purified: A substance or composition that is relatively free of impurities or substances that adulterate or contaminate the substance or composition. The term purified is a relative term and does not require absolute purity. Substantial purification denotes purification from impurities. A substantially purified substance or composition is at typically at least 60%, 70%, 80%, 90%, 95%, 98%, or 99% pure.

Soluble: Capable of becoming molecularly or ionically dispersed in a solvent to form a homogeneous solution. When referring to an amphiphile drug molecule conjugate and/or drug molecule, soluble is understood to be a single molecule in solution that does not assemble into multimers or other supramolecular structures through hydrophobic or other non-covalent interactions. A soluble molecule is understood to be freely dispersed as single molecules in solution. Hydrophobic blocks (H) described herein are insoluble or soluble only to concentrations of about 0.1 mg/mL or less. Solubility can be determined by visual inspection, turbidity measurements or dynamic light scattering.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Targeting molecules: Are broadly defined as molecules that direct drug molecules to a specific tissue or cell population. Targeting molecules are defined by their intended use and therefore include structurally diverse molecules including without limitation antibodies, Fabs, peptides, aptamers, saccharides (e.g., saccharides that bind to lectin receptors and/or are recognized by cellular transporters), amino acids, neurotransmitters, etc. As targeting molecules are often selected from molecules that bind cellular receptors that can activate downstream signaling cascades and/or impact the activity of other linked molecules, targeting molecules are often classified as drug molecules (D) in the present disclosure. Additionally, targeting molecules can also have solubilizing effects, and may be considered either or both drug molecules (D) and/or solubilizing (SG) groups.

T Cell: A type of white blood cell that is part of the immune system and may participate in an immune response. T cells include, but are not limited to, CD4 T cells and CD8 T cells. A CD4 T cell displays the CD4 glycoprotein on its surface and these cells are often referred to as helper T cells. These cells often coordinate immune responses, including antibody responses and cytotoxic T cell responses, however, CD4 T cells (e.g., regulatory T cells) can also suppress immune responses or CD4 T cells may act as cytotoxic T cells. A CD8 T cell displays the CD8 glycoprotein on its surface and these cells are often referred to as cytotoxic or killer T cells, however, CD8 T cells can also suppress immune responses.

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Reducing a sign or symptom or marker of a disease or pathological condition related to a disease, refers to any observable beneficial effect of the treatment and/or any observable effect on a proximal, surrogate endpoint, for example, tumor volume, whether symptomatic or not. Reducing a sign or symptom associated with a tumor or viral infection can be evidenced, for example, by a delayed onset of clinical symptoms of the disease in a susceptible subject (such as a subject having a tumor which has not yet metastasized, or a subject that may be exposed to a viral infection), a reduction in severity of some or all clinical symptoms of the disease, a slower progression of the disease (for example by prolonging the life of a subject having a tumor or viral infection), a reduction in the number of relapses of the disease, an improvement in the overall health or well-being of the subject, or by other parameters well known in the art (e.g., that are specific to a particular tumor or viral infection). A “prophylactic” treatment is a treatment administered to a subject who does not exhibit signs of a disease or exhibits only early signs for the purpose of decreasing the risk or severity of developing pathology.

Tumor or cancer or neoplasm: An abnormal growth of cells, which can be benign or malignant, often but not always causing clinical symptoms. “Neoplastic” cell growth refers to cell growth that is not responsive to physiologic cues, such as growth and inhibitory factors.

A “tumor” is a collection of neoplastic cells. In most cases, tumor refers to a collection of neoplastic cells that forms a solid mass. Such tumors may be referred to as solid tumors. In some cases, neoplastic cells may not form a solid mass, such as the case with some leukemias. In such cases, the collection of neoplastic cells may be referred to as a liquid cancer.

Cancer refers to a malignant growth of neoplastic cells, being either solid or liquid. Features of a cancer that define it as malignant include metastasis, interference with the normal functioning of neighboring cells, release of cytokines or other secretory products at abnormal levels and suppression or aggravation of inflammatory or immunological response(s), invasion of surrounding or distant tissues or organs, such as lymph nodes, etc.

A tumor that does not present substantial adverse clinical symptoms and/or is slow growing is referred to as “benign.”

“Malignant” means causing, or likely to cause in the future, significant clinical symptoms. A tumor that invades the surrounding tissue and/or metastasizes and/or produces substantial clinical symptoms through production and secretion of chemical mediators having an effect on nearby or distant body systems is referred to as “malignant.”

“Metastatic disease” refers to cancer cells that have left the original tumor site and migrated to other parts of the body, for example via the bloodstream, via the lymphatic system, or via body cavities, such as the peritoneal cavity or thoracic cavity.

The amount of a tumor in an individual is the “tumor burden”. The tumor burden can be measured as the number, volume, or mass of the tumor, and is often assessed by physical examination, radiological imaging, or pathological examination.

An “established” or “existing” tumor is a tumor that exists at the time a therapy is initiated. Often, an established tumor can be discerned by diagnostic tests. In some embodiments, an established tumor can be palpated. In some embodiments, an established tumor is at least 500 mm³, such as at least 600 mm³, at least 700 mm³, or at least 800 mm³ in size. In other embodiments, the tumor is at least 1 cm long. With regard to a solid tumor, an established tumor generally has a newly established and robust blood supply and may have induced the regulatory T cells (Tregs) and myeloid derived suppressor cells (MDSC).

Unit dose: A discrete amount of the pharmaceutical composition comprising a predetermined amount of the active ingredient.

Vesicle: A fluid filled sac. In some embodiments the vesicle is a sac comprising an amphiphilic substance. In some embodiments, the sac is a nanoparticle-based vesicle, which refers to a vesicle with a size or dimensions in the nanometer range. In some embodiments, a polymer vesicle is a vesicle that is formed from one or more polymers.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to an alkyl which may be substituted or not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)-, preferably alkylC(O)-.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

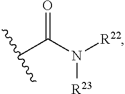

wherein R²² and R²³ each independently represent a hydrogen or hydrocarbyl group, or R²² and R²³ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

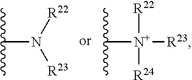

wherein R²², R²³, and R²⁴ each independently represent a hydrogen or a hydrocarbyl group, or R²² and R²³ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted aromatic carbocycles as well as heteroaryls. The term “aryl” is used interchangeably with the term “aromatic group” herein. Unless specifically stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(Ra)C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Aromatic carbocycles include single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

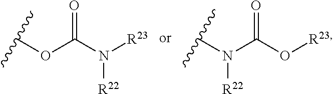

wherein R²² and R²³ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. For example, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR²² wherein R²² represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O-. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

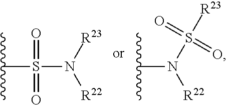

wherein R²² and R²³ independently represents hydrogen or hydrocarbyl.

The term “sulfoxide” is art-recognized and refers to the group-S(O)-.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR²² or —SC(O)R²² wherein R²² represents a hydrocarbyl.

The term “thioether”, as used herein, is equivalent to an ether, wherein the oxygen is replaced with a sulfur.

The term “urea” is art-recognized and may be represented by the general formula

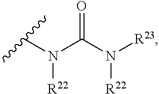

wherein R²² and R²³ independently represent hydrogen or a hydrocarbyl.

The term “aromatic amino acid” includes amino acids with a side chain comprising an aromatic group, such as phenylalanine, tyrosine, or tryptophan. Aromatic group refers to the portion of a molecule that comprises an aromatic ring. For example, phenylalanine is an aromatic amino acid that comprises an aromatic group, i.e., benzyl group. Phenylalanine (Phe) and Tryptophan (Trp) are prototypical aromatic amino acids.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting.

### Description of Embodiments

Provided herein are compositions of particles comprising amphiphiles and drug molecules useful for the treatment or prevention of a disease, e.g., cancer(s), autoimmune disease(s), allergy(ies) and/or infectious disease(s).

The present disclosure relates to a composition comprising a first amphiphile and optionally a second amphiphile each having the formula S-[B]-[U]-H, wherein S, independently for each occurrence, is a solubilizing block;
B, independently for each occurrence, is a spacer;
H, independently for each occurrence, is a hydrophobic block;
U, independently for each occurrence, is a linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof;
and at least one drug molecule D is noncovalently associated with or covalently bonded directly or via a suitable linker X1 to the first amphiphile and/or the optional second amphiphile.

In some embodiments, D is noncovalently associated with or covalently bonded directly or via a suitable linker X1 to the first amphiphile. In other embodiments, D is noncovalently associated with or covalently bonded directly or via a suitable linker X1 to the second amphiphile. In other embodiments, a D, independently selected for each occurrence, is noncovalently associated with or covalently bonded directly or via a suitable linker X1 to the first and the second amphiphile.

In some embodiments, the S of the first amphiphile comprises SGs selected from carboxylic acids.

In some embodiments, 4 to 8 SGs are connected to the S.

In some embodiments, the dendron amplifier comprises repeating monomer units of 1 to 10 generations having between 2 to 6 branches per generation.

In some embodiments, the dendron amplifier comprises repeating monomer units of 2 to 3 generations having between 2 to 3 branches per generation.

In some embodiments, the repeating monomer units are selected from -FG1-(CH₂)_{y2}CH(R¹)₂, -FG1-(CH₂)_{y2}C(R¹)₃, -FG1-(CH₂CH₂O)_{y2}CH(R¹)₂, -FG1-(CH₂CH₂O)_{y2}C(R¹)₃, -FG1-CH(R¹)₂, and -FG1-C(R¹)₃, wherein
R¹, independently for each occurrence, is selected from —(CH₂)_{y3}-FG2, —(OCH₂CH₂)_{y3}-FG2, and —CH₂(OCH₂CH₂)_{y3}-FG2;
y2 and y3, independently for each occurrence, are an integer of repeating units from 1 to 6;
FG1 is a first functional group; and
FG2 is a second functional group.

In some embodiments, FG1 is —NH₂ and FG2 is —CO₂H; or FG1 is —CO₂H and FG2 is —NH₂.

In some embodiments, the repeating monomer units are selected from hydroxy acids, amino acids, polyols, polyamines, and amino alcohols.

In some embodiments, the monomers comprise 3-hydroxypropanoic acid and serinol.

In some embodiments, the first amphiphile has the structure:

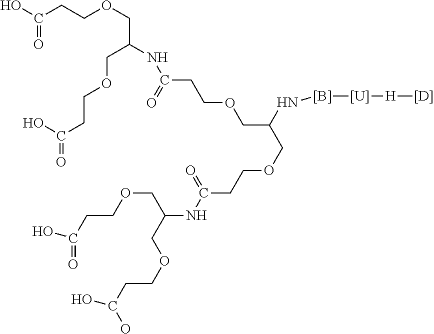

In some embodiments, the dendron amplifier comprises one or more polyethylene oxide (PEG) monomer units.

In some embodiments, the nanomedicine comprises a second amphiphile having the formula S-[B]-[U]-H.

In some embodiments, the S of the second amphiphile is a hydrophilic polymer.

In some embodiments, the hydrophilic polymer is a linear hydrophilic polymer.

In some embodiments, the hydrophilic polymer comprises monomeric units selected from HEMA, HPMA, PEG, and zwitterionic betaine monomers.

In some embodiments, the hydrophilic polymer comprises from between about 24 to about 300 monomer units, or from between about 48 to about 200 monomer units.

In some embodiments, the second amphiphile comprises a hydrophilic polymer comprising from between about 48 to about 200 monomer units and the first amphiphile and second amphiphile are at a molar ratio selected from between about 12:1 to about 6:1.

In some embodiments, the second amphiphile has the formula S-X6-B-[U]-H, wherein the linker X6 is selected from enzyme degradable peptides and pH sensitive linkers.

In some embodiments, the enzyme degradable peptide comprises 2 to 6 amino acids.

In some embodiments, the enzyme degradable peptide comprises an amino acid residue P1 selected from arginine, lysine, acetyl lysine, boc protected lysine, citrulline, glutamine, threonine, leucine, norleucine, alpha-aminobutyric acid, and methionine; and an amino acid residue a P2 selected from beta-alanine, glycine, serine, leucine, valine, and isoleucine.

In some embodiments, the B of the second amphiphile is selected from peptides or hydrophilic polymers comprising from between about 1 to about 36 monomer units, or between about 4 to about 24 monomer units.

In some embodiments, the first amphiphile comprises a spacer (B) selected from hydrophilic polymers comprising from between about 1 to about 36 monomer units.

In some embodiments, B of the first amphiphile comprises an equal or greater number of monomer units relative to the B of the second amphiphile.

In some embodiments, the first amphiphile and second amphiphile are at a molar ratio selected from between about 20:1 to about 1:20, or between about 10:1 to about 1:1.

In some embodiments, the H of the first amphiphile and/or second amphiphile comprises a higher alkane, an aromatic group, a fatty acid, a sterol, a polyunsaturated hydrocarbon, squalene, saponins, or a polymer.

In some embodiments, the H of the first amphiphile and/or second amphiphile comprises a polymer selected from a poly(amino acid).

In some embodiments, the H of the first amphiphile and/or second amphiphile comprises a dendron amplifier.

In some embodiments, each H independently comprises a poly(amino acid) comprising monomers selected from hydrophobic amino acids (M), reactive amino acids (N), spacer amino acids (O), charged amino acids (P) and combinations thereof provided that at least one of M or N is present.

In some embodiments, each H independently comprises a poly(amino acid) having the formula:

-(M)ₘ-(N)ₙ-(O)ₒ-(P)ₚ-R³,
wherein M, N, O and P are each independently present or absent, provided that at least one of M or N is present;
m, n, o and p each independently denote an integer of 1 to 100 with the sum of m, n, o and p less than or equal to 100;
R³ is selected from hydrogen, NH₂, NH—CH₃, NH—(CH₂)_{y5}CH₃, OH or a drug molecule (D) either connected directly or through a suitable linker X; and
y5 is an integer selected from 1 to 6.

In some embodiments, P is absent.

In some embodiments, N, O, and P are each absent.

In some embodiments, P, when present, is

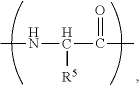

wherein each R⁵, independently, is a group that comprises 1 to 2 charged functional groups.

In some embodiments, O, when present, is

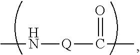

wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4.

In some embodiments, N, when present, is

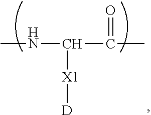

wherein each X1, independently, is a suitable linker; and each D, independently, is a drug molecule.

In some embodiments, M, when present, is

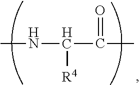

wherein each R⁴ is, independently, a hydrophobic group.

In some embodiments, R⁴ is

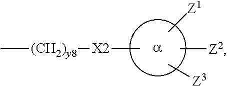

wherein,
α is aryl or heteroaryl;
X2 is present or absent and when present is a suitable linker;
y8 is selected from an integer from 0 and 6; and
Z¹, Z², and Z³ are each independently selected from hydrogen, fluorine, hydroxy, amino, alkyl, and fluoroalkyl.

In some embodiments, a is aryl. In other embodiments, a is heteroaryl.

In some embodiments, A is selected from an imidazolyl, phenyl, pyridinyl, naphthyl, quinolinyl, isoquinolinyl, indolyl, and benzimidazolyl.

In some embodiments, X2 is absent. In other embodiments, X2 is present and is selected from C(O), CO₂(CH₂)_{y9}, CO₂, C(O)NH(CH₂)_{y9}, NHC(O), and NHC(O)(CH₂)_{y9}, wherein y9 is an integer selected from 1 to 6. In other embodiments, X2 is present and is selected from alkyl and a PEG group.

In some embodiments, X1 is present and is selected from an enzyme cleavable linker, a pH sensitive linker, a self-immolative linker, a lower alkyl and a PEG group.

In some embodiments, each R⁴ is independently selected from:

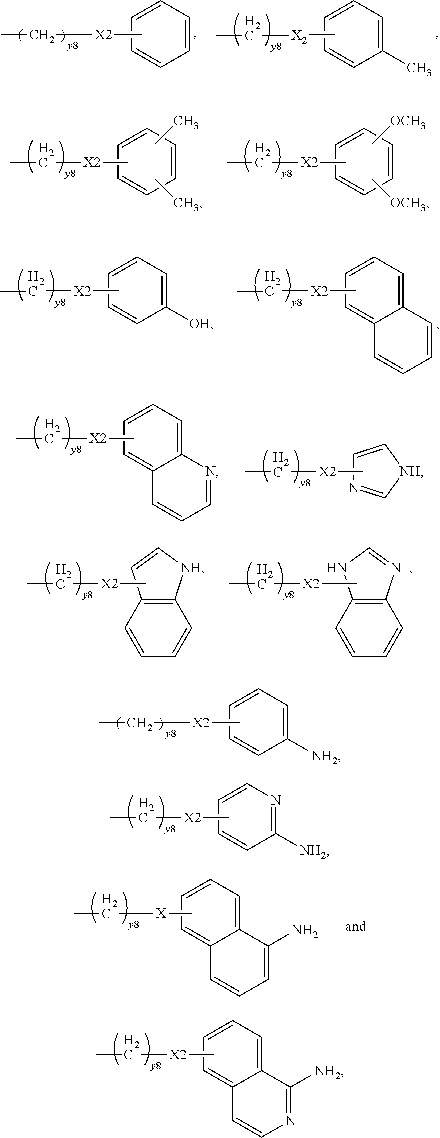

wherein each X2 is independently selected from a suitable linker and each y8 is independently selected from an integer from 0 and 6.

In some embodiments, each R⁴ is independently selected from:

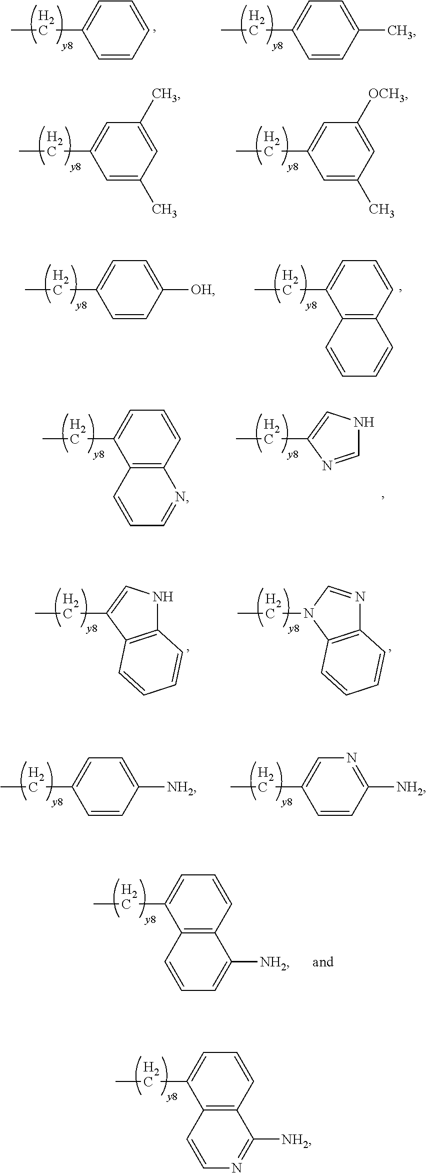

wherein each y8 is independently selected from an integer from 0 and 6.

In some embodiments, each R⁴ is independently selected from:

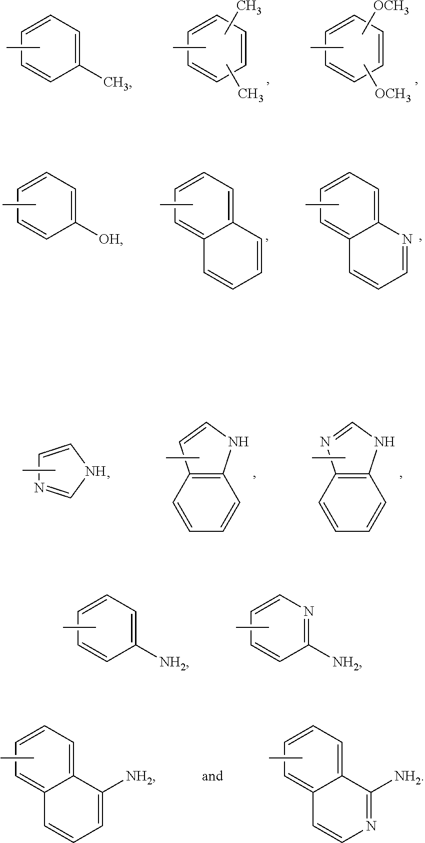

In some embodiments, each R⁴ is independently selected from:

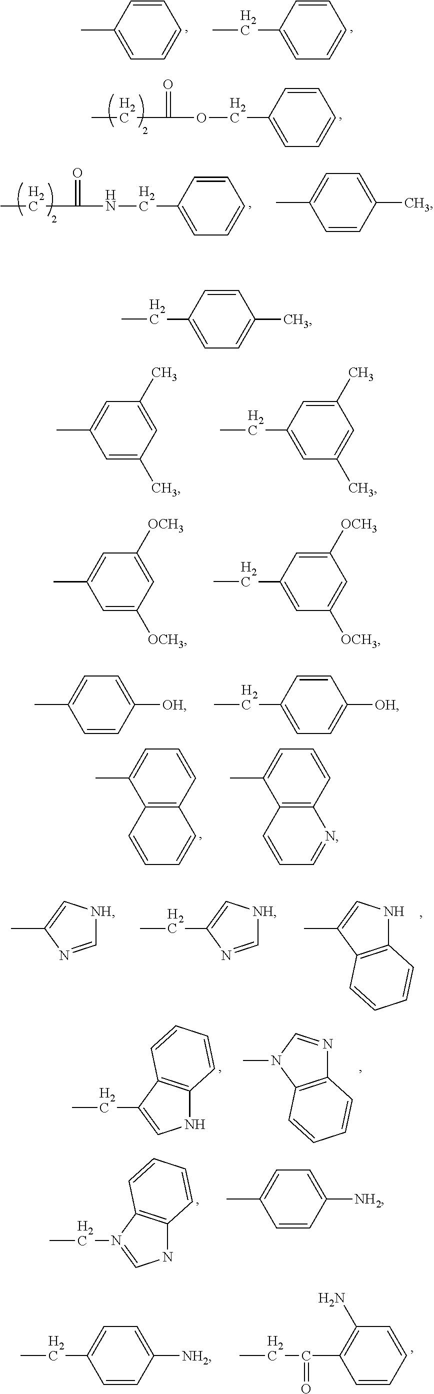

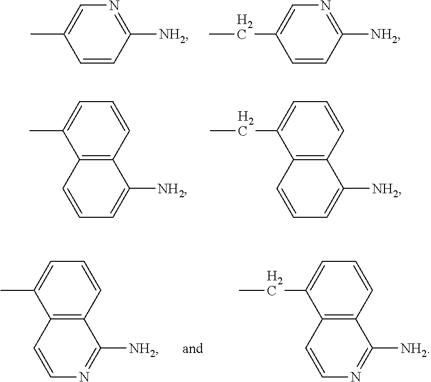

In some embodiments, each R⁴ is independently selected from:

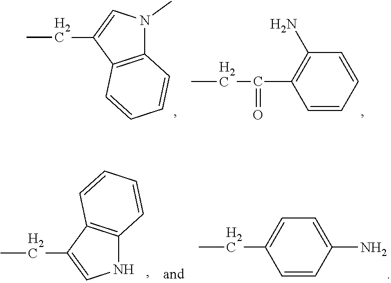

In some embodiments, each R⁴ is selected from:

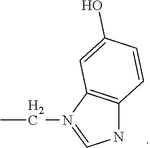

In some embodiments, the at least one D is selected from immunomodulatory and chemotherapeutic drugs.

In some embodiments, the at least one D is an immunostimulant selected from agonists of TLR-3, TLR-7, TLR-8, TLR-7/8, TLR-9 and STING.

In some embodiments, the D is a chemotherapeutic drug selected from anthracyclines, platins, androgens, such as calusterone; anti-adrenals, toxoids, taxanes, tyrosine kinase inhibitors and/or angiogenesis inhibitors, and PI3K inhibitors.

In some embodiments, the D is a chemotherapeutic drug selected from anthracyclines, such as doxorubicin and daunorubicin; platins, such as cisplatin, carboplatin and oxaliplatin; androgens, such as calusterone; anti-adrenals, such as mitotane or trilostane; members of taxoid or taxane family, such as paclitaxel (TAXOL®), docetaxel (TAXOTERE®) and analogues thereof; certain inhibitors of receptor tyrosine kinases and/or angiogenesis, including sorafenib (NEXAVAR®), and sunitinib (SUTENT®); and inhibitors of PI3K, such as alpelisib, and the like, and pharmaceutically acceptable salts, acids or derivatives of any of the above

In some embodiments, the at least one D is not covalently linked to the first and/or second amphiphile.

In some embodiments, the at least one D is covalently linked to the hydrophobic block (H) to the first and/or second amphiphile either directly or indirectly through a linker X1.

In some embodiments, the at least D is covalently linked to a hydrophobic block H either directly or via a linker U.

In some embodiments, the at least one D is covalently linked to the hydrophobic block of the first amphiphile, second amphiphile or both the first amphiphile and the second amphiphile either directly or through a linker X1.

In some embodiments, the linker X1 comprises an amide, carbamate, hydrazone, ketal or silyl ether moiety.

In some embodiments, the linker X1 comprises degradable peptide comprising 2 to 6 amino acids.

In some embodiments, the enzyme degradable peptide comprises an amino acid residue P1 selected from arginine, lysine, acetyl lysine, boc protected lysine, citrulline, glutamine, threonine, leucine, norleucine, alpha-aminobutyric acid, and methionine; and an amino acid residue a P2 selected from beta-alanine, glycine, serine, leucine, valine, and isoleucine.

In some embodiments, the composition comprises a second drug molecule (D2) selected from inhibitors of mTORC1.

In some embodiments, the inhibitors of mTORC1 are selected from rapamycin, everolimus, and temsirolimus.

In some embodiments, the at least one drug molecule is an inhibitor of mTORC1 and/or mTORC2.

In some embodiments, the inhibitor of mTORC1 and/or mTORC2 is selected from rapamycin, sirolimus, tacrolimus, INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458, RAD001, CCI-779, AP23573, BEZ235, AZD2014 and XL765.

In some embodiments, the inhibitor of mTORC1 and/or mTORC2 is selected from rapamycin, sirolimus, tacrolimus, everolimus, INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458, RAD001, CCI-779, AP23573, BEZ235, AZD2014, and XL765, and any derivatives or structural analogs thereof, including any prodrug forms thereof.

In some embodiments, the inhibitor of mTORC1 and/or mTORC2 is selected from INK128, AZD8055, Torin 1, and WYE-132.

In some embodiments, the inhibitor of mTORC1 and/or mTORC2 is selected from INK128, AZD8055, Torin 1, Torin 2, and WYE-132.

In some embodiments, the at least one drug molecule is a dual inhibitor of mTORC1 and mTORC2.

In some embodiments, the at least one drug molecule is a dual inhibitor of mTORC1 and mTORC2 selected from an ATP-competitive mTOR inhibitor. In some embodiments, the dual inhibitor of mTORC1 and mTORC2 is selected from INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458, BEZ235, AZD2014, and XL765, and any derivatives or structural analogs thereof, including any prodrug forms thereof.

In some embodiments, the molar ratio of amphiphile to the first drug molecule (D1) is from about 20:1 to about 1:20. In other embodiments, the molar ratio of amphiphile to the first drug molecule (D1) is from about 5:1 to about 1:5. In other embodiments, the molar ratio of amphiphile to the first drug molecule (D1) is from about 2:1 to about 1:4. In other embodiments, the molar ratio of amphiphile to the first drug molecule (D1) is from about 1:1 to about 1:4.

In some embodiments, the first amphiphile and/or the optional second amphiphile have the structure:

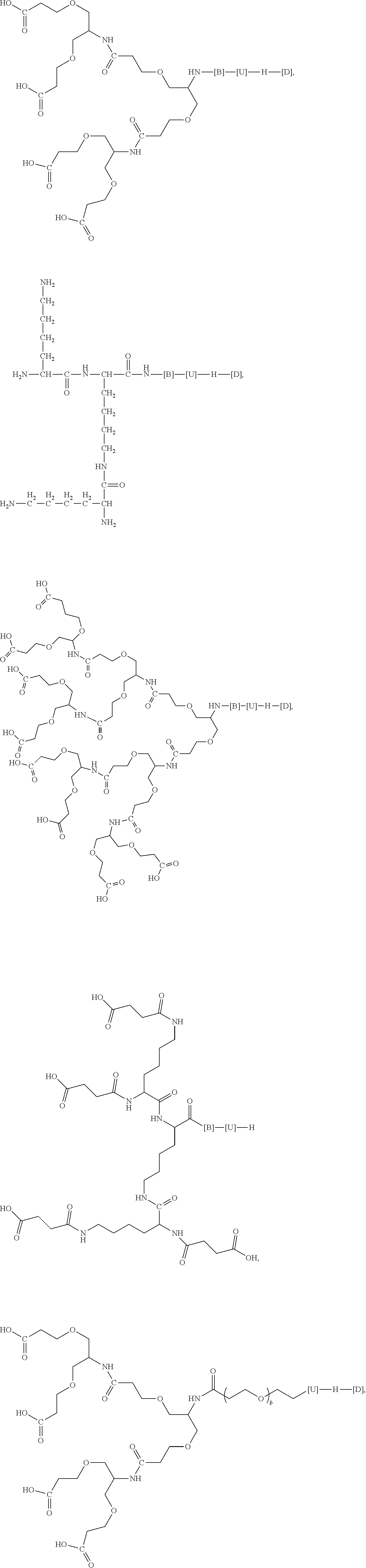

wherein b is an integer of repeating units from 1 to 48,

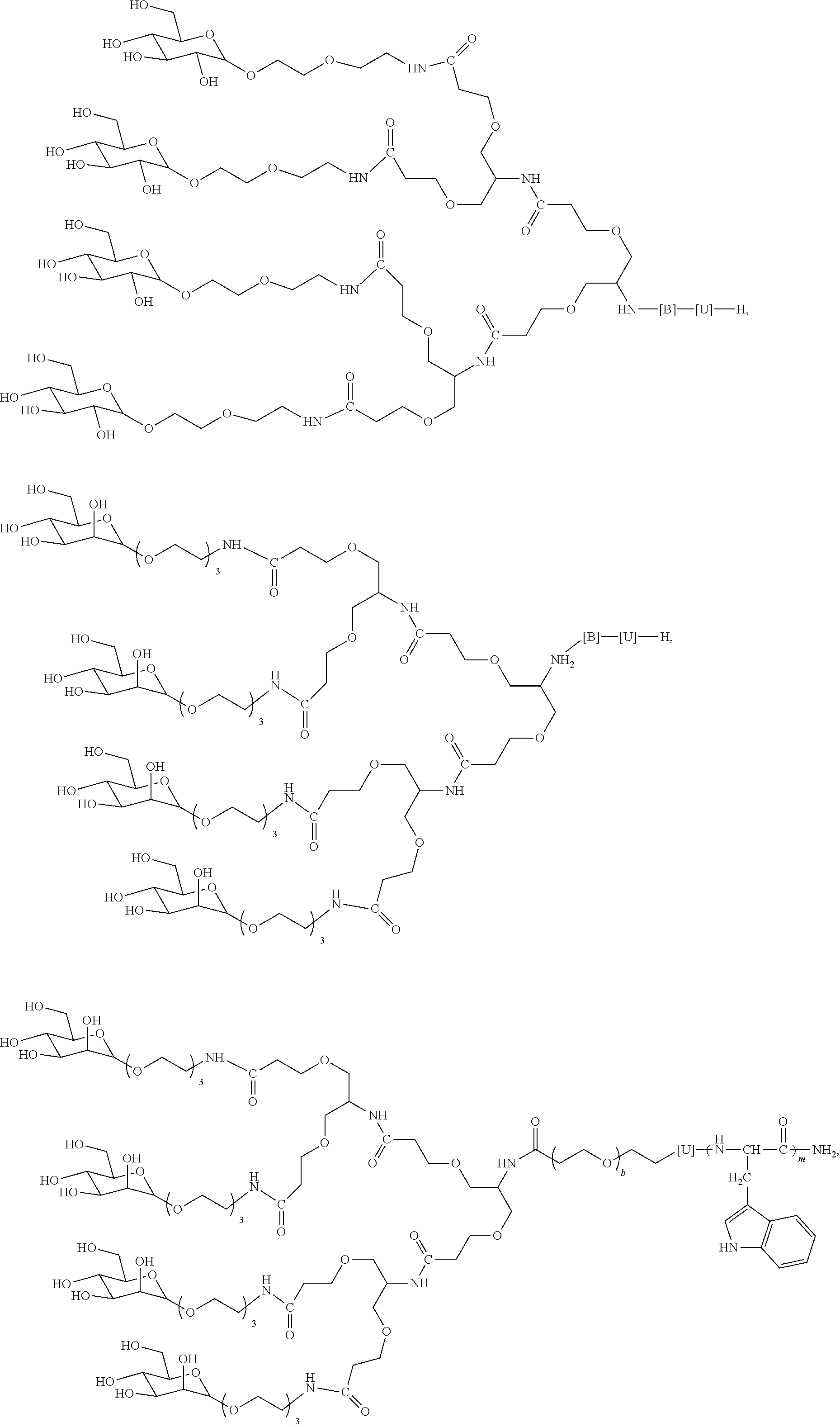

wherein b is an integer of repeating units from 1 to 36, or

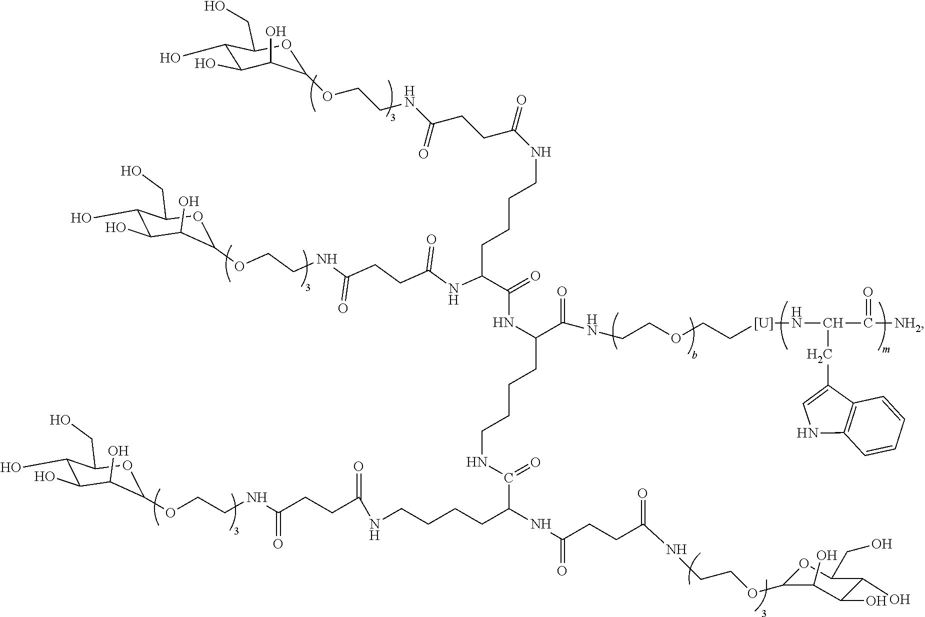

wherein b is an integer of repeating units from 1 to 36.

In some embodiments, the first amphiphile and/or the optional second amphiphile have the structure:

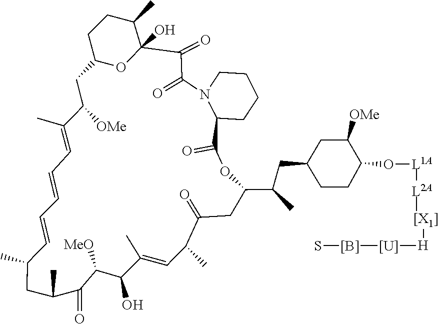

wherein
L^{1A} is an alkylene linker; and
L^{2A} is an amino, amido, ester, peptide, or nitrogen containing heterocyclyl linker.

In some embodiments, the first amphiphile and/or the optional second amphiphile have the structure:

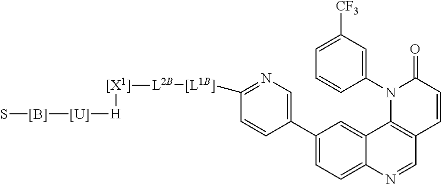

wherein
L^{1B} is an alkylene-C(O)—NH— linker; and
L^{2A} is an amino, amido, peptide, or nitrogen containing heterocyclyl linker; or

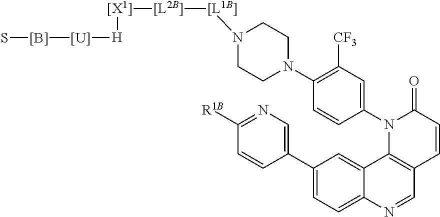

L^{1B} is a

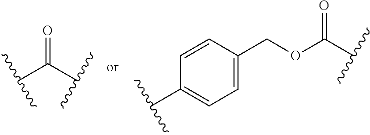

linker;
L^{2B} is an amino, amido, or peptide linker; and
R^{1B} is NH₂ or

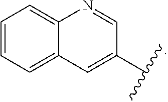 [SMILES: *c1cnc2ccccc2c1]

In some embodiments, the first amphiphile and/or the optional second amphiphile have the structure:

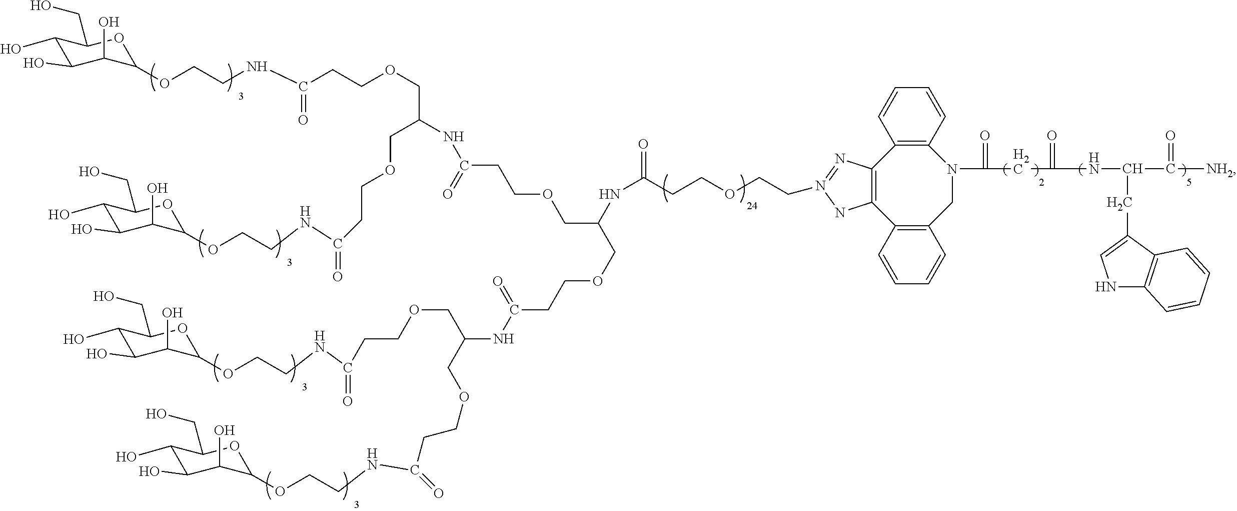

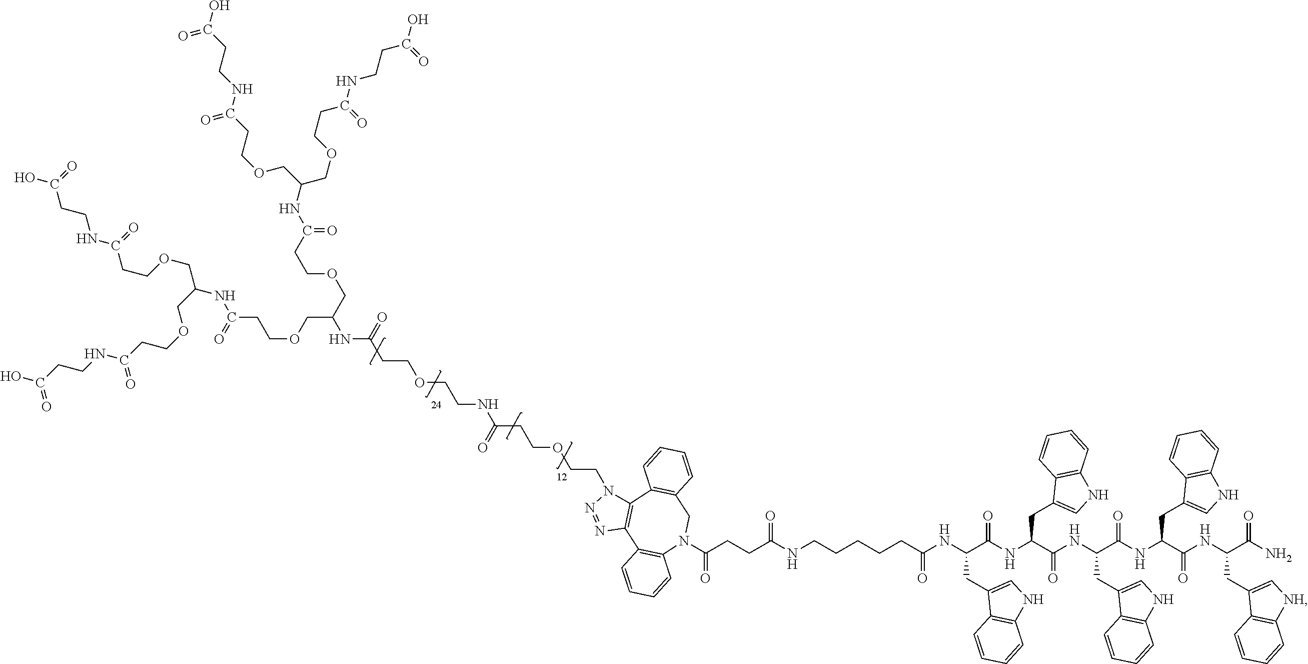

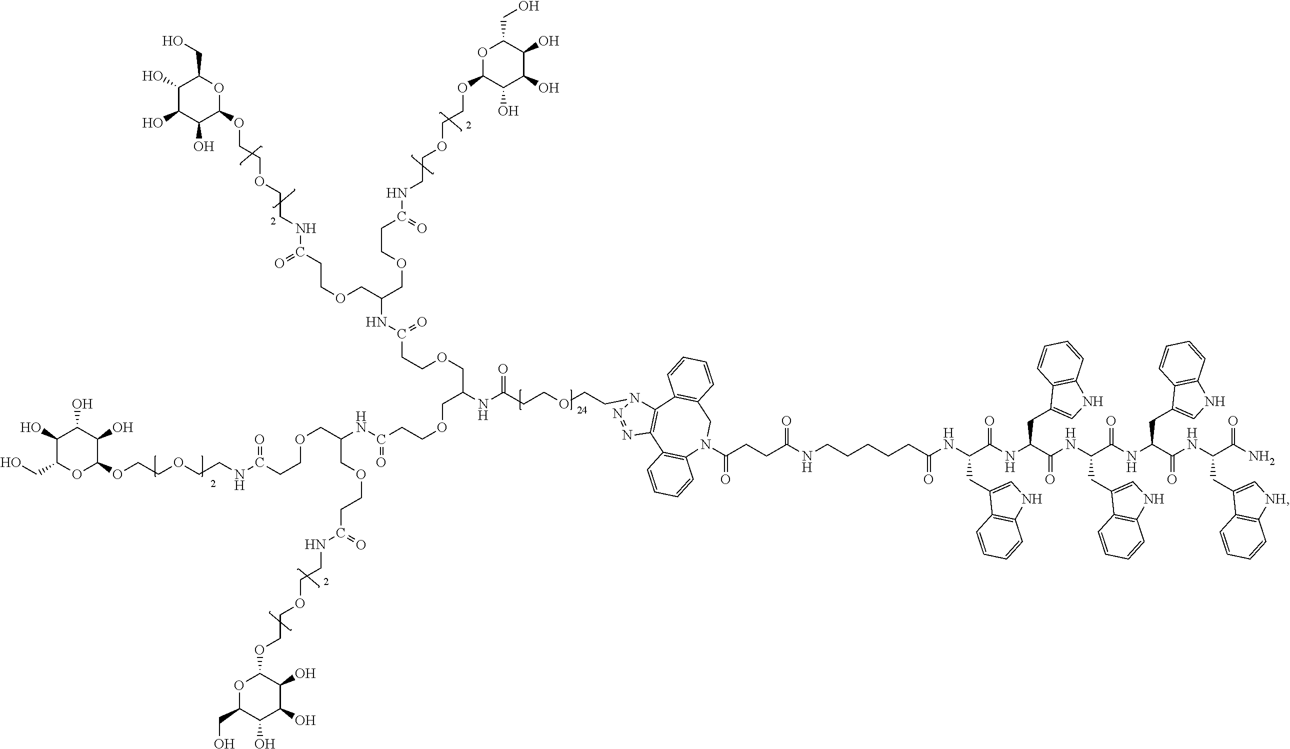

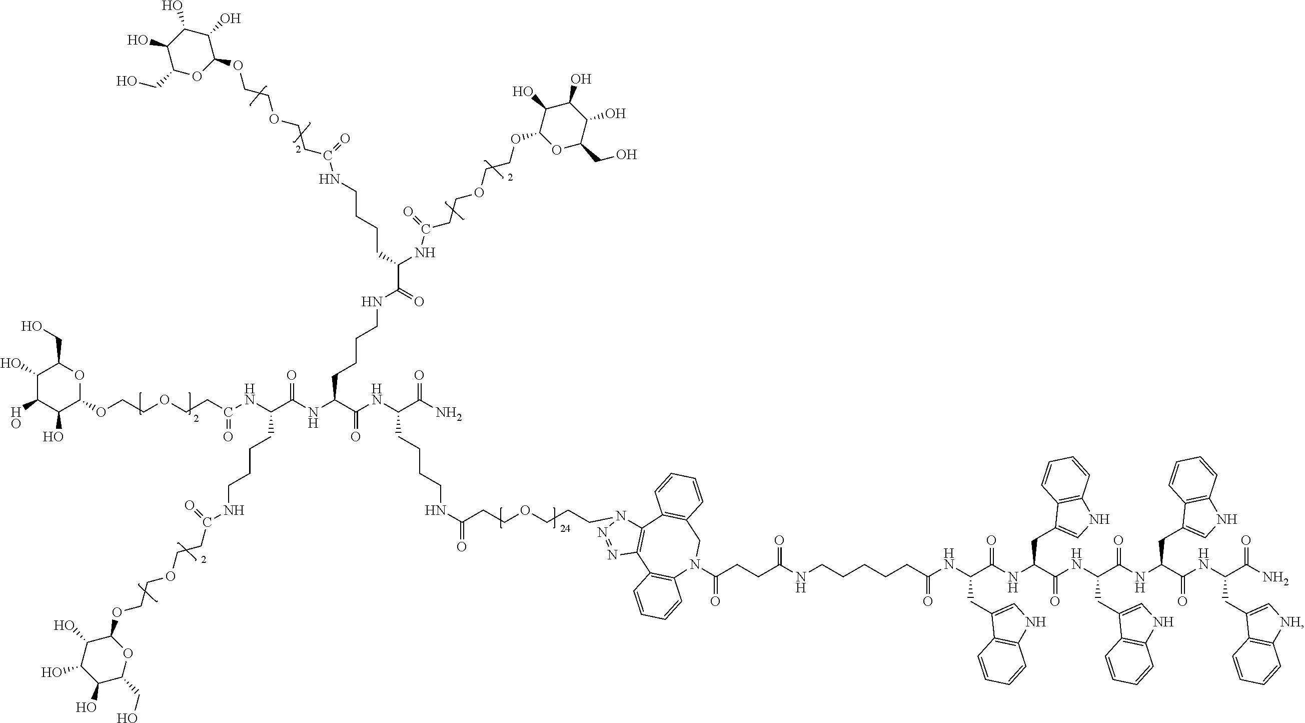

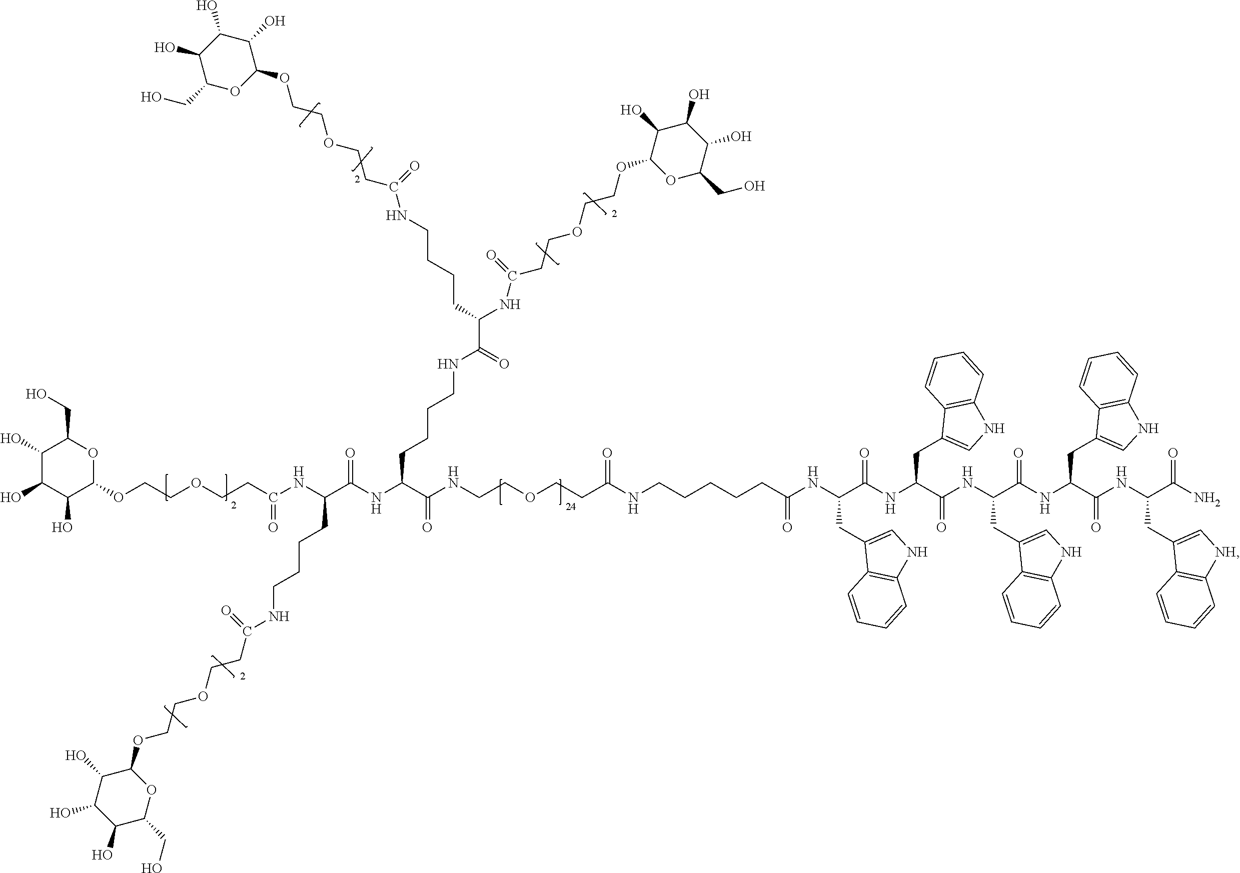

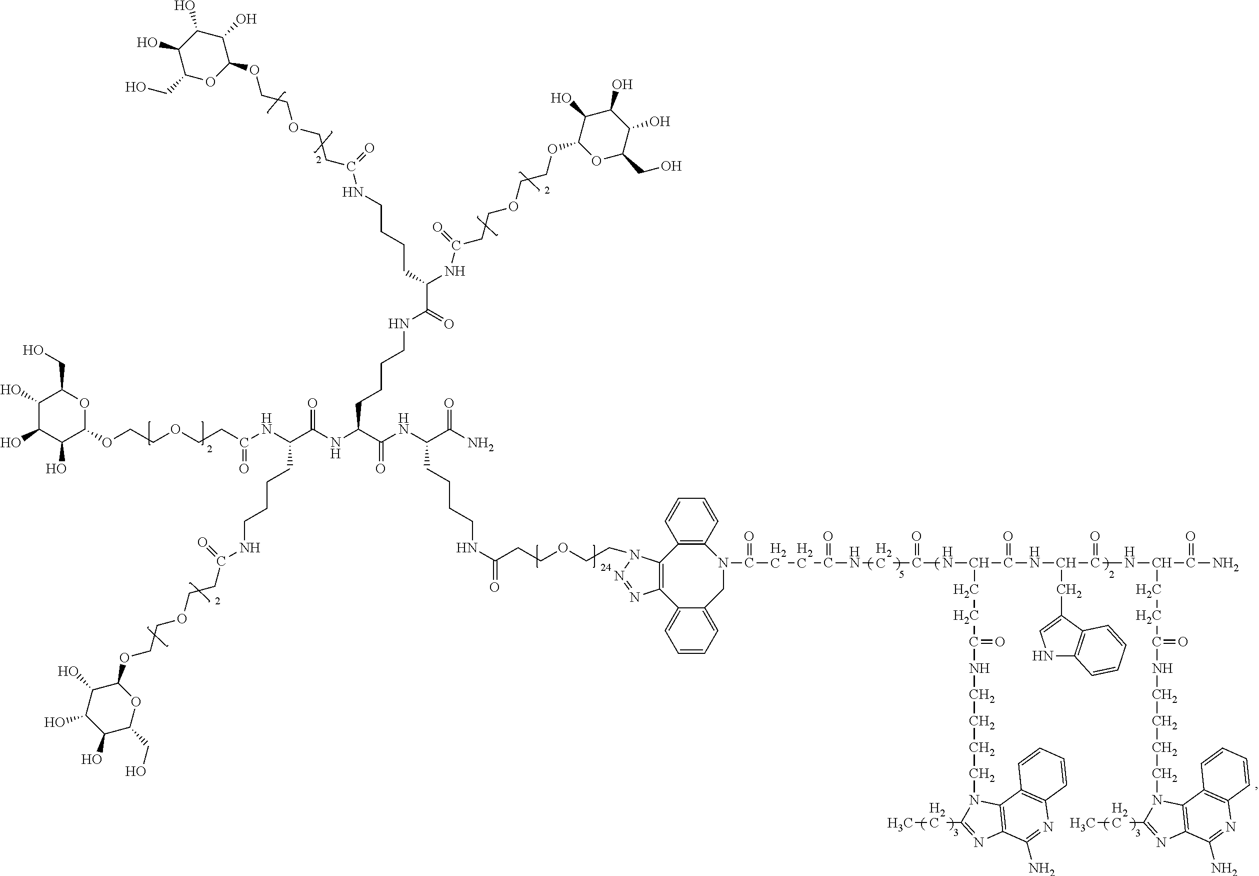

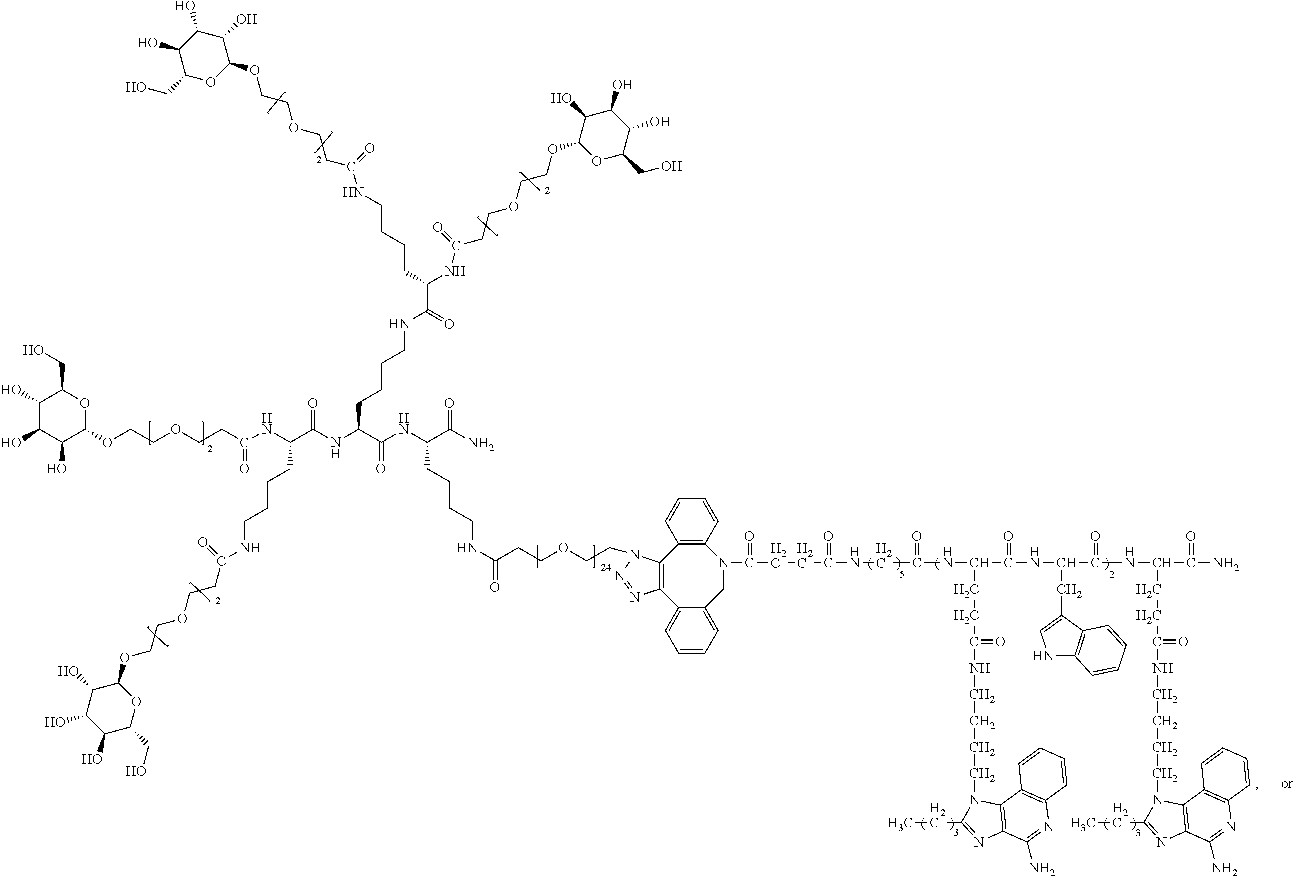

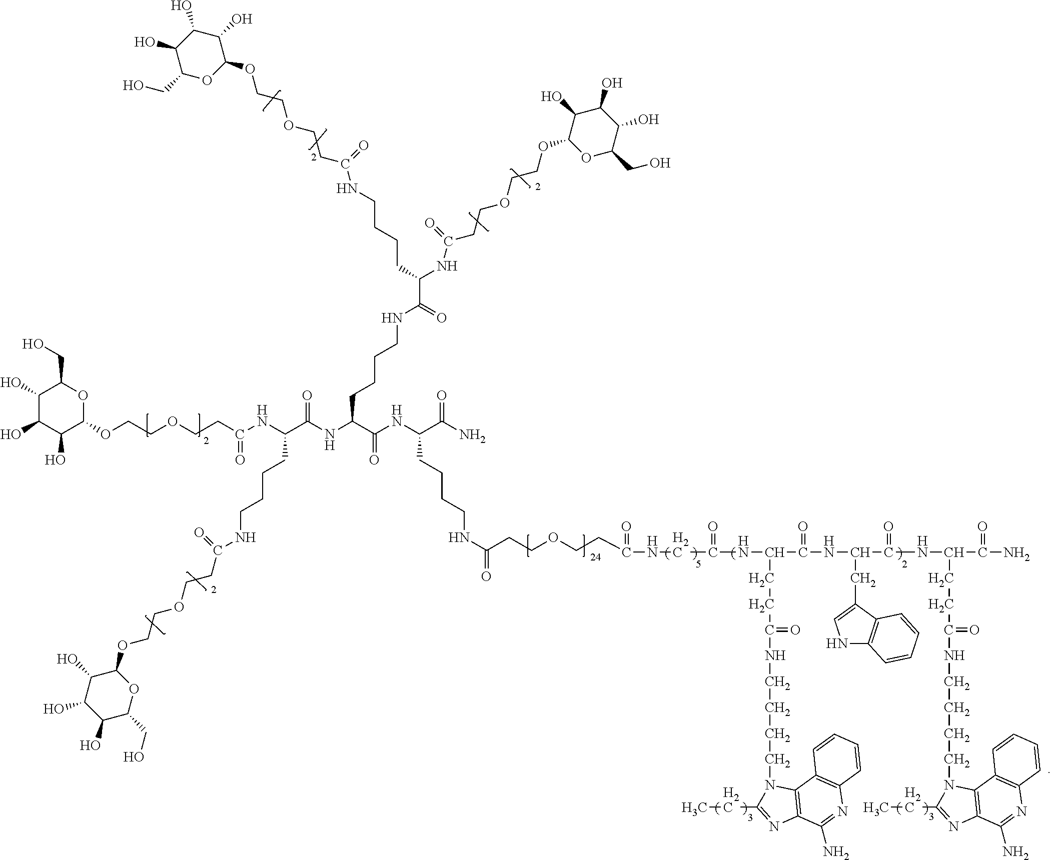

In some embodiments, the composition is in the form of micelles.

In some embodiments, the micelles are between about 5 nm to about 200 nm in diameter, or between about 5 nm and about 50 nm in diameter.

In some embodiments, the at least one drug molecule is noncovalently associated with the micelles. In some embodiments, the composition undergoes a change in overall charge and/or solubility when exposed to a pH less than 7.4, relative to its overall charge or solubility at physiologic pH of 7.4.

In some embodiments, the composition undergoes a change in overall charge and/or solubility when exposed to a pH less than 7.0, relative to its overall charge or solubility at physiologic pH of 7.4.

In some embodiments, the composition is a nanomedicine.

The present disclosure also provides a method of selectively delivering at least one drug molecule D to a target cell or tissue in a subject, comprising administering to the subject a composition of the invention.

In some embodiments, the pH of the target cell or tissue is less than 7.4.

In some embodiments, the pH of the target cell or tissue is about 6.0 to 7.3.

In some embodiments, the target cell or tissue is a tumor cell or tissue.

In some embodiments, the composition is administered intravenously.

The present disclosure also provides a method of treating a cancer, an infectious disease, or an inflammatory disease in a subject in need thereof, comprising administering to the subject the composition of the invention.

The present disclosure also provides a method of treating chronic viral infection in a subject in need thereof, comprising administering to the subject the composition of the invention.

The present disclosure also provides a method of preventing or inhibiting an immune response in a subject against a second drug molecule (D2) or an expression system (D2e), wherein the method comprises administering to the subject a composition comprising:
at least one amphiphile; and
a first drug molecule (D1) selected from inhibitors of mTORC1 and/or mTORC2,
wherein the composition is administered to the subject at a time 1 (Ti) and and the second drug molecule (D2) or the expression system (D2e) is administered to the subject at a time (T2).

In some embodiments, the first drug molecule (D1) is noncovalently associated with or covalently bonded directly or via a suitable linker X1 to the at least one amphiphile.

In some embodiments, the composition and the second drug molecule (D2) or the expression system (D2e) are each administered orally. In other embodiments, the composition and the second drug molecule (D2) or the expression system (D2e) are each administered by injection. In some embodiments, the composition and the second drug molecule (D2) or the expression system (D2e) are each administered by local injection. In other embodiments, the composition and the second drug molecule (D2) or the expression system (D2e) are each administered by intravascular injection.

In some embodiments, one of the composition and the second drug molecule (D2) or the expression system (D2e) is administered by intravascular injection and the other is administered by local injection.

In some embodiments, the composition is administered orally and the second drug molecule (D2) or the expression system (D2e) is administered by injection. In other embodiments, the second drug molecule (D2) or the expression system (D2e) is administered by local injection. In other embodiments, the second drug molecule (D2) or the expression system (D2e) is administered by intravascular injection.

In some embodiments, the composition is administered by injection and the second drug molecule (D2) or the expression system (D2e) is administered orally. In other embodiments, the composition is administered by local injection. In other embodiments, the composition is administered by intravascular injection.

In some embodiments, T1 and T2 are identical. In other embodiments, T1 occurs at least 6 hours before or at least 6 hours after T2. In other embodiments, T1 occurs between about 1 minute and about 120 minutes before T2.

In some embodiments, the first drug molecule is a dual inhibitor of mTORC1 and mTORC2.

In some embodiments, the first drug molecule is a dual inhibitor of mTORC1 and mTORC2 selected from an ATP-competitive mTOR inhibitor.

In some embodiments, the first drug molecule is selected from rapamycin, sirolimus, tacrolimus, INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458, BEZ235, AZD2014, and XL765, and any derivatives or structural analogs thereof, including any prodrug forms thereof.

In some embodiments, the first drug molecule is selected from rapamycin, sirolimus, tacrolimus, INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458, RAD001, CCI-779, AP23573, BEZ235, AZD2014 and XL765, and any derivatives or structural analogs thereof, including any prodrug forms thereof.

In some embodiments, the first drug molecule is everolimus.

In some embodiments, the first drug molecule is selected from Torin-1 and Torin-2.

In some embodiments, the second drug molecule (D2) is selected from any synthetic or recombinant peptide or protein-based drug molecule, or any modification or derivative thereof.

In some embodiments, the second drug molecule (D2) is an Anti-TNFα, Anti-VEGF, Anti-CD20, Anti-Her2, Anti-EGFR, Anti-α4/β1/7 integrin, Anti-CD3, Anti-GRPIIb/IIIa, Anti-CD20 or Anti-CD20 drug conjugates, Anti-IL2R, Anti-CD33, Anti-CD52, Anti-CD11, Anti-IgE, Anti-C5, Anti-IL-1b, Anti-EPCAM, Anti-IL12/23, Anti-IL6R, Anti-RANK-L, Anti-BLys, Anti-B. anthrasis PA, Anti-CTLA-4, Anti-PD1 or Anti-PDL1, Anti-CD30, Anti-Factor IX and X, or anti-viral drug molecule.

In some embodiments, the Anti-TNFα drug molecule is selected from Etanercept, Adalimumab, Infliximab, Certolizumab pegol, and Golimumab; the Anti-VEGF drug molecule is selected from Bevacizumab, Ranibizumab, and Ranibizumab; the Anti-CD20 drug molecule is selected from Rituximab and Ofatumumab; the Anti-Her2 drug molecule is Trastuzumab; the Anti-EGFR drug molecule is selected from Cetuximab and Panitumumab; the Anti-α4/β1/7 integrin drug molecule is Natalizumab; the Anti-CD3 drug molecule is Muromonomab; the Anti-GRPIIb/IIIa drug molecule is Abciximab; the Anti-CD20 or Anti-CD20 drug conjugate drug molecule is selected from Rituximab, Tositumomab, and Ibritumomab tiuxetan; the Anti-IL2R drug molecule is selected from Basiliximab and Daclizumab; the Anti-CD33 drug molecule is Gemtuzumab; the Anti-CD52 drug molecule is Alemtuzumab; the Anti-CD11 drug molecule is Efalizumab; the Anti-IgE drug molecule is Omalizumab; the Anti-C5 drug molecule is Eculizumab; the Anti-IL-1b drug molecule is Canakinumab; the Anti-EPCAM drug molecule is Catumaxomab; the Anti-IL12/23 drug molecule is Ustekinumab; the Anti-IL6R drug molecule is Toclizumab; the Anti-RANK-L drug molecule is Denosumab; the Anti-BLys drug molecule is Belimumab; the Anti-B. anthrasis PA drug molecule is Raxibacumab; the Anti-CTLA-4 drug molecule is Ipilimumab; the Anti-PD1 or Anti-PDL1 drug molecule is selected from Nivolumab, Pembrolizumab, and Atezolizumab; the Anti-CD30 drug molecule is Brentuxmiab vedotin; the Anti-Factor IX and X drug molecule is Emicizumab; and the anti-viral drug molecule is Palivizumab.

In some embodiments, the second drug molecule (D2) is insulin or modified insulin.

In some embodiments, the second drug molecule (D2) is Erythropoietin (EPO) or modified EPO.

In some embodiments, the second drug molecule (D2) is selected from Pramintide and Exenatide.

In some embodiments, the second drug molecule (D2) is GCSF or modified GCSF (e.g., Filgrastim, Pegfilgrastim, Sargramostim), Interferons or modified interferons (e.g., Avonex, Rebif, Peginterferon alfa-2a, Interferon beta-1b), IL-2 and modified IL-2 (e.g., Denileukin difitox), IL-11 (e.g., Oprelvekin), growth hormone, modified growth hormones and growth hormone antagonists (e.g., Pegvisoman), IGF1 (e.g., Mecasermin), follicle-stimulating hormone (FSH), human chorionic gonadotropin, Luteinizing hormone (e.g., Lutropin-a), calcitonin (e.g., Salmon calcitonin), parathyroid hormone or parts of parathyroid hormone (e.g., Teriparatide), Clotting cascade factors such as Factor VIIa, Factor VIII (e.g., Octocog alfa, Eptacog alfa, Rec antihemophilic factor), Factor IX, Protein C, al-proteinase inhibitor, Antithrombin III (serine protease inhibitor, desmopressin, Botulinum toxins (e.g., Botulinum toxin type A, OnabotulinumtoxinA, Botulinum toxin type B), P-Glucocerebrosidase, Alglucosidase-a, Laronidase, Idursulfase, Galsulfase, Agalsidase-P, Lactase, Pancreatic enzymes (lipase, amylase and other proteases), Adenosine deaminase, Tissue plasminogen activator, Drotrecogin-a, Trypsin, Collagenase, Human deoxyribonuclease I, Hyaluronidase, Papain, L-Asparaginase, Rasburicase, or Streptokinase.

In some embodiments, the second drug molecule (D2) is a small molecule.

In some embodiments, the small molecule is capable of forming haptens when administered to the subject.

In some embodiments, the small molecule is capable of inducing antibodies when administered to the subject.

In some embodiments, the small molecule is a β-lactam antibiotic.

In some embodiments, the small molecule is penicillin or cephalosporin.

In some embodiments, the small molecule is a chemotherapeutic drug.

In some embodiments, the second drug molecule (D2) is a blood product, a cell-based product, or a protein extract.

In some embodiments, the expression system (D2e) is selected from adenoviruses (Ad), adeno associated viruses (AAV), rhabdoviruses, poxviruses (e.g., MVA), herpesviruses, lentiviruses and DNA or RNA, which may be free or in the form of a complex with lipids (e.g., lipoplex) or polymer (polyplex).

In some embodiments, the first amphiphile of the composition has the formula S-[B]-[U]-H, wherein S, independently for each occurrence, is a solubilizing block;
B, independently for each occurrence, is a spacer;
H, independently for each occurrence, is a hydrophobic block;
U, independently for each occurrence, is a linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof; and
the first drug molecule (D1) is noncovalently associated with or covalently bonded directly or via a suitable linker X1 to the at least one amphiphile.

In some embodiments, the S of the first amphiphile comprises SGs selected from carboxylic acids.

In some embodiments, the S of the first amphiphile comprises SGs selected from sugar moieties.

In some embodiments, the SGs are mannose.

In some embodiments, 4 to 8 SGs are connected to the S.

In some embodiments, the dendron amplifier comprises repeating monomer units of 1 to 10 generations having between 2 to 6 branches per generation.

In some embodiments, the molar ratio of amphiphile to the first drug molecule (D1) is from about 20:1 to about 1:20. In other embodiments, the molar ratio of amphiphile to the first drug molecule (D1) is from about 5:1 to about 1:5. In other embodiments, the molar ratio of amphiphile to the first drug molecule (D1) is from about 2:1 to about 1:4. In other embodiments, the molar ratio of amphiphile to the first drug molecule (D1) is from about 1:1 to about 1:4.

In some embodiments, the composition is in the form of micelles.

In some embodiments, the micelles are between about 5 nm to about 200 nm in diameter, or between about 5 nm and about 50 nm in diameter.

In some embodiments, the first drug molecule is noncovalently associated with the micelles.

In some embodiments, further comprising administering a third drug molecule (D3) to the subject.

In some embodiments, the third drug molecule (D3) is TLR-7/8a (e.g., Compound 4), a TLR-3a (e.g., pICLC) or a TLR-9a (e.g., CpG). In other embodiments, the third drug molecule (D3) is a NOD-like receptor agonist (e.g., muramyl dipeptide (MDP)), a mincle receptor agonist (e.g., trehalose dibehenate (TDB)) and TLR4 agonists (e.g., LPS or MPL)

In some embodiments, any of the disclosed amphilies is employed in the method of method of preventing or inhibiting an immune response in a subject against a second drug molecule (D2) or an expression system (D2e).

### Linkers

The term linker refers to any molecule that joins together any two or more molecules (or “moieties”), such as any two or more components of amphiphiles or drug conjugates and may additionally perform any one or more of the following functions: I) increase or decrease water solubility; II) increase distance between any two components; III) impart rigidity or flexibility; or, IV) modulate the rate of degradation of the link between any two or more different molecules. As used herein, the term “linker” may be used to describe linkers (U), suitable linkers (X), such as X1, X2, X3, X4, X5 and X6.

Linkers that have particular utility are named, and specific, preferred compositions of those named linkers are described throughout the specification. The spacer (B) is a linker between the solubilizing block (S) and the hydrophobic block (H) on amphiphiles. The molecule that results from the reaction of Linker precursor 1 (“U1”), which is linked to the solubilizing block (S) or drug (D) either directly or via a spacer (B), with Linker precursor 2 (“U2”) on a hydrophobic block (H) is referred to as a Linker U. Suitable linker X refers to any linker suitable for linking two or more adjacent groups groups. Suitable linkers preferred for joining drug molecules (D) to hydrophobic blocks (H) are referred to as X1. Suitable linkers preferred for joining aryl or heteroaryl groups to the hydrophobic block are referred to as X2. Suitable linkers used to join reactive functional groups (“FG4”) to the pharmacophore of drug molecules (D) are referred to as X3. Suitable linkers preferred for joining charged groups to hydrophobic block (H) are referred to as X4. Suitable linkers preferred for joining SG to S are referred to as X5. Suitable linkers preferred for joining the solubilizing block (S) and spacer (B) of the amphiphile are referred to as X6.

The linker may use covalent or non-covalent means to join any two or more components. In preferred embodiments, a linker may join, i.e., link, any two components through a covalent bond. Covalent bonds are the preferred linkages used to join any two components and ensure that no component can immediately disperse from the other components following administration to a subject.

There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof, which, for covalent linkers, further comprise two or more functional groups, which may be the same or different, that are used to link any two molecules, e.g., any two components of amphiphiles and/or drug conjugates, through covalent bonds.

In some embodiments, the carbon linker can include a C1-C18 alkane linker, e.g., a lower alkyl linker, such as C1-C6 (i.e., from one to six methylene units), which can serve to increase the space between two or more molecules, i.e., different components, while longer chain alkane linkers can be used to impart hydrophobic characteristics. Alternatively, hydrophilic linkers, such as ethylene oxide linkers, may be used in place of alkane linkers to increase the space between any two or more heterologous molecules and increase water solubility. In other embodiments, the linker can be a cyclic and/or aromatic compound, or poly(aromatic) compound that imparts rigidity. The linker molecule may comprise a hydrophilic or hydrophobic linker. In several embodiments, the linker includes a degradable peptide sequence that is cleavable by an intracellular enzyme (such as a cathepsin or the immunoproteasome).

For linking two components of amphiphiles and drug conjugates, wherein at least one of the components comprises a peptide, it was found that linkers comprising between 2 and 7 methylene groups improved coupling of the two or components. In a non-limiting example, increasing the number of methylene units between the amide and the amine of the N-terminal amino acid of peptide-based hydrophobic blocks (H) led to improved coupling to other molecules, including U2, spacers (B) and solubilizing blocks (S). Therefore, in preferred embodiments, the N-terminal amino acid of poly(amino acid)-based hydrophobic blocks (H) comprises two or more, typically between 2 and 7, such as 1, 2, 3, 4, 5, 6, 7 methylene units. For clarity, an amino acid with 2 methylene units is beta-alanine and an amino acid with 5 methylene units is amino-hexanoic acid. In certain preferred embodiments, the N-terminal amino acid of peptide-based hydrophobic blocks (H) is amino-hexanoic acid (sometimes referred to as Ahx; CAS number 60-32-3). In other embodiments, the N-terminal amino acid of peptide-based hydrophobic blocks (H) is beta-alanine.

In some embodiments, the linker may comprise poly(ethylene oxide) (PEG). The length of the linker depends on the purpose of the linker. For example, the length of the linker, such as a PEG linker, can be increased to separate any two or more components, for example, to reduce steric hindrance, or in the case of a hydrophilic PEG linker can be used to improve water solubility. The linker, such as PEG, may be between about 1 and about 48 monomers in length, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 monomers in length or more. When used as a spacer (B), the PEG may be up to about 48 monomers in length or more, though, typically PEG spacers are been between about 1 and 36 monomers in length.

In some embodiments, wherein the linker comprises a carbon chain, the linker may comprise a chain of between about 1 or 2 and about 18 carbons, such as 1, 2, 3, 4, 5, 6, 7, 8, 9, 10, 11, 12, 13, 14, 15, 16, 17, 18 carbons in length or more. In some embodiments, wherein the linker comprises a carbon chain, the linker may comprise a chain of between about 12 and about 20 carbons. In some embodiments, wherein the linker comprises a carbon chain, the linker may comprise a chain of between no more than 18 carbons, typically between about 1 and 6 carbon atoms.

The linkage used to join any two or more molecules, e.g., any two or more components of amphiphiles and/or drug conjugates may comprise any suitable functional group, including but not limited to amides, esters, ethers, thioethers, silyl ethers, disulfides, carbamates, carbamides, hydrazides, hydrazones, acetals and triazoles.

In a non-limiting example of a covalent linkage, a click chemistry reaction may result in a triazole that links, i.e., joins together, any two components of the amphiphile or drug molecule conjugate. In several embodiments, the click chemistry reaction is a strain-promoted [3+2] azide-alkyne cyclo-addition reaction. An alkyne group and an azide group may be provided on respective molecules to be linked by “click chemistry”. In some embodiments, a drug (D) bearing an azide functional group is coupled to a hydrophobic block (H) having an appropriate reactive group, such as an alkyne, for example, a dibenzylcyclooctyne (DBCO).

In some embodiments, an amine is provided on one molecule and may be linked to another molecule by reacting the amine with any suitable electrophilic group such as carboxylic acids, acid chlorides, activated esters (for example, NHS ester), which results in an amide bond; the amine may be reacted with alkenes (via Michael addition); the amine may be reacted with aldehydes and ketones (via Schiff base); or, the amine may be reacted with activated carbonates or carbamates to yield a carbamate.

In some embodiments, the linker is cleavable under intracellular conditions, such that cleavage of the linker results in the release of any component linked to the linker, for example, a drug molecule (D).

For example, the linker can be cleavable by enzymes localized in intracellular vesicles (for example, within a lysosome or endosome or caveolae) or by enzymes, in the cytosol, such as the proteasome, or immunoproteasome. The linker can be, for example, a peptide linker that is cleaved by protease enzymes, including, but not limited to proteases that are localized in intracellular vesicles, such as cathepsins in the lysosomal or endosomal compartments of cells.

The peptide linker is typically between 1-10 amino acids, such as 1, 2, 3, 4, 5, 6, 7, 8, 9, 10, or more (such as up to 20) amino acids long, such as 11, 12, 13, 14, 15, 16, 17, 18, 19, 20 or more amino acids long. When used as a spacer (B), the peptide linker may be up to about 45 amino acids, though, when used as an enzyme degradable linker to control the rate of release of drug (D), or the shedding of the solubilizing block (S), linked to a spacer, e.g., via X6, the peptide linker is typically between 1 and 8 amino acids, such as 1, 2, 3, 4, 5, 6, 7 or 8 amino acids in length, though, typically no more than 6 amino acids in length.

Certain dipeptides are known to be hydrolyzed by proteases that include cathepsins, such as cathepsins B and D and plasmin, (see, for example, Dubowchik and Walker, 1999, Pharm. Therapeutics 83:67-123). For example, a peptide linker that is cleavable by the thiol-dependent protease cathepsin-B, can be used (for example, a Phe-Leu or a Gly-Phe-Leu-Gly (SEQ ID NO:1) linker). Other examples of such linkers are described, for example, in U.S. Pat. No. 6,214,345, incorporated herein by reference. In certain such embodiments, the peptide linker cleavable by an intracellular protease is a Val-Cit linker or a Phe-Lys linker (see, for example, U.S. Pat. No. 6,214,345, which describes the synthesis of doxorubicin with the Val-Cit linker). Note: for examples of amino acids and peptides provided throughout the specification (within the text of figures), unless otherwise specified, the peptides and amino acids are L-amino acids.

The cleavable peptide linker can be selected to promote processing (i.e., hydrolysis) of the peptide linker following intracellular uptake by immune cells. The sequence of the cleavable peptide linker can be selected to promote processing by proteases in the extracellular spaces, such as the extracellular environment of tumors, or in the intracellular spaces, such as the intracelluar environment of antigen presenting cells.

In several embodiments, linkers comprising peptide sequences of the formula Pn . . . P4-P3-P2-P1 are used to promote recognition by cathepsins, wherein P1 is selected from arginine, lysine, acetyl lysine (i.e., the epsilon amine is acetylated), boc protected lysine (i.e., the epsilon amine is boc protected), citrulline, glutamine, threonine, leucine, norleucine, alpha-aminobutyric acid (abbreviated as “a-But” herein) or methionine; P2 is selected from beta-alanine, glycine, serine, leucine, valine or isoleucine; P3 is selected rom beta-alanine, glycine, serine, alanine, proline, or leucine; and P4 is selected from beta-alanine, glycine, serine, arginine, lysine, acetyl lysine (i.e., the epsilon amine is acetylated), boc protected lysine, aspartic acid, glutamic acid or beta-alanine. In a non-limiting example, a tetrapeptide linker of the formula P4-P3-P2-P1 linked through an amide bond to another molecule and has the sequence Lys-Pro-Leu-Arg (SEQ ID NO:2). For clarity, the amino acid residues (Pn) are numbered from proximal to distal from the site of cleavage, which is C-terminal to the P1 residue, for example, the amide bond between P1-P1′ is hydrolyzed. Suitable peptide sequences that promote cleavage by endosomal and lysosomal proteases, such as cathepsin, are well described in the literature (see: Choe, et al., J. Biol. Chem., 281:12824-12832, 2006).

In several embodiments, linkers comprising peptide sequences are selected to promote recognition by the proteasome or immunoproteasome. Peptide sequences of the formula Pn . . . P4-P3-P2-P1 are selected to promote recognition by proteasome or immunoproteasome, wherein P1 is selected from basic residues and hydrophobic, branched residues, such as arginine, lysine, leucine, isoleucine and valine; P2, P3 and P4 are optionally selected from leucine, isoleucine, valine, lysine and tyrosine. In a non-limiting example, a cleavable linker of the formula P4-P3-P2-P1 that is recognized by the proteasome is linked through an amide bond at P1 to another molecule and has the sequence Tyr-Leu-Leu-Leu (SEQ ID NO:3). Sequences that promote degradation by the proteasome or immunoproteasome may be used alone or in combination with cathepsin cleavable linkers. In some embodiments, amino acids that promote immunoproteasome processing are linked to linkers that promote processing by endosomal proteases. A number of suitable sequences to promote cleavage by the immunoproteasome are well described in the literature (see: Kloetzel, et al., Nat. Rev. Mol. Cell Biol., 2:179-187), 2001, Huber, et al., Cell, 148:727-738, 2012, and Harris et al., Chem. Biol., 8:1131-1141, 2001).

In certain preferred embodiments, drug molecules (D) are linked to hydrophobic blocks (H) via linker X1 comprising an enzyme degradable peptide. A non-limiting example is shown here:

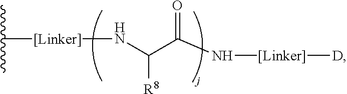

wherein D is a drug molecule; “Linker” is any suitable linker molecule; j denotes any integer, though, j is typically 1 to 6 amino acids, such as 1, 2, 3, 4, 5 or 6 amino acids; R⁸ is any suitable amino acid side group; the N-terminal amine of the peptide is linked either directly or via the ends, e.g., to the N- or C-termini of a hydrophobic block (H) comprising poly(amino acids), either directly or via U, or through reactive monomers comprising the hydrophobic block (H); and, brackets “[ ]” denote that the group is optional.

In certain preferred embodiments of drug molecules linked to hydrophobic blocks (H) via linker X1 comprising an enzyme degradable peptide, the drug molecule (D) is linked directly to the peptide through an amide bond as shown here:

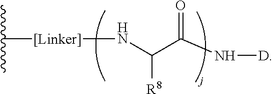

In a non-limiting example of the above structure, wherein the N-terminal Linker group is present and selected from beta alanine the structure is:

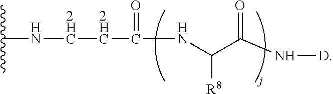

In some embodiments, the drug molecule (D) is linked to the peptide via a self-immolative carbamate linker. A non-limiting example is shown here:

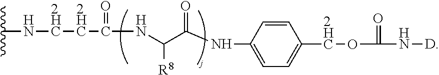

In the above example, wherein j is 4 and the amino acids are Serine-Lysine(Ac)-Valine-nor-Leucine, the structure is:

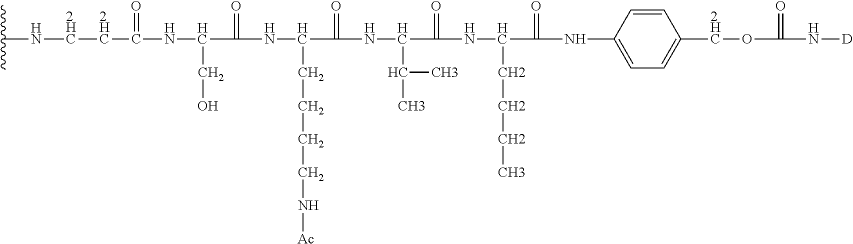

In some embodiments, drug molecules (D) are linked to hydrophobic blocks (H) through a sulfatase degradable linker X1, wherein hydrolysis of a sulfate by sulfatase results in release of the drug molecule from the linker. A number of arylsulfatase and alkysulfatase degradable linkers have recently been described (e.g., see: Bargh, et al., 2020, Chem. Sci. 11, 2375). In some embodiments of the present disclosure, drug molecules are linked to hydrophobic blocks (H) through sulfatase degradable linkers. Non-limiting examples are shown here for clarity:

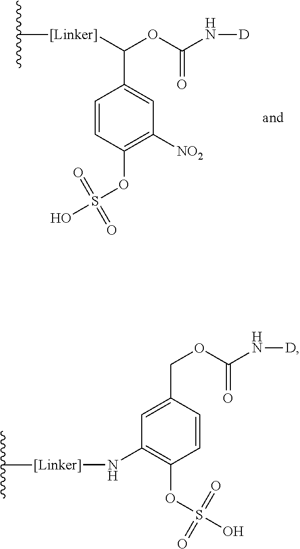

wherein D is a drug molecule; “Linker” is any suitable linker molecule linked either directly or via ends, e.g., to the N- or C-termini of a hydrophobic block (H) comprising poly(amino acids), either directly or via U, or through reactive monomers comprising the hydrophobic block (H); and, brackets “[ ]” denote that the group is optional.

Non-limiting examples of the above structures, wherein the “Linker” is present and selected from short alkyl linkers linked to the hydrophobic block through an amide are shown here for clarity:

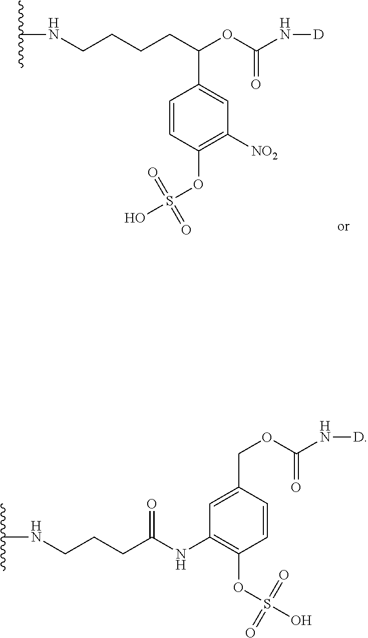

In other embodiments, any two or more components may be joined together through a pH-sensitive linker X that is sensitive to hydrolysis under acidic conditions. A number of pH-sensitive linkers are familiar to those skilled in the art and include for example, a hydrazone, carbohydrazone, semicarbazone, thiosemicarbazone, cis-aconitic amide, orthoester, acetal, ketal, silylether or the like (see, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661).

In certain embodiments, different components (e.g., drug molecule and hydrophobic block (H)) are linked together through pH-sensitive linkers that are stable at blood pH, e.g., at a pH of about 7.4, but undergo more rapid hydrolysis at endosomal/lysosomal pH, ˜pH 5-6.5. In certain, preferred embodiments, drug molecules (D) are linked to hydrophobic blocks (H) through reactive monomers via a pH-sensitive bonds, such as hydrazone bonds that result from the reaction between a ketone and a hydrazine. The functional group hydrazine linked to a carbonyl is sometimes referred to as hydrazide, though, hydrazine is meant to broadly refer to —NH—NH₂ groups, including when linked to carbonyl, e.g., C(O)—NH—NH₂. pH-sensitive linkages, such as a hydrazone, provide the advantage that the bond is stable at physiologic pH, at about pH 7.4, but is hydrolyzed at lower pH values, such as the pH of intracellular vesicles.

In certain preferred embodiments, drug molecules are linked by a linker X1 comprising a ketone and may be represented by the formula:

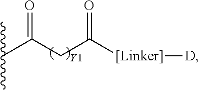

wherein D is any drug molecule; “Linker” is any suitable linker molecule; y1 denotes an integer between 1 to 6, preferably 4; brackets “[ ]” denote that the group is optional; and, wherein the ketone in the above example is used to link the linker linked drug molecule (D) to a reactive monomer through a hydrazone bond.

In the above example, wherein y1 is 4 and the drug molecule is linked directly (i.e., the “Linker” is absent) via an amide bond, the structure is:

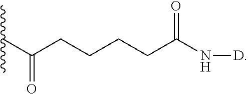

In preferred embodiments, drug molecules linked to ketones are linked to hydrophobic blocks (H) through hydrazone or carbohydrazone bonds. Non-limiting examples of drug molecules linked to a glutamic acid-based reactive monomer (N) through hydrazone and carbohydrazone bonds are shown here:

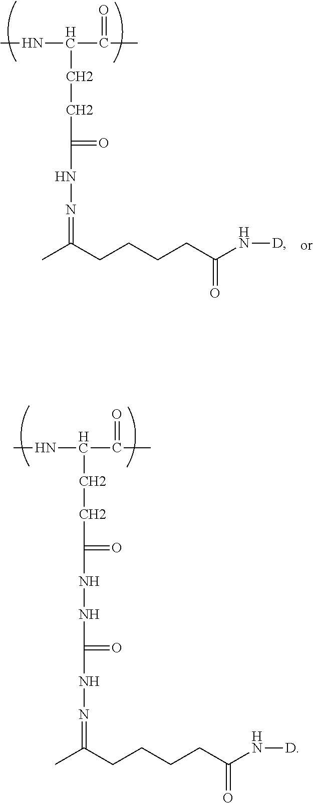

In some embodiments, the drug molecule comprises a ketone and may be linked directly to reactive monomers through hydrazone or carbohydrazone.

In other embodiments, the linker comprises a linkage that is cleavable under reducing conditions, such as a reducible disulfide bond. Many different linkers used to introduce disulfide linkages are known in the art (see, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.).

In preferred embodiments, the linker X1 linking a hydrophobic block (H) and one or more drug molecules (D) is a short alkyl or PEG linker. In other preferred embodiments, the linker X1 linking a hydrophobic block (H) and one or more drug molecules (D) is an enzyme degradable linker, such as a cathepsin degradable peptide or sulfatase degradable linker. In other preferred embodiments, the linker X1 linking a hydrophobic block (H) and one or more drug molecules (D) comprises an enzyme degradable peptide and a self-immolative linker.

X can be any suitable linker, though, in preferred embodiments, the linker X linking any two or more groups, is a short alkyl (i.e., lower alkyl) or PEG linker, e.g., a PEG linker with between about 1 to about 24 monomeric units.

### Spacer

The spacer (B) is a type of linker that links the solubilizing block (S) to the hydrophobic block (H) either directly or via a Linker (U), e.g., wherein the amphipile has the structure S-B-H or S-B-U-H. The spacer (B) may comprise any one or more of the following: amino acids, including non-natural amino acids; hydrophilic polymers, e.g., polymers based on ethylene oxide (PEG), acrylate, methacrylate, acrylamide or methacrylamide based monomers; alkane chains; or the like; or combinations thereof. The spacer (B) may be linked to the solubilizing block (S) and hydrophobic block (H) through any suitable means, e.g., directly, or indirectly via linkers, though the linkages typically comprise covalent bonds, e.g., amide bonds. Certain spacers linking the spacer (B) to the solubilizing block (S) are referred to as X6; preferred embodiments of X6 are described throughout the specification.

In some embodiments, the spacer (B) functions to provide distance, i.e., space, between the heterologous molecules, S and H. In other embodiments, the spacer (B) functions to impart hydrophobic or hydrophilic properties. In still other embodiments, the composition of the spacer may be selected to impart rigidity or flexibility. In other embodiments, the composition of the spacer may be selected for recognition by enzymes and promote degradation.

In some embodiments, the spacer (B) is a hydrophilic polymer, with monomer units selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof.

In some embodiments, the spacer (B) is a peptide sequence between about 1 to 45 amino acids in length, such as 1, 2, 3, 4, 5, 6, 7, 8, 9, 10, 11, 12, 13, 14, 15, 16, 17, 18, 19, 29, 21, 22, 23, 24, 25, 26, 27, 28, 29, 30, 31, 32, 33, 34, 35, 36, 37, 38, 39, 40, 41, 42, 43, 44 or 45 amino acids, typically no more than 45 amino acids in length, that is linked to the hydrophobic block (H) and solubilizing block (S) through, e.g., an amide bond formed between the N- and C-terminal carboxyl group of the spacer (B), respectively. The amide bond between the spacer (B) and the solubilizing block (S) and/or hydrophobic block (H) may be recognized by enzymes or may be selected for resistance to enzyme-mediated hydrolysis.

In other embodiments, the spacer (B) is a hydrophilic polymer comprising monomer units selected from non-natural, hydrophilic monomers, e.g., ethylene oxide (PEG), HPMA, HEMA, or the like, and is about 1 to 48 monomers in length (i.e. degree of polymerization), such as 1, 2, 3, 4, 5, 6, 7, 8, 9, 10, 11, 12, 13, 14, 15, 16, 17, 18, 19, 29, 21, 22, 23, 24, 25, 26, 27, 28, 29, 30, 31, 32, 33, 34, 35, 36, 37, 38, 39, 40, 41, 42, 43, 44, 45, 46, 47 or 48 monomers, typically no more than 48 monomers in length, that is linked to the hydrophobic block (H) and solubilizing block (S) either directly or through linkers.

Specific compositions of spacers that lead to unexpected improvements in biological activity are described throughout the specification. Note: spacer groups (B) and solubilizing blocks (S) may both comprise hydrophilic polymers (e.g., hydrophilic poly(amino acids); hydrophilic methacrylate-based polymers, such as HEMA; hydrophilic methacrylamide-based polymers, such as HPMA, PEG, etc.); however, the distinction between S and B is based in part on function and called attention to in specific examples of amphiphiles.

### Linker (U)

A linker (U) optionally joins solubilizing block (S) fragments (S-[B]-U1) to hydrophobic block (H) fragments (U2-H) through the reaction of U1 with U2 to form amphiphiles (S-[B]-U-H), or the linker U joins drug molecule (D) fragments (D-[B]-U1) to hydrophobic block (H) fragments (U2-H) through the reaction of U1 with U2 to form drug molecule conjugates (D-[B]-U-H)

While solubilizing blocks (S) on the amphiphile may be joined directly to hydrophobic blocks (H), i.e., S-H, or via a spacer, i.e., S-B-H, it may be beneficial under certain circumstances to produce the solubilizing block (S) and hydrophobic block (H) as separate fragments comprising Linker Precursor U1 (S-[B]-U1) and Linker Precursor U2 (U2-H), which may be joined on-resin or in solution to yield S-[B]-U-H. Similarly, while drug molecules may be joined directly to hydrophobic blocks (H), i.e., D-H, or via a spacer, it may be beneficial under certain circumstances to produce the solubilizing block (S) and hydrophobic block (H) as separate fragments comprising Linker Precursor U1 (S-[B]-U1) and Linker Precursor U2 (U2-H), which may be joined on-resin or in solution to yield S-[B]-U-H.

In preferred embodiments, the Linker Precursors used to form Linker U are selected for site-selectivity, i.e., a reaction only takes place between U1 and U2 and between no other groups. In some embodiments, Linker Precursor U1 comprises an activated carboxylic acid and is reacted with a Linker Precursor U2 that comprises an amine to form Linker U comprising an amide; or, U1 comprises an amine and is reacted with U2 that comprises an activated carboxylic acid to form Linker U comprising an amide. In some embodiments, Linker Precursor U1 comprises a maleimide and is reacted with Linker Precursor U2 that comprises a thiol to form a Linker U comprising a thioether bond; or, U1 comprises a thiol and is reacted with U2 that comprises a maleimide to form a Linker U comprising a thioether bond. In some embodiments, Linker Precursor U1 comprises an azide and is reacted with Linker Precursor U2 that comprises an alkyne to form a Linker U that comprises a triazole; or, U1 comprises an alkyne and is reacted with a U2 that comprises an azide to form a Linker US comprising a triazole.

In preferred embodiments, the amphiphile of formula S-[B]-U-H is joined together by linking a solubilizing block fragment (S-[B]-U1) to a hydrophobic block fragment (U2-H), wherein the Linker Precursor U2 comprises a strained alkyne (e.g., dibenzocyclooctyne (DBCO), bicyclononyne (BCN) or the like) that is reacted with Linker Precursor U1 which comprises an azide to form the Linker U that comprises a triazole. In other preferred embodiments, Linker Precursor U2 comprises an azide that is reacted with the Linker Precursor U1 that comprises a strained alkyne (e.g., dibenzocyclooctyne (DBCO), bicyclononyne (BCN) or the like) to form the Linker U which comprises a triazole. In a non-limiting example, the Linker Precursor U2 comprising DBCO is linked to the hydrophobic block (H) via a suitable linker X (e.g., DBCO-NHS, CAS number 1353016-71-3) and the Linker Precursor U1 (e.g. azido acid, such as azidopentanoic acid; azido amino acid, such as azido-lysine (abbreviated Lys(N3), CAS number 159610-92-1; or, azido amine, such as azido-butylamine) is linked to the solubilizing block fragment (S-[B]-U1) via a suitable linker X.

In preferred embodiments, the Linker U preferably comprises an amide, thioether or triazole.

### Dendron Amplifier

Dendron amplifiers are a specific type of linker moiety that functions to increase the valency (i.e., the number) of groups present on any components of amphiphiles or drug molecule conjugates described herein. For instance, in preferred embodiments of solubilizing blocks (S), dendron amplifiers are used to increase the valency of solubilizing groups (referred to as “SG” in formulae) that are present on the surface of the solubilizing block (S). In other embodiments, the hydrophobic block (H) of an amphiphile or drug molecule conjugate comprises a dendron amplifier and is used to increase the valency of attached drug molecules (D).

Dendron amplifiers (also referred to as “dendrons”) are regularly branched molecules that are often symmetric and typically comprise repeating units of monomers that comprise three or more functional groups (FG) and a branch point. Dendron amplifiers may be expressed by the formula, (FG′)-T-(FGt)d, wherein FG′ and FGt are the focal point and terminal functional groups, respectively, which are selected from any suitable functional group; T is any suitable linker and “d” is any integer greater than 1, typically between 2 to 32, though, more preferably between 2 and 8, such as 2, 3, 4, 5, 6, 7, and 8. The multiple by which dendron amplifiers increase the terminal functional group (FGt) can be expressed as FGt=β^{γ}, wherein β is the number of branches that occur for each generation of the dendron and the symbol γ is the number of generations, wherein the number of branches is any integer, though, typically between 2 to 6, and the number of generations is any integer, though, typically between 1 to 10. Terminal functional groups present on solubilizing blocks that are free (i.e., unreacted), may also be referred to as solubilizing groups (SG).

Dendron amplifiers may comprise repeats of a monomer comprising a first functional group (FG1) and a second functional group (FG2), wherein the first functional group is reactive towards the second functional group. For instance, a non-limiting example of a 2^{nd} generation dendron amplifier with β=2 comprising repeats of a monomer comprising a first functional group (FG1) and a second functional group (FG2), wherein the first functional group is reactive towards the second functional group, is shown here for clarity:

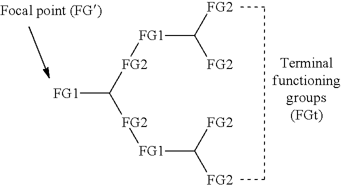

Wherein, the first functional group at the starting point is also referred to as the focal point functional group (FG′) and the terminal FG2 are referred to as the terminal functional groups or FGt.

A non-limiting example of a 3^{rd} generation dendron formed from monomers comprising a first and second functional group wherein β=2 is shown here for clarity:

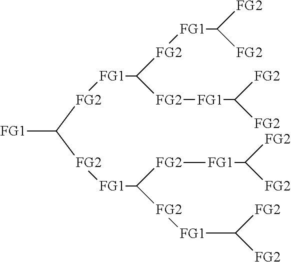

A non-limiting example of a 2^{nd} generation dendron amplifier with β=3 comprising repeats of a first monomer comprising a first functional group (FG1) and a second functional group (FG2), wherein the first functional group is reactive towards the second functional group, is shown here for clarity:

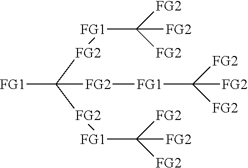

Monomers comprising a first functional group and a second functional group, wherein the first functional group is reactive towards the second functional group, and the monomer comprises at least one first functional group and two or more second functional groups may be selected from any suitable monomer. Non-limiting examples include FG1-(CH₂)_{y2}CH(R¹)₂, FG1-(CH₂)_{y2}C(R¹)₃, FG1-(CH₂CH₂O)_{y2}CH(R¹)₂, FG1-(CH₂CH₂O)_{y2}C(R¹)₃, FG1-CH(R¹)₂, FG1-C(R¹)₃, wherein R¹ is independently selected from (CH₂)_{y3}-FG2, (OCH₂CH₂)_{y3}-FG2 or CH₂(OCH₂CH₂)_{y3}-FG2) and y2 and y3 are each an integer number of repeating units selected from between 1 to 6.

A non-limiting example of FG1-CH(R′)₂, wherein FG1 is NH₂, R¹ is CH₂(OCH₂CH₂)_{y3}-FG2, y3 is 1 and FG2 is COOH is shown here for clarity:

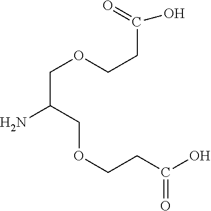 [SMILES: NC(COCCC(=O)O)COCCC(=O)O]

Wherein the above monomer is used to produce a 2^{nd} generation amplifying linker, the structure is:

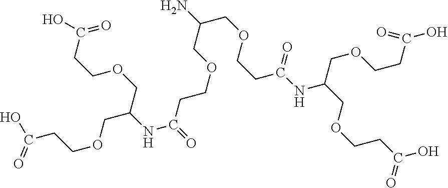 [SMILES: NC(COCCC(=O)NC(COCCC(=O)O)COCCC(=O)O)COCCC(=O)NC(COCCC(=O)O)COCCC(=O)O]

Additional non-limiting examples of monomers comprising a first functional group and a second functional group, wherein the first functional group is reactive towards the second functional group, and the monomer comprises at least one first functional group and two or more second functional groups include FG1-(CH₂)_{y2}N(R²)₂, FG1-(CH₂CH₂O)_{y2}CH₂CH₂N(R²)₂, wherein R² is independently selected from (CH₂)_{y3}-FG2, (CH₂CH₂O)_{y3}(CH₂)_{y4}-FG2, (CH₂OCH₂CH₂)_{y3}-FG2) and y2, y3 and y4 are each an integer of repeating units selected from between 1 to 6. Note: in the above example, FG′ is an amine and the 4 FGt are carboxylic acids.

A non-limiting example of FG1-(CH₂CH₂O)_{y1}CH₂CH₂N(R²)₂, wherein FG1 is NH₂, R² is (CH₂CH₂O)_{y3}(CH₂)_{y4}-FG2, y2 is 2, y3 is 1, y4 is 2 and FG2 is COOH is shown here for clarity:

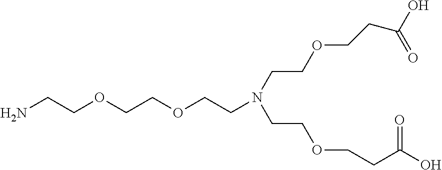 [SMILES: NCCOCCOCCN(CCOCCC(=O)O)CCOCCC(=O)O]

In still additional non-limiting examples of monomers comprising a first functional group and a second functional group, wherein the first functional group is reactive towards the second functional group, and the monomer comprises at least one first functional group and two or more second functional groups include certain amino acids, such as glutamic acid, aspartic acid, lysine or ornithine. A non-limiting example of a 3^{rd} generation lysine dendron is shown here for clarity:

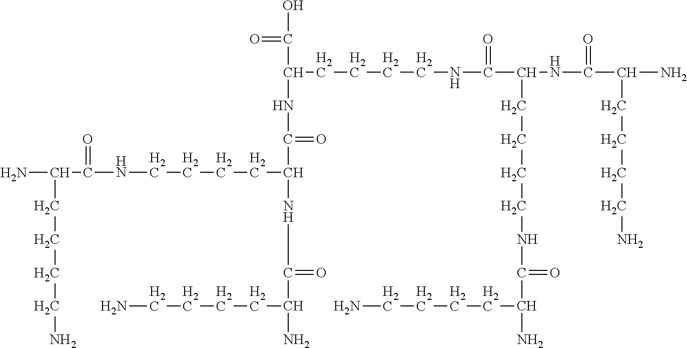 [SMILES: NCCCCC(N)C(=O)CNC(CCCCNC(=O)C(N)CCCCN)C(=O)NC(CCCCNC(=O)C(CCCCNC(=O)C(N)CCCCN)NC(=O)C(N)CCCCN)C(=O)O]

Dendron amplifiers may comprise repeats of two monomers, wherein a first monomer comprises three or more first functional groups (FG1) and the second monomer comprises two or more second functional groups (FG2), wherein the first functional group is reactive towards the second functional group. For instance, a non-limiting example of a 2^{nd} generation dendron amplifier with β=2 comprising repeats of a first and second monomer, wherein the first monomer comprises three first functional groups (FG1) and the second monomer comprises two second functional groups (FG2), wherein the first functional group is reactive towards the second functional group, is shown here for clarity:

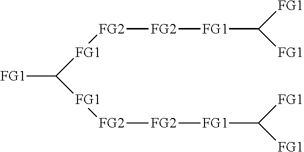

A non-limiting example of a 1^{st} generation dendron amplifier with β=2 comprising repeats of a first and second monomer, wherein the first monomer comprises three first functional groups (FG1) and the second monomer comprises three second functional groups (FG2), wherein the first functional group is reactive towards the second functional group, is shown here for clarity:

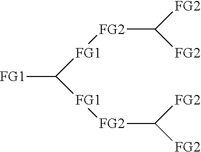

Dendron amplifiers may be used to join any three or more components of amphiphiles and drug molecule conjugates. The focal point functional group (FG′) and the terminal functional groups (FGt) may be further functionalized, i.e., reacted to fit a particular purpose.

In preferred embodiments of amphiphiles of formula S-[B]-[U]-H, the solubilizing block (S) comprises a dendron amplifier wherein the focal point is linked to the hydrophobic block (H) either directly or indirectly via a spacer (B) and/or Linker U and the terminal functional groups (FGt) are either unlinked and serve as the solubilizing groups (SG) or are linked to solubilizing groups (SG) to distinct from the FGt. Solubilizing groups (SG) are any molecules that are hydrophilic and/or charged; preferred solubilizing groups (SG) are described throughout the specification.

In some embodiments of amphiphiles of formula S-[B]-[U]-H-D and drug molecule conjugates of formula H-D, the hydrophobic block (H) comprises a dendron amplifier wherein the focal point is linked to either (i) a solubilizing block (S) either directly or indirectly via a spacer (B) and/or Linker U; or (ii) a drug molecule either directly or via a Linker X1. In other embodiments of drug molecule conjugates, the hydrophobic block is linked to two or more drug molecules via a dendron amplifier.

In some embodiments, the hydrophobic block (H) comprises a dendron amplifier and the terminal functional groups (FGt) are linked to hydrophobic drug molecules. In such embodiments, the focal point is linked to either (i) a solubilizing block (S) either directly or indirectly via a spacer (B) and/or Linker U; or (iii) is unreacted or capped with a terminal group, such as an acetyl group. Capped or capping refers to the modification of a functional group, such as FGt, to make it less reactive and/or have neutral charge at pH 7.4. For example, an amine may be capped with an activated carboxylic acid (e.g., acetyl chloride) to result in a relatively less reactive amide; or, e.g., a strained alkyne may be capped with an alkyl-azide to result in a relatively less reactive triazole.

### Hydrophobic Block (H)

The hydrophobic block (sometimes designated “H” in formulae) is a molecule with substantially limited water solubility, or is amphiphilic in properties, and capable of assembling into supramolecular structures, e.g., micellar, nano- or micro-particles in aqueous solutions. In certain embodiments, the hydrophobic block (H) is insoluble, or forms micelles, in aqueous solutions at concentrations of about 1.0 mg/mL or less, e.g., about 0.1 mg/mL or about 0.01 mg/mL. In some embodiments, the hydrophobic block is soluble in aqueous solutions at certain concentrations, temperatures and/or pH ranges but becomes insoluble in response to a change in concentration, temperature and/or pH. For instance, in some embodiments, the hydrophobic block is a hydrophobic polymer that is temperature-responsive, i.e., the hydrophobic polymer is soluble in aqueous solutions at temperatures below a transition temperature (Tₜᵣ) but becomes insoluble at temperatures above the transition temperature. Preferred hydrophobic blocks (H) are molecules that have a solubility of at least less than about 1.0 mg/mL, such as less than about 0.1 mg/mL or less than about 0.01 mg/mL, at or near physiologic pH (˜pH 7.4), between about pH 6.5 to pH 8.5 or between about pH 6.0 and pH 9.0, and at or near physiologic temperature (˜37° C.) and physiologic salt concentrations (˜10 g/L) and salt composition.

The hydrophobic block (H) may be chosen from any molecule comprising higher alkanes, cyclic aromatics, fatty acids, compounds deriving from terpenes/isoprenes, or polymers or oligomers that have limited water solubility and/or amphiphilic characteristics.

Exemplary higher alkanes include but are not limited to octane, nonane, decane, undecane, dodecane, tridecane, tetradecane, pentadecane, hexadecane, heptadecane and octadecane. Exemplary cyclic aromatics include but are not limited to phenyl. Exemplary saturated and unsaturated fatty acids include but are not limited to myristic acid, palmitic acid, stearic acid or oleic acid. In some embodiments, the hydrophobic block (H) is a fatty acid, for example myristic acid. In other embodiments, the hydrophobic block (H) comprises a diacyl lipid, such as 1,2-dioleoyl-sn-glycero-3-phosphoethanolamine or 1,2-distearoyl-sn-glycero-3-phosphoethanolamine or a lipopeptide, e.g., Pam2Cys. In some embodiments, the fatty acid or lipid based hydrophobic block (H) may further comprise a PEG. Exemplary compounds deriving from terpenes/isoprene include sterol derivatives, such as cholesterol, and squalene. In some embodiments, the hydrophobic block (H) comprises cholesterol. In some embodiments, the hydrophobic block (H) comprises a saponin, e.g., QS-21.

In some embodiments the hydrophobic block (H) is a linear, branched or brush polymer (or oligomer). The hydrophobic block (H) can be a homopolymer or copolymer. The hydrophobic block (H) can comprise one or many different types of monomer units. The hydrophobic block (H) can be a statistical copolymer or alternating copolymer. The hydrophobic block (H) can be a block copolymer, such as the A-B type, or the polymer can comprise a grafted copolymer, whereby two or more polymers are linked through polymer analogous reaction.

The hydrophobic block (H) may comprise polymers comprising naturally occurring and/or non-natural monomers and combinations thereof.

In some embodiments, the hydrophobic block (H) is selected from natural biopolymers. Natural biopolymers may include peptides (sometimes referred to as poly(amino acids)) which comprise hydrophobic amino acids. Non-limiting examples of hydrophobic amino acids include leucine, isoleucine, norleucine, valine, tryptophan, phenylamine, tyrosine and methionine, as well as hydrophilic amino acids that have been modified, such as by acetylation or benzoylation to have hydrophobic characteristics. Natural biopolymers that are water soluble in their native form may be used but must be modified chemically to make such natural biopolymers water insoluble and suitable for use as hydrophobic block (H). For example, biopolymers which comprise of hydrophilic amino acids, such as glutamic acid or lysine residues may be modified at the gamma carboxyl or epsilon amine groups, respectively, for the attachment of a hydrophobic molecule, such as a hydrophobic drug molecule, to increase the hydrophobicity of the resulting modified biopolymer. Similarly, biopolymers can be selected from hydrophilic polysaccharides, which may include but are not limited to glycogen, cellulose, dextran, alginate and chitosan, but such polysaccharides should be modified chemically, for example via acetylation or benzoylation of hydrophilic functional groups to render the resulting modified polysaccharide water insoluble. In still further embodiments the hydrophobic block comprises monomers selected from lactic acid and/or glycolic acid.

Monomers comprising the hydrophobic block (H) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylates and (meth)acrylamides include benzyl methacrylamide (BnMAM) and benzyl methacrylate (BnMA), respectively.

Certain monomers described herein as hydrophobic monomers may be water soluble under certain conditions but are hydrophobic and water insoluble at certain conditions in aqueous solutions. Non-limiting examples include temperature-responsive monomers, such as N-isopropylmethacrylamide (NIPMAM); a homopolymer comprising entirely of NIPMAM may be water soluble at room temperature but may become insoluble and form particles at elevated temperatures. Such distinctions are made to facilitate description of certain embodiments. In some embodiments, the hydrophobic block comprises a majority of monomer units selected from hydrophobic monomers that are temperature-responsive (sometimes referred to as “temperature-responsive monomers”), such as NIPAM, NIPMAM, N,N′-diethylacrylamide (DEAAM), N-(L)-(1-hydroxymethyl)propyl methacrylamide (HMPMAM), N,N′-dimethylaminoethylmethacrylate (DMEMA), N-(N-ethylcarbamido)propylmethacrylamide, N-vinylisobutyramide (PNVIBA), N-vinyl-n-butyramide (PNVBA), N-acryloyl-N-propylpiperazine (PNANPP), N-vinylcaprolactam (PVCa), DEGMA, TEGMA, or poly(amino acids) or γ-(2-methoxyethoxy)esteryl-L-glutamate. In still other embodiments, the hydrophobic block (H) may comprise monomers of ethylene oxide, propylene oxide or combinations thereof

Hydrophobic blocks (H) comprising a polymer typically comprise hydrophobic monomers and one or more other types of monomers, such as reactive monomers optionally linked to a drug molecule, spacer monomers and/or charged monomers. In some embodiments of hydrophobic blocks (H) comprising a polymer (or oligomer), a majority of monomer units are selected from hydrophobic monomers. In other embodiments of hydrophobic blocks (H) comprising a polymer (or oligomer), a majority of monomer units are selected from reactive monomers linked to hydrophobic drug molecules. In still other embodiments of hydrophobic blocks (H) comprising a polymer (or oligomer), the polymer comprises hydrophobic monomers and reactive monomers linked to hydrophobic drug molecules. In still further embodiments of hydrophobic blocks (H) comprising a polymer (or oligomer), the polymer comprises hydrophobic monomers and charged monomers and optionally reactive monomers linked to hydrophobic drug molecules.

In preferred embodiments, the hydrophobic block (H) comprises a polymer (or oligomer) that comprises hydrophobic monomers that further comprise aryl groups. In certain embodiments, the hydrophobic block (H) comprises heteroaryl groups. In still other embodiments, the aryl or heteroaryl groups of the hydrophobic block (H) comprise an amino substituent. The present inventors found that hydrophobic blocks (H) comprising aminoaryl or aminoheteroaryl groups lead to improved manufacturability and solubility in water-miscible solvents. The present inventors also found that amphiphiles with hydrophobic blocks (H) comprising aromatic amines lead to formation of stable particles with low CMC.

In preferred embodiments, the hydrophobic block (H) comprises monomers that comprise aryl or heteroaryl groups. Exemplary aryl groups (sometimes referred to as “aromatics” or “aromatic rings”) include but are not limited to phenyl, naphthyl, and quinolinyl. Non-limiting examples include:

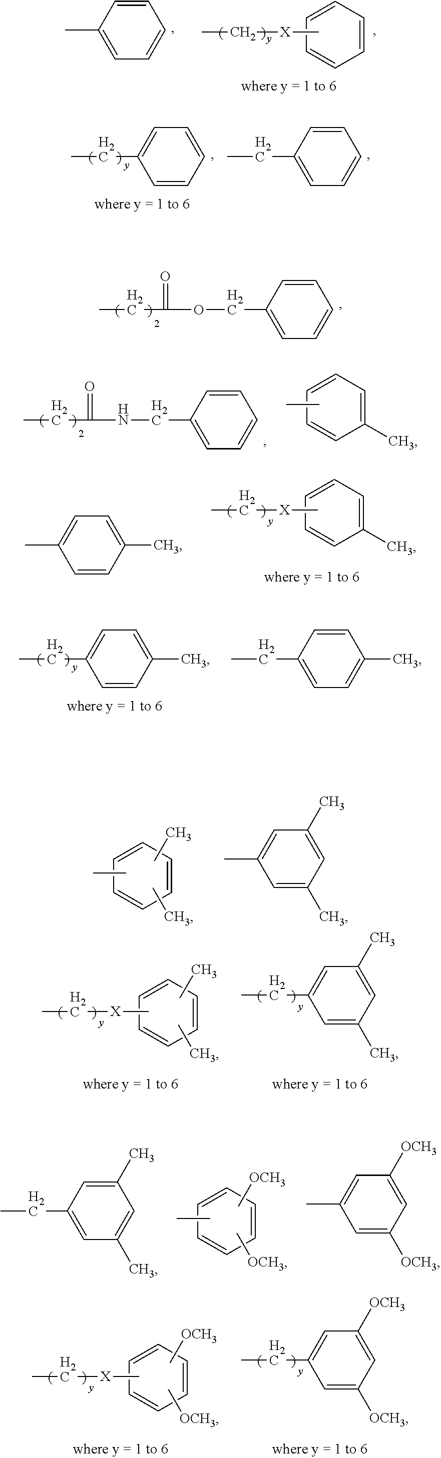

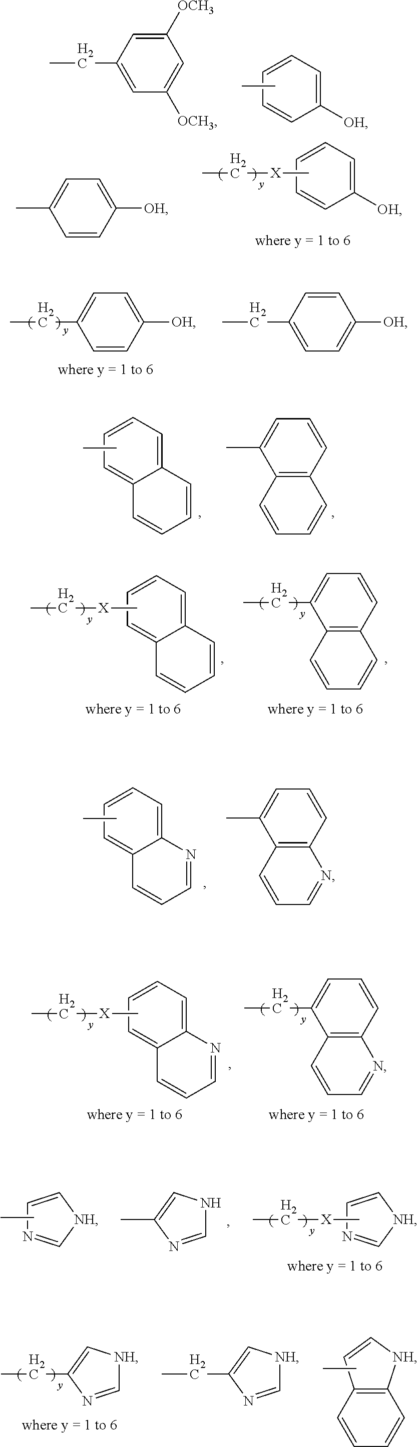

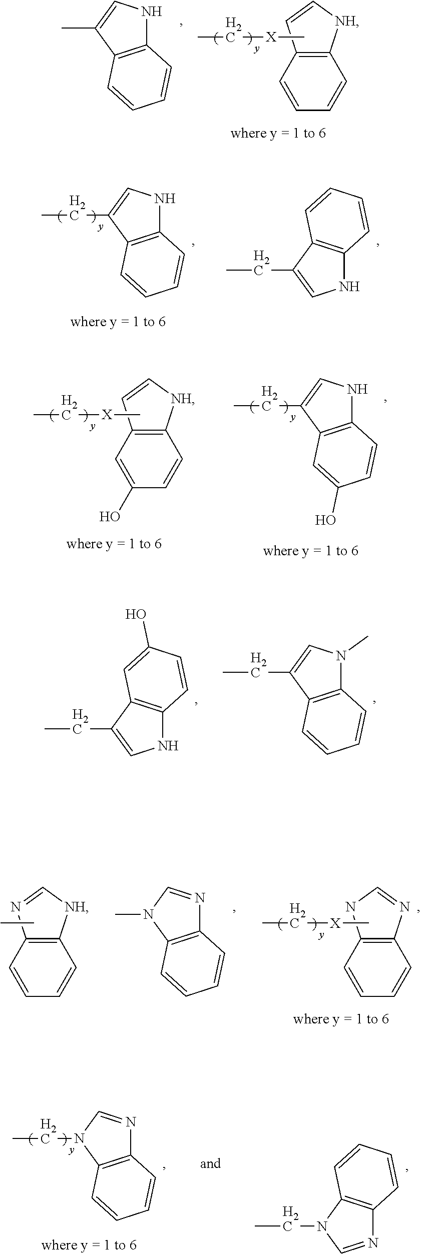

wherein X is any suitable linker molecule and y is an integer value, typically between 1 and 6.

In preferred embodiments, aryl or heteroaryl groups include but are not limited to

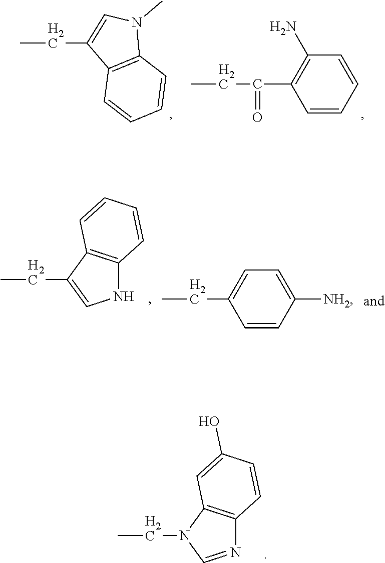

Furthermore, in the aforementioned aryl or heteroaryl groups one or more hydrogen atoms may be substituted for one or more fluorine atoms. In certain embodiments, the hydrophobic block comprises fluorinated aliphatic, aryl or heteroaryl groups, wherein one or more hydrogen atoms of the aforementioned groups comprising the hydrophobic monomer may be substituted for one or more fluorine atoms. The following non-limiting examples of fluorinated aryl groups may be present in hydrophobic monomers:

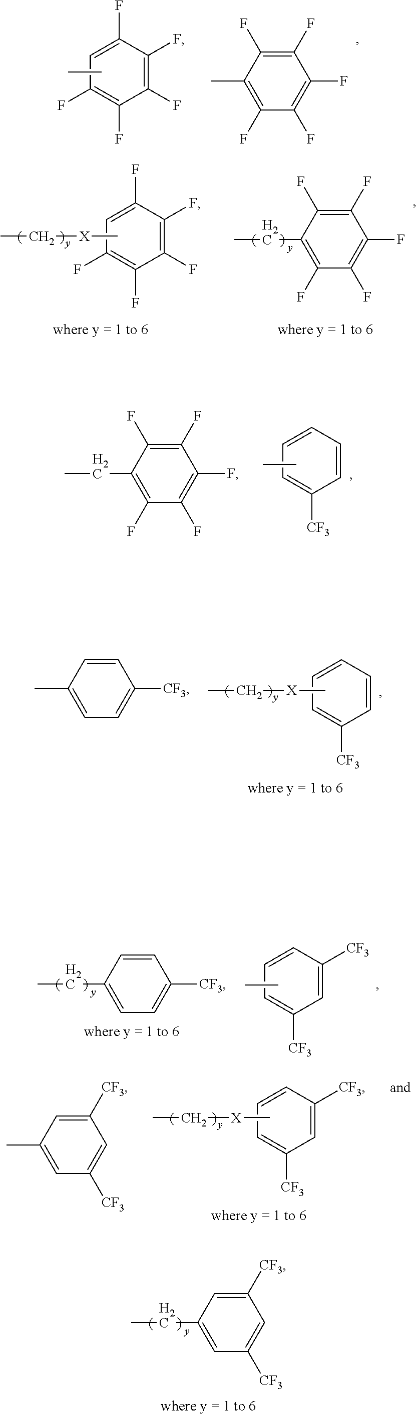

wherein X is any suitable linker molecule and y is an integer value, typically between 1 and 6.

The present inventors have unexpectedly found that hydrophobic blocks (H) comprising aminoaryl or aminoheteroaryl groups lead to improved manufacturing and solubility in polar aprotic solvents and alcohols. Therefore, in certain preferred embodiments, the hydrophobic block (H) comprises moieties of the formula —Ar—NHR, where Ar can be a aryl or heteroaryl, and R is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Non-limiting examples of aminoaryl or aminoheteroaryl groups include but are not limited to:

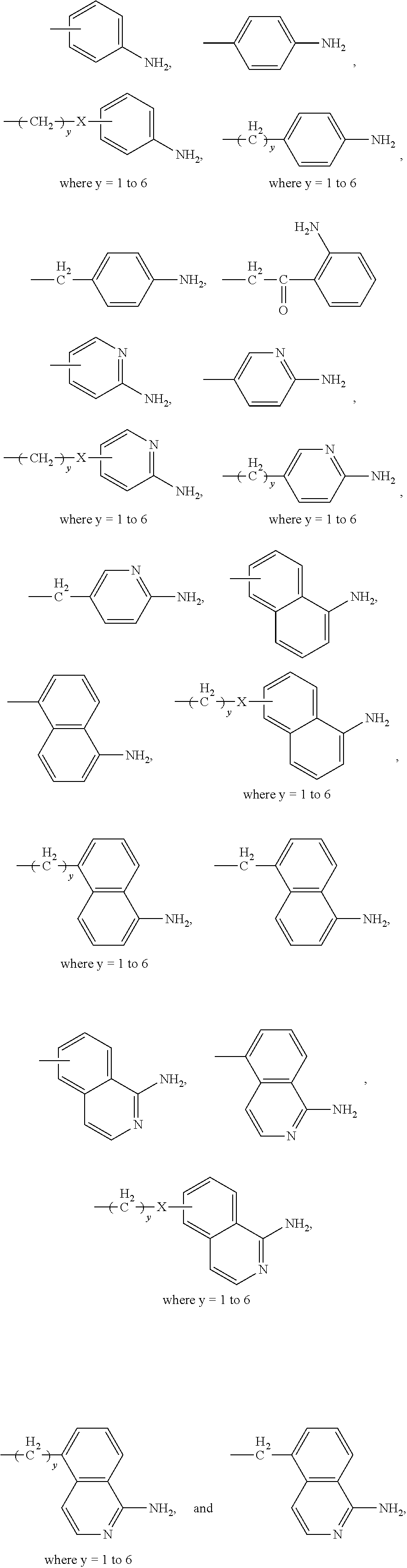

wherein X is any suitable linker molecule and y is an integer value, typically between 1 and 6.

In some embodiments, the hydrophobic block (H) comprises polymers (or oligomers) that further comprise hydrophobic monomers with fused aryl groups (e.g., naphthyl) or fused heteroaryl groups (e.g., xanthenyl or quinolinyl). In some embodiments, the hydrophobic block (H) comprises reactive monomers linked to hydrophobic drug molecules. In some embodiments, the hydrophobic drug molecules (e.g., imidazoquinolines) are aromatic and thus the reactive monomers linked to hydrophobic drug molecules comprising aromatic groups may also be described as hydrophobic monomers comprising aromatic groups or reactive monomers linked to drugs.

In some embodiments, the hydrophobic block (H) comprises a poly(amino acid) of Formula I:

-(M)ₘ-(N)ₙ-(O)ₒ-(P)ₚ-R³

wherein the poly(amino acid) of Formula I comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; and R₃ is typically selected from hydrogen, NH₂, NH₂—CH₃, NH₂—(CH₂)_{y5}CH₃, OH, or drug molecules (D) either linked directly or through X1.

In some embodiments, P is absent. In other embodiments, N, O, and P are each absent.

In some embodiments, P is

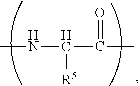

wherein each R⁵, independently, is a group that comprises 1 to 2 charged functional groups.

In some embodiments, O is

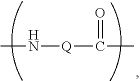

wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4.

In some embodiments, N is

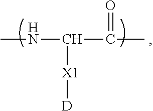

wherein each X1, independently, is a suitable linker; and each D, independently, is a drug molecule.

In some embodiments, M is

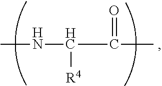

wherein each R⁴ is, independently, a hydrophobic group.

In some embodiments, the hydrophobic block (H) comprises a poly(amino acid) of Formula I:

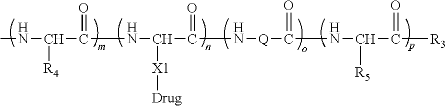

wherein the poly(amino acid) of Formula I comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; R³ is typically selected from hydrogen, NH₂, NH₂—CH₃, NH₂—(CH₂)_{y5}CH₃, OH, or drug molecules (D) either linked directly or through X1; R⁴ is any hydrophobic group typically selected from aryl or heteroaryl groups; R⁵ is any group that comprises one or more functional groups that are charged in aqueous solutions or are pH-responsive and charged in aqueous solutions at certain pH ranges; Q is typically selected from any lower alkyl or heteroalkyl including but not limited to (CH₂)_{y6} and (CH₂CH₂O)_{y7}CH₂CH₂, where y6 is any integer from 1 to 6 and y7 is an integer typically selected from 1 to 4; and, the N-terminus is linked to either (i) a solubilizing block (S) directly or indirectly via a spacer (B) and/or a Linker U; or (iii) a drug molecule either directly or via X1. Note: hydrophobic amino acids, reactive amino acids, spacer amino acids and charged amino acids are sometimes described more generally as hydrophobic monomers, reactive monomers, spacer monomers and charged monomers.

In preferred embodiments of poly(amino acids) of Formula I, R⁴ is

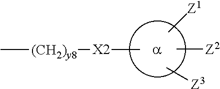

wherein,
α is aryl or heteroaryl;
X2 is present or absent and when present is is a suitable linker;
y8 is selected from an integer from 0 and 6; and
Z¹, Z², and Z³ are each independently selected from H, F, hydroxy, amino, alkyl, and fluoroalkyl.

In preferred embodiments of poly(amino acids) of Formula I, a is aryl, e.g., phenyl or naphthyl. In other embodiments, a is heteroaryl, e.g., imidazolyl, pyridinyl, quinolinyl, isoquinolinyl, indolyl, and benzimidazolyl.

In preferred embodiments of poly(amino acids) of Formula I, X2 is absent. In other embodiments, X2 is present and is selected from C(O), CO₂(CH₂)_{y9}, and C(O)NH(CH₂)_{y9}, NHC(O) and NHC(O)(CH₂)_{y9}, wherein y9 is an integer typically selected from 1 to 6. In other embodiments, X2 is present and is selected from lower alkyl and PEG groups.

In preferred embodiments of poly(amino acids) of Formula I, the poly(amino acid) of Formula I comprises hydrophobic amino acids, M, selected from any natural or non-natural amino acid that comprises a hydrophobic group, R⁴. In preferred embodiments, R⁴ is selected from hydrophobic groups comprising aryl groups, heteroaryl groups, aminoaryl, and/or aminoheteroaryl. Non-limiting examples of R⁴ include but are not limited to:

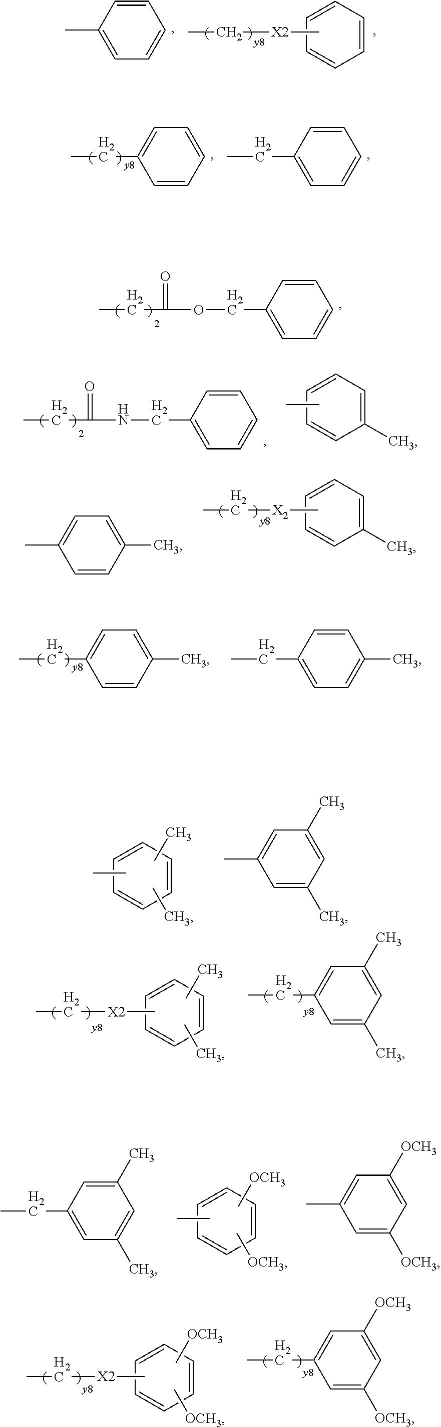

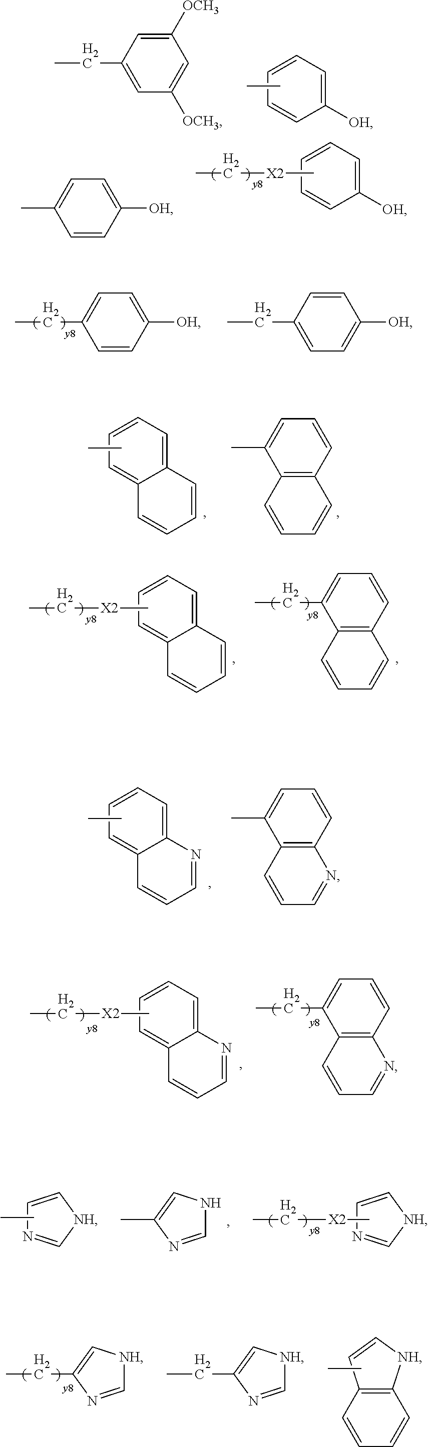

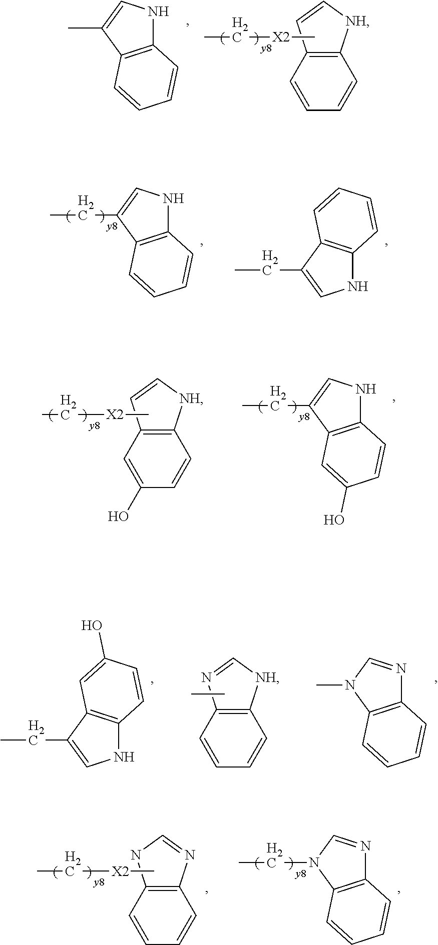

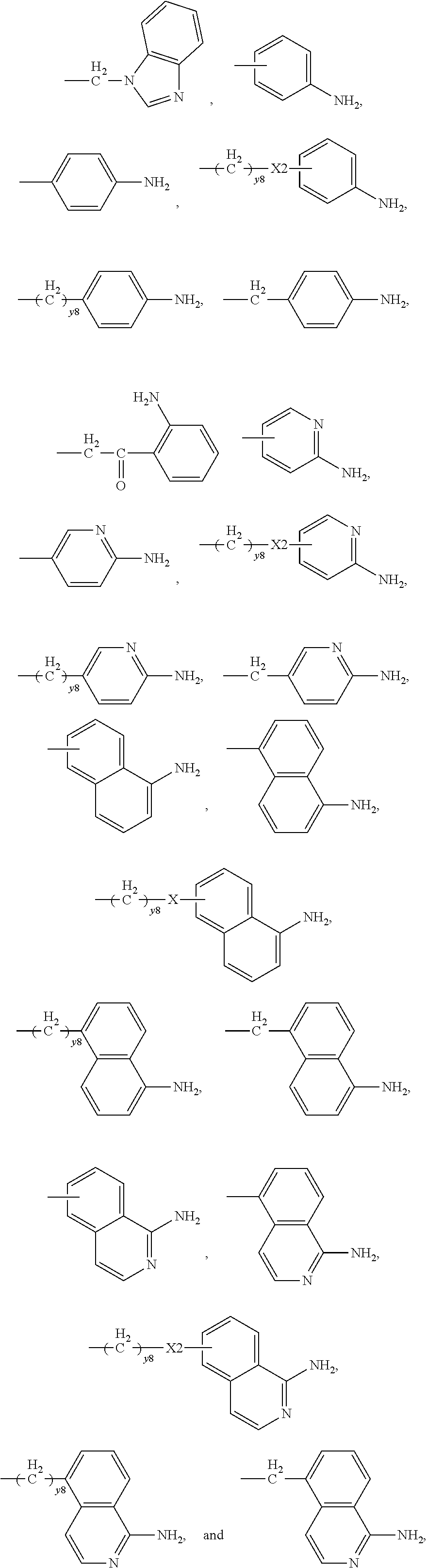

wherein X2 is any suitable linker molecule and y8 is an integer value, typically between 0 and 6. In preferred embodiments y8 is 1.

In a non-limiting example, wherein R⁴ is

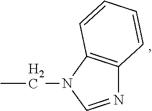

monomer M is:

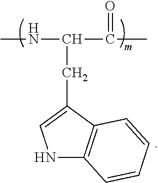

In some embodiments, the poly(amino acid)-based hydrophobic block (H) of Formula I comprises reactive amino acids, N, that are selected from any natural or non-natural amino acid, wherein a drug molecule (D) is linked directly or through X1 to the monomer. Suitable reactive amino acids include but are not limited to any amino acids bearing a group suitable for attachment of drug molecules, including amino acids with azide, alkyne, tetrazine, transcyclooctyne (TCO), protected hydrazine, ketone, aldehyde, certain hydroxyl groups, isocyanate, isothiocyanate, carboxylic acids, activated carboxylic acids, activated carbamates, protected maleimide, thiol and/or amine groups.

X1 is any suitable linker for linking drug molecules, D, to the hydrophobic block (H), including to the reactive amino acid, N, of poly(amino acids). X1 used to join D to reactive amino acids, N, is typically selected from —(CH₂)_{y10}-FG3 and —(CH₂)_{y10}-R⁶ (or —C(O)—(CH₂)_{y10}-FG3 and —C(O)—(CH₂)_{y10}-R⁶ when drugs are linked at the N-terminus or off of amine groups, or —NH—(CH₂)_{y10}-FG3 and —NH—(CH₂)_{y10}—R⁶ when drugs are linked at the C-terminus or off of carbonyl groups), wherein y10 is any integer, typically selected from 1 to 6, and R⁶ is typically selected from any one or more of —C(O)—NH—R⁷, —NH—C(O)—R⁷, —NH—C(O)—O—R⁷, —O—C(O)—NH—R⁷, —O—C(O)—R⁷, —C(O)—O—R⁷, —O—R⁷, O—C(O)—W, or —C(O)—W, wherein R⁷ is typically selected from any one or more of —(CH₂)_{y11}—W, —(CH₂)_{y11}—(OCH₂CH₂)_{y12}—W, —(CH₂)_{y11}—(OCH₂CH₂)_{y12}—(CH₂)_{y13}—W, —CHR⁸—C(O)—W, —CHR⁸—C(O)—(NH—CHR⁸—C(O))ⱼ—W, —(CH₂)_{y11}—C(O)—NH—CHR⁸—C(O)—W, —(CH₂)_{y11}—C(O)—NH—CHR⁸—C(O)—(NH—CHR⁸—C(O))ⱼ—W, —(CH₂)_{y11}—(OCH₂CH₂)_{y12}—C(O)—NH—CHR⁸—C(O)—W, —(CH₂)_{y11}—(OCH₂CH₂)_{y12}—(CH₂)_{y13}C(O)—NH—CHR⁸—C(O)—W, —(CH₂)_{y11}—(OCH₂CH₂)_{y12}—C(O)—NH—CHR⁸—C(O)—(NH—CHR⁸—C(O))ⱼ—W, —(CH₂)_{y11}—(OCH₂CH₂)_{y12}—(CH₂)_{y13}—C(O)—NH—CHR⁸—C(O)—(NH—CHR⁸—C(O))ⱼ—W, —CHR⁸—C(O)—NH—C₆H₄—CH₂—O—C(O)—W, —CHR⁸—C(O)—NH(CH₃)(CH₂)₂—O—C(O)—W, —CHR⁸—C(O)—(NH—CHR⁸—C(O))ⱼ—NH—C₆H₄—CH₂—O—C(O)—W, —CHR⁸—C(O)—(NH—CHR⁸—C(O))ⱼ—NH(CH₃)(CH₂)₂—O—C(O)—W, —(CH₂)_{y11}—C(O)—(NH—CHR⁸—C(O))ⱼ—NH—C₆H₄—CH₂—O—C(O)—W, —(CH₂)_{y11}—C(O)—(NH—CHR⁸—C(O))—NH(CH₃)(CH₂)₂—O—C(O)—W, —(CH₂)_{y11}—(OCH₂CH₂)_{y12}—C(O)—(NH—CHR⁸—C(O))ⱼ—NH—C₆H₄—CH₂—O—C(O)—W, —(CH₂)_{y11}—(OCH₂CH₂)_{y12}—C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—(NH—CHR⁸—C(O))ⱼ—NH(CH₃)(CH₂)₂—O—C(O)—W, —(CH₂)_{y11}—(OCH₂CH₂)_{y12}—(CH₂)_{y13}—C(O)—NH—(CH₂)_{y14}—C(O)—(NH—CHR⁸—C(O))ⱼ—NH—C₆H₄—CH₂—O—C(O)—W, —(CH₂)_{y11}—(OCH₂CH₂)_{y12}—(CH₂)_{y13}C(O)—NH—(CH₂)_{y14}—C(O)—(NH—CHR⁸—C(O))ⱼ—NH(CH₃)(CH₂)₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH—C₆H₄—CH₂—O—C(O)—W, —(CH₂)_{y11}—(OCH₂CH₂)_{y12}—C(O)—NH—(CH₂)_{y14}—C(O)—(NH—CHR⁸—C(O))ⱼ—NH(CH₃)(CH₂)₂—O—C(O)—W, —CHR⁸—C(O)—NH—(CH₂)_{y15}—W, —CHR⁸—NH—C(O)—(CH₂)_{y15}—W, —CHR⁸—C(O)—(NH—CHR⁸—C(O))ⱼ—NH—(CH₂)_{y15}—W, —CHR⁸—NH—(C(O)—CHR⁸—NH)ⱼ—C(O)—(CH₂)_{y15}—W, where y11, y12, y13, y14, y15 and j are each independently selected from any integer typically selected from 1 to 6, R⁸ is any amino acid side group, and W can be independently selected from H (hydrogen), FG3, LG and w; wherein FG3 is any suitable functional group for attachment to the drug molecule, which may be selected from, but not limited to, carboxylic acid, activated carboxylic acids (e.g., carbonylthiazolidine-2-thione (“TT”), NHS or nitrophenol esters), carboxylic acid anhydrides, amine and protected amines (e.g., tert-butyloxycarbonyl protected amine), OSi(CH₃), alkene, azide, alkyne, stained-alkyne, halogen (e.g., fluoride, chloride), olefins and endo cyclic olefins (e.g., allyl), CN, OH, and epoxy, hydrazines (including hydrazides), carbohydrazides, aldehydes, ketones, carbamates and activated carbamates, LG is any suitable leaving group, which may be selected from, but not limited to any suitable leaving group (e.g., NHS, TT, nitrophenol, etc.), and, w is a group that results from either the reaction of FG4 with FG3 or the displacement of LG with FG4, and is typically selected from NH—C(O)—, NH—C(O)—, C(O)—NH—, O—C(O)—NH—, C(O)—NH—N═C(CH₃)—, NH—N═C(CH₃)— or —C(CH3)=N—NH—C(O)—, wherein w is always linked to D either directly (i.e., w-D) or indirectly via X3 (i.e., w-X3-D).

Drug molecules (D) may be attached to the reactive amino acid, N, directly or via X1 through reaction of FG4 with FG3, wherein FG4 is any suitable functional group on the drug (D) that is reactive with FG3. Alternatively, drug molecules (D) may be linked to the reactive amino acid, N, via X1 through displacement of LG with any suitable FG4 comprising a nucleophile, e.g., a primary amine, or drug molecules (D) may be linked to the reactive amino acid, N, via X1 through displacement of an LG present on the drug molecule with any suitable FG3 comprising a nucleophile.

In preferred embodiments, FG3 is a carboxylic acid and FG4 is an amine, which react to form an amide. In a non-limiting example, X1 is selected from —(CH₂)_{y10}-FG3, y10 is 2, FG3 is a carboxylic acid, and FG4 present on the drug is an amine (i.e., NH₂-D), which react to form an amide, which may be represented as —(CH₂)₂-C(O)-D (amine not shown) or —(CH₂)₂—C(O)—NH-D (amine shown), indicating that the drug is linked via an amide bond at the carbonyl of X1, which (after amide bond formation) may be described as —(CH₂)_{y10}—R⁶, wherein y10 is 2, R⁶═C(O)—W, and W is the group w, which is NH- and is linked to D to give —(CH₂)₂—C(O)—NH-D.

The drug may additionally comprise a linker, X3, between the reactive functional group FG4 and the pharmacophore, e.g., FG4-X3-D. Specific, preferred compositions of X3 are described elsewhere.

In other embodiments, FG3 is an amine and FG4 is a carboxylic acid, which react to form an amide. In a non-limiting example, X1 is —(CH₂)_{y10}-FG3, y10 is 4, FG3 is an amine, and FG4 present on the drug is a carboxylic acid (i.e., COOH-D), which react to form an amide, which may be represented as —(CH₂)₄—NH-D (carbonyl not shown) or —(CH₂)₄—NH—C(O)-D (carbonyl shown), indicating that the drug is linked via an amide bond at the amine of X1.

In still other embodiments, FG3 is a ketone or aldehyde and FG4 is a hydrazide or carbohydrazide, which react to form a hydrazone. In a non-limiting example, X1 is —(CH₂)_{y10}-R⁶, y10 is 4, R⁶ is —NH—C(O)—R⁷, R⁷ is (CH₂)_{y11}, y11 is 2 and W is C(O)—CH3, and FG4 present on the drug molecule is a hydrazide (NH₂—NH₂-C(O)-D), which reacts with X1, i.e., —(CH₂)₄—NH—C(O)—(CH₂)₂—C(O)—CH₃ to form a hydrazone bond, i.e, —(CH₂)₄—NH—C(O)—(CH₂)₂-C(CH3)=N—NH—C(O)-D. In still other embodiments, FG3 is a hydrazide or carbohydrazide and FG4 is a ketone or aldehyde that reacts to form a hydrazone. In a non-limiting example, X1 is —(CH₂)_{y10}-R⁶, y10 is 2, R⁶ is —C(O)—W, W is FG3 and FG3 is —NH—NH₂ and FG4 present on the drug molecule is a ketone CH₃C(O)-D (or optionally CH₃C(O)-X3-D), which reacts with X1 to form —(CH₂)₄—C(O)—NH—NH₂ to form a hydrazone bind, i.e., form —(CH₂)₄—C(O)—NH—N═C(CH₃)-D.

In certain preferred compositions, drug molecules (D) are linked directly to the reactive amino acid, N. A non-limiting example of a reactive amino acid comprising a linker selected from —(CH₂)y₁₀-FG3, wherein y10=2, FG3 is carboxylic acid (i.e., the reactive amino acid is glutamic acid) linked to a drug molecule is shown below for clarity:

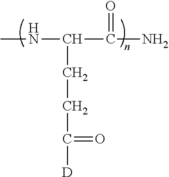

In certain other preferred embodiments, drug molecules (D) are linked to the reactive amino acid (N) via an enzyme degradable peptide and/or self-immolative linker, wherein the self-immolative linker is typically selected from —NH—C₆H₄—CH₂—O—C(O)— or —NH(CH₃)(CH₂)₂—O—C(O)— and FG4 present on the drug is an amine, e.g., NH₂-D or NH₂—X3-D, which results in a carbamate bond between the linker and the drug. In a non-limiting example, the reactive monomer comprises a linker selected from (CH₂)_{y10}-R⁶, wherein y10=2, R⁶ is —C(O)—NH—R⁷ and R⁷ is (CH₂)_{y11}—C(O)—(NH—CHR⁸—C(O))ⱼ—NH—C₆H₄—CH₂—O—C(O)—W, wherein y11 is 2, R⁸ is any amino acid group, j is an integer typically selected from 1 to 6, W is selected from the group w, which is NH-linked to the drug (D), as shown here:

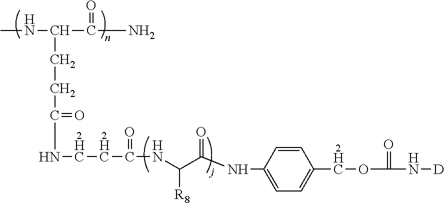

In preferred compositions of X1 comprising enzyme degradable linkers, the enzyme degradable linker typically comprises between 1 and 6 amino acids, such as 1, 2, 3, 4, 5 or 6 amino acids selected from single amino acids, dipeptides, tripeptides, tetrapeptides, pentapeptides and hexapeptides recognized and cleaved by enzymes, such as cathpesins and/or the immunoproteasome.

Reactive amino acids (N) may comprise functional groups that can impart charge; however, the classification of an amino acid as a reactive amino acid monomer is context-dependent and based on its intended use. For example, monomers comprising carboxylic acids may be referred to as charged monomers if the carboxylic acid is not used for drug attachment, whereas the same monomers linked to an amine bearing drug molecule, e.g., via an amide bind, would be considered a reactive monomer.

In some embodiments, the poly(amino acid)-based polymer of Formula I comprises spacer amino acids, O, that are selected from any natural or non-natural amino acid that are non-bulky and near neutral, such as a PEG amino acid spacer, e.g., Q of monomer O is a lower alkyl or PEG, e.g., —(CH₂)_{y6}—, —CH₂—CH₂—O- or —(CH₂—CH₂—O)_{y7}CH₂—CH₂—, wherein y6 and y7 are each independently an integer typically between 1 and 6. Alternatively, monomer 0, is selected from amino acids with a small, i.e., non-bulky, substituent selected from hydrogen, lower alkyl or a lower alkyl comprising a hydroxyl and is provided to increase the spacing or flexibility of the polymer backbone.

Non-limiting examples include:

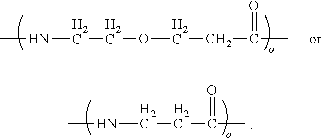

In some embodiments, the poly(amino acid)-based polymer of Formula I comprises optional co-monomer(s), P, that are selected from any natural or non-natural amino acid, wherein R₅ is selected from any group comprising a functional group that carriers charge either permanently or at a specific pH in aqueous solutions. Non-limiting examples of charged amino acids include any natural or non-natural amino acid that comprise amine, quaternary ammonium, sulfonic acid, sulfuric acid, sulfonium, phosphoric acid, phosphonic acid, phosphonium, carboxylic acid, boronic acid functional groups and/or combination thereof, including zwitterions, which may be linked either directly or via a suitable linker molecule, as well as any composition of salts thereof. Non-limiting examples of salts include, e.g., positively charged functional groups, e.g., ammonium ions paired with halide (e.g., chloride) ions. Other non-limiting examples of suitable salts of charged amino acids include conjugate bases of carboxylic, sulfonic and phosphonic acids, paired with group 1 metals, such as sodium, or ammonium or guanidinium ions.

In some preferred embodiments of amphiphiles for nucleic acid delivery, e.g., wherein the drug molecule (D), is nucleic acid, the amphiphile comprises a hydrophobic block (H) further comprising a poly(amino acid)-based polymer of Formula I that includes R⁵ selected from groups that have net positive charge, which include but are not limited to:

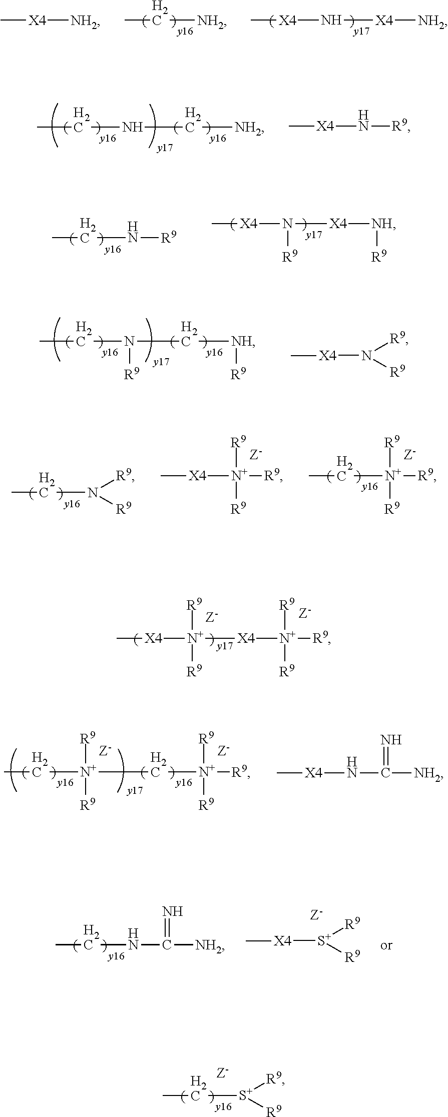

wherein X4 is any suitable linker, y16 and y17 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z⁻ is any suitable counter anion, which is typically selected from conjugate bases of weak acids or halide ions, such as Cl⁻, I⁻, or Br⁻.

The hydrophobic block (H) functions to drive particle assembly in aqueous solutions and therefore, in preferred embodiments of amphiphiles or drug molecule conjugates, the hydrophobic block (H) comprises hydrophobic amino acids and/or reactive amino acids linked to hydrophobic drug molecules. In preferred embodiments of poly(amino acid)-based polymers of Formula I, the poly(amino acid)-based polymer (or oligomer) of Formula I comprises hydrophobic amino acids (M) and/or reactive amino acids (N) linked to hydrophobic drug molecules, and optionally spacer amino acids (O) and/or charged amino acids (P). In preferred embodiments of amphiphiles or drug molecule conjugates used for the delivery of neutral drug molecules, the hydrophobic block (H) is typically selected from poly(amino acid)-based polymers of Formula I comprising hydrophobic amino acids (M) and/or reactive amino acids (N) linked to hydrophobic drug molecules, and optionally spacer amino acids (O), but not charged amino acids (P). In contrast, wherein the amphiphiles or drug molecule conjugates are used for nucleic acid delivery or for the delivery of charged drug molecules, the hydrophobic block (H) is typically selected from poly(amino acid)-based polymers of Formula I comprising hydrophobic amino acids (M) and/or charged amino acids (P) and optionally reactive amino acids (N) linked to hydrophobic drug molecules and spacer amino acids (O), wherein the charge of the charged amino acid is opposite that of the nucleic acid or charged drug molecule. Particular compositions of hydrophobic blocks (H) based on poly(amino acid)-based polymers or oligomers of Formula I that led to unexpected improvements in biological activity are described throughout the specification.

In some embodiments, the hydrophobic block (H) is a poly(amino acid) of Formula I comprising entirely hydrophobic monomers (m):

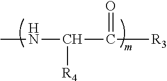

Non-limiting examples include:

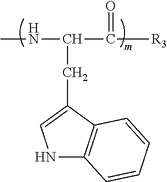

A non-limiting example of a poly(amino acid) of Formula I composed entirely of hydrophobic monomers (M) selected from tryptophan, wherein m is equal to 5 (i.e., 5 monomeric units), R³ is an amine and the N-terminal amine is linked to a solubilizing block (S) either directly or indirectly through a spacer (B) and/or linker U, is shown here for clarity:

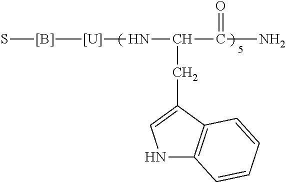

In some embodiments drug molecules (D) are linked via the N-terminus or C-terminus of hydrophobic blocks (H) comprising poly(amino acids) of Formula I. A non-limiting example is shown here for clarity:

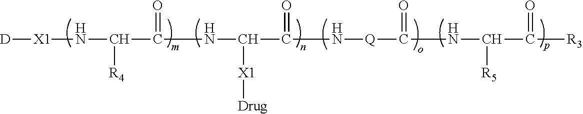

Wherein the poly(amino acid) comprises hydrophobic amino acids selected from tryptophan and R³ is NH₂ the structure is:

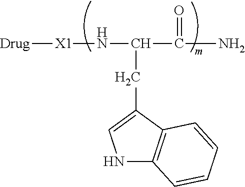

Wherein when X1 comprises a PAB-Cit-Val linked to the poly(amino acid) via a succinate linker the structure is:

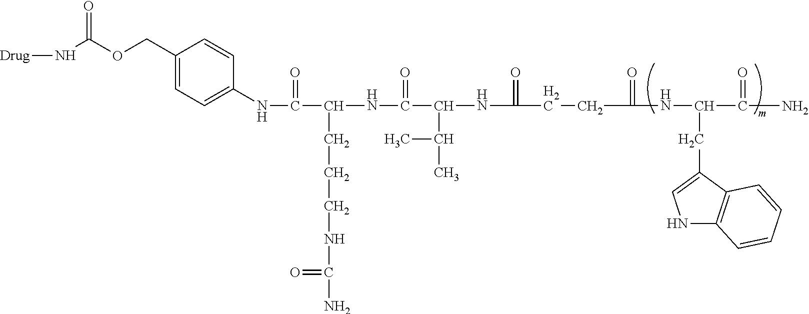

Alternatively, wherein X1, comprises a PAB-Cit-Val linked to the poly(amino acid) via Linker U resulting from the reaction between azide and DBCO, an exemplary strained alkyne, wherein the DBCO moiety is linked to poly(amino acid) via Ahx, the structure is:

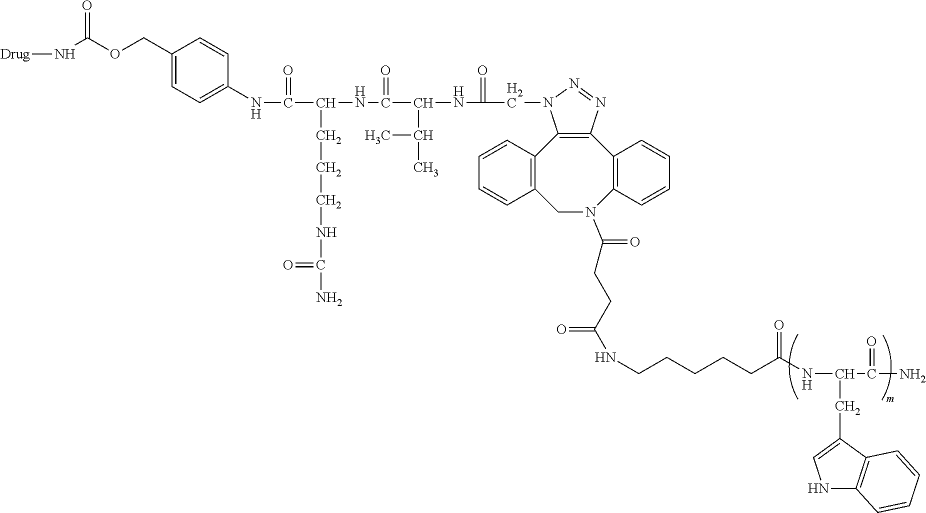

Herein, we report the unexpected finding that amphiphilic copolymers with hydrophobic polymers or oligomers (H) which comprise poly(amino acid)-based copolymers that include aromatic amino acids (e.g., phenylalanine, amino phenylalanine, histidine, tryptophan, tyrosine, benzyl glutamate) and/or aromatic drug molecules (e.g., imidazoquinolines), have unexpected improvements in manufacturability through improved solubility in polar aprotic solvents and alcohols as well as improved particle stability as compared with poly(amino acids) comprising hydrophobic amino acids selected from aliphatic amino acids.

An additional notable finding relates to how the number of monomer units comprising the hydrophobic block (H) impacts particle formation. For example, poly(amino acid)-based hydrophobic blocks (H) which comprise at least 5 hydrophobic amino acids were typically needed to ensure stable assembly of particles comprising amphiphiles of formula S-[B]-[U]-H (optionally further comprising a drug molecule, e.g., S-[B]-[U]-H-D). Though, unexpectedly, poly(amino acid)-based hydrophobic blocks (H) which comprise oligomers with as few as 3 monomers that included aromatic rings were found to be sufficient to drive stable particle assembly. Notably, increasing the number of hydrophobic monomers comprising the poly(amino acid)-based hydrophobic block (H) from 3 to 5 and from 5 to 10 hydrophobic monomers led to increased particle stability. While increasing the total number of monomers comprising hydrophobic blocks (H) (i.e. total number of monomers or degree of polymerization) led to improved particle stability, both the total number of monomers and composition of the poly(amino acids) of Formula I also impacted manufacturability. Though, the nature of the hydrophobic monomers also impacted manufacturability. For example, poly(amino acids) of Formula I comprising between 10-30 consecutive monomers selected from hydrophobic amino acids comprising aryl groups and/or heteroaryl groups were found to be more reliably manufactured than poly(amino acids) of Formula I comprising between 10-30 consecutive monomers selected from hydrophobic amino acids comprising aliphatic groups.

Therefore, in preferred embodiments of poly(amino acid)-based hydrophobic blocks (H), the hydrophobic block (H) comprises 3 or more, preferably about 3 to about 100 hydrophobic amino acids (M) and/or reactive amino acids linked to drug molecules (D), though, more preferably between about 3 to 30 hydrophobic amino acids (M) and/or reactive amino acids linked to drug molecules (D), more preferably wherein the hydrophobic amino acids and/or reactive amino acids linked to drug molecules (D) further comprise aryl, heteroaryl, aminoaryl and/or aminoheteroaryl groups.

Hydrophobic Blocks (H) with Branched Architecture

In some embodiments, the amphiphile comprises a hydrophobic block (H) that is branched. In certain preferred embodiments, the hydrophobic block (H) comprises a dendron, wherein the focal point is linked to either (i) a solubilizing block (S) either directly or indirectly via a spacer (B) and/or Linker U; (ii) a drug molecule either directly or via a Linker U; or (iii) a capping group, and the terminal functional groups (FGt) are linked to hydrophobic molecules, e.g., hydrophobic drug molecules, more preferably hydrophobic molecules comprising aromatic groups, e.g., hydrophobic drug molecules comprising aromatic groups.

Non-limiting examples of amphiphiles or drug molecule conjugates comprising hydrophobic blocks (H) with dendron architecture, wherein the terminal functional groups (FGt) are linked to hydrophobic drug molecules are provided below for clarity:

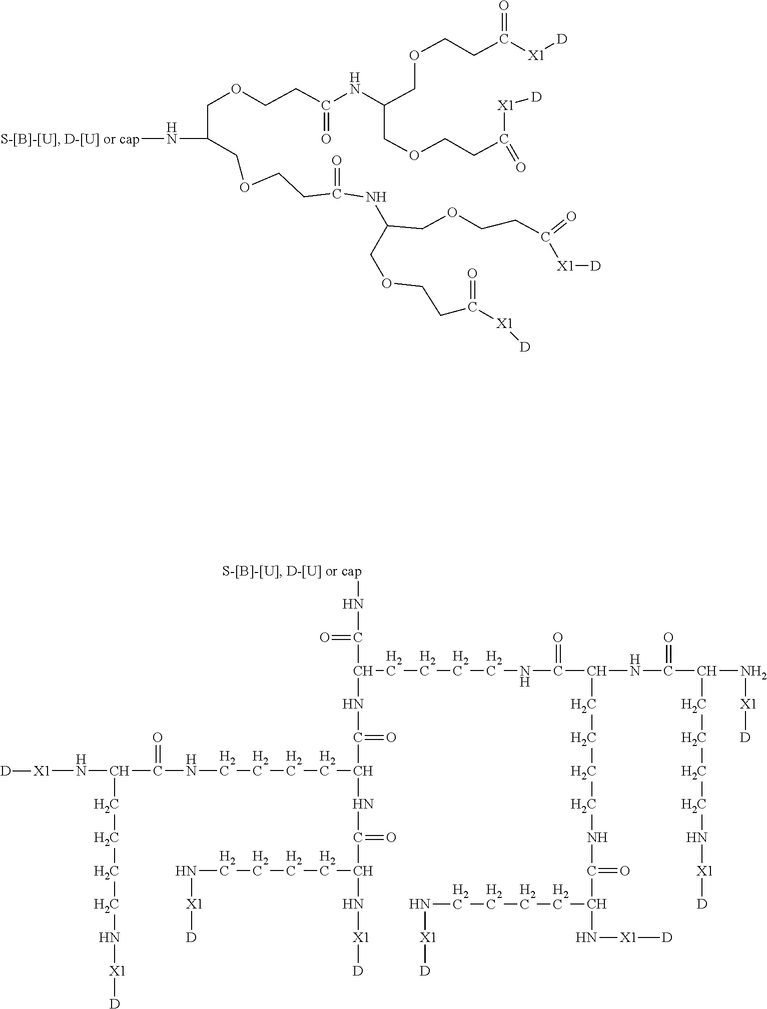

wherein X1 is either present or absent and when present is any suitable linker and D is any suitable drug molecule, preferably selected from hydrophobic drug molecules comprising aromatic groups, and the focal point is attached to either (i) a solubilizing block (S) either directly or indirectly via a spacer (B) and/or Linker U; (ii) a drug molecule either directly or via a Linker U; or, (iii) a capping group.

Additional examples of hydrophobic blocks (H) with dendron architecture that have particular utility for certain applications and/or lead to unexpected improvements in manufacturing and/or biological activity are provided throughout the specification.

Density (Mol %) of Hydrophobic Groups and/or Drug Molecules

The density (i.e., mol %) of the hydrophobic monomers (e.g., hydrophobic amino acids or reactive monomers linked to hydrophobic drug molecules) incorporated into polymer-based hydrophobic blocks (H), e.g., poly(amino acids) of Formula I, were found by the inventors of the present disclosure to impact particle stability and biological activity. In general, the density (mol %) of hydrophobic monomers (e.g., hydrophobic amino acids or reactive monomers linked to hydrophobic drug molecules) required is inversely proportional to the length (i.e., degree of polymerization) of the polymer.

For instance, the preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) is typically 100 mol % for polymers (or “oligomers”) with 3 monomers; 75-100 mol % for polymers (or “oligomers”) with 4 monomers, such as 75 mol % or 100 mol % for polymers with 4 monomers; 60-100 mol % for polymers (or “oligomers”) with 5 monomers, such as 60 mol %, 80 mol % or 100 mol %; 50-100 mol % for polymers (or “oligomers”) with 6 monomers, such as 50 mol %, 66.6 mol %, 83.3 mol % and 100 mol %; 42-100 mol % for polymers (or “oligomers”) with 7 monomers, such as 42 mol %, 57 mol %, 71 mol %, 85.7 mol % and 100 mol %; 37.5-100 mol % for polymers (or “oligomers”) with 8 monomers, such as 37.5 mol %, 50 mol %, 75 mol %, 87.5 mol % and 100 mol %; 33.3-100 mol % for polymers (or “oligomers”) with 9 monomers, such as 33.3 mol %, 44.4 mol %, 55.6 mol %, 66.6 mol %, 77.9 mol %, 88.9 mol % and 100 mol %; 30-100 mol % for polymers (or “oligomers”) with 10 monomers, such as 30 mol %, 40 mol %, 50 mol %, 60 mol %, 70 mol %, 80 mol %, 90 mol % and 100 mol %. The preferred density (mol %) of hydrophobic monomers (e.g., hydrophobic amino acids, M) and/or reactive monomers linked to hydrophobic drug molecules (e.g., reactive amino acids (N) linked to hydrophobic drug molecules) for polymers with between 11 and 20 monomers is typically between 20 mol % to 100 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol %, 80 mol %, 81 mol %, 82 mol %, 83 mol %, 84 mol %, 85 mol %, 86 mol %, 87 mol %, 88 mol %, 89 mol %, 90 mol %, 91 mol %, 92 mol %, 93 mol %, 94 mol %, 95 mol %, 96 mol %, 97 mol %, 98 mol %, 99 mol % or 100 mol %, provided that at least 3 hydrophobic monomers (M) or reactive monomers (N) linked to hydrophobic drugs are present; 10-100 mol %, more preferably 20-80 mol %, such as 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, 60 mol %, 61 mol %, 62 mol %, 63 mol %, 64 mol %, 65 mol %, 66 mol %, 67 mol %, 68 mol %, 69 mol %, 70 mol %, 71 mol %, 72 mol %, 73 mol %, 74 mol %, 75 mol %, 76 mol %, 77 mol %, 78 mol %, 79 mol % or 80 mol % for polymers with between 21 and 30 monomers, provided that at least 3 hydrophobic monomers (M) or reactive monomers (N) linked to hydrophobic drugs are present; and, 5-60 mol %, more preferably, 10-40 mol % for polymers with >30 monomers, such as 10 mol %, 11 mol %, 12, mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol % and 40 mol % for polymers with >30 monomers.

In the above examples, in preferred embodiments, the polymer is a poly(amino acid) and the monomer is selected from hydrophobic monomers (e.g., hydrophobic amino acid and/or reactive monomers linked to hydrophobic drug molecules) that comprise an aryl group, and, more preferably, a heteroaryl, aminoaryl, and/or aminoheteroaryl. Additionally, in the above examples, the hydrophobic monomer may be selected from two or more monomers, e.g., two or more distinct hydrophobic monomers (e.g., hydrophobic amino acids), or one or more hydrophobic monomers and one or more reactive monomers (e.g., reactive amino acids) linked to hydrophobic drugs, such that the total mol % of hydrophobic monomers falls within the preferred ranges.

### General Properties of Polymer-Based Hydrophobic Blocks (H)

The average molecular weight of polymer-based hydrophobic blocks (H) can be readily estimated based on the number and composition of monomers (e.g., amino acids for poly(amino acids)) and is typically between about 500 g/mol to about 60,000 g/mol.

The polydispersity, Mw/Mn, of the hydrophobic polymer or oligomer (H) typically ranges from about 1.0 to 2.0 and depends on the polymerization technique used. For instance, poly(amino acid)-based hydrophobic polymers or oligomers (H) are typically prepared by solid phase peptide synthesis and will have polydispersity of 1.0 as the polymers are molecularly defined. Polymers formed by chain growth polymerization will have polydispersities >1.0. The hydrophobic polymer or oligomer (H) may also comprise polymers based on cyclic monomers, such as poly(amino acid)-based hydrophobic polymers or oligomers (H) based on amino acid N-carboxyanhydrides (NCAs).

The size of the polymer-based hydrophobic block (H) may either be expressed by the molecular weight or degree of polymerization. For molecularly defined, monodisperse polymers, the length (or degree of polymerization) of the polymer can be calculated by dividing the molecular weight (e.g., theoretical or experimentally determined) by the average molecular weight of the monomer unit(s) comprising the polymer. For polydisperse polymers, the number-average molecular weight, abbreviated Mn, is preferred for estimating the degree of polymerization. As a non-limiting example, a polydisperse polymer with a Mn of 25 kDa and an average monomer molecular weight of 250 g/mol would have a degree of polymerization of 100. The molecular weight of a polymer can also be calculated by multiplying the degree of polymerization by the average monomer molecular weight.

In preferred embodiments of hydrophobic blocks (H), the molecular weight or Mn, is preferably between about 0.5 kDa and 60 kDa, such as about 0.5 kDa, 1 kDa, 1.5 kDa, 2 kDa, 2.5 kDa, 3 kDa, 3.5 kDa, 4 kDa, 4.5 kDa, 5 kDa, 6 kDa, 7 kDa, 8 kDa, 9 kDa, 10 kDa, 11 kDa, 12 kDa, 13, kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa or 60 kDa. More preferably, the molecular weight of the hydrophobic block is between about 0.5 kDa to about 20 kDa. In certain embodiments, the hydrophobic block (H) is a poly(amino acid) and has a molecular weight of between about 0.5 kDa and about 10 kDa or about 1.5 kDa to about 5 kDa.

Polymers described herein can be synthesized by any suitable means and should preferably have low or no polydispersity. For instance, poly(amino acids) described herein are typically produced by solid-phase peptide synthesis and are molecularly defined with no polydispersity. Similarly, PEG based spacers, solubilizing blocks and dendrons described herein have little to no polydispersity. In contrast, polymers produced by radical polymerization will have some degree of polydispersity, which may be calculated by dividing the weight-average molecular weight Mw by Mn, i.e., polydispersity index (PDI)=Mw/Mn. Though, the polydispersity of polymers produced by radical polymerization may be controlled by the polymerization technique utilized. Therefore, in preferred embodiments, living polymerization, e.g., RAFT polymerization, is used to synthesize polymers with PDI less than 2.0, typically between about 1.01 and 1.2.

### Solubilizing Block

The amphiphiles disclosed herein comprise a solubilizing block (S) that functions to impart solubility in aqueous solutions at certain temperature, pH and salt concentration. In certain embodiments, the solubilizing block (S) is soluble in aqueous solutions up to about 1-1,000 mg/mL, e.g., up to about 1 mg/mL, about 10 mg/mL, about 100 mg/mL, about 200 mg/mL, or about 500 mg/mL, though, typically not more than 1,000 mg/mL. In some embodiments, the solubilizing block (S) is soluble in aqueous solutions at certain concentrations, temperatures and/or pH ranges but becomes insoluble or less soluble in response to a change in concentration, temperature and/or pH. Preferred solubilizing blocks (S) are molecules that are soluble at concentrations up to at least 1 mg/mL or up to at least about 10 mg/mL or up to at least about 100 mg/mL at or near physiologic pH (˜pH 7.4), and at or near physiologic temperature (˜37° C.), such as between about 32-40° C., and at or near physiologic salt concentrations (˜10 g/L) and salt composition.

The solubilizing block may be chosen from any molecule that is water soluble and/or has hydrophilic characteristics. In some embodiments, the solubilizing block (S) is selected from a linear, branched or brush polymer (or oligomer). The solubilizing block (S) can be a homopolymer or copolymer. The solubilizing block (S) can comprise one or many different types of monomer units. The solubilizing block (S) can be a statistical copolymer or alternating copolymer. The solubilizing block (S) can be a block copolymer, such as the A-B type, or the polymer can comprise a grafted copolymer, whereby two or more polymers are linked through a polymerization-type reaction.

The solubilizing block (S) may comprise polymers comprising naturally occurring and/or non-natural monomers and combinations thereof.

In some embodiments, the solubilizing block (S) is selected from natural biopolymers. Natural biopolymers selected as solubilizing blocks (S) may include peptides (sometimes referred to as poly(amino acids)) comprising hydrophilic amino acids. Non-limiting examples of hydrophilic amino acids include serine, sulfo-serine, glutamic acid, aspartic acid, lysine, ornithine, arginine. Biopolymers can be selected from hydrophilic polysaccharides, which may include but are not limited to glycogen, cellulose, dextran, alginate and chitosan. In certain preferred embodiments, the solubilizing block comprises a linear or dendron-based poly(amino acid). In some embodiments, the solubilizing block comprising a linear or dendron-based poly(amino acid) comprises negatively charged amino acids typically selected from aspartic acid and/or glutamic acid, or lysine or ornithine residues converted to negatively charged groups through reaction with malonic acid, succinic acid, glutaric acid, adipic acid or the like.

In certain preferred embodiments, the solubilizing block comprises between about 2-12 negatively charged amino acids or negatively charged solubilizing groups linked to amino acids. In a non-limiting example, a poly(amino acid) comprising 12 aspartic acid monomers, e.g., Asp-Asp-Asp-Asp-Asp-Asp-Asp-Asp-Asp-Asp-Asp-Asp (SEQ ID NO:4), is used to prepare a solubilizing block (S) with a net negative charge of −12; a poly(amino acid) comprising 11 aspartic acid monomers, e.g., Asp-Asp-Asp-Asp-Asp-Asp-Asp-Asp-Asp-Asp-Asp (SEQ ID NO:5), is used to prepare a solubilizing block (S) with a net negative charge of −11; a poly(amino acid) comprising 10 aspartic acid monomers, e.g., Asp-Asp-Asp-Asp-Asp-Asp-Asp-Asp-Asp-Asp (SEQ ID NO:6), is used to prepare a solubilizing block (S) with a net negative charge of −10; a poly(amino acid) comprising 9 aspartic acid monomers, e.g., Asp-Asp-Asp-Asp-Asp-Asp-Asp-Asp-Asp (SEQ ID NO:7), is used to prepare a solubilizing block (S) with a net negative charge of −9; a poly(amino acid)) comprising 8 aspartic acid monomers, e.g., Asp-Asp-Asp-Asp-Asp-Asp-Asp-Asp (SEQ ID NO:8), is used to prepare a solubilizing block (S) with a net negative charge of −8; a poly(amino acid) comprising 7 aspartic acid monomers, e.g., Asp-Asp-Asp-Asp-Asp-Asp-Asp (SEQ ID NO:9), is used to prepare a solubilizing block (S) with a net negative charge of −7; a poly(amino acid) comprising 6 aspartic acid monomers, e.g., Asp-Asp-Asp-Asp-Asp-Asp (SEQ ID NO:10:), is used to prepare a solubilizing block (S) with a net negative charge of −6; a poly(amino acid) comprising 5 aspartic acid monomers, e.g., Asp-Asp-Asp-Asp-Asp (SEQ ID NO:11), is used to prepare a solubilizing block (S) with a net negative charge of −5; a poly(amino acid) comprising 4 aspartic acid monomers, e.g., Asp-Asp-Asp-Asp (SEQ ID NO:12), is used to prepare a solubilizing block (S) with a net negative charge of −4; a poly(amino acid) comprising 3 aspartic acid monomers, e.g., Asp-Asp-Asp, is used to prepare a solubilizing block (S) with a net negative charge of −3; a poly(amino acid) comprising 2 aspartic acid monomers, e.g., Asp-Asp, is used to prepare a solubilizing block (S) with a net negative charge of −2. In the above examples, aspartic acid (Asp) may be replaced with any suitable negatively charged amino acid, including but not limited to glutamic acid, sulfo-serine, or phospho-serine, or lysine or ornithine residues converted to negatively charged groups through reaction of a primary amine with malonic acid, succinic acid, glutaric acid, adipic acid or the like, wherein the negatively charged amino acids may be the same or different.

Monomers comprising the solubilizing block (S) can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. Specific examples of (meth)acrylate and (meth)acrylamide monomers include N-2-hydroxypropyl(methacrylamide) (HPMA) and hydroxyethyl(methacrylate) (HEMA). Various monomers suitable for the solubilizing block (S) are described below.

In certain embodiments, the solubilizing block (S) comprises hydrophilic polymers selected from synthetic or natural poly(saccharides), such as glycogen, cellulose, dextran, alginate and chitosan. Hydrophilic polymers used as the solubilizing block (S) should have sufficient length to provide adequate surface coverage to stabilize particles formed by amphiphiles, e.g., amphiphiles of formula S-[B]-[U]-H. In preferred embodiments of solubilizing blocks comprising hydrophilic polymers with linear architecture, the hydrophilic polymer comprises 50 or more monomer units, such as between 50 to 300, though, preferably between 50 and 200 monomer units.

Solubilizing blocks (S) comprising linear polymers may comprise homopolymers comprising a single monomer composition or copolymers having two or more distinct compositions of monomers. In some embodiments, the homopolymer comprises neutral, hydrophilic monomers or charged monomers, e.g., positive, negative or zwitterion monomers. In other embodiments, the copolymer comprises neutral, hydrophilic monomers, and positive, negative or zwitterion monomers, or any combination thereof. Solubilizing blocks comprising linear polymers may comprise monomers linked to any solubilizing groups (SG) (or “moieties”), which generally refers to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof

In some embodiments, the solubilizing block (S) comprises neutral hydrophilic monomers, which may be described generically as hydrophilic monomers. In some embodiments, the hydrophilic monomers are selected from (meth)acrylates or (meth)acrylamides (inclusive of acrylates, methacrylates, acrylamides and methacrylamides) of the chemical formula CH₂═CR¹¹—C(O)—R¹⁰ (“Formula II”), wherein the acryl side group R¹⁰ may be selected from one or more of —OR², —NHR¹² or —N(CH₃)R¹², where R¹¹ can be H or CH₃, and R¹² is independently selected from any hydrophilic substituent. Non-limiting examples of R¹² include but are not limited to H (except for OR¹³), CH₃, CH₂CH₃, CH₂CH₂OH, CH₂(CH₂)₂OH, CH₂CH(OH)CH₃, CHCH₃CH₂OH or (CH₂CH₂O)_{y}H, where y is an integer number of repeating units, typically 1 to 6, such as 1, 2, 3, 4, 5 or 6.

A non-limiting example of a neutral hydrophilic monomer of Formula II wherein R¹⁰=NHR¹², R¹¹=CH₃, and R¹³=CH₂CH(OH)CH₃ is N-2-hydroxypropyl(methacrylamide) (HPMA):

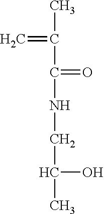

The above example, N-(2-hydroxypropyl(methacrylamide)) (HPMA), is an example of a neutral hydrophilic monomer of Formula II.

In some embodiments, the solubilizing block (S) comprises charged monomers that contain one or more functional groups (“charged functional group”) that either have a fixed charge or have net charge under certain physiological conditions. Non-limiting examples of charged monomers include any monomer that comprises amine, quaternary ammonium, sulfonic acid, sulfuric acid, sulfonium, phosphoric acid, phosphonic acid, phosphonium, carboxylic acid and/or boronic acid functional groups, as well as any combinations or salt forms thereof.

In some embodiments, charged monomers are selected from (meth)acrylates and (meth)acrylamides with chemical formula CH₂═CR¹⁴—C(O)—R¹³ (“Formula III”). The acryl side group R¹³ may be selected from one or more of the groups consisting of —OR¹⁵, —NHR¹⁵ or —N(CH₃)R¹⁵, where R¹⁴ can be H or CH₃ and R¹⁵ can be selected from, but is not limited to, H, linear alkyl structures such as (CH₂)_{y}NH₂, (CH₂)_{y}-imidazole, (CH₂)_{y}-pyridine amine, (CH₂)_{y}-(quinoline-amine), (CH₂)_{y}-pyridine amine, (CH₂)_{y}-naphthalene amine, (CH₂)_{y}CH(NH₂)COOH, (CH₂)_{y}COOH, (CH₂)_{y}CH(CH₃)COOH, (CH₂)_{y}C(CH₃)₂COOH, (CH₂)_{y}PO₃H₂, (CH₂)_{y}OPO₃H₂, (CH₂)_{y}SO₃H, (CH₂)_{y}OSO₃H, (CH₂)_{y}B(OH)₂, CH₂N(CH₃)₂, CH₂CH₂N(CH₃)₂, CH₂CH₂CH₂N(CH₃)₂, CH₂N(CH₂CH₃)₂, CH₂CH₂N(CH₂CH₃)₂, CH₂CH₂CH₂N(CH₂CH₃)₂, CH₂N(CH(CH₃)₂), CH₂CH₂N((CH(CH₃)₂), CH₂CH₂CH₂N(CH(CH₃)₂), CH[CH₂N(CH₃)₂]₂, CH(COOH)CHCH₂COOH, (CH₂)_{y}NH(CH₂)ⱼCOOH, (CH₂)_{y}N(CH₃)(CH₂)_{y}COOH, (CH₂)_{y}N⁺(CH₃)₂(CH₂)_{y}COOH, (CH₂)_{y}N⁺(CH₂—CH₃)₂(CH₂)_{y}COOH, [CH₂CH(CH₃)O]₅PO₃H₂, C(CH₃)₂CH₂SO₃H, C₆H₄B(OH)₂, (CH₂)y(PO₄⁻)(CH₂)_{y}—N⁺(CH₃)₃, (CH₂)_{y}(PO₄H)(CH₂)_{y}—N(CH₃)₂, (CH₂)y(PO₄)(CH₂)_{y}—N⁺(CH₂CH₃)₃, (CH₂)_{y}(PO₄H)(CH₂)_{y}—N(CH₂CH₃)₂, (CH₂)_{y}NH(CH₂)ⱼSO₃H, (CH₂)_{y}N(CH₃)(CH₂)_{y}SO₃H, (CH₂)_{y}N⁺(CH₃)₂(CH₂)_{y}SO₃⁻, or (CH₂)_{y}N⁺(CH₂—CH₃)₂(CH₂)_{y}SO₃⁻, where y, independently for each occurrence, is an integer number of a repeating units, typically between 1 to 6, such as 1, 2, 3, 4, 5 or 6. In some embodiments of (meth)acrylates and (meth)acrylamides of Formula III, the acryl side group comprises tetraalkyl ammonium salts, nitrogen containing heterocycles, aminoaryl, or aminoheteroaryl, which may be linked to the monomer through any suitable means either directly or via a linker. Non-limiting examples of aryls, nitrogen containing heteroaryls and/or aminoheteroaryls include pyrrolyl, imidazolyl, pyridinyl, pyrimidinyl, pyrazinyl, diazepinyl, indolyl, quinolinyl, amino quinolinyl, amino pyridinyl, purinyl, pteridinyl, anilinyl, amino naphthyl or the like. In certain preferred embodiments of (meth)acrylates and (meth)acrylamides of Formula III, the acryl side group comprises carboxylic acid(s), which may be linked to the monomer through any suitable means either directly or via a linker. A non-limiting example of a charged monomer of Formula III wherein R¹³=—OR¹⁵, R⁴=CH₃ and R¹⁵=H is:

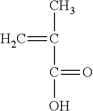

### Dendron-Based Solubilizing Blocks

Certain preferred embodiments of solubilizing blocks (S) comprise dendron amplifiers (“dendrons”), wherein the focal point of the solubilizing block (S) is linked either directly or indirectly via a spacer (B) and/or Linker U to a hydrophobic block (H), and the terminal groups (FGt) are either blind ended (unlinked) and function as solubilizing groups, or the terminal functional groups (FGt) are linked to solubilizing groups, wherein the solubilizing groups (SG) (or “moieties”) generally refer to any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof. In some embodiments, the solubilizing block (B) comprises dendron architecture and the terminal functional groups (FGt) are unlinked and therefore FGt are the solubilizing groups (SG). In other embodiments, the solubilizing block (B) comprises dendron architecture and the terminal functional groups (FGt) are linked either directly or via a linker to a solubilizing group (SG).

An unexpected finding reported herein is that the architecture and composition of amphiphiles of formula S-[B]-[U]-H had a marked impact on particle stability and drug loading into such particles. Accordingly, it was observed by the authors of the present disclosure that amphiphiles of formula S-[B]-[U]-H comprising solubilizing blocks with dendron architecture formed nanoparticles with improved hydrodynamic stability, higher drug loading and increased biological activity as compared with amphiphiles of formula S-[B]-[U]-H comprising solubilizing blocks (S) with linear architecture. Therefore, in preferred embodiments of amphiphiles, the amphiphile comprises a solubilizing block (S) further comprising a dendron amplifier, with a single (“core” or “focal point”) functional group linked either directly or indirectly via a spacer (B) and/or linker (U) to a hydrophobic block (H), additionally wherein the dendron has 2 or more solubilizing groups (SG), preferably, between 2 and 32 solubilizing groups, though more preferably between 4 and 8 solubilizing groups. Preferred compositions of dendron-based solubilizing blocks (S) are described throughout the specification.

The solubilizing groups (SG) of the solubilizing block (S) function to improve solubility and therefore stability of particles formed by amphiphiles but also impact blood protein interactions, cellular uptake and intracellular trafficking. Therefore, solubilizing groups (SG) should be carefully selected to meet the demands of the application. It was identified that particular solubilizing group (SG) compositions that led to unexpected improvements in biological activity. Accordingly, particles comprising amphiphiles with solubilizing groups comprising dendrons with solubilizing groups (SG) selected from carboxylic acids with net negative charge (at pH 7.4) were found to be inefficiently phagocytosed by most cell types. Similarly, particles comprising amphiphiles with solubilizing groups comprising linear polymers or dendrons with net neutral or near neutral charge were generally found to be poorly phagocytosed by immune cells, e.g., antigen presenting cells, and other cell populations, unless the linear polymers or dendrons comprise neutral sugar molecules that bind C-type lectin receptors that promote uptake by immune cell populations or other sugar molecules, such as glucose or galactose, which promote uptake via GLUT1 and asialglycoprotein, respectively, by various cell populations. Furthermore, particles comprising amphiphiles with solubilizing groups comprising linear polymers or dendrons with net positive charge were found to be broadly taken up by various cell populations, particularly by antigen presenting cells. Thus, the solubilizing block (S) charge and composition can be tuned by varying the solubilizing groups (SG) to modulate biological activity. Preferred compositions of solubilizing groups are described below and throughout the specification.

### Solubilizing Groups (SG)

Solubilizing groups (SG) (or “moieties”) are defined broadly as any hydrophilic groups, including neutral hydrophilic groups that do not carry a full integer value of charge; zwitterions, which are neutral but carry a whole number value of positive charge and a whole number value of negative charge; positively charged groups; and negatively charged groups; or a combination thereof.

In certain preferred embodiments, the solubilizing block (B) comprises solubilizing groups (SG) selected from sugar molecules comprising one or more sugar monomers, e.g., monosaccharides, disaccharides, trisaccharides, oligosaccharides and the like. Non-limiting examples of solubilizing groups selected from sugar molecules include but are not limited to glucose, glucosamine, N-acetyl glucosamine, galactose, galactosamine, N-acetyl galactosamine, mannose and sialyl lewis^{X} (sLeX), which may be linked to solubilizing blocks through any suitable linker at any suitable attachment point, e.g.:

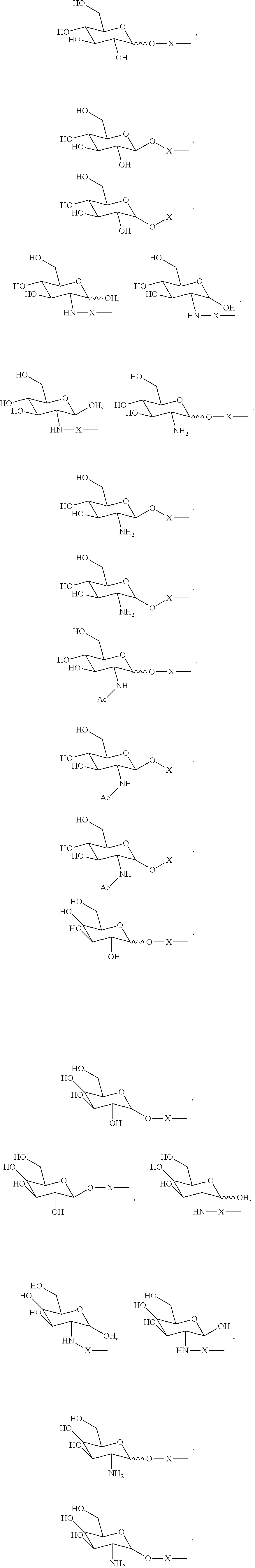

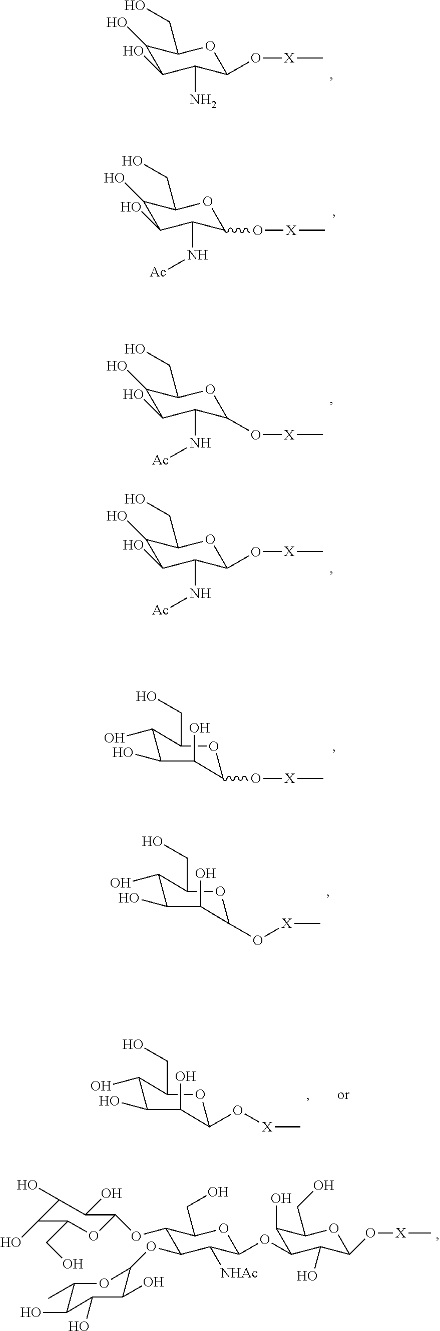

wherein X is any suitable linker molecule, which may be present or absent, and when present is typically selected from lower alkyl or PEG groups.

In some embodiments, the solubilizing block (S) comprises solubilizing groups (SG) that have net positive or net negative charge in aqueous buffers at a pH of about 7.4. The charge of the solubilizing groups (SG) may be dependent or independent of the pH of the solution in which the solubilizing block (S) is dispersed, such is the case, for example, for tertiary amines and quaternary ammonium compounds that are pH dependent and pH independent, respectively. Non-limiting examples of solubilizing groups that have net positive or net negative charge at certain pH in aqueous solutions or have pH independent charge are provided here for clarity:

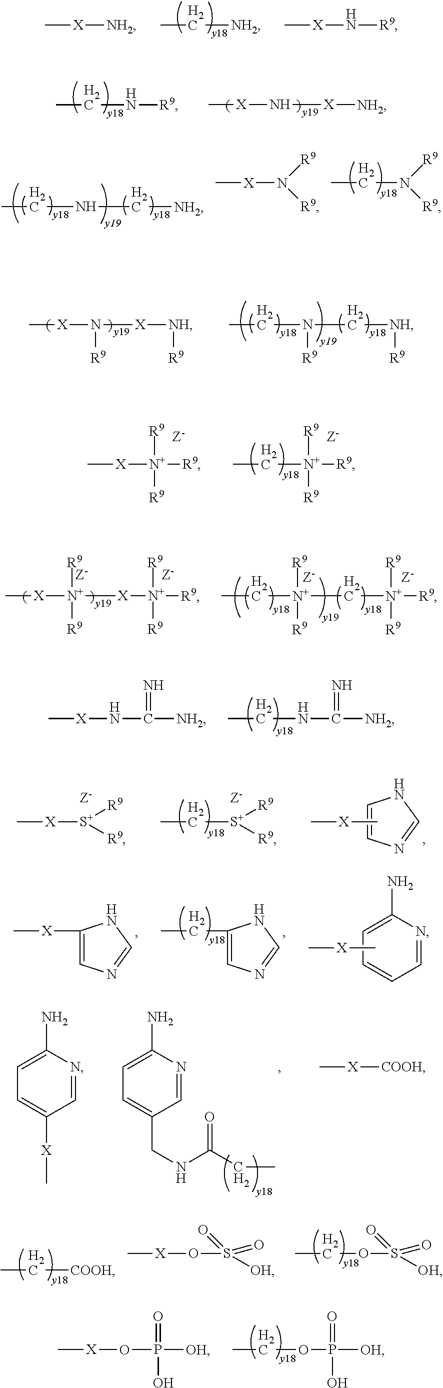

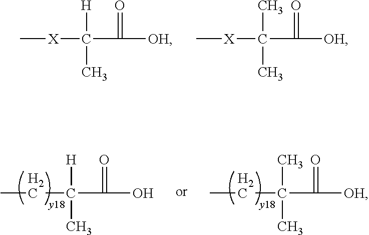

wherein X is any suitable linker molecule, which may be present or absent, and when present is typically selected from lower alkyl or PEG, y18 and y19 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, and Z⁻ is any suitable counter anion, which is typically selected from conjugate bases of weak acids or halide ions, such as Cl⁻, I⁻, or Br⁻.

In certain preferred embodiments, the solubilizing block (S) comprises solubilizing groups (SG) selected from zwitterions that have 0 net charge, or net 0 charge in aqueous conditions at certain pH. In some embodiments, the solubilizing block (S) comprises solubilizing groups (SG) selected from zwitterions that have 0 net charge at pH 7.4, but have net positive charge at reduced pH, e.g., tumor pH between about 5.5 to 7.0. Non-limiting examples of solubilizing groups comprising zwitterions are provided here for clarity:

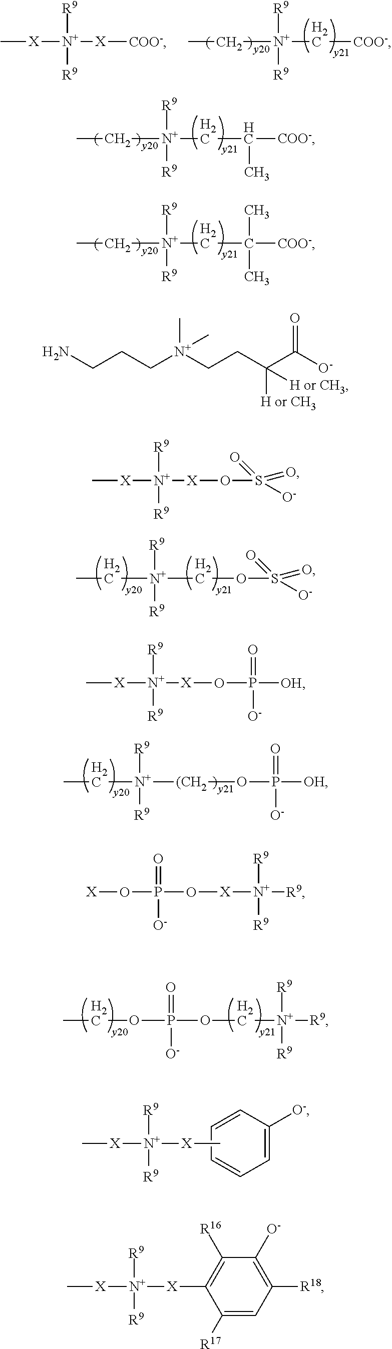

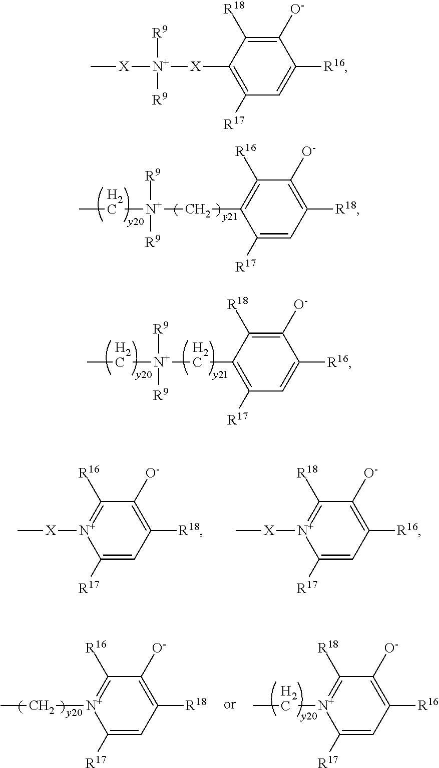

wherein X is any suitable linker, which may be present or absent, and when present is typically selected from lower alkyl or PEG groups, y20 and y21 are each independently any integer, typically selected from between 1 to 6, R⁹ is selected from lower alkyl or branched alkyl groups, such as CH₃, CH₂CH₃, CH₂CH₂CH₃, CH(CH₃)₂, H₂CH(CH₃)₂ or the like, R¹⁶, R¹⁷ and R¹⁸ are each independently selected from —H, CH₃, F and —NO₂.

In some embodiments, the solubilizing group (SG) may further comprise a targeting moiety and/or drug molecule. As a non-limiting example, certain sugar molecules may improve solubility and therefore function as a solubilizing group; additionally, the sugar molecule may bind to cell surface receptors and/or exert a physiological effect and therefore also function as a targeting moiety and/or drug molecule (D). Accordingly, solubilizing groups (SG) comprising mannose bind to mannose receptors and therefore target cells and tissues expressing such receptors; additionally, binding to the mannose receptor can promote phagocytosis and may therefore exert a physiological effect. Additional non-limiting examples of solubilizing groups (SG) that may perform two or more functions include targeting molecules comprising hydrophilic peptides, glycopeptides, antibodies, fragments of antibodies, nanobodies, nucleic acid aptamers and related molecules that are both hydrophilic and bind to specific cells or tissues.

### Linkage of Solubilizing Group (SG) to the Solubilizing Block

Solubilizing groups (SG) may be linked to the solubilizing block (S) through any suitable means, including any suitable linker molecule. In certain preferred embodiments of dendron-based solubilizing blocks (S), the terminal functional group is a carboxylic acid, and the solubilizing group is linked via an ester or, more preferably, an amide bond. In certain other preferred embodiments of dendron-based solubilizing blocks (S), the terminal functional group is an amine, and the solubilizing group is linked to the terminal functional group via an amide or carbamate bond.

In preferred embodiments, solubilizing groups (SG) are linked to the solubilizing block (S) through a covalent bond via a suitable linker X, which is typically selected from lower alkyl or PEG groups. Particular suitable linkers X that are preferred for joining SG to S are referred to as X5. In non-limiting examples, solubilizing blocks (S) selected from either polymers comprising monomers comprising amines or dendrons comprising terminal functional groups (FGt) comprising amines, e.g., —NH₂, are covalently linked to solubilizing groups (SG) via a suitable linker, X5, through reaction with activated carboxylic acids (LG—C(O)—R¹⁹) to yield —NH—C(O)—R¹⁹; activated mixed carbonates (LG—C(O)—O—R¹⁹) or chloroformates (Cl—C(O)—O—R¹⁹) to yield NH—C(O)—O—R¹⁹; aldehydes or ketones (CR²²(O)—R¹⁹) to yield Schiff base of formula CR²²(—NH)-R¹⁹; alkenes (C(R²²)(R²³)═C(R²⁴)(R¹⁹) to yield Michael-addition products (e.g., NH-C(R²²)(R²³)—CH(R²⁴)(R¹⁹) or —N(C(R²²)(R²³)—CH(R²⁴)(R¹⁹))₂); or, alkyl or aryl halide (LG-R¹⁹, wherein LG=Cl, Br or I), to yield —NH—R¹⁹, —N(—R¹⁹)₂ and/or —N+(—R¹⁹)₃. In additional non-limiting examples, solubilizing blocks (S) selected from either polymers comprising monomers comprising carboxylic acids or dendrons comprising terminal functional groups (FGt) comprising carboxylic acids, e.g., —COOH (or —C(O)-LG), are covalently linked to solubilizing groups (SG) via a suitable linker, X5, through reaction with an amine (NH₂—R¹⁹) to yield —C(O)—NH—R¹⁹ or methylamine (R¹⁹—N(CH₃)(H) or R¹⁹—NHMe) to yield —C(O)—N(CH₃)(R¹⁹)

In the above non-limiting examples, LG is any suitable leaving group, and R¹⁹ may be selected from but is not limited to —(CH₂)ₜ-SG, —(CH₂CH₂O)ₜ—CH₂CH₂—SG, —(CH₂)ₜ—C(O)—NH—(CH₂)ᵤ-SG, —(CH₂CH₂O)ₜCH₂CH₂C(O)—NH—(CH₂)ᵤ-SG, —(CH₂)ₜ—NH—C(O)—NH—(CH₂)ᵤ-SG and (CH₂CH₂O)ₜCH₂CH₂NH—C(O)—(CH₂)ᵤ-SG where t and u are each independently an integer typically selected from between 1 to 6, such as 1, 2, 3, 4, 5 or 6. Preferred X5 for linking S to SG (i.e., S-X5-SG) are typically selected from —NH—(CH₂)ₜ—, —NH—(CH₂CH₂O)ₜ—CH₂CH₂—, —NH—(CH₂)ₜ—C(O)—NH—(CH₂)ᵤ—, —NH—(CH₂CH₂O)ₜCH₂CH₂C(O)—NH—(CH₂)ᵤ—, NH—(CH₂)ₜ—NH—C(O)—NH—(CH₂)ᵤ—, —NH(CH₂CH₂O)ₜCH₂CH₂NH—C(O)—(CH₂)ᵤ—, —C(O)—(CH₂)ₜ—, —C(O)—(CH₂CH₂O)ₜ—CH₂CH₂—, —C(O)—(CH₂)ₜ—C(O)—NH—(CH₂)ᵤ—, C(O)—(CH₂CH₂O)ₜCH₂CH₂C(O)—NH—(CH₂)ᵤ—, C(O)—(CH₂)ₜ—NH—C(O)—NH—(CH₂)ᵤ— or —C(O)—(CH₂CH₂O)ₜCH₂CH₂NH—C(O)—(CH₂)ᵤ—, where t and u are each independently an integer typically selected from between 1 to 6, such as 1, 2, 3, 4, 5 or 6.

A non-limiting example of an amphiphile comprising a solubilizing block (S) with dendron architecture, wherein the dendron is second generation and comprises monomeric units selected from FG1-CH(R′)₂, wherein FG1 (and the focal point) is NH₂, R¹ is (OCH₂CH₂)_{y}-FG2, y is 1 and FG2 (and FGt) is COOH, wherein the terminal functional group (FGt) carboxylic acids are linked to NH₂—R¹⁹ to yield —C(O)—NH₂—R¹⁹ wherein R¹⁹ is —(CH₂CH₂O)ₜ—CH₂CH₂—SG, t=1 and the solubilizing group is selected from a glucose is provided below for clarity:

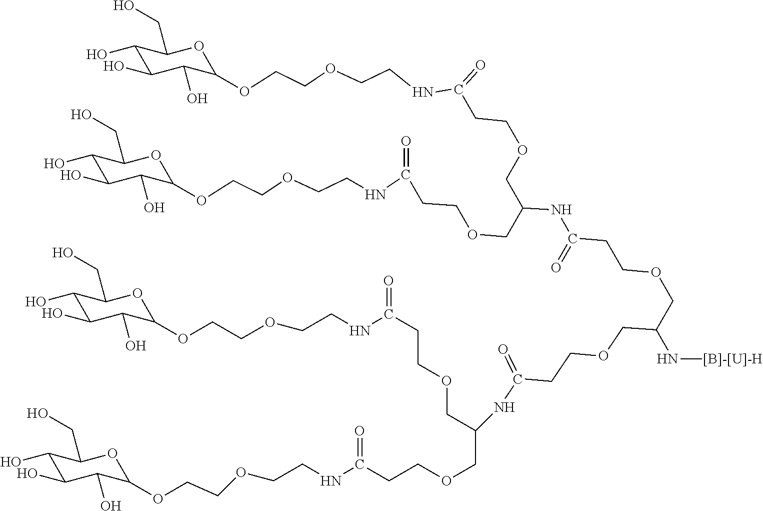

Wherein the solubilizing block (S) is linked either directly or indirectly via a spacer (B) and/or Linker U to the hydrophobic block (H), which may further comprise a drug molecule (e.g., H-D). In the above example, X5 is —NH—R¹⁹ and R¹⁹ is —(CH₂CH₂O)ₜ—CH₂CH₂—SG, which may be written as —NH—(CH₂CH₂O)ₜ—CH₂CH₂—(SG not shown), wherein t=1 and SG is a glucose.

Additional examples of solubilizing blocks (S) with dendron architecture that have particular utility for certain applications and/or lead to unexpected improvements in manufacturing and/or biological activity are provided throughout the specification.

### Impact of the Number of Charged Functional Groups

In some embodiments, the solubilizing block (S) has a net negative charge and comprises one or more functional groups that carry a negative charge at pH 7.4. Suitable solubilizing blocks (S) that carry a net negative charge include molecules bearing functional groups (e.g., functional groups with a pKa of about 7.4 or less) that occur as the conjugate base of an acid at physiologic pH, at a pH of about 7.4 or less. These include but are not limited to molecules bearing carboxylates, sulfates, phosphates, phosphoramidates, and phosphonates. The solubilizing block (S) bearing a carboxylate may be selected from but is not limited to carboxylic acids selected from glutamic acid, aspartic acid, pyruvic acid, lactic acid, glycolic acid, glucuronic acid, citrate, isocitrate, alpha-keto-glutarate, succinate, fumarate, malate, oxaloacetate, butyrate, methylbutyrate, dimethylbutyrate and derivatives thereof. In certain embodiments, the solubilizing block (S) comprises a molecule with between 1 to 20 negatively charged functional groups, such as 1, 2, 3, 4, 5, 6, 7, 8, 9, 10, 11, 12, 13, 14, 15, 16, 17, 18, 19 or 20 negatively charged functional groups, though, typically no more than 8 negatively charged functional groups, preferably between 4 and 8 negatively charged functional groups.

In some embodiments, the solubilizing block (S) has a net positive charge and comprises positively charged functional groups. Suitable solubilizing blocks (S) that carry a net positive charge include molecules that occur as the conjugate acid of weak bases at pH 7.4, wherein the pKa of the conjugate acid of the base is greater than 7.4. These include but are not limited to molecules bearing primary, secondary and tertiary amines, as well as quaternary ammonium, guanidinium, phosphonium and sulfonium functional groups. Suitable molecules bearing ammonium functional groups include, for example, imidazolium, and tetra-alkyl ammonium compounds. In some embodiments, the solubilizing block comprises quaternary ammonium or sulfonium compounds that carry a permanent positive charge that is independent of pH.

In some embodiments, the solubilizing group (S) comprises between 1-20 positively charged functional groups, such as 1, 2, 3, 4, 5, 6, 7, 8, 9, 10, 11, 12, 13, 14, 15, 16, 17, 18, 19 or 20 positively charged functional groups. For amphiphiles, the solubilizing block (S) typically has no no more than 8 charged functional groups, preferably between 4 and 8 positively charged functional groups.

### Counter-Ion Selection

An additional consideration regarding charged molecules (C) is the counterion selected. Non-limiting examples of charged molecules (C) bearing functional groups with positive charge include but are not limited to halides, including chloride, bromide and iodide anions, and conjugate bases of acids, including, phosphate, sulfates, sulfites and carboxylate anions including formate, succinate, acetate and trifluoroacetate. Suitable counterions for charged molecules (C) bearing functional groups with negative charge include but are not limited to hydrogen and alkali and alkaline earth metals, including, for example, sodium, potassium, magnesium and calcium, or conjugate acids of weak bases, such as ammonium compounds. Suitable amines used to form the ammonium salt include but are not limited to ammonium, primary amines, such as tris(hydroxymethyl)aminomethane (“TRIS”), secondary amines based on di-alkyl amines, such as dimethyl amine and diethyl amine, tertiary amines based on tri-alkyl amines, such as trimethylamine, di-isopropryl ethylamine (DIPEA) and triethylamine (TEA), as well as quaternary ammonium compounds. Unexpectedly, tris(hydroxymethyl)aminomethane (or Tris) as the ammonium salt of acids as the counterion of amphiphilic block copolymers with negative charge has improved solubility in both water-miscible organic solvents, such as DMSO, DMF, acetone and ethanol, and aqueous solutions. For these reasons, the protonated form of tris(hydroxymethyl)aminomethane is a preferred counter-ion to use in the preparation of salts of conjugate bases of acids present on the amphiphilic block copolymers of the present disclosure.

### Preferred Compositions of Amphiphiles

The composition of the amphiphile is selected to meet the specific demands of the application. The surface properties of the amphiphile, e.g., the properties of the solubilizing block(s) (S), primarily control the pharmacokinetics, including tissue targeting. Accordingly, for amphiphiles delivered by the intravenous route, the surface properties of the amphiphile strongly dictate blood clearance and tissue distribution: amphiphiles with solubilizing groups comprising high net positive charge are rapidly cleared from the blood and are predominantly taken up by phyagocytic cells in the liver, spleen and lung; amphiphiles with solubilizing groups comprising sugar molecules that bind asialoglycoprotein receptor, e.g., GalNAc, are predominantly taken up by phyagocytic cells in the liver; and amphiphiles with solubilizing groups comprising hydrophilic polymers with net neutral or net negative charge that have random coil architecture typically have relatively longer half-life in the blood and are preferred for targeting tissues other than liver, spleen and lung.

The architecture of the amphiphile was also found to impact the hydrodynamic behavior, including particle size and particle size stability in aqueous buffers. Specifically, amphiphiles comprising solubilizing blocks with dendron architecture comprising 2 or more solubilizing groups, typically no more than 16, for example, between 2 and 8 solubilizing groups, typically promoted nanoparticle micellization even at neutral or near neutral charge.

Therefore, in certain preferred embodiments of nanomedicines for intravenous drug delivery, the nanomedicine comprises an amphiphile with a solubilizing block (S) comprising a dendron amplifier (i.e., has dendron architecture) with two or more solubilizing groups that are either linked to the hydrophobic block (H) directly or indirectly via a spacer (B) and/or linker (U). A non-limiting example of an amphiphile with a solubilizing block (S) comprising a dendron amplifier (i.e., has dendron architecture) linked to a hydrophobic block (H) directly or indirectly via a spacer (B) and/or linker (U) is provided below for clarity:

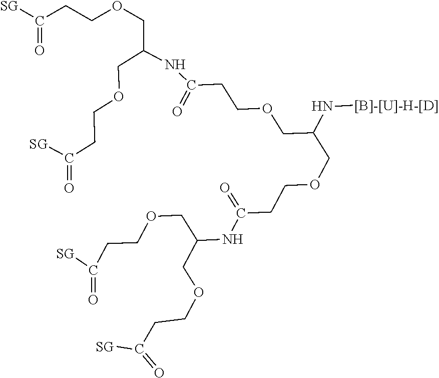

wherein SG is selected from sugar molecules, carboxylic acids, amines and/or hydroxyls that are linked to S either directly or via a suitable linker X, or, more preferably, X5; B is a spacer; U is a linker; H is a hydrophobic block typically selected from poly(amino acids) of Formula I; D is a drug molecule; [ ] denotes that the group is optional; and - denotes that the two adjacent groups are directly attached to one another by a covalent bond or indirectly to one another via a suitable linker X.

In certain preferred embodiments the spacer (B) is present and selected from PEG and the above structure becomes:

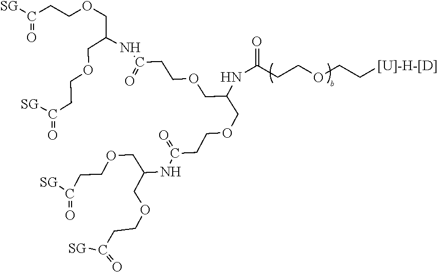

wherein b is an integer number of monomeric units comprising the spacer and is typically between 1 and 48, such as 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 22, 43, 44, 45, 46, 47 or 48 monomeric units, preferably between about 1 and 36 monomer units, or between about 4 and 36 monomeric units, most preferably 24 monomeric units.

In some embodiments the amphiphile comprises a dendron amplifier comprising amino acids. A non-limiting example of an amphiphile with a solubilizing block (S) comprising a dendron amplifier (i.e., has dendron architecture) comprising lysine that is linked to a hydrophobic block (H) directly or indirectly via a spacer (B) and/or linker (U) is provided below for clarity:

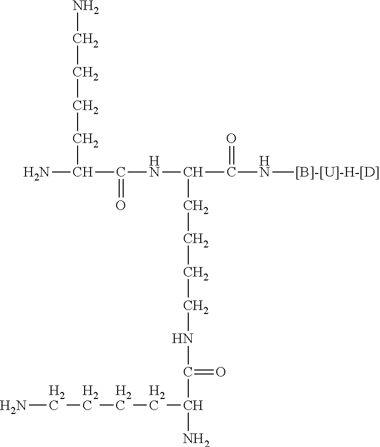

wherein SG in the above example is an amine, i.e., the terminal functional group (FGt) is the SG; B is a spacer; U is a linker; H is a hydrophobic block typically selected from poly(amino acids) of Formula I; D is a drug molecule; [ ] denotes that the group is optional; and - denotes that the two adjacent groups are directly attached to one another by a covalent bond or indirectly to one another via a suitable linker X.

In some embodiments of amphiphiles comprising amino acid based dendron amplifiers, the solubilizing groups (SG) are linked to S either directly or via a suitable linker X, or, more preferably, X5. In a non-limiting example, wherein the SG are linked to the lysine based dendron amplifier of the above structure through the linker X5, the structure becomes:

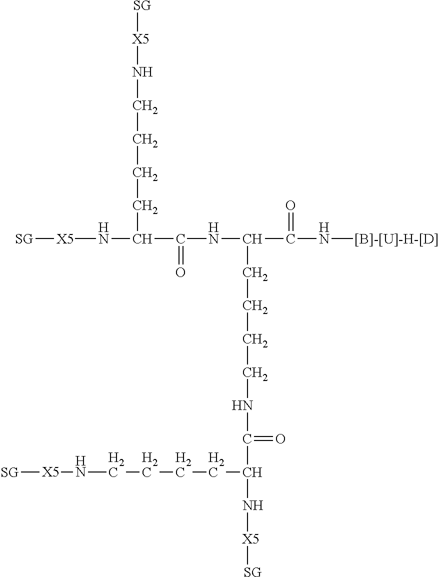

wherein SG is selected from sugar molecules, carboxylic acids, amines and/or hydroxyls that are linked to S either directly or via a suitable linker X, or, more preferably, X5; B is a spacer; U is a linker; H is a hydrophobic block typically selected from poly(amino acids) of Formula I; D is a drug molecule; [ ] denotes that the group is optional; and - denotes that the two adjacent groups are directly attached to one another by a covalent bond or indirectly to one another via a suitable linker X.

In contrast, amphiphiles comprising poly(amino acid) based solubilizing blocks with linear architecture typically required net charge greater than or equal to +2 or less than or equal to −2 to promote, or greater than or equal to +4 or less than or equal to −4 to promote nanoparticle micellzation. In preferred embodiments of amphiphiles comprising poly(amino acid) based solubilizing blocks with linear architecture, the solubilizing block typically comprises between 2 and 12 charged amino acids, such as 2, 3, 4, 5, 6, 7, 8, 9, 10, 12 charged amino acids. Non-limiting examples of amphiphiles comprising poly(amino acid) based solubilizing blocks with linear architecture are shown below for clarity:

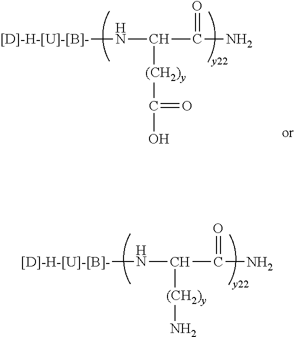

wherein the solubilizing block (S) comprises an integer number of amino acids, y22; y is an integer typically selected from between 1 to 6; B is a spacer; U is a linker; H is a hydrophobic block typically selected from poly(amino acids) of Formula I; D is a drug molecule; [ ] denotes that the group is optional; and - denotes that the two adjacent groups are directly attached to one another by a covalent bond or indirectly to one another via a suitable linker X. Note: in the above example, the solubilizing block comprising amino acids may be linked to the hydrophobic block directly or indirectly via B and/or U at the N-terminus (shown) or C-terminus (not shown), and the other end may be capped, e.g., the C-terminus may be capped with an amide (shown) or the N-terminus may be capped with an acetyl group.

In certain preferred embodiments of amphiphiles comprising poly(amino acid) based solubilizing blocks with linear architecture, the SG is linked to S via the linker X5. Non-limiting examples are shown below for clarity:

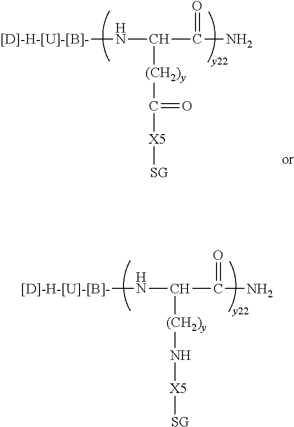

wherein SG is selected from sugar molecules, carboxylic acids, amines and/or hydroxyls that are linked to S either directly or via a suitable linker X, or, more preferably, X5.

### Selection of Drug Molecules (D) for Cancer Treatment and Chronic Viral Infections

The nanomedicine compositions disclosed herein comprise at least one amphiphile and at least one drug molecule (D). The drug molecule (D) is selected based on the intended use of the nanomedicine. The nanomedicines disclosed herein for treating cancer and chronic viral infections comprise drug molecules selected from immunostimulants and/or chemotherapeutic agents.

Suitable immunostimulants include various agonists of pattern recognition receptors (PRRs), particularly agonists of Stimulator of Interferon Genes (STING), TLR-3, TLR-4, TLR-7, TLR-8, TLR-7/8 and TLR-9. Non-limiting examples of TLR-3 agonists include dsRNA, such as PolyI:C, and nucleotide base analogs; TLR-4 agonists include lipopolysaccharide (LPS) derivatives, for example, monophosphoryl lipid A (MPL) small molecules such as pyrimidoindole; TLR-7 & -8 agonists include ssRNA and nucleotide base analogs, including derivatives of imidazoquinolines, hydroxy-adenine, benzonaphthyridine and loxoribine; TLR-9 agonists include unmethylated CpG and small molecules that bind to TLR-9; STING agonists include cyclic dinucleotides, and synthetic small molecules, such as alpha-mangostin and its derivatives as well as linked amidobenzimidazole (“diABZI”) and related molecules (see: Ramanjulu et al., Nature, 20:439-443, 2018).

In several embodiments, the nanomedicine comprises an immunostimulant selected from a TLR agonist, such as an imidazoquinoline-based TLR-7/8 agonist. For example, the immunostimulant can be Imiquimod (R2137) or Resiquimod (R2148), which are approved by the FDA for human use for certain indications and uses. In other embodiments, the immunostimulant comprises a TLR-7 agonist, a TLR-8 agonist and/or a TLR-7/8 agonist. Numerous such agonists are known, including many different imidazoquinoline compounds.

Imidazoquinolines are synthetic immunomodulatory drugs that act by binding Toll-like receptors-7 and/or -8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton and are described here in as hydrophobic molecules or sometimes as comprising heteroaryl or amino-heteroayl groups. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod or resiquimod.

In some embodiments, the immunostimulant is a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the immunostimulant is an imidazoquinoline with the formula:

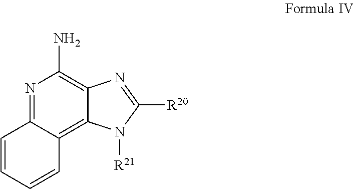

In Formula IV, R²⁰ is selected from one of hydrogen, optionally-substituted lower alkyl, or optionally-substituted lower ether; and R²¹ is selected from one of optionally substituted arylamine, or optionally substituted lower alkylamine. R²¹ may be optionally substituted to a linker that links to a polymer.

In some embodiments, the R²⁰ included in Formula IV can be selected from hydrogen,

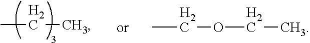

In some embodiments, R²¹ can be selected from,

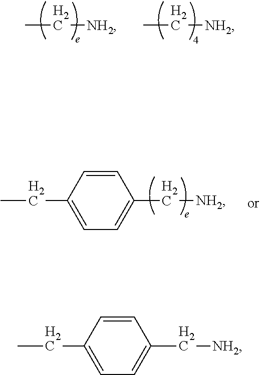

wherein e denotes the number of methylene unites is an integer from 1 to 4.

In some embodiments, R²¹ can be

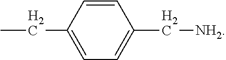

In some embodiments, R²¹ can be

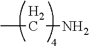

In some embodiments, R²⁰ can be

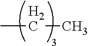

### and R²¹ can be

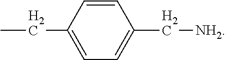

In some embodiments, at least one D is

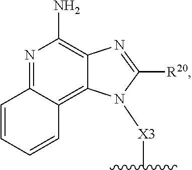

wherein R²⁰ is selected from H, alkyl, alkoxyalkyl, aryl, heteroaryl, aminoalkyl, amide and ester; and X3 is selected from alkyl, alkoxyalkyl, aralkyl, heteroaralkyl, aryl, heteroaryl and carboxy.

In some embodiments, wherein, R²⁰ is selected from H, alkyl and alkoxyalkyl; and X3 is selected from alkyl and aralkyl. In other embodiments, R²⁰ is butyl.

In some embodiments, X3 is alkyl.

In some embodiments, the drug (D) used for cancer treatment is an immunostimulant selected from agonists of STING. In some embodiments, the agonist of STING is selected from amidobenzimidazole based molecules. A non-limiting example is shown here for clarity, wherein the piperazine ring is optionally used as a reactive handle for linkage either directly or via a linker to the amphiphile, e.g., to reactive monomers of poly(amino acid)-based hydrophobic blocks (H).

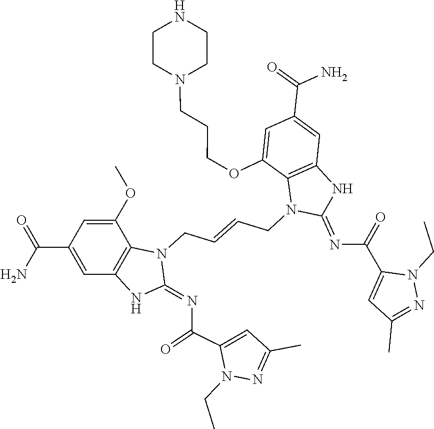 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

In other embodiments, the drug used for cancer treatment is selected from chemotherapeutics.

In some embodiments, chemotherapeutic is selected from alkylating agents (cisplatin, cyclophosphamide & temozolomide as an example), mitotic inhibitors (taxanes and Vinca alkaloids) or antimetabolites (5-fluorouracil, capecitabine & methotrexate as an example). In some embodiments the chemotherapeutic drug is an immunomodulator that reverse immune suppression, including inhibitors of adenosine receptors and aryl hydrocarbon receptors. In other embodiments, the chemotherapeutic is selected from topoisomerase inhibitors (Topoisomerase I inhibitors and Topoisomerase II inhibitors). A non-limiting example is shown here for clarity, wherein the tertiary amine of topotecan is optionally modified to enable linkage either directly or via a linker to the amphiphile, e.g., to reactive monomers of poly(amino acid)-based hydrophobic blocks (H):

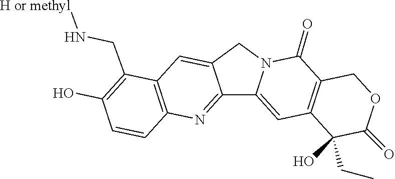

In other embodiments, the chemotherapeutic is selected from tyrosine kinase inhibitors. A non-limiting example is shown here for clarity, wherein the morpholine group of gefitinib is optionally replaced with a piperazine group to enable linkage either directly or via a linker to the amphiphile, e.g., to reactive monomers of poly(amino acid)-based hydrophobic blocks (H).

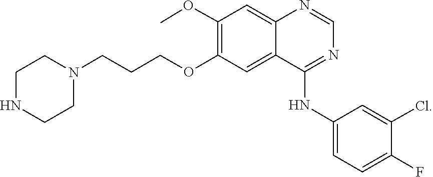 [SMILES: COc1cc2ncnc(Nc3ccc(F)c(Cl)c3)c2cc1OCCCN1CCNCC1]

In other embodiments, the chemotherapeutic is selected from angiogenesis (e.g., anti-VEGF receptor) inhibitors. A non-limiting example is shown here for clarity, wherein the tertiary amine of sunitinib can be optionally modified to enable linkage either directly or via a linker to the amphiphile, e.g., to reactive monomers of poly(amino acid)-based hydrophobic blocks (H):

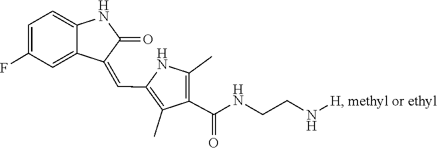

In other embodiments, the chemotherapeutic is selected from tumor antibiotics (anthracycline family, actinomycin-D and bleomycin as an example). In a non-limiting example, the anthracycline is doxorubicin and has the structure, wherein the amine or ketone may optionally be used for linkage either directly or via a linker to the amphiphile, e.g., to reactive monomers of poly(amino acid)-based hydrophobic blocks (H).

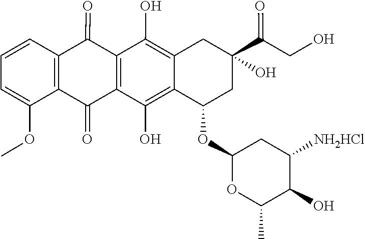

While any class of chemotherapeutic could be used, it was found, unexpectedly, that certain classes of chemotherapeutics used in combination with immunostimulants lead to unexpectedly enhanced tumor clearance. Herein, it is disclosed that preferred chemotherapeutics are those that induce either or both reversal of immune-suppression and/or the induction of immunogenic cell death. Thus, in certain embodiments of nanomedicines for cancer treatment, the nanomedicine includes immunostimulants and/or chemotherapeutics, wherein the chemotherapeutics are selected from anthracyclines, taxanes, platinum compounds, 5-fluorouracil, cytaribine and other such molecules that are useful for eliminating or altering the phenotype of suppressor cells in the tumor microenvironment.

Drug molecule(s) may be covalently attached to the amphiphile(s) or associated with the amphiphile(s) through non-covalent interactions, e.g., hydrophobic or electrostatic interactions. In some embodiments, the drug molecule is covalently attached to the amphiphile, e.g., the drug molecule is attached to to the hydrophobic block of the amphiphile through a degradable linker. In other embodiments, the drug molecule (D) is hydrophobic and is incorporated in the hydrophobic core of particles comprising amphiphile(s). In still other embodiments, the drug molecule carries a net positive or net negative charge and associates with a portion of the amphiphile that carrier opposite charge. The preferred method of incorporating the drug molecule depends on the chemical composition of the drug molecule.

In some embodiments of nanomedicines for cancer treatment, at least one drug molecule (D) is selected from immunostimulants that are hydrophobic and/or amphiphilic and are incorporated into the nanoparticles comprising amphiphiles through non-covalent interactions, such as hydrophobic interactions with the hydrophobic blocks comprising the core of the nanoparticles. Non-limiting examples of hydrophobic inmmunostimulants include, squalene-based immunostimulants; lipid-based PRR agonists, such as mincle receptor agonists (e.g., trehalose dimycolate and trehalose dibehenate) lipopolysaccharide-based agonists of TLR-4, and lipopeptide-based agonists of TLR-1/2 and TLR-2/6; heteroaryl-based agonists of TLR-4 (e.g., pyrimidoindole); and agonists of TLR-7/8 (e.g., imidazoquinolines and benzonaphthyridines) and STING (e.g., diABZI).

In some embodiments of nanomedicines for chronic viral infections, at least one drug molecule (D) is selected from immunostimulants that are hydrophobic and/or amphiphilic and are incorporated into the nanoparticles comprising amphiphiles through non-covalent interactions, such as hydrophobic interactions with the hydrophobic blocks comprising the core of the nanoparticles. Non-limiting examples of hydrophobic inmmunostimulants include, squalene-based immunostimulants; lipid-based PRR agonists, such as mincle receptor agonists (e.g., trehalose dimycolate and trehalose dibehenate) lipopolysaccharide-based agonists of TLR-4, and lipopeptide-based agonists of TLR-1/2 and TLR-2/6; heteroaryl-based agonists of TLR-4 (e.g., pyrimidoindole); and agonists of TLR-7/8 (e.g., imidazoquinolines and benzonaphthyridines) and STING (e.g., diABZI). In some embodiments, the chronic viral infection is a hepatitis infection. In some embodiments, the chronic viral infection is a hepatitis A, hepatitis B, hepatitis C, hepatitis D, or hepatitis E infection. In other embodiments the virus if human papilloma virus. In other embodiments the virus is a retrovirus, such as human immunodeficiency virus. In other embodiments the virus is a polyoma virus.

In some embodiments of nanomedicines for cancer treatment, at least one drug molecule (D) is selected from chemotherapeutic drugs that are hydrophobic and/or amphiphilic and are incorporated into the nanoparticles comprising amphiphiles through non-covalent interactions. Many chemotherapeutic drugs are highly hydrophobic and may be incorporated into the hydrophobic core of nanoparticles assembled from the amphiphiles disclosed herein. Non-limiting examples include anthracyclines, such as doxorubicin and daunorubicin; androgens, such as calusterone; anti-adrenals, such as mitotane or trilostane; members of taxoid or taxane family, such as paclitaxel (TAXOL®), docetaxel (TAXOTERE®) and analogues thereof; certain inhibitors of receptor tyrosine kinases and/or angiogenesis, including sorafenib (NEXAVAR®), and sunitinib (SUTENT®); and inhibitors of PI3K, such as alpelisib, and the like, and pharmaceutically acceptable salts, acids or derivatives of any of the above.

Nanomedicine comprising immunostimulants and/or chemotherapeutics may be used to treat any cancer. Non-limiting examples include hematological tumors, such as leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, including fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers (including adenocarcinoma, a bronchiolaveolar carcinoma, a large cell carcinoma, or a small cell carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma); skin cancer, such as a basal cell carcinoma, a squamous cell carcinoma, a Kaposi's sarcoma, or a melanoma; and, premalignant conditions, such as variants of carcinoma in situ, or vulvar intraepithelial neoplasia, cervical intraepithelial neoplasia, or vaginal intraepithelial neoplasia.

### Drug Molecules (D) for Tolerance

Disclosed herein are compositions of particles comprising amphiphiles further comprising a drug molecule selected from immunosuppressants, including but not limited to aryl hydrocarbon receptor (AHR) agonists; certain steroids, including glucocorticoids including synthetic analogs, such as prednisone (and its metabolite prednisolone); certain histone deacetylase inhibitors (HDACS), such as inhibitors of HDAC9; retinoic acid receptor agonists; mammalian target of rapamycin (mTOR) inhibitors, such as rapamycin; certain cyclin dependent kinase (CDK) inhibitors; certain adenosine receptor agonists; agonists of PD1; calcineurin inhibitors, such as cyclosporine; and other molecules that suppress proinflammatory or cytotoxic activity by immune cells or antibodies.

Compositions comprising amphiphiles and drugs molecules (D) selected from inhibitors of mTORC1 and/or mTORC2 were found to be particularly effective for tolerance applications, including for treating inflammatory conditions, such as allergies, autoimmunity, transplant rejection, and other inflammatory processes resulting from, e.g., infection, trauma, toxic and/or ischemic injury (e.g., following cerebrovascular attack, myocardial infection).

In certain embodiments, the drug molecule is selected from inhibitors of mTORC1 and/or mTORC2, including but not limited to rapamycin and rapalogs (such as sirolimus, tacrolimus, everolimus, CCI-779, AP23573 and RAD001) and ATP-competitive mTOR inhibitors, including but not limited to INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458 (omipalisib), BEZ235, AZD2014 and XL765, including those described in US2011/0195966A1, US2008/0081809A1, U.S. Pat. No. 8,796,455B2, U.S. Pat. No. 8,492,381B2, U.S. Pat. No. 8,394,818B2, US2008/0234262A1 and related publications (e.g., Liang Y., et al., Structure-Activity Relationship Study of QL47: A Broad-Spectrum Antiviral Agent, Med. Chem. Lett. (2017) and Liu Q., et al., Discovery of 1-(4-(4-Propionylpiperazin-1-yl)-3-(trifluoromethyl)phenyl)-9-(quinolin-3-yl)benzo[h][1,6]naphthyridin-2(1H)-one as a Highly Potent, Selective Mammalian Target of Rapamycin (mTOR) Inhibitor for the Treatment of Cancer, J. Med. Chem. (2010)), and any derivatives, analogs, prodrug or salt forms thereof, each of which are herein incorporated by reference.

An unexpected finding disclosed herein is that certain mTOR inhibitors, particularly those that inhibit both mTORC1 and mTORC2, i.e., dual mTORC1/mTORC2 inhibitors, including ATP-competitive mTOR inhibitors, were more effective for inducing tolerance (e.g., reverse or suppress autoimmunity, allergies, transplant rejection and other inflammatory processes) than mTOR inhibitors that solely inhibit mTORC1. In some embodiments, the mTOR inhibitor has an mTORC1 inhibitory activity of <1 μM and/or an mTORC2 inhibitory activity of <1 μM. In some embodiments, the mTOR inhibitor has an mTORC1 inhibitory activity of <0.5 μM and/or an mTORC2 inhibitory activity of <0.5 μM. In some embodiments, the mTOR inhibitor has an mTORC1 inhibitory activity of <0.1 μM and/or an mTORC2 inhibitory activity of <0.1 μM. Inhibitory activity may be expressed as an IC₅₀ and be based on in vitro or cell-based assays. The inhibitory activity may be assessed directly, e.g., in a competitive binding assay or determined based on a functional read out, e.g., fluorescence reporter probe or cell growth.

In some embodiments comprising amphiphiles and drug molecules selected from mTOR inhibitors, the mTOR inhibitor is admixed with the amphiphile and associates with the hydrophobic core of micelles formed by amphiphiles in aqueous solution (e.g., aqueous buffer).

In other embodiments, the mTOR inhibitor is linked to the hydrophobic block (H) of the amphiphile through a linker. In some embodiments, the mTOR inhibitor linked to the hydrophobic block (H) of the amphiphile is an analog of everolimus, for example an analog of everolimus bearing a free amine for reaction to the H block either directly or via a linker, such as:

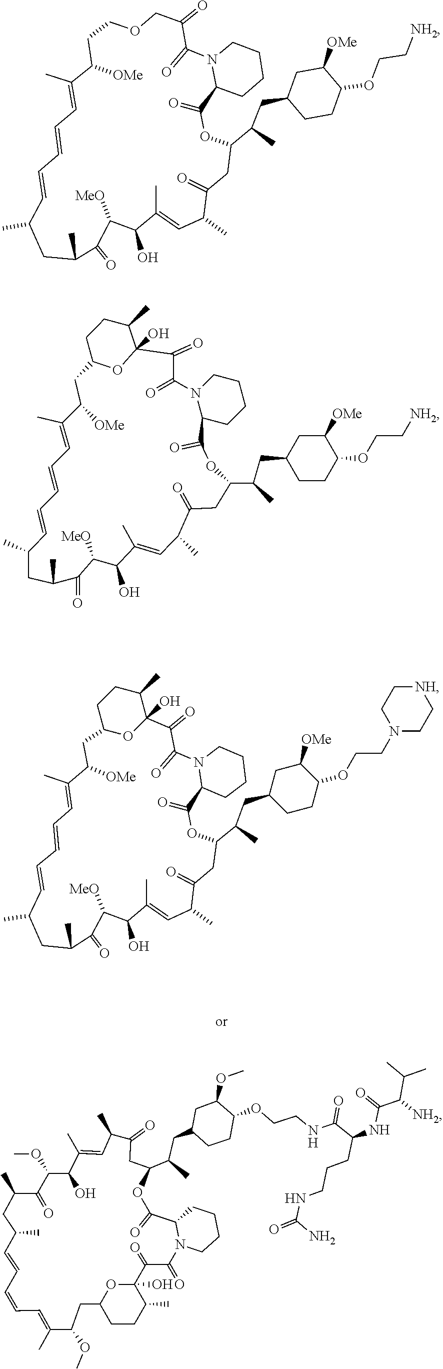

or, alternatively, the analog may comprise a carboxylic acid group for attaching to the H block either directly or via a linker, such as:

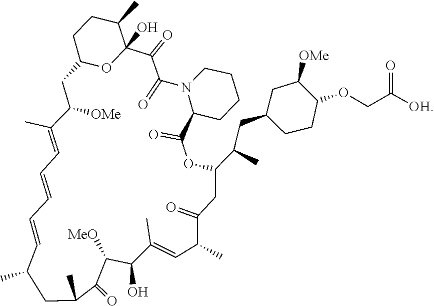

In some embodiments, the mTOR inhibitor linked to the hydrophobic block (H) of the amphiphile is Torin 2, or an analog of Torin 1 or Torin 2, for example, Torin 2, or an analog of Torin 1 or Torin 2, bearing a free amine for reaction to the H block either directly or via a linker, such as

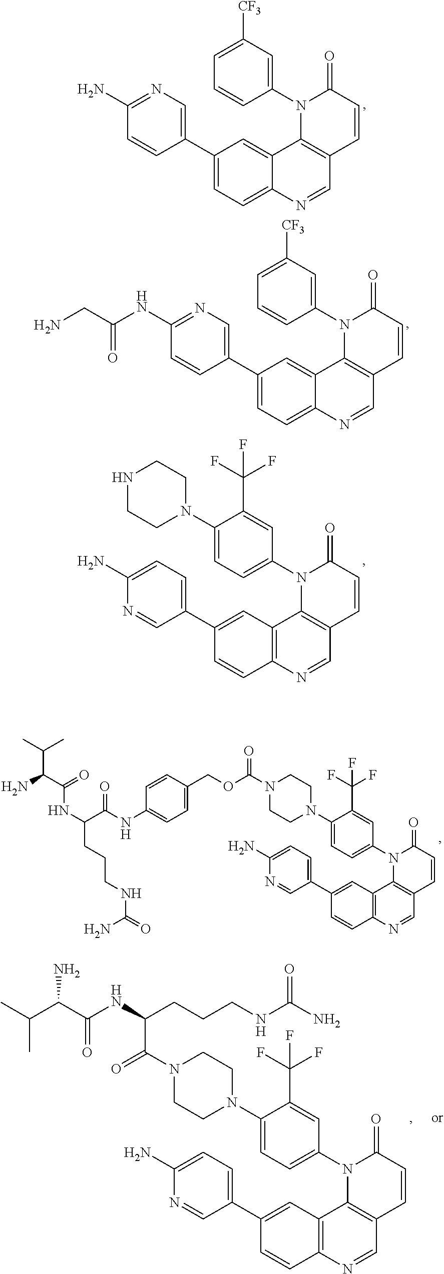

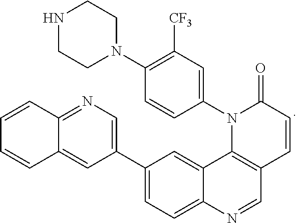 [SMILES: O=c1ccc2cnc3ccc(-c4cnc5ccccc5c4)cc3c2n1-c1ccc(N2CCNCC2)c(C(F)(F)F)c1]

In some embodiments, the amphiphile has the following structure

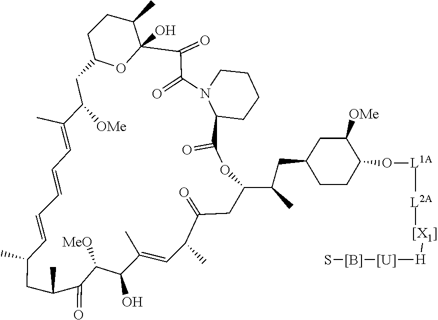

wherein S is a solubilizing block;
B is a spacer;
H is a hydrophobic block;
U is a linker;
L^{1A} is an alkylene linker;
L^{2A} is an amino, amido, ester, peptide, or nitrogen containing heterocyclyl linker;
X1 is a suitable linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof.

In some embodiments, the amphiphile has the following structure

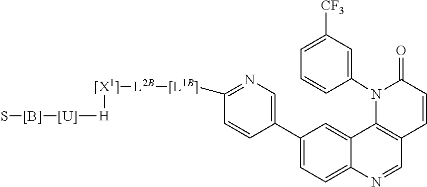

wherein S is a solubilizing block;
B is a spacer;
H is a hydrophobic block;
U is a linker;
L^{1B} is an alkylene—C(O)—NH— linker;
L^{2B} is an amino, amido, peptide, or nitrogen containing heterocyclyl linker;
X1 is a suitable linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof.

In some embodiments, the amphiphile has the following structure

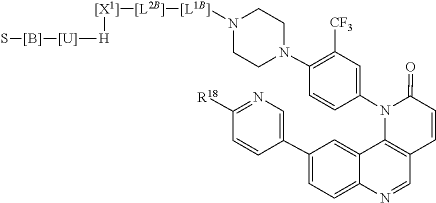

wherein S is a solubilizing block;
B is a spacer;
H is a hydrophobic block;
U is a linker;
L^{1B} is a

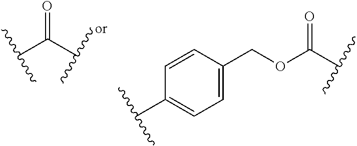

or linker;
L^{2B} is an amino, amido, or peptide linker;
R^{1B} is NH₂ or

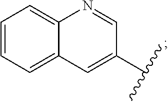 [SMILES: *c1cnc2ccccc2c1]

X1 is a suitable linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof.

### General Compositions of Amphiphile(s) and Drug Molecule(s)

The amphiphiles describes herein have general utility for various drug delivery applications, particularly delivery of immunostimulatory or chemotherapeutic drugs for treating cancer or viral infections, and for treating treating inflammatory conditions, such as allergies, autoimmunity, transplant rejection, and other inflammatory processes resulting from, e.g., infection, trauma, toxic and/or ischemic injury (e.g., following cerebrovascular attack, myocardial infection).

Amphiphiles with a solubilizing block (S) comprising a dendron amplifier (i.e., has dendron architecture) linked to a hydrophobic block (H) directly or indirectly via a spacer (B) and/or linker (U), wherein the solubilizing block is selected from solubilizing groups selected from sugar molecules and/or carboxylic acids, B is selected from hydrophilic polymers (e.g., PEG) and H is selected from poly(amino acids) of Formula I comprising aromatic groups, were found to be particularly effective for formulation of drug molecules, particularly hydrophobic drug molecules.

In certain preferred embodiments of amphiphile(s) and drug molecules, the amphiphile has the below structure, referred to as Formula V:

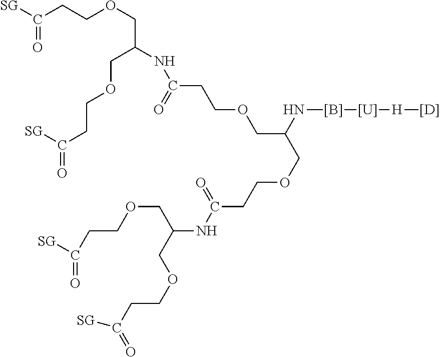

wherein SG is preferably selected from sugar molecules (e.g., mannose, N-acetyl galactosamine, or glucose) or carboxylic acids that are linked to S either directly or via a suitable linker X, or, more preferably, X5; B is a spacer preferably selected from hydrophilic polymers, more preferably PEG with between 4 and 36 monomeric units, most preferably 24 monomeric units; U is a linker; H is a hydrophobic block selected from poly(amino acids) of Formula I; the one or more drug molecules are typically selected from immunostimulants (e.g., PRR agonists, such as agonists of STING and TLR-7/8 of Formula IV), chemotherapeutic agents (e.g., anthracyclines) and immunosuppressants (such as mTOR inhibitors, e.g., everolimus, Torin 1 and Torin 2) that are either admixed with the amphiphile or attached directly or via a linker to H, wherein any unlinked (i.e., the drug molecules are not covalently linked to the amphiphile) drug molecules are admixed at a ratio of about 20:1 to about 1:20, or about 5:1 to about 1:5, though, more preferably about 1:2 to about 1:4 moles of amphiphile to moles of drug; D is a drug molecule; [ ] denotes that the group is optional; and - denotes that the two adjacent groups are directly attached to one another by a covalent bond or indirectly to one another via a suitable linker X.

In some embodiments the amphiphile of Formula V has the structure:

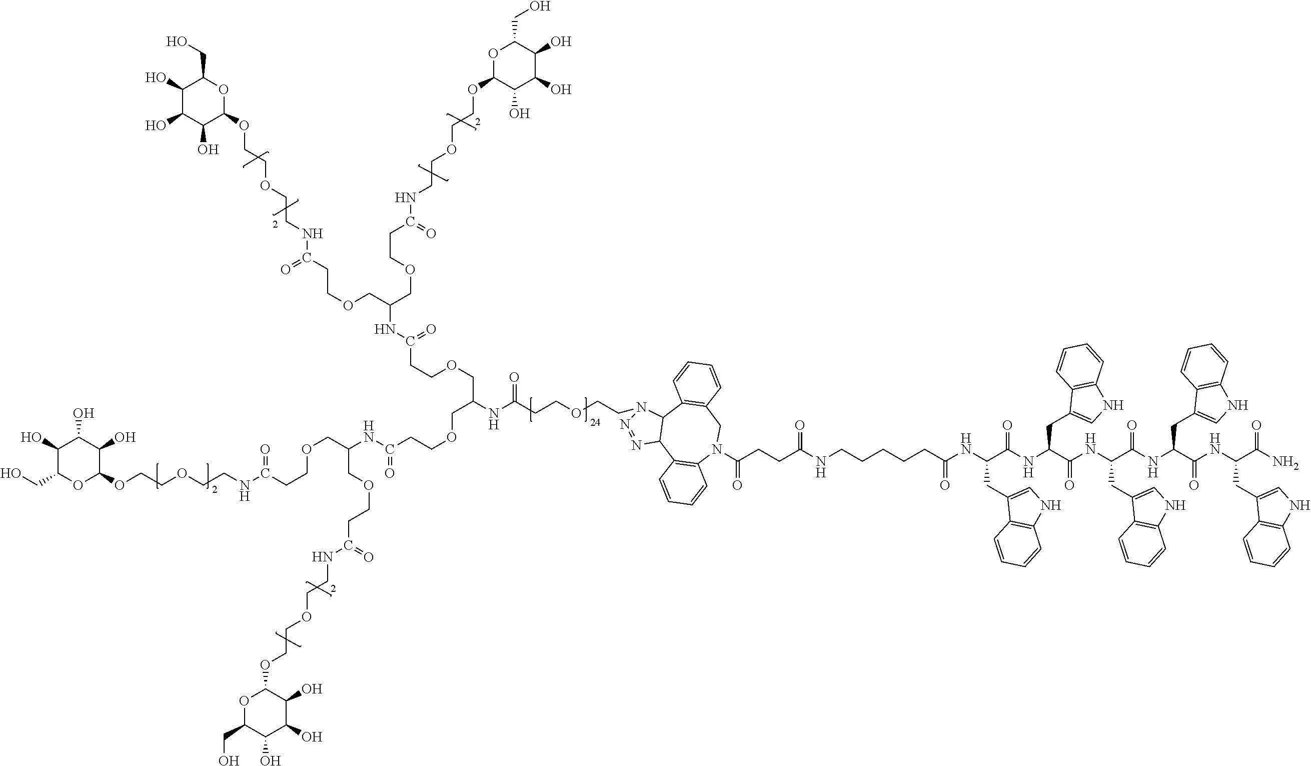

In still other embodiments, wherein the amphiphile comprises a solubilizing block (S) comprising a dendron amplifier (i.e., has dendron architecture) linked to a hydrophobic block (H) directly or indirectly via a spacer (B) and/or linker (U), wherein the solubilizing block is selected from solubilizing groups selected from sugar molecules and/or carboxylic acids, B is selected from hydrophilic polymers (e.g., PEG) and H is selected from poly(amino acids) of Formula I comprising aromatic groups, and the dendron amplifier is lysine, the structure is referred to as Formula VI and is shown below for clarity:

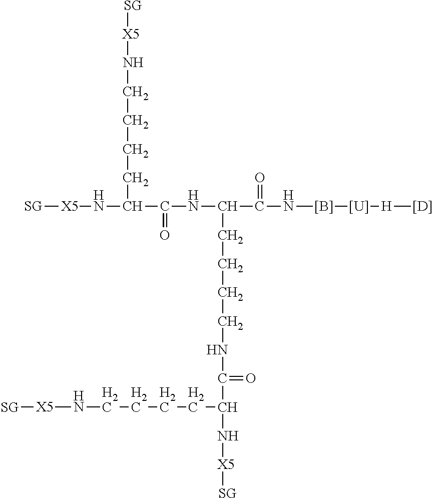

wherein SG is preferably selected from sugar molecules (e.g., mannose, N-acetyl galactosamine, or glucose) or carboxylic acids that are linked to S either directly or via a suitable linker X, or, more preferably, X5; B is a spacer preferably selected from hydrophilic polymers, more preferably PEG with between 4 and 36 monomeric units, most preferably 24 monomeric units; U is a linker; H is a hydrophobic block selected from poly(amino acids) of Formula I; the one or more drug molecules are typically selected from immunostimulants (e.g., PRR agonists, such as agonists of STING and TLR-7/8 of Formula IV), chemotherapeutic agents (e.g., anthracyclines) and immunosuppressants (such as mTOR inhibitors, e.g., everolimus, Torin 1 and Torin 2) that are either admixed with the amphiphile or attached directly or via a linker to H, wherein any unlinked (i.e., the drug molecules are not covalently linked to the amphiphile) drug molecules are admixed at a ratio of about 20:1 to about 1:20, or about 5:1 to about 1:5, though, more preferably about 1:2 to about 1:4 moles of amphiphile to moles of drug; D is a drug molecule; [ ] denotes that the group is optional; and - denotes that the two adjacent groups are directly attached to one another by a covalent bond or indirectly to one another via a suitable linker X.

In some embodiments, the amphiphile of Formula VI has the structure:

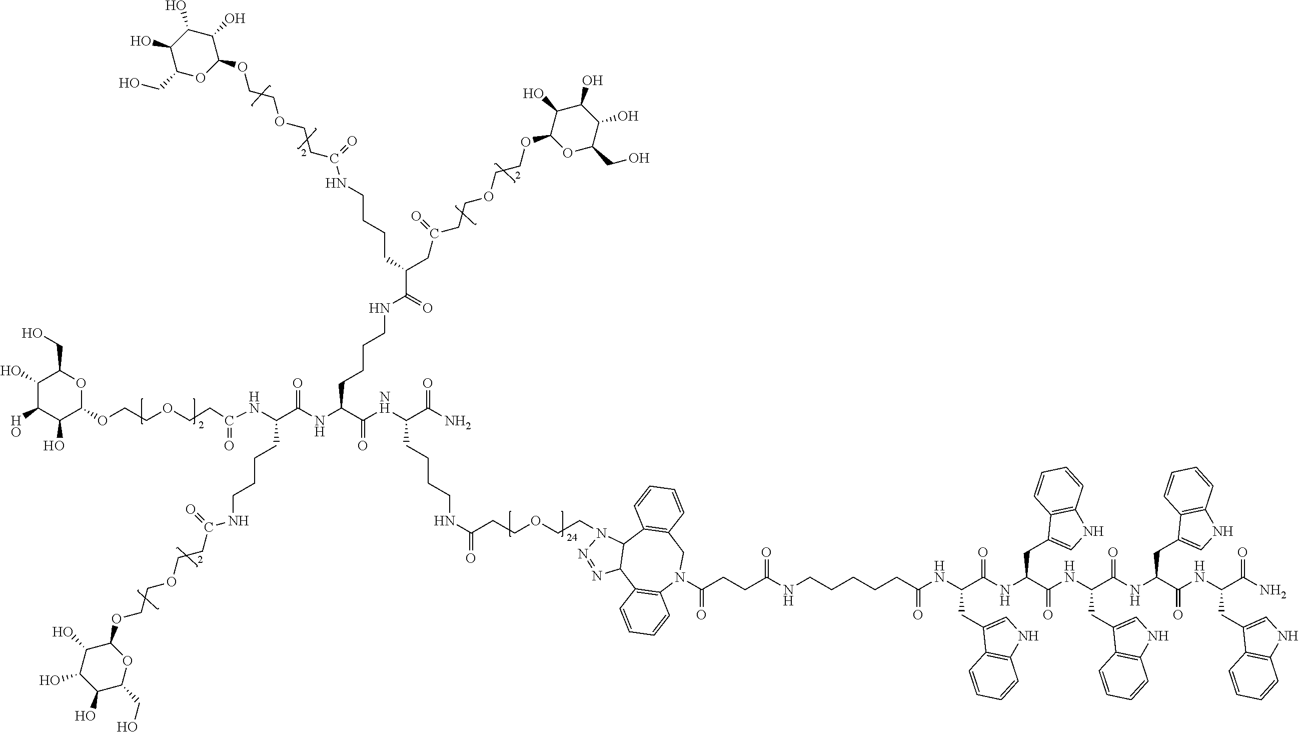

or, alternatively, in the above structure, the mannose is replaced with N-acetyl-galactoseamine (GalNAc), and optionally the triazole linker and/or Ahx linkers are absent.

In some embodiments, the amphiphile of Formula VI has the structure:

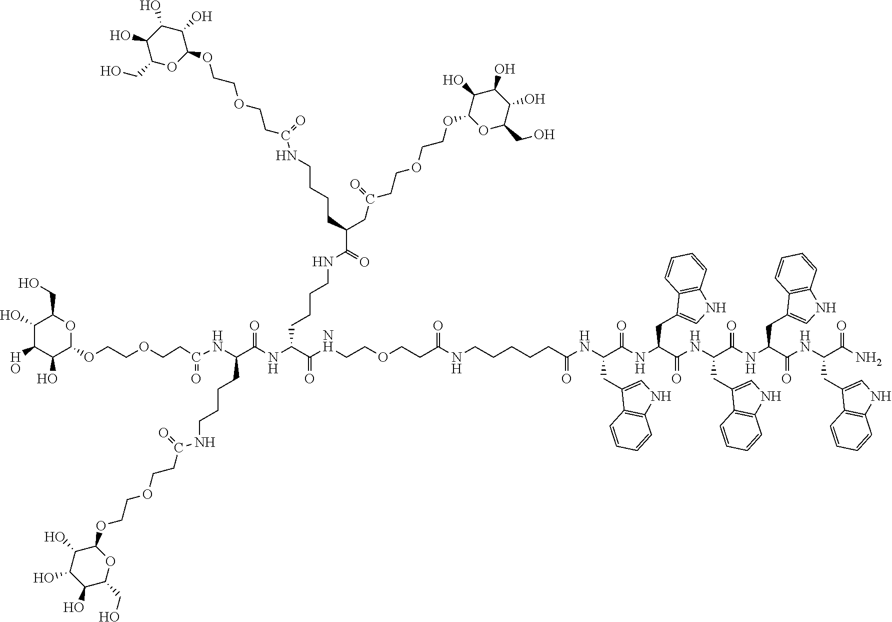 [SMILES: NC(=O)[C@H](Cc1c[nH]c2ccccc12)NC(=O)[C@H](Cc1c[nH]c2ccccc12)NC(=O)[C@H](Cc1c[nH]c2ccccc12)NC(=O)[C@H](Cc1c[nH]c2ccccc12)NC(=O)[C@H](Cc1c[nH]c2ccccc12)NC(=O)CCCCCNC(=O)CCOCCNC(=O)[C@@H](CCCCNC(=O)[C@@H](CCCCNC(=O)CCOCCO[C@H]1O[C@H](CO)[C@@H](O)[C@H](O)[C@@H]1O)CC(=O)CCOCCO[C@H]1O[C@H](CO)[C@@H](O)[C@H](O)[C@@H]1O)NC(=O)[C@@H](CCCCNC(=O)CCOCCO[C@H]1O[C@H](CO)[C@@H](O)[C@H](O)[C@@H]1O)NC(=O)CCOCCO[C@H]1O[C@H](CO)[C@@H](O)[C@H](O)[C@@H]1O]

or, alternatively, in the above structures, the mannose is replaced with N-acetyl-galactoseamine (GalNAc).

Particles comprising amphiphiles and at least one drug molecule that assembled into micelles of about 5 to 200 nm in diameter in aqueous solutions (e.g., aqueous buffers, such as PBS pH 7.4), particularly particles between about 5 to 50 nm diameter were found to be preferred for manufacturing (e.g., improved recovery following sterile filtration) and have improved biological activity as compared with particles >50 nm diameter. In some embodiments, the micelles are from about 5 nm to about 200 nm in diameter, such as between about 5 nm to about 100 nm in diameter and more preferably between about 5 nm to about 50 nm in diameter, such as 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm or about 50 nm.

The ratio of amphiphile to drug molecules (e.g., the at least one drug molecule) was also found to be a critical factor that impacted particle hydrodynamic behavior and biological activity. Historically, high amphiphile to drug molecule ratios, e.g., >10:1 ratio of amphiphile to drug molecule, are more readily achieved, whereas lower ratios of amphiphile to drug molecule (or, conversely, higher ratios of drug molecule to amphiphile) are often more challenging, particularly for large drug molecules (e.g., >500 daltons) or hydrophobic drug molecules (e.g., drug molecules with water solubility less than 1 mg/mL, or less such as less than 0.1 mg/mL). An unexpected finding disclosed herein is that preferred embodiments of amphiphiles, such as amphiphiles of Formula V and Formula VI, enabled low amphiphile to drug molecule molar ratios (or, conversely, high drug molecule to amphiphile molar ratios). Based on these findings, for the amphiphiles described herein, such as the amphiphiles of Formula V and Formula VI, the molar ratio of amphiphile to drug molecule is typically selected to maximize drug loading without negatively impacting stability of the particles formed by the amphiphiles.

Accordingly, a molar ratio of an amphiphile (e.g., an amphiphile for formula S-B-[U]-H) to drug molecule (D) of between about 20:1 to about 1:20, such as about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 and 1:20, was generally found to lead to compositions of stable nanoparticle micelles with desired size between about 5 nm to about 200 nm, such as 5 to about 50 nm. Though, wherein the drug molecule has molecular weight >500 daltons and/or is hydrophobic, the molar ratio of amphiphile to D is selected to be between about 5:1 to about 1:5, or more preferably between about 2:1 to about 1:4 were even more preferred and ratios of about about 1:1 to about 1:4 were most preferred.

pH-Responsive Amphiphiles for Improved Tumor Targeting

Amphiphiles comprising negatively charged functional groups and having net negative charge were found to have reduced uptake by antigen presenting cells as compared with amphiphiles with neutral or net positive charge. Such properties can be beneficial for reducing uptake by antigen presenting cells in blood, liver, and spleen and promoting increased uptake in other tissues, such as tumors. Moreover, certain amphiphiles that are negatively charged at pH 7.4 but undergo a change in properties in response to a change in pH (i.e., the amphiphiles are pH-responsive), e.g., a change in properties, such as charge and/or solubility, at pH less than pH 7.2, were found to reduce uptake by antigen presenting cells in blood, liver, and spleen and increase uptake in tumors. A non-limiting explanation is that the extracellular environment of the tumor is a lower pH than blood and the change in properties of the pH-responsive amphiphile at reduced pH in the tumor resulted in increased retention of the amphiphile within the tumor.

Therefore, in preferred embodiments of amphiphiles for tumor targeting the amphiphile comprises net negative charge at blood pH 7.4 but is pH-responsive and undergoes a change in properties at pH less than 7.4, or at pH less than 7.3, 7.2, 7.1, 7.0, 6.9, 6.8, 6.7, 6.6, 6.5, 6.4, 6.3, 6.2, or 6.1, preferably at a pH between about pH 6.0 to 7.3, such as pH 6.0, 6.1, 6.2, 6.3, 6.4, 6.5, 6.6, 6.7, 6.8, 6.9, 7.0, 7.1. 7.2 or 7.2.

Disclosed herein are structures of amphiphiles that exhibited pH-responsiveness and led to improved tumor targeting. An unexpected finding was that amphiphiles comprising solubilizing blocks with dendron amplifiers comprising solubilizing groups selected from carboxylic acids typically exhibited pH-responsiveness at pH values near physiologic pH 7.4. Therefore, in preferred embodiments, the amphiphile is pH-responsive and comprises a solubilizing block comprising carboxylic acid groups linked to a dendron amplifier.

In certain preferred embodiments, the pH-responsive amphiphile comprising a solubilizing block with dendron architecture and carboxylic acid solubilizing groups is:

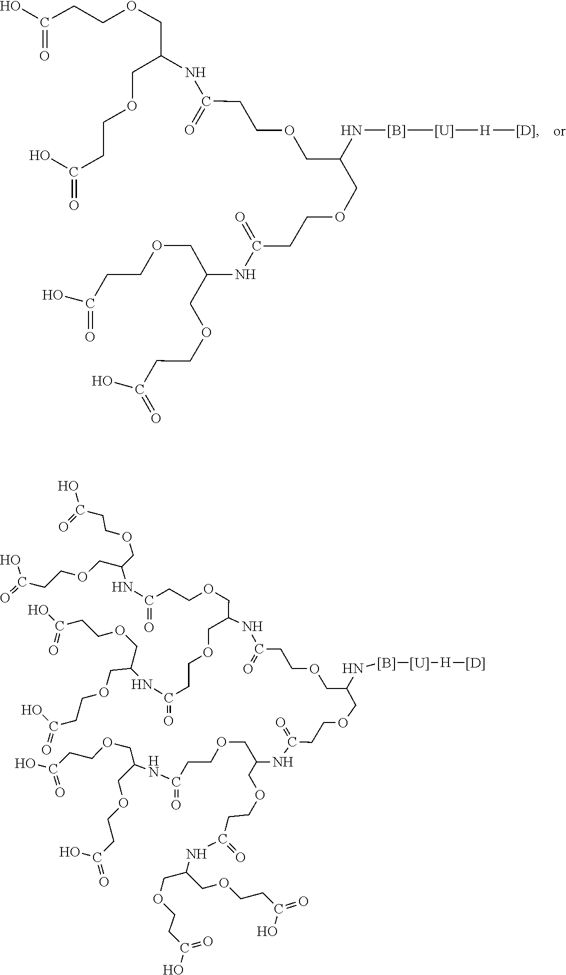

wherein B is a spacer; U is a linker; H is a hydrophobic block typically selected from poly(amino acids) of Formula I or H comprises a dendron amplifier and the terminal functional groups are linked directly or via a linker to hydrophobic groups or hydrophobic drug molecules that preferably comprise aryl or heteroaryl groups; D is a drug molecule; [ ] denotes that the group is optional; - denotes that the two adjacent groups are directly attached to one another by a covalent bond or indirectly to one another via a suitable linker X. In other embodiments, the terminal functional groups (FGt), i.e., the carboxylic acid groups, are linked to SG comprising carboxylic acids via X5, e.g., the carboxylic acids shown above may be reacted with glycine or beta-alanine to yield a structure with SG comprising alpha or beta carboxylic acids, respectively.

In certain preferred embodiments of the above structure, the amphiphile comprises a spacer (B) selected from PEG and the structure is:

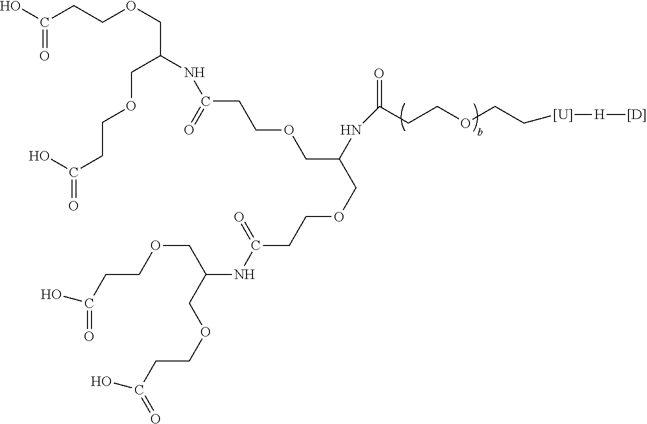

wherein b is an integer number of monomeric units comprising the spacer and is typically between 1 and 48, such as 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 22, 43, 44, 45, 46, 47 or 48 monomeric units, preferably between about 1 and 36 monomer units, or between about 4 and 36 monomeric units, most preferably 24 monomeric units.

In certain preferred embodiments of the above structure, the amphiphile comprises a hydrophobic block (H) selected from poly(amino acids) of Formula I and the structure becomes:

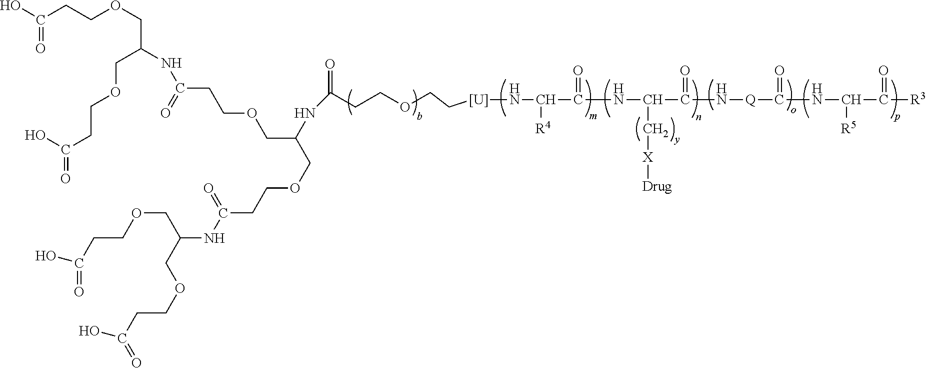

wherein the poly(amino acid) of Formula I optionally comprises monomers selected from hydrophobic amino acids (M), reactive amino acids (N), spacer amino acids (O), charged amino acids (P) and combinations thereof provided that at least monomer M or N are present; m, n, o and p denote that there are an integer of repeat units of monomers M, N, O and P, respectively, which may be distributed along the polymer in a specific or random order; R³ is typically selected from hydrogen, NH₂, NH₂—CH₃, NH₂—(CH₂)_{y5}CH₃, OH, or drug molecules (D) either linked directly or through X1; R⁴ is any hydrophobic group typically selected from aryl or heteroaryl groups; R⁵ is any group that comprises one or more functional groups that are charged in aqueous solutions or are pH-responsive and charged in aqueous solutions at certain pH ranges; Q is typically selected from any lower alkyl or heteroalkyl including but not limited to (CH₂)_{y6} and (CH₂CH₂O)_{y7}CH₂CH₂, where y6 is any integer from 1 to 6 and y7 is an integer typically selected from 1 to 4.

In certain preferred embodiments of the above structure, the amphiphile comprises a hydrophobic block (H) selected from poly(amino acids) of Formula I, wherein monomer M and N are present and monomers O and P are absent, and the structure is:

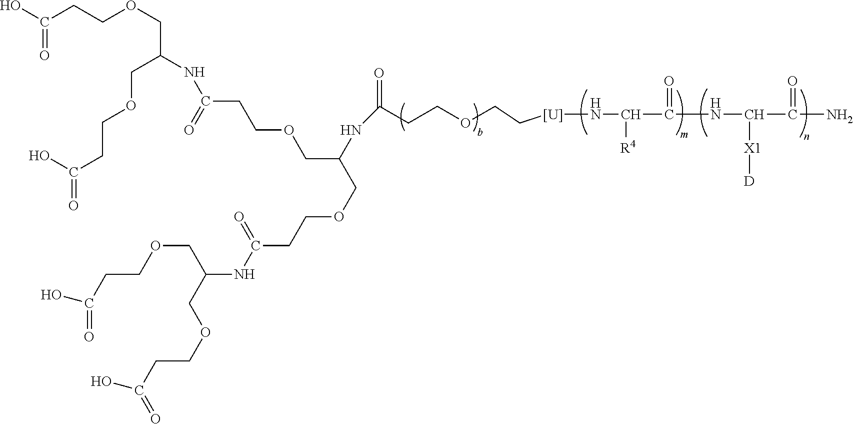

wherein the sum of m and n is an integer typically selected from 3 to 30, or alternatively m is absent and n is any integer typically selected from between 3 to 30 or n is absent and m is any integer typically selected from between 3 and 30.

In certain preferred embodiments, monomer M of the amphiphile of the above structure is absent, and the hydrophobic block (H) is linked to the spacer (B) through a Linker U comprising a triazole and the structure is:

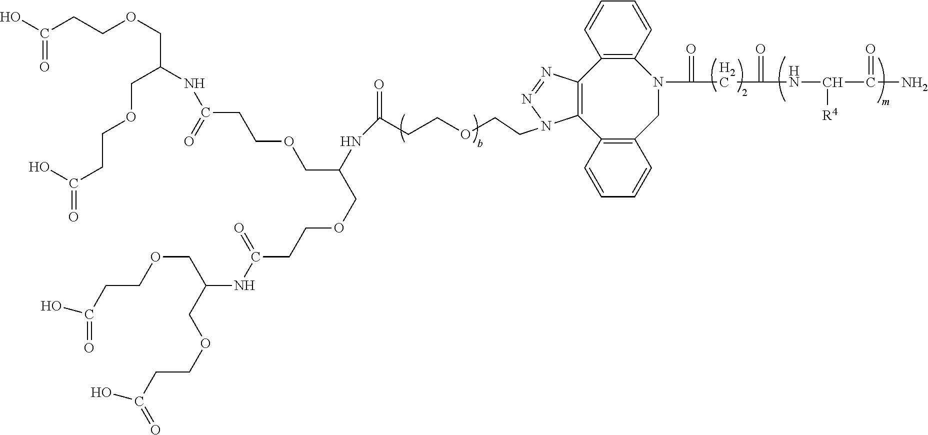

wherein m is an integer typically selected from between 3 and 30 and R⁴ is typically selected from aryl group or heteroaryl groups.

In certain preferred embodiments, monomer M of the amphiphile of the above structure is selected from tryptophan and the structure is:

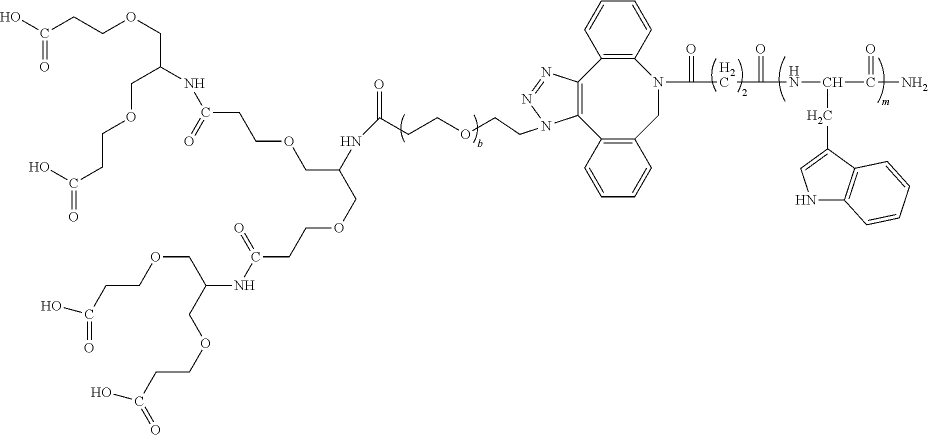

In certain preferred embodiments, the pH-responsive amphiphile comprises a solubilizing block that comprises a poly(amino acid)-based dendron amplifier, e.g., a lysine, ornithine, glutamic or aspartic acid based dendron amplifier. A non-limiting example of a pH-responsive amphiphile comprising a lysine-based dendron wherein the primary amines of the lysine, i.e., FGt, are substituted with succinic acid is shown here for clarity:

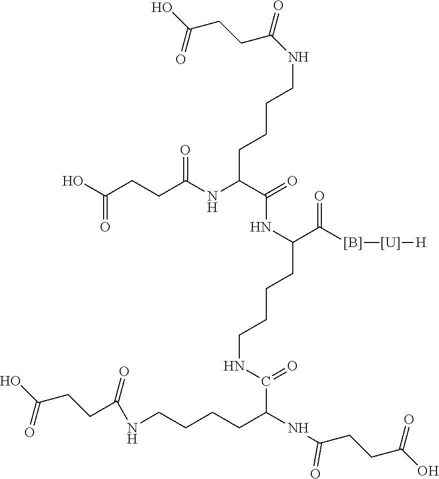

wherein B is a spacer; U is a linker; H is a hydrophobic block typically selected from poly(amino acids) of Formula I, or H is a dendron amplifier and the terminal functional groups are linked directly or via a linker to hydrophobic groups or hydrophobic drug molecules that preferably comprise aryl or heteroaryl groups; D is a drug molecule; [ ] denotes that the group is optional; - denotes that the two adjacent groups are directly attached to one another by a covalent bond or indirectly to one another via a suitable linker X. In other embodiments, the terminal functional groups (FGt), i.e., the amine groups, are linked malonic acid, glutaric acid or adipic acid.

### Nanomedicines Comprising a First Amphiphile and a Second Amphiphile

Preferred embodiments of nanomedicines disclosed herein comprise a first amphiphile comprising a solubilizing block with dendron architecture, wherein the solubilizing groups (SG) of the first amphiphile are typically selected from amines, sugar molecules or carboxylic acids.

In certain preferred embodiments of nanomedicines for cancer treatment, the first amphiphile is pH-responsive and comprises a solubilizing block with dendron architecture and solubilizing groups (SG) selected from carboxylic acids. In certain other preferred embodiments of nanomedicines for cancer treatment, the first amphiphile comprises a solubilizing block with dendron architecture and solubilizing groups (SG) selected from sugar molecules, preferably sugar molecules that bind to receptors on APCs, including certain C-type lectin receptors, such as the mannose receptor and DC-SIGN.

An unexpected finding disclosed herein is that tumor targeting and efficacy of nanomedicines for cancer treatment, particularly nanomedicines for cancer treatment administered by the intravenous route, could be improved by the addition of a second amphiphile. Specifically, for nanomedicines comprising a first amphiphile with a solubilizing block comprising a dendron amplifier and solubilizing groups selected from carboxylic acids or sugar molecules, addition of a second amphiphile with a solubilizing block comprising a hydrophilic polymer and having linear architecture was found to improve tumor uptake. A non-limiting explanation is that the second amphiphile shields the nanomedicine in the blood and reduces clearance by APCs in liver and spleen.

Therefore, in preferred embodiments of nanomedicines for cancer treatment, the nanomedicine comprises a first amphiphile and a second amphiphile wherein the first amphiphile is pH-responsive and comprises a solubilizing block with dendron architecture and solubilizing groups (SG) selected from carboxylic acids, and the second amphiphile comprises a solubilizing block further comprising a hydrophilic polymer and having linear architecture. In certain other preferred embodiments of nanomedicines for cancer treatment, the nanomedicine comprises a first amphiphile and a second amphiphile wherein the first amphiphile comprises a solubilizing block with dendron architecture and solubilizing groups (SG) selected from sugar molecules, such as mannose, and the second amphiphile comprises a solubilizing block further comprising a hydrophilic polymer and having linear architecture.

Several properties, including the length and monomer composition, of the hydrophilic polymer based solubilizing block of the second amphiphile were found to impact the capacity of the nanomedicine to target tumor tissue. Accordingly, hydrophilic polymers selected from neutral hydrophilic polymers comprising monomers selected from PEG, HPMA and HEMA, as well as zwitterionic betaine monomers, such as phosphorylcholine methacrylate (PCMA), sulfobetaine methacrylate (SBMA), carboxybetaine methacrylate (CBMA-2), carboxybetaine acrylamide (CBAA-3) and carboxybetaine methacrylamide (CBMAA-3) and the like, were found to be preferred for use as the solubilizing block of the second amphiphile. In addition to the monomer composition, the length of the hydrophilic polymer-based solubilizing block of the second amphiphile also required optimization to ensure shielding of the nanomedicine from APCs in the blood without hindering activity of the associated drug molecule. Accordingly, the results disclosed herein shown that as solubilizing blocks of the second amphiphile, hydrophilic polymers with between about 24 to 300 monomer units, or more preferably about 48 to about 200 monomer units, are optimal for tumor targeting.

An additional consideration is the interplay between the molar ratio of the first amphiphile and second amphiphile and the optimal length of the hydrophilic polymer-based solubilizing block of the second amphiphile. The optimal ratio of first and second amphiphile for a given length of the hydrophilic polymer-based solubilizing block of the second amphiphile is provided herein, wherein:

For a hydrophilic polymer-based solubilizing block of the second amphiphile comprising less than 48 monomer units, the ratio of first and second amphiphile should be selected from between about 4:1 or less, such as between 4:1 to 1:4, or more preferably about 2:1 to about 1:2; for a hydrophilic polymer-based solubilizing block of the second amphiphile comprising between about 48 monomer units and about 200 monomer units, the ratio of first and second amphiphile should be selected from between about 16:1 to about 2:1, such as between about 12:1 to about 6:1, or, more preferably about 9:1; and, for a hydrophilic polymer-based solubilizing block of the second amphiphile comprising greater than 200 monomer units, the ratio of first and second amphiphile is typically greater than about 10:1, such as about 12:1 or higher.

### Linker X6

Nanomedicines comprising particles further comprising a first amphiphile and optionally a second amphiphile are designed to shield drug molecules during transit through the body, but the solubilizing blocks of the first and/or second amphiphile can hinder drug molecule activity after the nanomedicine reaches its target tissue, such as tumors. Therefore, a linker X6, which is a specific linker used to link solubilizing blocks to hydrophobic blocks either directly or via a spacer (B) and/or linker U, was developed by the inventors of the present disclosure to enable controlled shedding of the solubilizing block (S).

In certain preferred embodiments of nanomedicines that comprise a second amphiphile, the second amphiphile comprises a solubilizing block (S) that is linked to X6, which is linked to a hydrophobic block (H) either directly or via a spacer (B) and/or linker U, and the X6 comprises a degradable bond preferably selected from a pH-sensitive bond or enzyme degradable bond. In certain preferred embodiments, X6 comprises an enzyme degradable peptide that is between about 1 to 8 amino acids in length, preferably between about 2 to 6 amino acids in length. In other preferred embodiments, X6 comprises a pH sensitive bond typically selected from hydrazones, silyl ethers and ketals.

### Compositions for Treating Cancer and Viral Infections

In certain compositions of amphiphiles and drug molecules for treating cancer or viral infections, the amphiphile is selected from amphiphiles for Formula V, wherein SG is preferably selected from mannose linked to the dendron via an X5 preferably selected from a short carbon or PEG linker, B is preferably selected from a hydrophilic polymer, and H is preferably selected from a poly(amino acid) of formula I comprising the monomers m and n. In such an example, wherein X5 is CH₂(CH₂OCH₂)₂CH₂—NH- and B is selected from PEG24 (i.e., a PEG with 24 monomer units), the structure is:

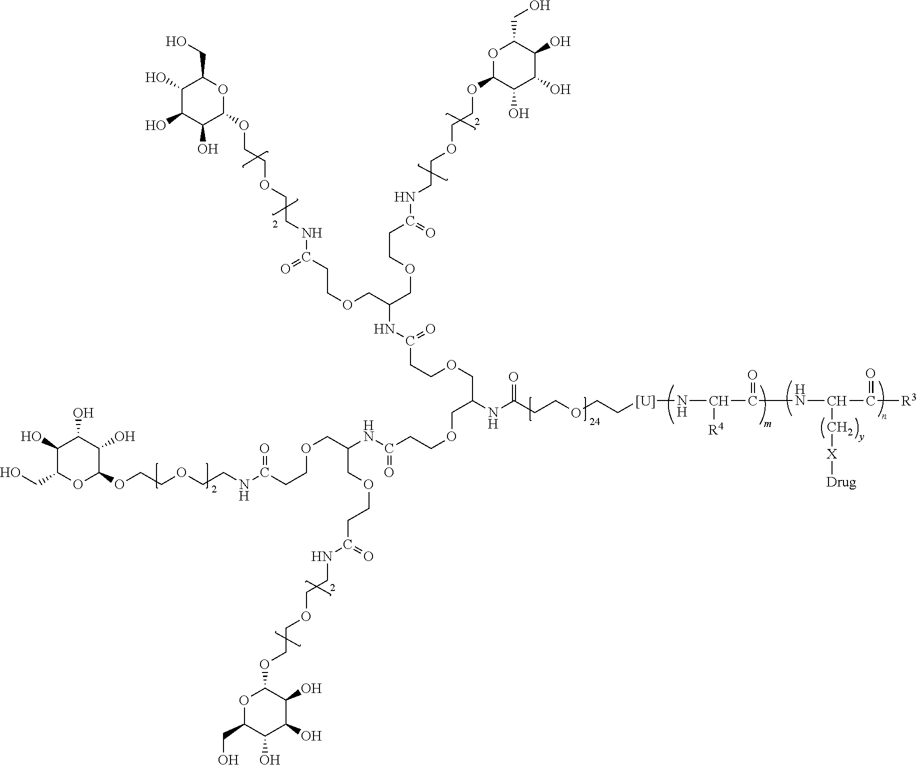

Wherein, the drug molecule is preferably selected from chemotherapeutic drugs (e.g., anthracyclines) or immunostimulants, such as PRR agonists, or more preferably agonists of TLR-7/8 and STING; the sum of m and n is typically between 3 and 30 and R³ is typically NH₂.

In a non-limiting example, wherein in the above structure the monomer M is selected from tryptophan and the drug molecule is an imidazoquinoline of Formula IV, the structure is:

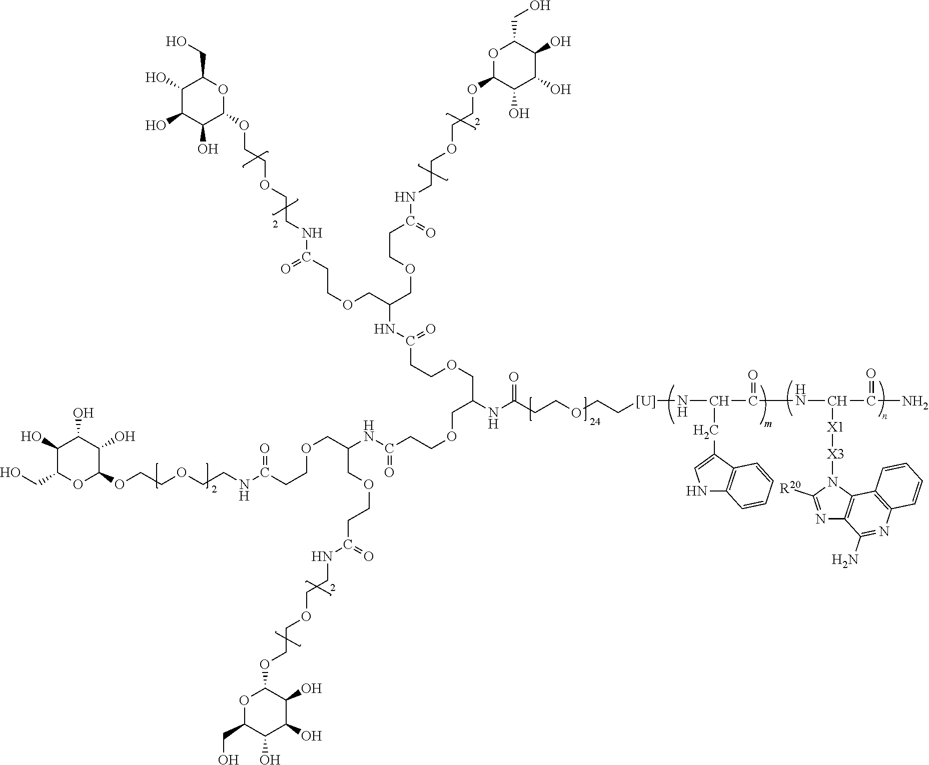

wherein, the sum of m and n is typically between 3 and 30, X1 is typically —(CH₂)_{y}—C(O)— and X3 is typically NH—(CH₂)_{y}— or NH—(C₆H₄)—CH₂— and R²⁰ is a short alkyl (such as CH₃—(CH₂)_{y}—) or ether, when y is an integer between 1 and 6; and, wherein U is present, U is typically comprises an amide or a triazole, such as a triazole resulting from the reaction between azide and DBCO.

In some embodiments the structure is:

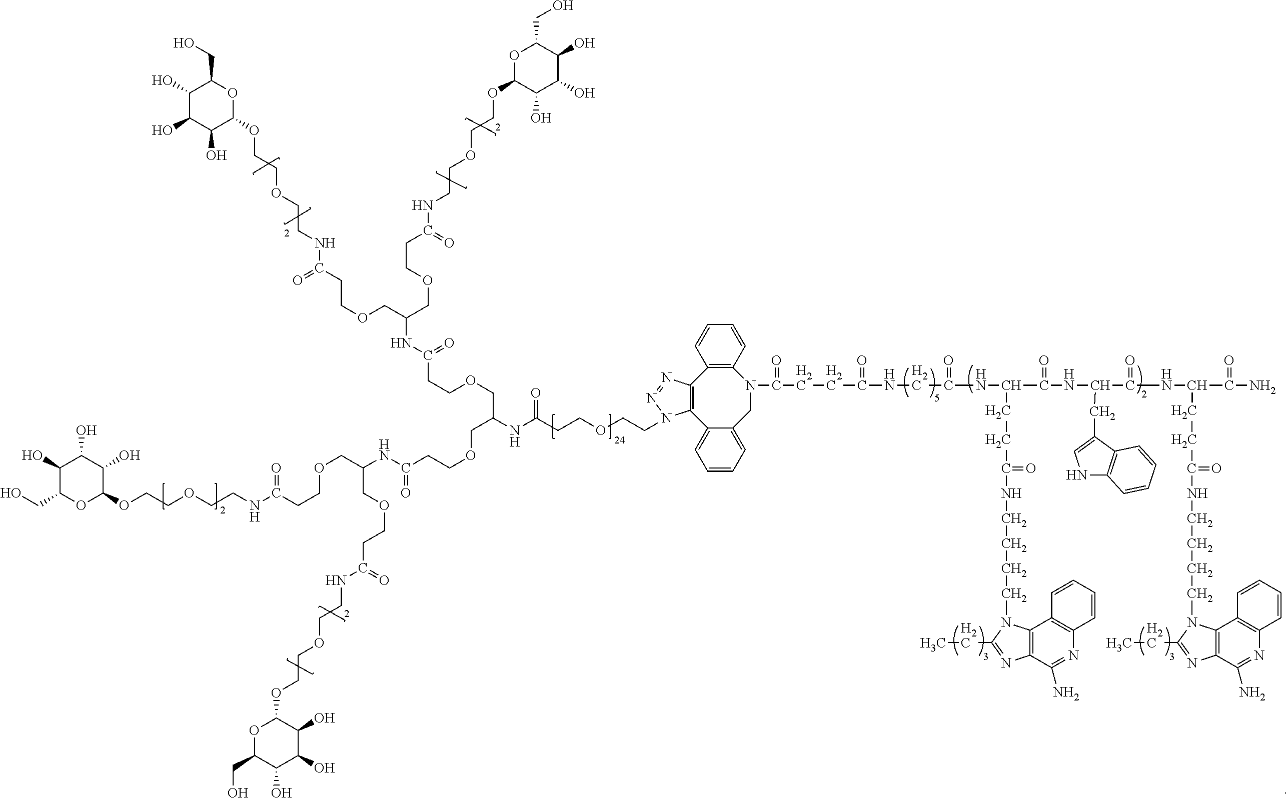

In certain embodiments of amphiphiles and drug molecules for treating cancer or viral infections, the amphiphile is selected from amphiphiles for Formula VI, wherein SG is preferably selected from mannose linked to the dendron via a X5 preferably selected from a short carbon or PEG linker, B is preferably selected from a hydrophilic polymer, and H is preferably selected from a poly(amino acid) of formula I comprising the monomers m and n. In certain preferred embodiments the structure is:

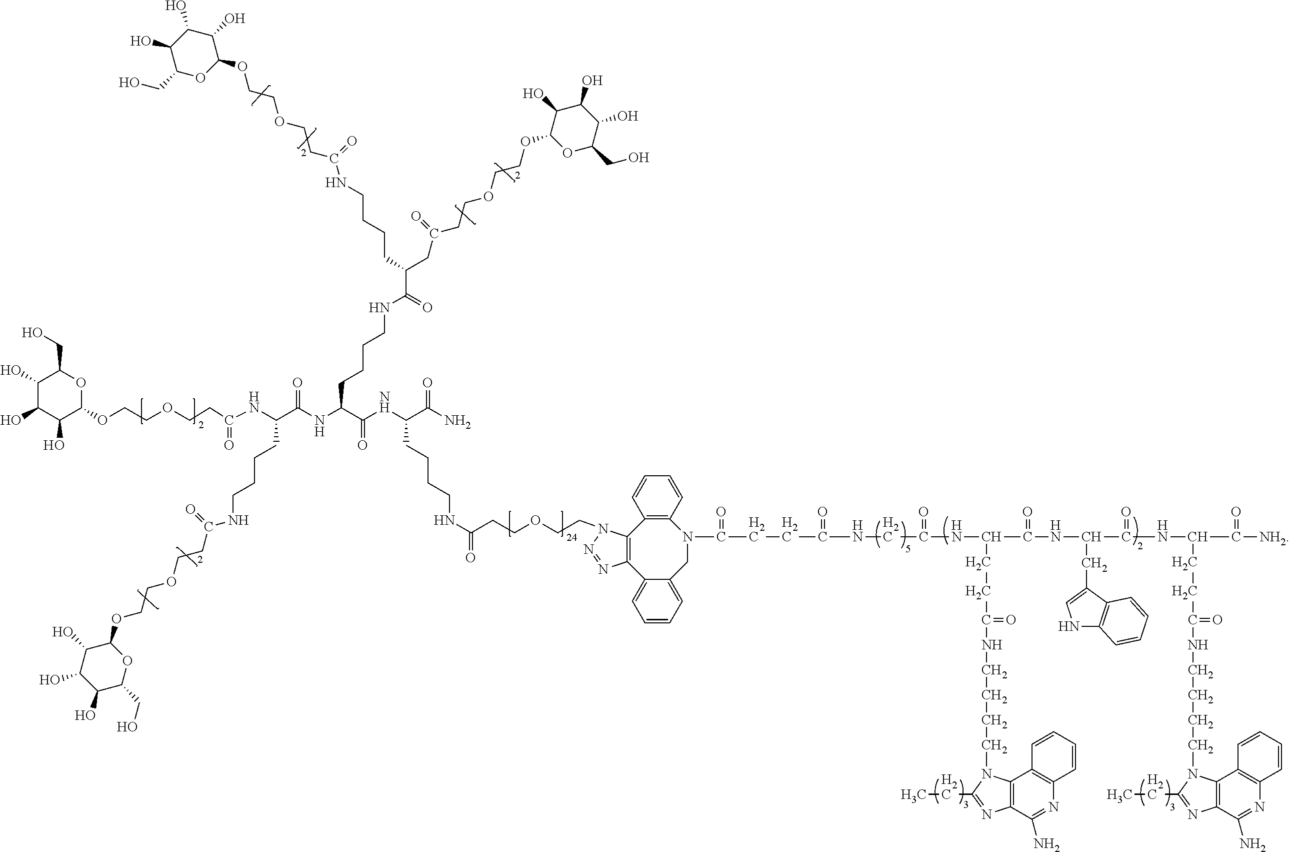

In certain other embodiments the structure is:

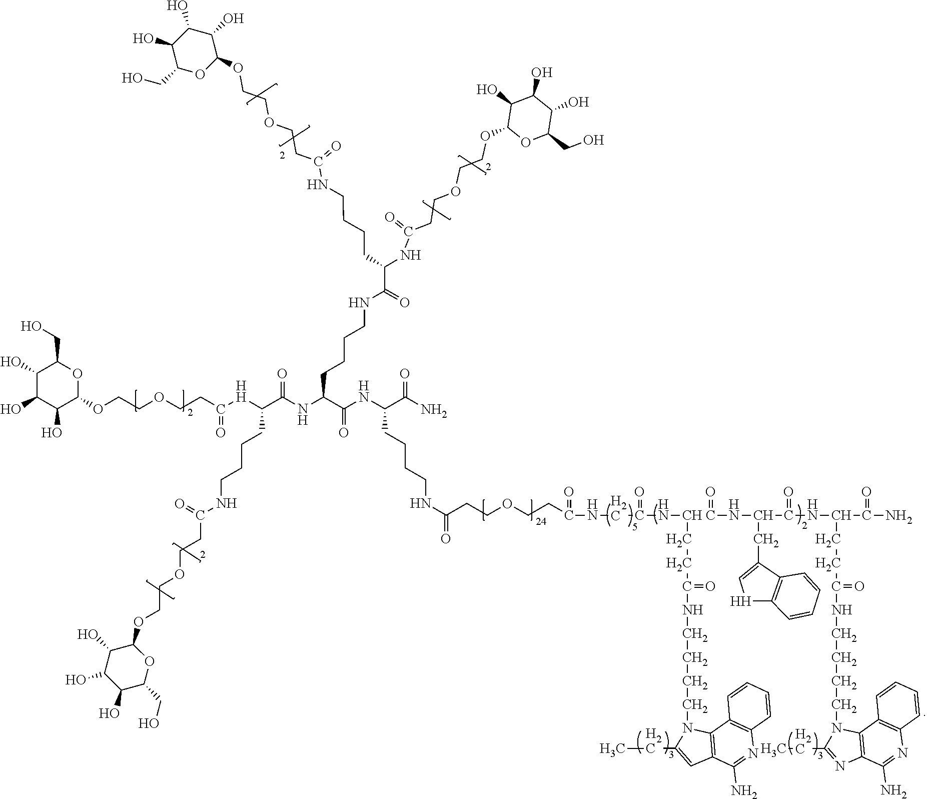

In certain preferred embodiments for treating viral infections of the liver, e.g., Hepatitis B, Hepatitis C, or cancer of the liver, the mannose residues shown in the above structures are replaced with N-acetylgalactosamine (GalNAc).

### Drug Molecules to Prevent ABC

Accelerated blood clearance (ABC), which can be caused by anti-drug or anti-nanoparticle antibodies, can reduce the activity of drugs, particularly drugs packaged in nanoparticles, delivered by the intravenous route upon repeat dosing.

An unexpected finding disclosed herein is that anti-drug antibodies as well as ABC can be mitigated by including certain immunomodulatory drugs in nanomedicines comprising amphiphiles. For instance, for nanomedicines used for cancer treatment that comprise at least one amphiphile and at least one drug molecule selected from immunostimulants and/or chemotherapeutics, such as an immunostimulant selected from agonists of TLR-7, TLR-8, TLR-7/8 or STING, it was observed that addition of another drug molecule that inhibits mTORC1, such as rapamycin, prevents unwanted anti-drug and/or anti-nanoparticle antibody responses and ABC without adversely affecting the efficacy of treatment. Therefore, in certain preferred embodiments of nanomedicines for cancer treatment, the nanomedicine comprises an immunostimulant and/or chemotherapeutic drug and an mTORC1 inhibitor, such as rapamycin.

### Compositions for Preventing, Inhibiting or Reducing Unwanted Immune Responses

In some embodiments of compositions of particles comprising amphiphiles further comprising a first drug molecule (D1) selected from inhibitors of mTORC1 and/or mTORC2, it was found that such compositions could be administered at a first time (Ti) to a subject and prevent or inhibit antibodies or antibodies and T cells from being generated against a second drug molecule (D2) or viral, DNA or RNA expression system (abbreviated “D2e”) administered at a second time (T2), which may the same or different from T1.

In some embodiments of compositions of particles comprising amphiphiles further comprising a first drug molecule (D1) selected from inhibitors of mTORC1 and/or mTORC2, it was found that such compositions could be administered at a first time (Ti) and administration route (I1) to a subject and prevent or inhibit antibodies or antibodies and T cells from being generated against a second drug molecule (D2) or viral, DNA or RNA expression system administered at a second time (T2), which may the same or different from Ti, and by a second administration route (I2), which may be the same for different from I1.

Disclosed herein are compositions of particles comprising amphiphiles further comprising a first drug molecule (D1) selected from inhibitors of mTORC1 and/or mTORC2 that were found to reduce or eliminate antibody responses against the second drug molecule (D2) or expression system (D2e).

In certain embodiments, the first drug molecule (D1) is selected from inhibitors of mTORC1 and/or mTORC2, including but not limited to rapamycin and rapalogs (such as sirolimus, tacrolimus, everolimus, CCI-779, AP23573 and RAD001) and ATP-competitive mTOR inhibitors, including but not limited to INK128 (MLN0128), AZD-8055, AZD-2016, KU-0063794, CC223, Torin-1, Torin-2, WYE354, WYE132, OSI-027, OXA-01, PI-103, NVP-BEZ235, GNE-493, GSK2126458 (omipalisib), BEZ235, AZD2014 and XL765, including those described in US2011/0195966A1, US2008/0081809A1, U.S. Pat. No. 8,796,455B2, U.S. Pat. No. 8,492,381B2, U.S. Pat. No. 8,394,818B2, US2008/0234262A1 and related publications (e.g., Liang Y., et al., Structure-Activity Relationship Study of QL47: A Broad-Spectrum Antiviral Agent, Med. Chem. Lett. (2017) and Liu Q., et al., Discovery of 1-(4-(4-Propionylpiperazin-1-yl)-3-(trifluoromethyl)phenyl)-9-(quinolin-3-yl)benzo[h][1,6]naphthyridin-2(1H)-one as a Highly Potent, Selective Mammalian Target of Rapamycin (mTOR) Inhibitor for the Treatment of Cancer, J. Med. Chem. (2010)), and any derivatives, analogs, prodrug, or salt forms thereof, each of which are herein incorporated by reference.

An unexpected finding disclosed herein is that certain mTOR inhibitors, particularly those that inhibit both mTORC1 and mTORC2, i.e., dual mTORC1/mTORC2 inhibitors, including ATP-competitive mTOR inhibitors, were more effective for preventing or inhibiting antibody responses and other unwanted immune responses against a second drug molecule or expression system (D2 or D2e) than mTOR inhibitors that solely inhibit mTORC1. In some embodiments, the mTOR inhibitor has an mTORC1 inhibitory activity of <1 μM and/or an mTORC2 inhibitory activity of <1 μM. In some embodiments, the mTOR inhibitor has an mTORC1 inhibitory activity of <0.5 μM and/or an mTORC2 inhibitory activity of <0.5 μM. In some embodiments, the mTOR inhibitor has an mTORC1 inhibitory activity of <0.1p M and/or an mTORC2 inhibitory activity of <0.1p M. Inhibitory activity may be expressed as an IC₅₀ and be based on in vitro or cell-based assays. The inhibitory activity may be assessed directly, e.g., in a competitive binding assay or determined based on a functional read out, e.g., fluorescence reporter probe or cell growth.

In certain embodiments the first drug molecule selected from inhibitors of mTORC1 and/or mTORC2 inhibits both mTORC1 and mTORC2 and is preferably selected from INK128 (MLN0128), AZD-8055, AZD-2016, KU-0063794, CC223, tricyclic benzonaphthyridinones (e.g., Torin-1, Torin-2, etc.), WYE354, WYE132, OSI-027, OXA-01. In particularly preferred embodiments, the first drug molecule is WYE132, AZD8055, Torin1, Torin 2, or INK128, or any derivative or salt form thereof.

Particles comprising amphiphiles and a first drug molecule (D1) that assembled into micelles of about 5 to 200 nm in diameter in aqueous solutions (e.g., aqueous buffers, such as PBS pH 7.4), particularly particles between about 5 to 50 nm diameter were found to be preferred for manufacturing (e.g., improved recovery following sterile filtration) and have improved biological activity as compared with particles >50 nm diameter. In some embodiments, the micelles are from about 5 nm to about 200 nm in diameter, such as between about 5 nm to about 100 nm in diameter and more preferably between about 5 nm to about 50 nm in diameter, such as 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm or about 50 nm.

The ratio of amphiphile to the first drug molecule (D1) was also found to be a critical factor that impacted particle hydrodynamic behavior and biological activity. For the amphiphiles described herein, such as the amphiphiles of Formula V and Formula VI, the molar ratio of amphiphile to first drug molecule (D1) is typically selected to maximize drug loading without negatively impacting stability of the particles formed by the amphiphiles. Accordingly, a molar ratio of an amphiphile (e.g., an amphiphile for formula S-B-[U]-H) to a first drug molecule (D1) of between about 20:1 to about 1:20, such as about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 and 1:20, was generally found to lead to compositions of stable nanoparticle micelles with desired size between about 5 nm to about 200 nm, such as 5 to about 50 nm. Though, when the first drug molecule (D1) has molecular weight >500 daltons and/or is hydrophobic, the molar ratio of amphiphile to first drug molecule (D1) is selected to be between about 5:1 to about 1:5, or more preferably between about 2:1 to about 1:4 were even more preferred and ratios of about about 1:1 to about 1:4 were most preferred.

For particles comprising a first drug molecule (D1) selected from inhibitors of mTORC1 and/or mTORC2 for reducing, inhibiting or eliminating antibody responses against a second drug molecule (D2) or expression system (D2e), the chemical composition of the amphiphile was found to strongly affect formation of (i) stable nanoparticle micelles of between about 5 nm to 200 nm, such as between about 5 nm to about 100 nm in diameter and more preferably between about 5 nm to about 50 nm in diameter; (ii) adequate drug loading; and (iii) optimized therapeutic index in vivo. Accordingly, amphiphiles comprising solubilizing blocks with dendron architecture each comprising between 2 to 32 solubilizing groups, more preferably between 4 and 8 solubilizing groups, selected from sugar molecules (e.g., mannose or GalNAc) and carboxylic acids, and the hydrophobic block is typically selected from fatty acids, lipids, cholesterol, hydrophobic polymers such as peptides of Formula I that are hydrophobic.

A non-limiting example of an amphiphile comprising a solubilizing block (S) with dendron architecture with 4 solubilizing groups, wherein the dendron is second generation, is provided below for clarity:

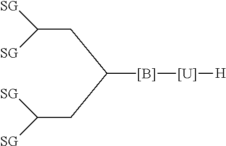

wherein, in preferred embodiments, the solubilizing groups are selected from sugar molecules (e.g., mannose) and carboxylic acids; the dendron comprises monomeric units comprising ethylene oxide or amino acids; the spacer (B) is absent or selected from hydrophilic polymers, such as PEG with between about 4 to about 24 monomeric units, preferably about 24 monomeric units; the Linker U is present or absent; and the hydrophobic block is typically selected from fatty acids, lipids, cholesterol or hydrophobic polymers, such as peptides of Formula I that are hydrophobic.

In the above structure, wherein the dendron is second generation and comprises monomeric units selected from FG1-CH(R′)₂, wherein FG1 (and the focal point) is NH₂, R¹ is (OCH₂CH₂)_{y}-FG2, y is 1 and FG2 (and FGt) is COOH, wherein the terminal functional group (FGt) carboxylic acids are linked via X5 to solubilizing groups selected from mannose, the structure is provided below for clarity:

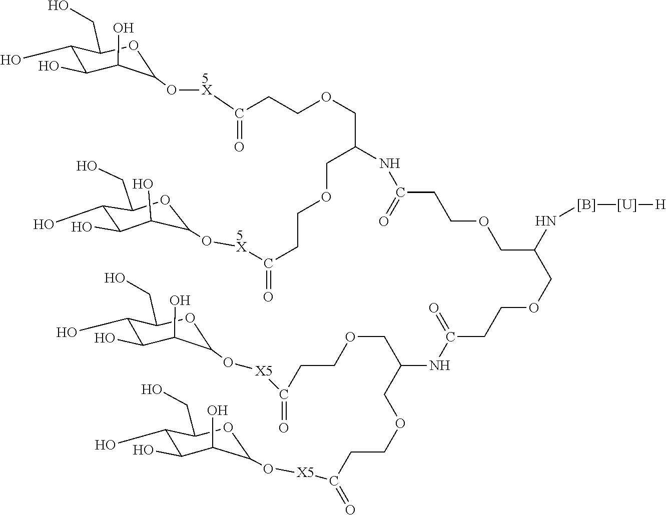

In the above structure, wherein X5 is —(CH₂CH₂O)ₜ—CH₂CH₂— and t=2, the structure becomes:

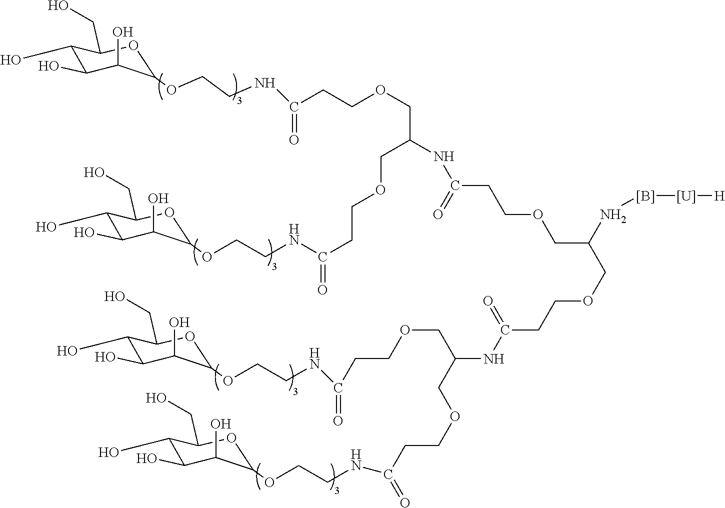

In the above structure, wherein the spacer is PEG and the hydrophobic block is a polymer of Formula I comprising tryptophan the structure is:

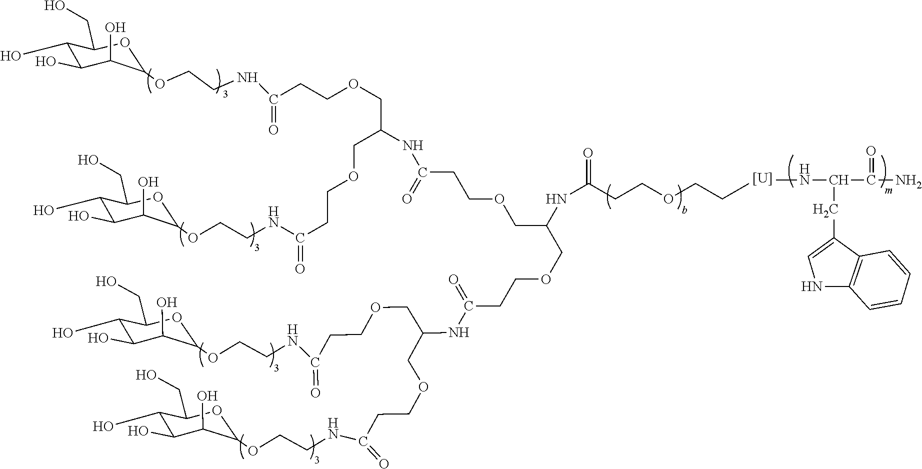

Wherein b is typically selected from 1 to about 36 monomeric units, preferably between about 4 to about 24 monomeric units, or more preferably about 24 monomeric units, and m is an integer typically selected from between about 3 to about 30 monomeric units, more preferably between about 5 and 10 monomeric units.

In the above structure, wherein the dendron is replaced with a second-generation lysine dendron with terminal succinate groups, the structure is:

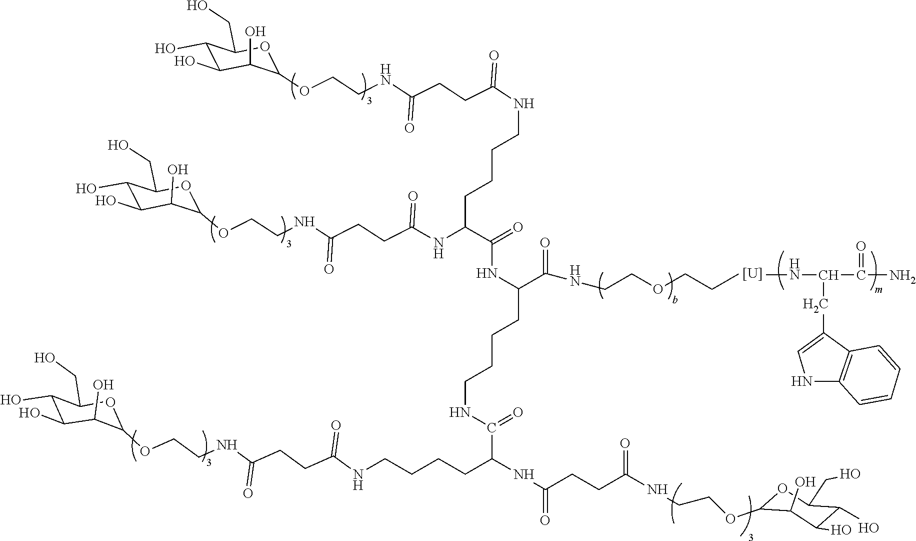

In some embodiments the amphiphile has the structure:

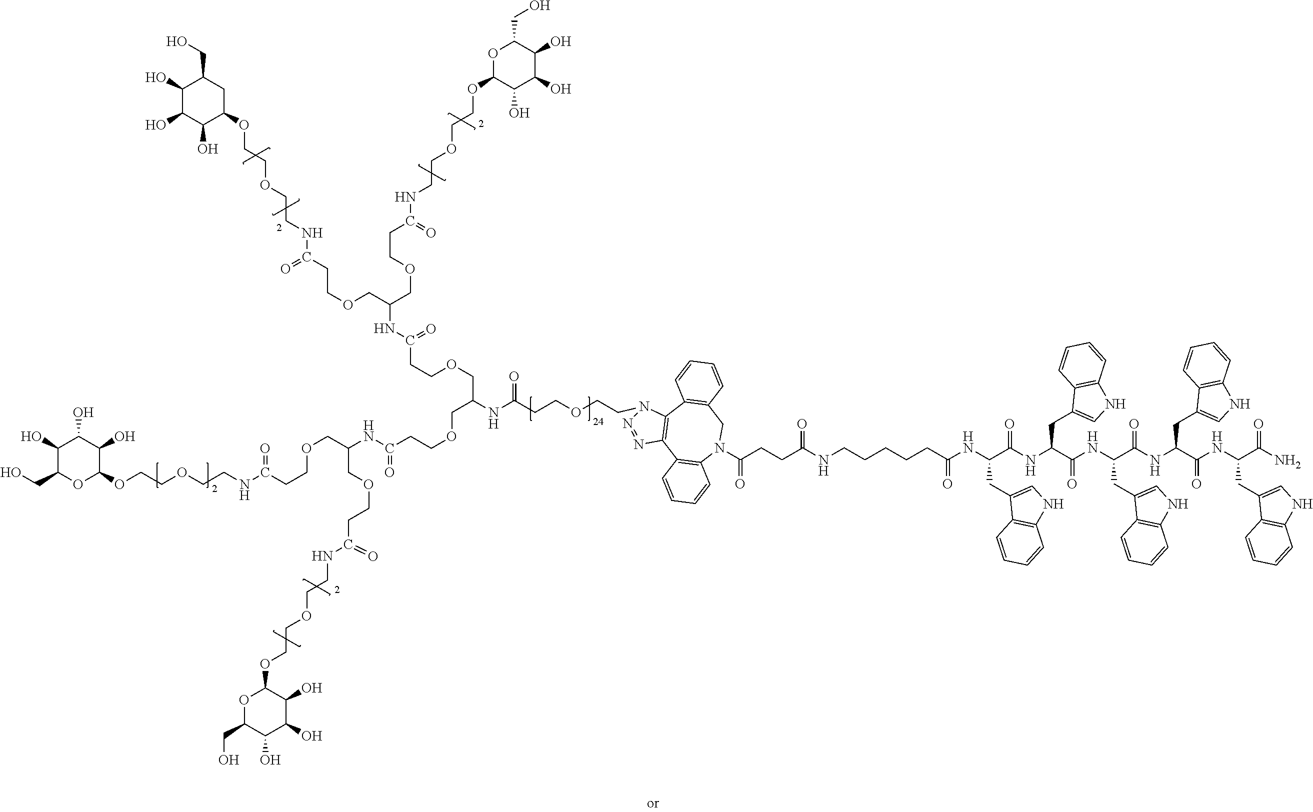

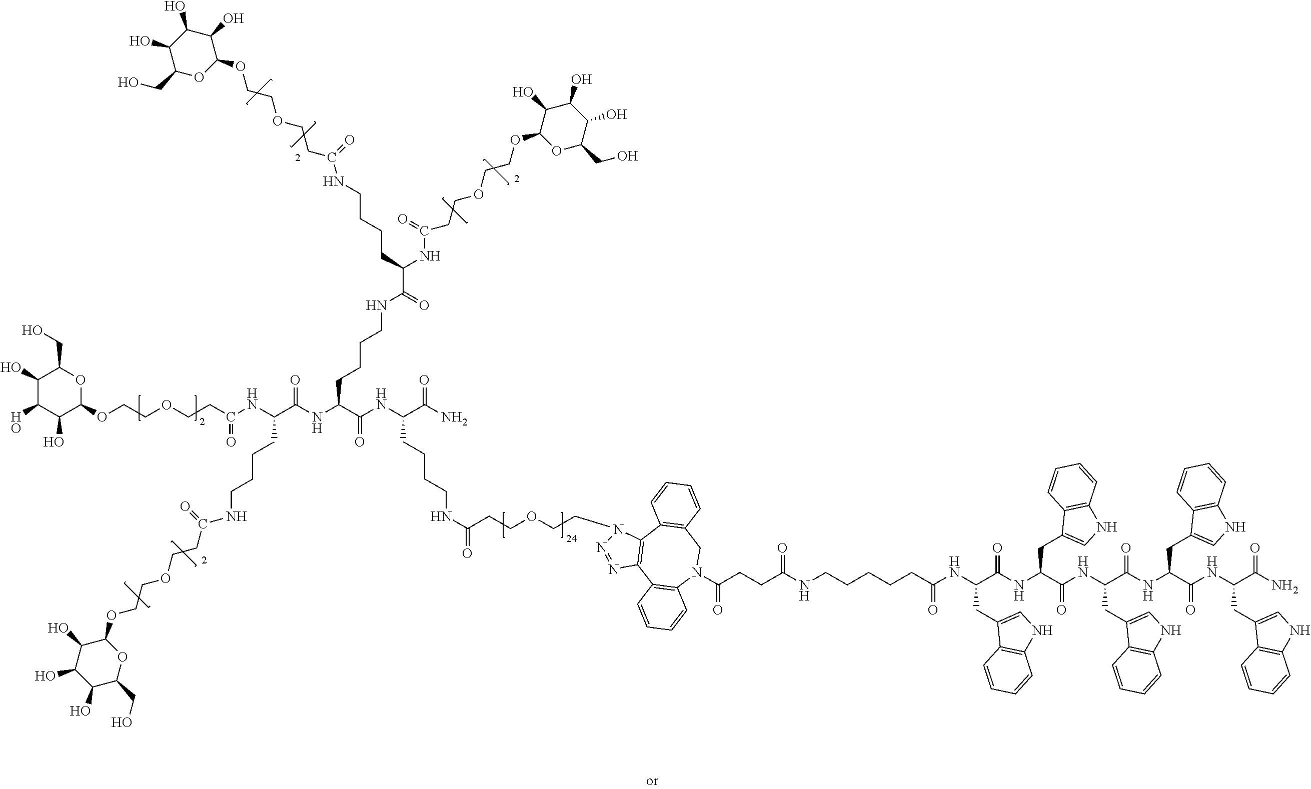

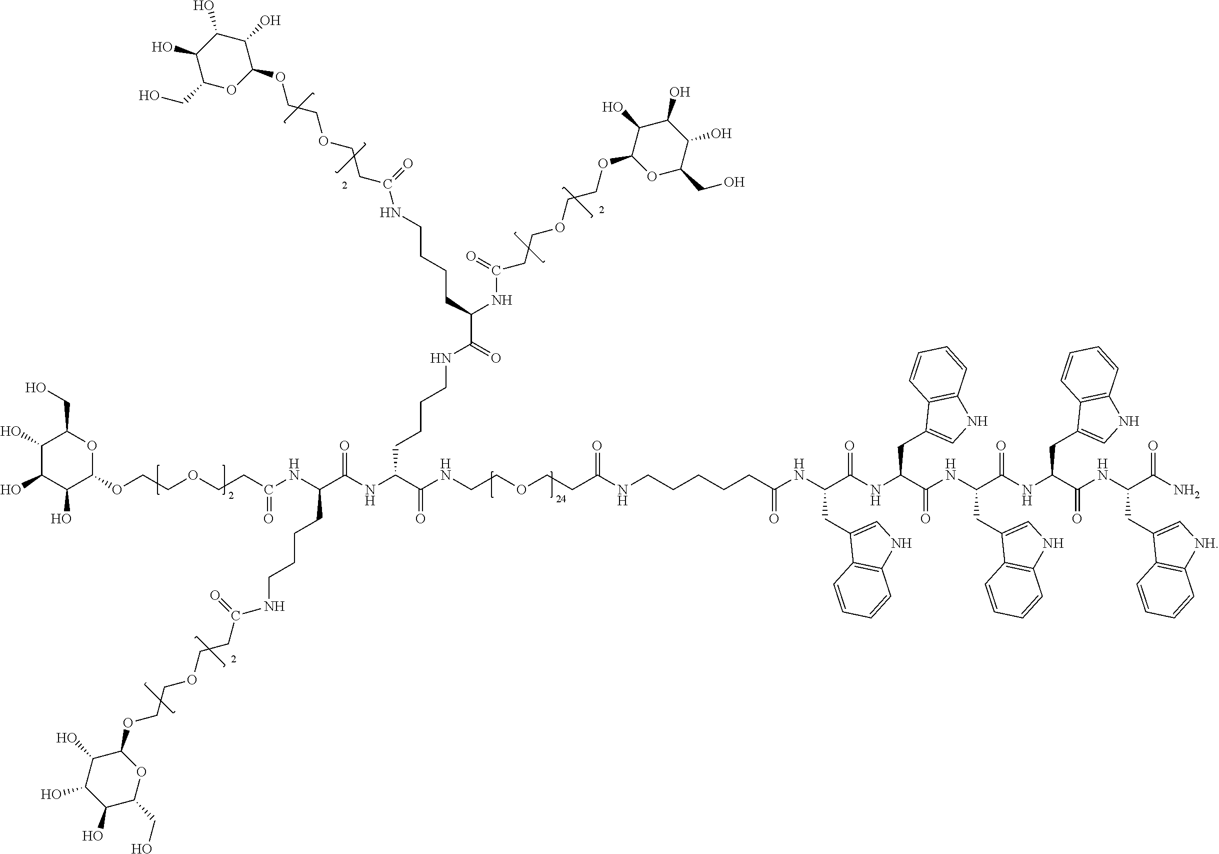

Such formulations were found to be effective for reducing, inhibiting or eliminating antibody responses against a broad variety of different drug molecules and viral, DNA and RNA expression systems that, when used in formulations, are referred to as D2 and D2e, respectively.

Without limiting the foregoing, particles comprising amphiphiles further comprising a first drug molecule (D1) selected from inhibitors of mTORC1 and/or mTORC2 are effective for reducing, inhibiting or preventing antibody responses against second drug molecules (D2) selected from any synthetic or recombinant peptide or protein-based drug molecules and any modifications or derivatives thereof, including but not limited to, any protein or peptide-based antigens, or Anti-TNFα (e.g., Etanercept, Adalimumab, Infliximab, Certolizumab pegol, Golimumab), anti-VEGF (e.g., Bevacizumab, Ranibizumab, Ranibizumab), anti-CD20 (e.g., Rituximab, Ofatumumab), anti-Her2 (e.g., Trastuzumab), anti-EGF-R (e.g., Cetuximab, Panitumumab), anti-α4/β1/7 integrin (e.g., Natalizumab), anti-CD3 (e.g., Muromonomab), Anti-GRPIIb/IIIa (e.g., Abciximab), Anti-CD20 and Anti-CD20 drug conjugates (e.g., Rituximab, Tositumomab, Ibritumomab tiuxetan), Anti-IL2R (e.g., Basiliximab, Daclizumab), anti-CD33 (e.g., Gemtuzumab), anti-CD52 (e.g., Alemtuzumab), anti-CD11 (e.g., Efalizumab), anti-IgE (e.g., Omalizumab), anti-C5 (e.g., Eculizumab), anti-IL-1b (e.g., Canakinumab), Anti-EPCAM (e.g., Catumaxomab), Anti-IL12/23 (e.g., Ustekinumab), Anti-IL6R (e.g., Toclizumab), Anti-RANK-L (e.g., Denosumab), Anti-BLys (e.g., Belimumab), Anti-B. anthrasis PA (e.g., Raxibacumab), Anti-CTLA-4 (e.g., Ipilimumab), anti-PD1 or anti-PDL1 (e.g., Nivolumab, Pembrolizumab, Atezolizumab) anti-CD30 (e.g., Brentuxmiab vedotin), anti-Factor IX and X (e.g., Emicizumab) anti-viral antibodies (e.g., Palivizumab), Insulin and modified insulin (e.g., Insulin, Insulin Glargine, Insulin Aspart, Rhu insulin, Insulin lispro, Insulin detemir, Humulin), EPO and modified EPO (e.g., Epoetin alfa, Darbepoetin alfa, Epoeticn beta), Pramintide, Exenatide, G-CSF and modified GCF (e.g., Filgrastim, Pegfilgrastim, Sargramostim), Interferons and modified interferons (e.g., Avonex, Rebif, Peginterferon alfa-2a, Interferon beta-1b), IL-2 and modified IL-2 (e.g., Denileukin difitox), IL-11 (e.g., Oprelvekin), growth hormone, modified growth hormones and growth hormone antagonists (e.g., Pegvisoman), IGF1 (e.g., Mecasermin), follicle-stimulating hormone (FSH), human chorionic gonadotropin, Luteinizing hormone (e.g., Lutropin-α), calcitonin (e.g., Salmon calcitonin), parathyroid hormone or parts of parathyroid hormone (e.g., Teriparatide), Clotting cascade factors such as Factor VIIa, Factor VIII (e.g., Octocog alfa, Eptacog alfa, Rec antihemophilic factor), Factor IX, Protein C, al-proteinase inhibitor, Antithrombin III (serine protease inhibitor, desmopressin, Botulinum toxins (e.g., Botulinum toxin type A, OnabotulinumtoxinA, Botulinum toxin type B), P-Glucocerebrosidase, Alglucosidase-α, Laronidase, Idursulfase, Galsulfase, Agalsidase-β, Lactase, Pancreatic enzymes (lipase, amylase and other proteases), Adenosine deaminase, Tissue plasminogen activator, Drotrecogin-α, Trypsin, Collagenase, Human deoxyribonuclease I, Hyaluronidase, Papain, L-Asparaginase, Rasburicase, Streptokinase; any small molecule drugs, particularly those that can form haptens and are prone to inducing antibodies such as penicillin and cephalosporin antibiotics and chemotherapeutic drugs; and blood products, cell-based products, nucleic acids (e.g., including siRNA) and protein extracts.

It was found that particles comprising amphiphiles further comprising a first drug molecule (D1) selected from inhibitors of mTORC1 and/or mTORC2 are particularly effective for reducing or preventing antibody responses against foreign proteins or peptides, i.e., proteins or peptides that are not endogenously produced by the subject. For example, patients who have enzyme deficiencies or clotting factor deficiencies may require enzyme replacement or receipt of clotting factors that are otherwise not produced endogenously, and such patients are prone to inducing unwanted immune responses against enzymes or clotting factors that make them resistant to such therapies. Additionally, patients receiving antibody therapies, particularly non-naturally occurring antibodies, such as fusion antibodies, antibody-drug conjugates, bi-specific antibodies, humanized antibodies, etc., that are otherwise not produced endogenously, often develop unwanted immune responses against such antibodies that render the antibodies ineffective. Therefore, preferred D2 are selected from proteins or peptides that are not produced endogenously by the subject, such as enzymes used in enzyme replacement therapy, clotting factors used in patients with clotting deficiencies (e.g., hemophiliacs) and non-naturally occurring antibodies, particularly such as fusion antibodies, antibody-drug conjugates, bi-specific antibodies, and humanized antibodies (e.g., humanized mouse or primate antibodies).

Without limiting the foregoing, particles comprising amphiphiles further comprising a first drug molecule (D1) selected from inhibitors of mTORC1 and/or mTORC2 are effective for reducing, inhibiting or preventing antibody responses against second drug molecules selected from expressions systems (D2e), including but not limited to adenoviruses (Ad), adeno associated viruses (AAV), rhabdoviruses, poxviruses (e.g., MVA), herpesviruses and lentiviruses as well as non-viral forms of DNA and RNA, which may be free or in the form of a complex with lipids (e.g., lipoplex) or polymer (polyplex).

In some methods, the first drug and second drug are co-administered, i.e., they are mixed together and administered to a subject at the same time and at the same injection site, i.e., T1=T2 and I1=I2. In other methods, the first drug and second drug are administered to a subject at different times and/or different administration routes, i.e., T1≠T2 and/or I1≠I2. In preferred embodiments, T1=T2 and I1=I2. Though, it is not always practical to administer D1 and D2 (or D2e) at the same time; in such instances, Ti should occur at least 6 hours before or at least 6 hours after T2, though, preferably at least 6 hours before T2, and more preferably between about 1 minute and 120 minutes prior T2, such as at least about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 35 minutes, 40 minutes, 45 minutes, 50 minutes, 55 minutes, 60 minutes, 70 minutes, 80 minutes, 90 minutes, 100 minutes, 110 minutes or 120 minutes. In preferred methods I1=I2, i.e. I1 is selected to match the route of administration of I2, such as an intravascular injection (e.g., intravenous or intraarterial), local injection (e.g., subcutaneous, transcutaneous, intramuscular, intrathecal, intraocular, intraarticular) or oral route of administration. In certain other methods, the first drug is administered multiple times, such as 1 or more time before administration of D2 or D2e and/or 1 or more times after administration of D1 or D2e.

### Compositions for Reducing or Preventing Graft or Device Rejection

Compositions of particles comprising amphiphiles further comprising a first drug molecule (D1) selected from inhibitors of mTORC1 and/or mTORC2, can similarly be administered at a first time (T1) and administration route (I1) to a subject and prevent antibodies or antibodies and T cells from being generated against a graft or implanted device.

In general, preferred compositions of particles comprising an amphiphile and a first drug molecule (D1) selected from inhibitors of mTORC1 and/or mTORC2 used for preventing antibodies or antibodies and T cells from being generated against a graft or implanted device are the same or like those used for preventing, inhibiting or reducing antibodies against a second drug molecule (D2) or expression system (D2e).

### Examples

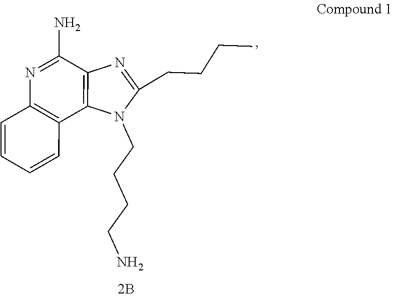 [SMILES: CCCCc1nc2c(N)nc3ccccc3c2n1CCCCN]

Compound 1, 1-(4-aminobutyl)-2-butyl-1H-imidazo[4,5-c]quinolin-4-amine, also referred to as 2B, was synthesized starting from 3-nitro-2,4-dichloroquinoline, 1-b, which was prepared as previously described (Lynn G M, et al., Nat Biotechnol 33(11):1201-1210, 2015). To 21 g of 1-b (87.8 mmol, 1 eq) in 210 mL of triethylamine (TEA) (10% w/w) was added 16.34 g (87.8 mmol, 1 eq) of N-boc-1,4-butanediamine while stirring vigorously. The reaction mixture was heated to 70° C. and monitored by HPLC, which confirmed that the reaction was complete after 2 hours. The triethylamine was removed under vacuum and the resulting oil was dissolved in 200 mL of dichloromethane and then washed with 3×100 mL DI H₂O. The organic layer was dried with Na₂SO₄ and then removed under vacuum and the resulting oil was triturated with 1:1 (v:v) hexane and diethyl ether to yield 30.7 g of yellow crystals of intermediate 1-c. MS (APCI) calculated for C₁₈H₂₃ClN₄O₄, m/z 394.1 found, 394.9.

1-d. 30.7 g (76.4 mmol) of intermediate 1-c was dissolved in 300 mL of ethyl acetate in a Parr Reactor vessel that was bubbled with argon, followed by the addition of 3 g of 10% platinum on carbon. The reaction vessel was kept under argon and then evacuated and pressurized with H₂(g) several times before pressurizing to 55 PSI H₂(g) while shaking vigorously. The H₂(g) was continually added until the pressure stabilized at 55 PSI, at which point the reaction was determined to be complete. The reaction mixture from the Parr Reactor was then filtered through celite end evaporated to dryness to obtain a yellow oil that was triturated with 1:1 hexanes/ether to yield white crystals that were collected by filtration to obtain 27.4 g of spectroscopically pure white crystals of 1-d. MS (APCI) calculated for C₁₈H₂₅ClN₄O₂, m/z 364.2, found 365.2.

1-e. To 10 g (27.4 mmol, 1 eq) of 1-d in 50 mL of THF was added 7.7 mL of triethylamine (54.8 mmol, 2 eq) followed by the dropwise addition of 3.6 g of valeroyl chloride (30.1 mmol, 1.1 eq) in 30 mL of THF while stirring vigorously while the reaction mixture was on ice. After 90 minutes, the ice bath was removed and the THF was removed under vacuum, resulting in a yellow oil that was dissolved in 100 mL of dichloromethane (DCM) that was washed with 3×50 mL of pH 5.5 100 mM acetate buffer. The DCM was removed under vacuum in an oil that was triturated with ethyl acetate to obtain 10.4 g of a white solid that was dissolved in methanol with 1 g of CaO (s), which was heated at 100° C. for 5 hours while stirring vigorously. The reaction mixture was filtered and dried to yield 10.2 g of an off-white solid, intermediate, 1-e. MS (ESI) calculated for C₂₃H₃₁ClN₄O₂, m/z 430.21, found 431.2.

1-f. To 10.2 g (23.7 mmol, 1 eq) of 1-e was added 30.4 g (284 mmol, 12 eq) of benzylamine liquid, which was heated to 110° C. while stirring vigorously. The reaction was complete after 10 hours and the reaction mixture was added to 200 mL ethyl acetate and washed 4×100 mL with 1 M HCl. The organic layer was dried with Na₂SO₄ and then removed under vacuum and the resulting oil was recrystallized from ethyl acetate to obtain 10.8 g of spectroscopically pure white crystals of intermediate, 1-f. MS (ESI) calculated for C₃₀H₃₉N₅O₂, m/z 501.31, found 502.3.

Compound 1. 10.8 g (21.5 mmol) of 1-f was dissolved in 54 mL of concentrated (>98%) H₂SO₄ and the reaction mixture was stirred vigorously for 3 hours. After 3 hours, viscous red reaction mixture was slowly added to 500 mL of DI H₂O while stirring vigorously. The reaction mixture was stirred for 30 minutes and then filtered through Celite, followed by the addition of 10 M NaOH until the pH of the solution was ˜pH 10. The aqueous layer was then extracted with 6×200 mL of DCM and the resulting organic layer was dried with Na₂SO₄ and reduced under vacuum to yield a spectroscopically pure white solid. ¹H NMR (400 MHZ, DMSO-d₆) δ 8.03 (d, J=8.1 HZ, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

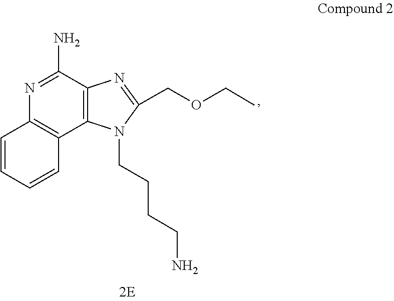 [SMILES: CCOCc1nc2c(N)nc3ccccc3c2n1CCCCN]

Compound 2, referred to as 2E, was prepared as previously described (Lynn G M, et al., Nat Biotechnol 33(11):1201-1210, 2015). ¹H NMR (400 MHz, DMSO-d6) δ 8.02 (dd, J=16.6, 8.2 Hz, 1H), 7.63-7.56 (m, 1H), 7.47-7.38 (m, 1H), 7.30-7.21 (m, 1H), 6.55 (s, 2H), 4.76 (s, 2H), 4.54 (q, J=6.3, 4.4 Hz, 2H), 3.54 (q, J=7.0 Hz, 2H), 2.58 (t, J=6.9 Hz, 2H), 1.93-1.81 (m, 2H), 1.52 (m, 2H), 1.15 (t, J=7.0 Hz, 3H). MS (APCI) calculated for C₁₇H₂₃N₅O m/z 313.2, found 314.2 (M+H)⁺.

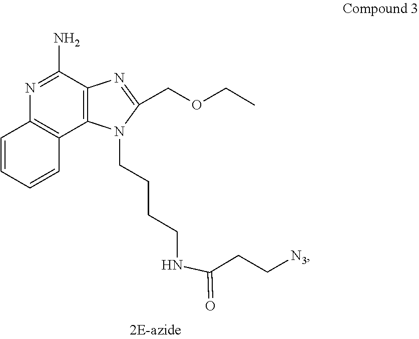 [SMILES: CCOCc1nc2c(N)nc3ccccc3c2n1CCCCNC(=O)CCN=[N+]=[N-]]

Compound 3, referred to as 2E-azide, was prepared as previously described (Lynn G M, et al., Nat Biotechnol 33(11):1201-1210, 2015). MS (APCI) calculated for C₂₀H₂₆N₈O₂ m/z 410.2, found 411.2 (M+H)⁺.

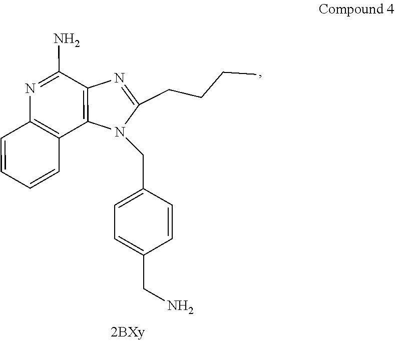 [SMILES: CCCCc1nc2c(N)nc3ccccc3c2n1Cc1ccc(CN)cc1]

Compound 4, 1-(4-(aminomethyl)benzyl)-2-butyl-1H-imidazo[4,5-c]quinolin-4-amine, referred to as 2BXy, was previously described (see: Lynn G M, et al., In vivo characterization of the physicochemical properties of polymer-linked TLR agonists that enhance vaccine immunogenicity. Nat Biotechnol 33(11):1201-1210, 2015, and Shukla N M, et al. Syntheses of fluorescent imidazoquinoline conjugates as probes of Toll-like receptor 7. Bioorg Med Chem Lett 20(22):6384-6386, 2010). 1H NMR (400 MHz, DMSO-d₆) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3 (M+H)⁺.

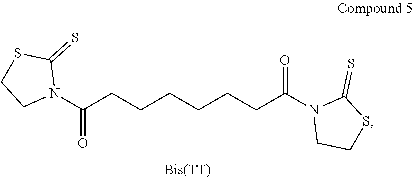 [SMILES: O=C(CCCCCCC(=O)N1CCSC1=S)N1CCSC1=S]

Compound 5, referred to as Bis(TT), was synthesized using Suberic acid and 2-thiazoline-2-thiol (TT) as starting materials. Briefly, 500 mg of Suberic acid (2.87 mmol, 1 eq), 752.7 mg of TT (6.31 mmol, 2.2 eq) and 1.431 g of EDC (7.46 mmol, 2.6 eq) were dissolved in 17.5 mL of dry DMSO. 70.15 mg of DMAP (0.57 mmol, 0.2 eq) was added and the reaction mixture was stirred at room temperature for 1 hour. The reaction mixture was diluted with DCM and washed twice with 1 M HCl and once with DI water. The organic fractions were dried with sodium sulfate and evaporated under reduced pressure to provide a yellow solid in quantitative yield.

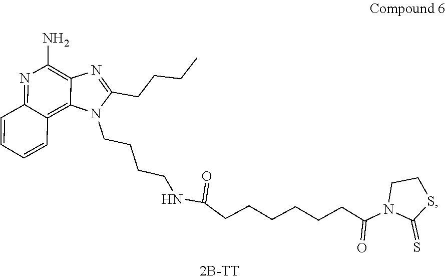 [SMILES: CCCCc1nc2c(N)nc3ccccc3c2n1CCCCNC(=O)CCCCCCC(=O)N1CCSC1=S]

Compound 6, referred to as 2B-TT, was synthesized using Compound 5 and Compound 1 as starting materials. Briefly, 50 mg (0.16 mmol, 1 eq) of Compound 1 was dissolved in 0.6 mL of methanol and added dropwise to a vigorously stirring solution of 301.1 mg of Compound 5 (0.8 mmol, 5 eq) in 1.93 mL of DCM. After 30 minutes, the reaction mixture was injected directly onto a column and purified by flash chromatography using a 2-step gradient: 5% methanol in DCM over 5 column volumes (CVs), followed by a 5-50% methanol in DCM gradient over 20 CVs. The fractions were combined and the solvent was removed under vacuum. MS (ESI) calculated for C₂₉H₄₀N₆O₂S₂ m/z 568.27 found 569.3 (M+H)⁺.

To screen for potential enzyme degradable linkers, a series of peptide-AMC conjugates were synthesized (see: Compounds 8-25 listed in Table 1) by solid-phase peptide synthesis.

TABLE 1
Substrates for evaluating enzyme specificity.

| | | SEQ | Theo. | Found |
|---|---|---|---|---|
| Cmpd # | Sequence* | ID NO: | MW | MW |
| 8 | Ac-A′VZ-AMC | 13 | 544.62 | conforms |
| 9 | Ac-A′SPVZ-AMC | 14 | 728.82 | conforms |
| 10 | Ac-A′SK(Ac)VZ-AMC | 15 | 801.90 | conforms |
| 11 | Ac-A′SKVZ-AMC | 16 | 759.87 | conforms |
| 12 | Ac-A′VK(Ac)-AMC | 17 | 557.65 | conforms |
| 13 | Ac-A′VK-AMC | 18 | 515.63 | conforms |
| 14 | Ac-A′VB-AMC | 19 | 472.56 | conforms |
| 15 | Ac-A′SPVB-AMC | 20 | 656.75 | conforms |
| 16 | Ac-A′SK(Ac)VB-AMC | 21 | 729.84 | conforms |
| 17 | Ac-A′SKVB-AMC | 22 | 687.81 | conforms |
| 18 | Ac-A′SK(Ac)SB-AMC | 23 | 717.78 | conforms |
| 19 | Ac-A′SKSB-AMC | 24 | 675.76 | conforms |
| 20 | Ac-A′VnL-AMC | 25 | 500.61 | conforms |
| 21 | Ac-A′SPVnL-AMC | 26 | 684.81 | conforms |
| 22 | Ac-A′SK(Ac)VnL-AMC | 27 | 757.89 | conforms |
| 23 | Ac-A′SKVnL-AMC | 28 | 715.86 | conforms |
| 24 | Ac-A′SK(Ac)SnL-AMC | 29 | 745.84 | conforms |
| 25 | Ac-A′SKSnL-AMC | 30 | 703.81 | conforms |

*Single letter code is used for the amino acid sequences listed in the above table;
A′ = β-alanine,
B = α-aminobutyric acid,
K = lysine,
Ac = acyl,
nL = norleucine,
S = serine,
V = valine,
AMC = 7-Amino-4-methylcoumarin.
All peptides in the above table were manufactured by solid-phase peptide synthesis by Genscript (Piscataway, NJ)

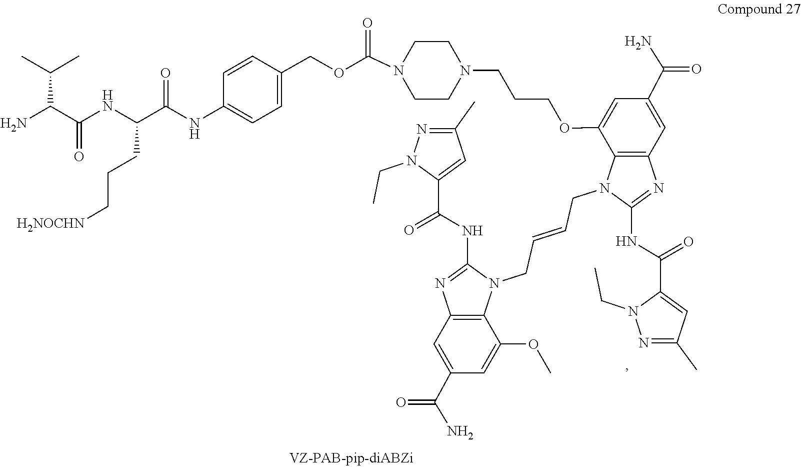

Compound 27, referred to as VZ-PAB-pip-diABZi was synthesized by first adding Fmoc-Val-Cit-PAB-PNP (104 mg, 0.14 mmol) followed by DIEA (44 mg, 0.34 mmol) to a solution of pip-diABZi, Compound 7, (120 mg, 0.14 mmol) in DMAC (6.0 mL). The solution was stirred for 16 hours at room temperature and the desired product was precipitated by the addition of cold diethyl ether (100 mL). Isolation of the solid by centrifugation afforded the desired Fmoc-protected intermediate as an off-white solid (70 mg, 34% yield). This Fmoc-protected intermediate was used in the next synthetic step without additional purification or characterization. The Fmoc protected intermediate (70 mg, 0.047 mmol) was dissolved in 20% piperdine in DMF (0.6 mL). The solution was stirred for 30 minutes at room temperature and then 12 mL of cold diethyl ether was added to precipitate the product. The cold diethyl ether was decanted from the product-containing solid pellet. This crude solid was washed with additional cold diethyl ether (3×12 mL). The resultant solid was dried overnight to afford 50 mg (85% yield) of a pure (98.2% AUC at 220 nm) off-white solid. MS (EI) calculated for C₆₁H₇₉N₁₉O₁₁, m/z 1253.6, found, 1255 (M+H)⁺.

Compounds 26, 28, 29, 162, and 163 were produced in a similar manner as that described for Compound 17. Table 2 provides a summary of the synthesis and characterization of compounds 26, 27, 28, 29, 162 and 163.

TABLE 2
drug-peptide compounds

| Cmpd # | Sequence* | Theo, MW | Found |
|---|---|---|---|
| 26 | VZ-PAB-2BXy | 986.48 | conforms |
| 28 | VZ-PAB-napthylene | 562.29 | conforms |
| 29 | VZ-diABZi | 1104.57 | conforms |
| 162 | VZ-PAB-2B | 716.89 | conforms |
| 163 | VZ-PAB-Kyn | 613.67 | conforms |

*Single letter code is used for the amino acid sequences listed in the above table;
A′ = β-alanine,
B = α-aminobutyric acid,
K = lysine,
Ac = acyl,
nL = norleucine,
S = serine,
V = valine,
AMC = 7-Amino-4-methylcoumarin.
Kyn = Kynurenine.

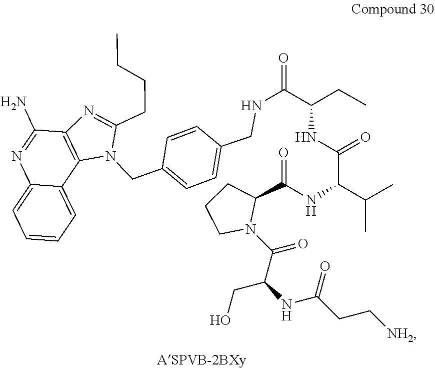 [SMILES: CCCCc1nc2c(N)nc3ccccc3c2n1Cc1ccc(CNC(=O)[C@H](CC)NC(=O)[C@@H](NC(=O)[C@@H]2CCCN2C(=O)[C@H](CO)NC(=O)CCN)C(C)C)cc1]

Compound 30, referred to as A′SPVB-2BXy was synthesized by combining Compound 4 (40 mg, 0.11 mmol), A′SPVB, (68 mg, 0.11 mmol) (SEQ ID NO:31), which was synthesized by solid phase synthesis, DIEA (86 mg, 0.67 mmol) in DMF (2.0 mL). HATU (42 mg, 0.11 mmol) was added and the yellow solution was stirred for 16 hours at room temperature. The solvent was removed and the material was partitioned between EtOAC (10 mL) and sat'd NaHCO₃ (10 mL). The EtOAc layer was removed and then washed with 10% KHSO₄, dried over MgSO₄, filtered, and purified by flash chromatography using a 2-step gradient: 0% methanol in DCM, followed by a 0-10% methanol in DCM gradient. The desired protected-intermediate was obtained as a white solid (37 mg, 35% yield). To the protected intermediate of Compound 30 was added DCM (0.5 mL) followed by TFA (0.5 mL). The solution was stirred at room temperature for 30 minutes, was evaporated to dryness, and the desired product was obtained as an off-white solid (34 mg, 99% yield, 96.2% AUC at 220 nm). MS (EI) calculated for C₄₂H₅₈N₁₀O₆, m/z 798.5, found 799.5 (M+H)⁺.

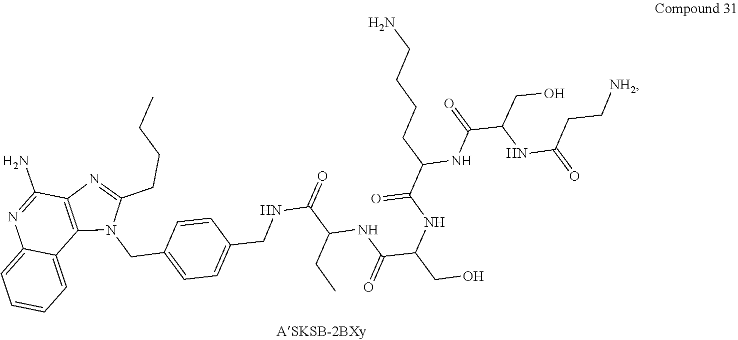 [SMILES: CCCCc1nc2c(N)nc3ccccc3c2n1Cc1ccc(CNC(=O)C(CC)NC(=O)C(CO)NC(=O)C(CCCCN)NC(=O)C(CO)NC(=O)CCN)cc1]

Compound 31, referred to as A′SKSB-2BXy, was synthesized using the same procedure as described for Compound 30, except A′SKSB (SEQ ID NO:32), which was synthesized by solid phase peptide synthesis, was used in place of A′SPVB. The product was obtained as a white solid (70% yield, 90.4% AUC at 220 nm). MS (EI) calculated for C₄₁H₅₉N₁₁O₇, m/z 817.5, found 818.4 (M+H)⁺.

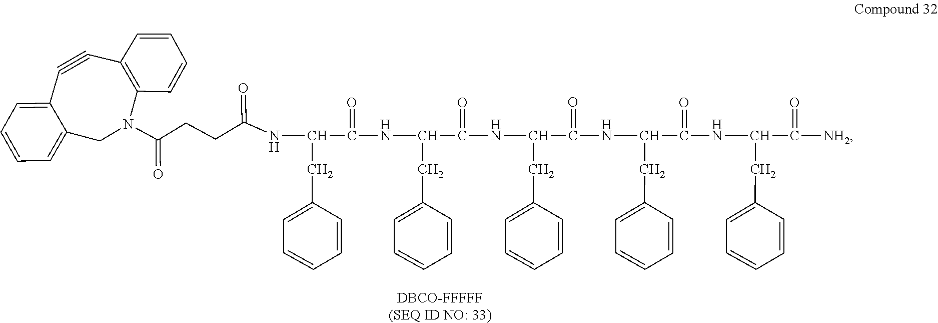

Compound 32, referred to as DBCO-F₅, F₅ or DBCO-(Phe)₅ was synthesized by reacting 50.0 mg (0.066 mmol, 1 eq) of the precursor NH₂-(Phe)₅-NH₂ (SEQ ID:34), which was prepared by solid phase peptide synthesis, with 29.4 mg of DBCO-NHS (0.073 mmol, 1.1 eq) and 7.4 mg of triethylamine (0.073 mmol, 1.1 eq) in 1.0 mL of DMSO. Compound 32 was purified on a preparatory HPLC system using a gradient of 30-95% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain a spectroscopically pure (>95% AUC at 254 nm) white powder. MS (ESI) calculated for C₆₄H₆₁N₇O₇ m/z 1039.46, found 1040.6 (M+H)⁺.

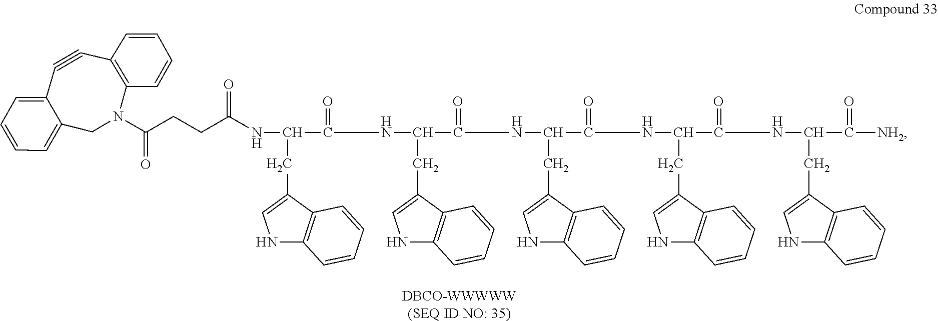

Compound 33, referred to as DBCO-W₅, W₅ or DBCO-(Trp)₅ was synthesized by reacting 137.6 mg (0.15 mmol, 1 eq) of the precursor NH₂-(Trp)₅-NH₂(SEQ ID NO:36), which was prepared by solid phase peptide synthesis, with 146.1 mg of DBCO-NHS (0.057 mmol, 2.5 eq) and 14.7 mg of triethylamine (0.15 mmol, 1.1 eq) in 3.0 mL of DMSO. Compound 33 was purified on a preparatory HPLC system using a gradient of 52-72% acetonitrile/H₂O (0.05% TFA) over 12 minutes on an Agilent Prep-C18 column, 50×100 mm, 5 μm. The product eluted at ˜10 minutes and the resulting fractions were collected, frozen and then lyophilized to obtain 75.1 mg (42% yield) of a spectroscopically pure (>95% AUC at 254 nm) white powder. MS (ESI) calculated for C₇₄H₆₆N₁₂O₇ m/z 1234.52, found 1235.6 (M+H)⁺.

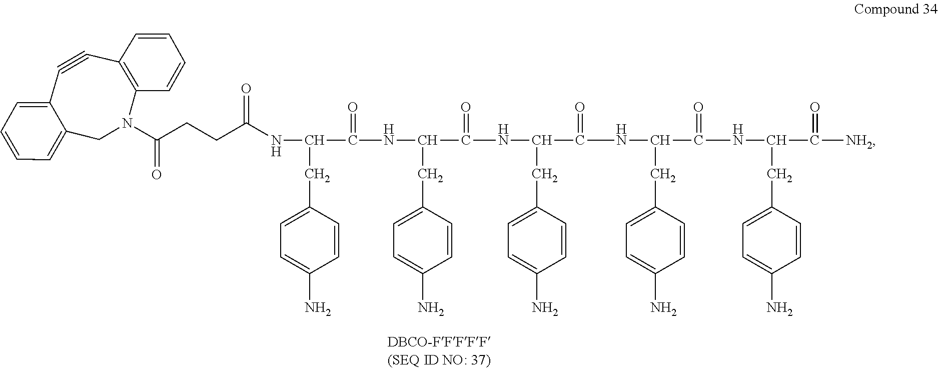 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCC(=O)N1Cc2ccccc2C#Cc2ccccc21]

Compound 34, referred to as DBCO-F′₅ or F′₅ was synthesized by reacting 49.8 mg (0.06 mmol, 1 eq) of the precursor NH₂-(F′)₅-NH₂ (SEQ ID NO:38), which was prepared by solid phase peptide synthesis, with 24.5 mg of DBCO-TT (0.057 mmol, 1.0 eq) and 30.3 mg of NaHCO₃ (0.36 mmol, 6.0 eq) in 1.0 mL of DMF. The reaction was run overnight at room temperature and HPLC indicated that the reaction was complete by 24 hours. Compound 34 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜3.4 minutes and the resulting fractions were collected, frozen and then lyophilized to obtain 25.8 mg (38.4% yield) of a spectroscopically pure (>95% AUC at 254 nm) white powder. MS (ESI) calculated for C₆₄H₆₆N₁₂O₇ m/z 1114.52, found 1116.1 (M+H)⁺.

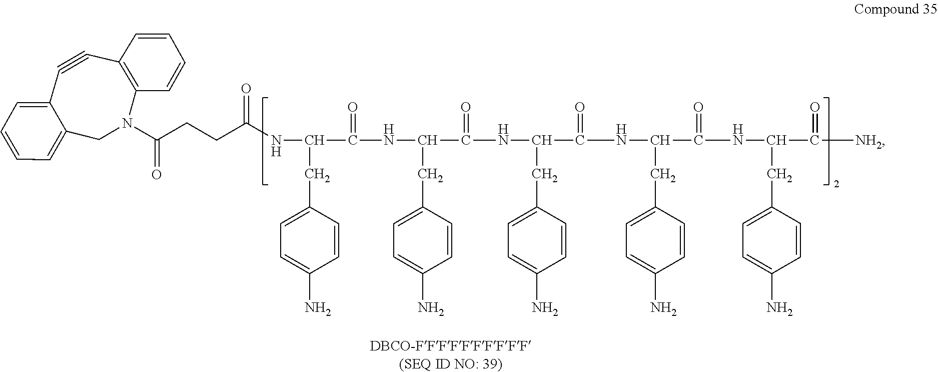

Compound 35, referred to as DBCO-F′₁₀ or F′₁₀ was synthesized by reacting 30 mg (0.0183 mmol, 1 eq) of the precursor NH₂-(F′)₁₀-NH₂ (SEQ ID NO:40), which was prepared by solid phase peptide synthesis, with 7.4 mg of DBCO-TT (0.018 mmol, 1.0 eq) and 16.9 mg of NaHCO₃ (0.20 mmol, 11 eq) in 1.0 mL of DMF. The reaction was run overnight at room temperature and HPLC indicated that the reaction was complete by 24 hours. Compound 35 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜6.3 minutes and the resulting fractions were collected, frozen and then lyophilized to obtain 14 mg (39.5% yield) of a spectroscopically pure (>95% AUC at 254 nm) white powder. MS (ESI) calculated for C₁₀₉H₁₁₆N₂₂O₁₂ m/z 1924.91, found 963.9(M/2+H)⁺.

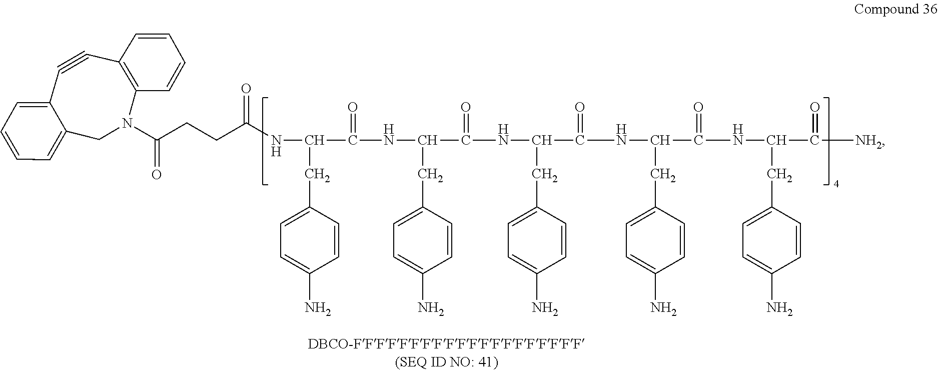

Compound 36, referred to as DBCO-F′₂₀ or F′₂₀ was synthesized by reacting 30 mg (0.009 mmol, 1 eq) of the precursor NH₂-(F′)₂₀-NH₂ (SEQ ID NO:42), which was prepared by solid phase peptide synthesis, with 3.7 mg of DBCO-TT (0.009 mmol, 1.0 eq) and 16.2 mg of NaHCO₃ (0.20 mmol, 21 eq) in 1.0 mL of DMF. The reaction was run overnight at room temperature and HPLC indicated that the reaction was complete by 24 hours. Compound 36 was purified on a preparatory HPLC system using a gradient of 10-30% acetonitrile/H₂O (0.05% TFA) over 10 minutes on an Agilent Prep-C18 column, 30×100 mm, 5 μm. The product eluted at ˜6.3 minutes and the resulting fractions were collected, frozen and then lyophilized to obtain 10.6 mg (32.4% yield) of a spectroscopically pure (>95% AUC at 254 nm) white powder. MS (ESI) calculated for C₁₉₉H₂₁₆N₄₂O₂₂ m/z 3545.71 found 1183.6 (M/3+H)⁺ and 887 (M+4H)⁺.

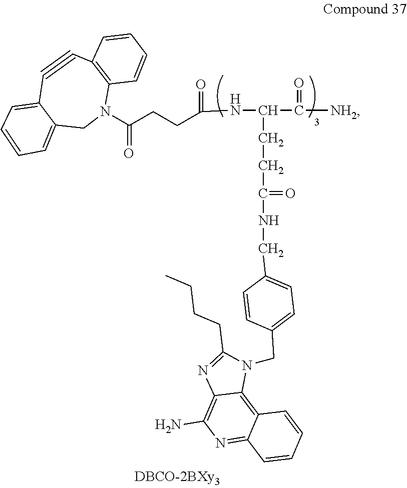

Compound 37, referred to as DBCO-2BXy₃, 2BXy₃ or DBCO-(Glu(2BXy)₃), was synthesized starting from an Fmoc-(Glu)₃-NH₂ precursor, which was prepared by solid-phase peptide synthesis. 50 mg of Fmoc-(Glu)₃-NH₂ (SEQ ID NO:43) (0.08 mmol, 1 eq), 143 mg of Compound 4 (0.40 mmol, 5 eq), 84 mg of 2-chloro-4,6-dimethoxy-1,3,5-triazine (CDMT) (0.48 mmol, 6 eq) and 48.5 mg of 4-methylmorpholine (NMM) (0.48 mmol, 6 eq) were added to 3.25 mL of DMSO while stirring vigorously at room temperature under ambient air. The reaction progress was monitored by HPLC (AUC 254 nm). 1 additional equivalent of Compound 4 and 2 additional equivalents of both CDMT and NMM were added after 30 minutes. After 2 hours, the reaction was complete and the reaction mixture was added to 50 mL of a 1M HCl solution to precipitate the Fmoc-protected intermediate, which was collected by centrifuging the solution at 3000 g at 4° C. for 10 minutes. The HCl solution was discarded and the Fmoc protected intermediate was collected as a solid white pellet. The white solid was re-suspended in 50 mL of a 1M HCl solution and spun at 3000 g at 4° C. for 5 minutes; the 1 M HCl solution was discarded and the product was collected as a solid pellet. This process was repeated and then the solid was collected and dried under vacuum to yield 156.1 mg of the Fmoc protected intermediate in quantitative yield. The Fmoc protected product was then added to 1.5 mL of a 20% piperidine in DMF solution for 30 minutes at room temperature to yield the deprotected product that was then precipitated from 50 mL of ether and centrifuged at 3000 g at 4° C. for 30 minutes. The product was collected as a solid pellet and then washed twice more with ether, followed by drying under vacuum to yield 126.4 mg of the intermediate. 60 mg of the resulting intermediate, NH₂-(Glu-2BXy)₃-NH₂, (0.042 mmol, 1 eq) was then reacted with 18.6 mg (0.046 mmol, 1.1 eq) of DBCO-NHS ester (Scottsdale, Arizona, USA) and 8.5 uL of triethylamine (0.084 mmol, 2 eq) in 1 mL of DMSO for 6 hours at room temperature. The resulting product, Compound 37, was purified on a preparatory HPLC system using a gradient of 30-70% acetonitrile/H₂O (0.05% TFA) over 12 minutes on an Agilent Prep-C18 column, 50×100 mm, 5 μm. The product eluted at 7.0 minutes and the resulting fractions were collected, frozen and then lyophilized to obtain 40.12 mg (55.7% yield) of a spectroscopically pure (>95% AUC at 254 nm) white powder. MS (ESI) calculated for C₁₀₀H₁₀₆N₂₀O₈ m/z 1714.85, found 858.9 (M/2)⁺.

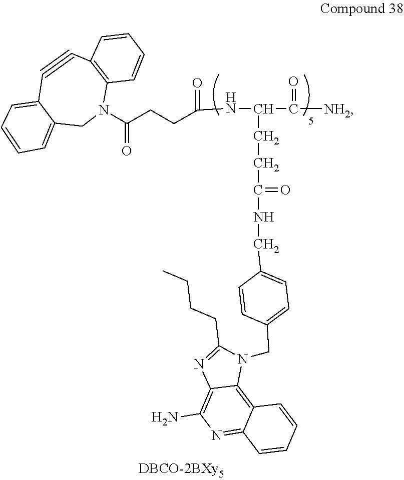

Compound 38, referred to as DBCO-2BXy₅, 2BXy₅ or DBCO-(Glu(2BXy)₅), was synthesized using the same procedure as described for Compound 37, except Fmoc-(Glu)₅-NH₂ (SEQ ID NO:44) was used as the starting material for conjugation of Compound 4. Compound 38 was purified on a preparatory HPLC system using a gradient of 38-48% acetonitrile/H₂O (0.05% TFA) over 12 minutes on an Agilent Prep-C18 column, 50×100 mm, 5 μm. The product eluted at 8.0 minutes and the resulting fractions were collected, frozen and then lyophilized to obtain 45.9 mg (63.4% yield) of a spectroscopically pure (>95% AUC at 254 nm) white powder. MS (ESI) calculated for C₁₅₄H₁₆₆N₃₂O₁₂ m/z 2655.34, found 886.6 (M/3)⁺.

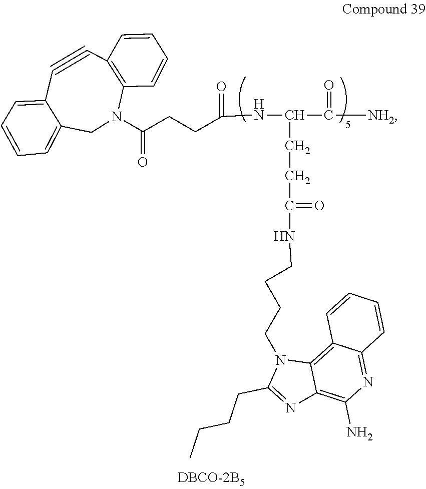

Compound 39, referred to as DBCO-2B₅, 2B₅ or DBCO-(Glu(2B)₅), was synthesized using the same procedure as described for Compound 37, except Fmoc-(Glu)₅-NH₂ (SEQ ID NO:45) was used as the starting material for conjugation of Compound 1. Compound 39 was purified on a preparatory HPLC system using a gradient of 33-45% acetonitrile/H₂O (0.05% TFA) over 12 minutes on an Agilent Prep-C18 column, 50×100 mm, 5 μm. The product eluted at ˜10.0 minutes and the resulting fractions were collected, frozen and then lyophilized to obtain 25.2 mg (62.6% yield) of a spectroscopically pure (>95% AUC at 254 nm) white powder. MS (ESI) calculated for C₁₃₄H₁₆₆N₃₂O₁₂ m/z 2415.34, found 1209.3 (M/2)⁺.

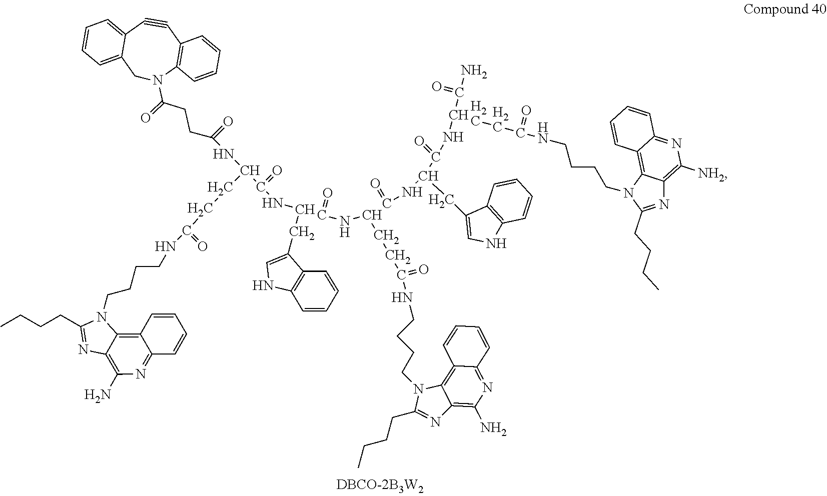 [SMILES: CCCCc1nc2c(N)nc3ccccc3c2n1CCCCNC(=O)CCC(NC(=O)C(Cc1c[nH]c2ccccc12)NC(=O)C(CCC(=O)NCCCCn1c(CCCC)nc2c(N)nc3ccccc3c21)NC(=O)C(Cc1c[nH]c2ccccc12)NC(=O)C(CCC(=O)NCCCCn1c(CCCC)nc2c(N)nc3ccccc3c21)NC(=O)CCC(=O)N1Cc2ccccc2C#Cc2ccccc21)C(N)=O]

Compound 40, referred to as DBCO-2B₃W₂, 2B₃W₂ or DBCO-(Glu(2B)₃(Trp)₂), was synthesized using the same procedure as described for Compound 37, except Fmoc-Glu-Trp-Glu-Trp-Glu-NH₂ (SEQ ID NO:46) was used as the starting material for conjugation of Compound 1. Compound 40 was purified on a preparatory HPLC system using a gradient of 33-47% acetonitrile/H₂O (0.05% TFA) over 12 minutes on an Agilent Prep-C18 column, 50×100 mm, 5 m. The product eluted at ˜8 minutes and the resulting fractions were collected, frozen and then lyophilized to obtain 197 mg (50.6% yield) of a spectroscopically pure (>95% AUC at 254 nm) white powder. MS (ESI) calculated for C₁₁₀H₁₂₆N₂₄O₁₀ m/z 1943.01, found 973.0 (M/2)⁺.

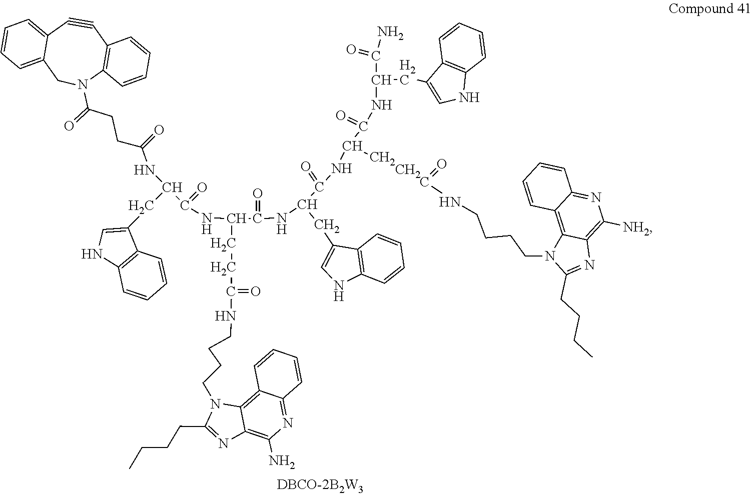 [SMILES: CCCCc1nc2c(N)nc3ccccc3c2n1CCCCNC(=O)CCC(NC(=O)C(Cc1c[nH]c2ccccc12)NC(=O)C(CCC(=O)NCCCCn1c(CCCC)nc2c(N)nc3ccccc3c21)NC(=O)C(Cc1c[nH]c2ccccc12)NC(=O)CCC(=O)N1Cc2ccccc2C#Cc2ccccc21)C(=O)NC(Cc1c[nH]c2ccccc12)C(N)=O]

Compound 41, referred to as DBCO-2B₂W₃, 2B₂W₃ or DBCO-(Glu(2B)₂(Trp)₃), was synthesized using the same procedure as described for Compound 37, except Fmoc-Trp-Glu-Trp-Glu-Trp-NH₂ (SEQ ID NO:47), was used as the starting material for conjugation of Compound 1. Compound 41 was purified on a preparatory HPLC system using a gradient of 35-65% acetonitrile/H₂O (0.05% TFA) over 12 minutes on an Agilent Prep-C18 column, 50×100 mm, 5 μm. The product eluted at ˜9 minutes and the resulting fractions were collected, frozen and then lyophilized to obtain 11.6 mg (62.5% yield) of a spectroscopically pure (>95% AUC at 254 nm) white powder. MS (ESI) calculated for C₉₈H₁₀₆N₂₀O₉ m/z 1706.85, found 854.9 (M/2)⁺.

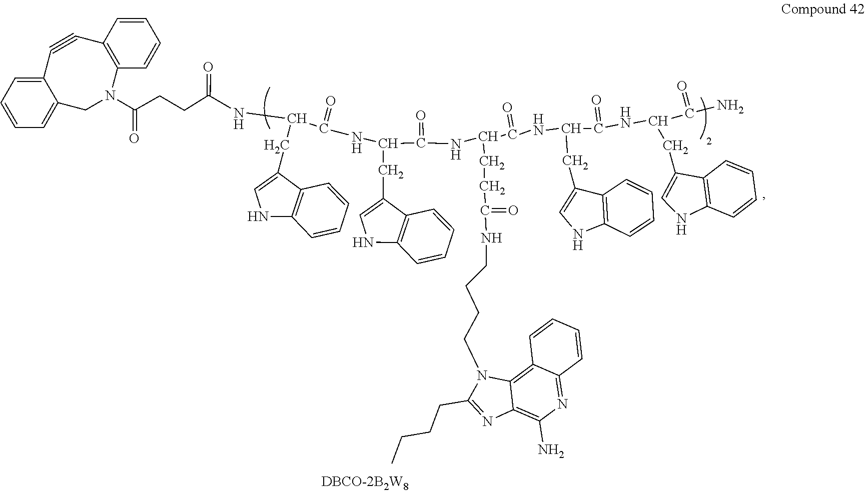

Compound 42, referred to as DBCO-2B₂W₈, 2B₂W₈ or DBCO-(Glu(2B)₂(Trp)₈), was synthesized using the same procedure as described for Compound 37, except Fmoc-Trp-Trp-Glu-Trp-Trp-Trp-Trp-Glu-Trp-Trp-NH₂ (SEQ ID NO:48), was used as the starting material for conjugation of Compound 1. Compound 42 was purified on a preparatory HPLC system using a gradient of 35-85% acetonitrile/H₂O (0.05% TFA) over 12 minutes on an Agilent Prep-C18 column, 50×100 mm, 5 μm. The product eluted at ˜8.0 minutes and the resulting fractions were collected, frozen and then lyophilized to obtain 3.3 mg (16.3% yield) of a spectroscopically pure (>95% AUC at 254 nm) white powder. MS (ESI) calculated for C₁₅₃H₁₅₆N₃₀O₁₄ m/z 2637.24, found 1320.2 (M/2)⁺.

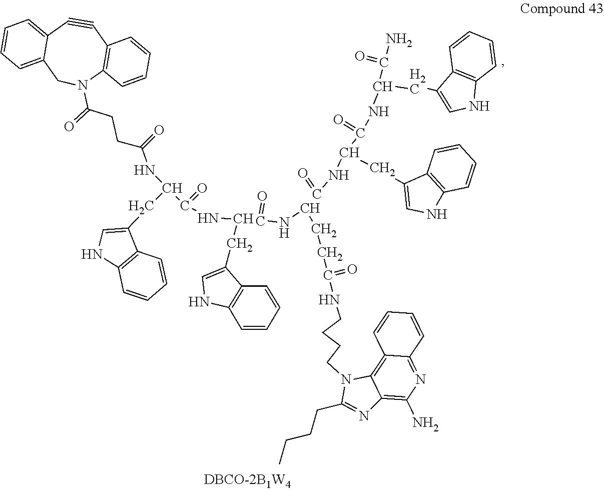 [SMILES: CCCCc1nc2c(N)nc3ccccc3c2n1CCCCNC(=O)CCC(NC(=O)C(Cc1c[nH]c2ccccc12)NC(=O)C(Cc1c[nH]c2ccccc12)NC(=O)CCC(=O)N1Cc2ccccc2C#Cc2ccccc21)C(=O)NC(Cc1c[nH]c2ccccc12)C(=O)NC(Cc1c[nH]c2ccccc12)C(N)=O]

Compound 43 referred to as DBCO-2B₁W₄, 2B₁W₄ or DBCO-(Glu(2B)₁(Trp)₄), was synthesized using the same procedure as described for Compound 37, except Fmoc-Trp-Trp-Glu-Trp-Trp-NH₂ (SEQ ID NO:49), was used as the starting material for conjugation of Compound 1. Compound 43 was purified on a preparatory HPLC system using a gradient of 50-55% acetonitrile/H₂O (0.05% TFA) over 12 minutes on an Agilent Prep-C18 column, 50×100 mm, 5 m. The product eluted at 8.9 minutes and the resulting fractions were collected, frozen and then lyophilized to obtain 9.7 mg (55.4% yield) of a spectroscopically pure (>95% AUC at 254 nm) white powder. MS (ESI) calculated for C₈₆H₈₆N₁₆O₈ m/z 1470.68, found 736.6 (M/2)⁺.

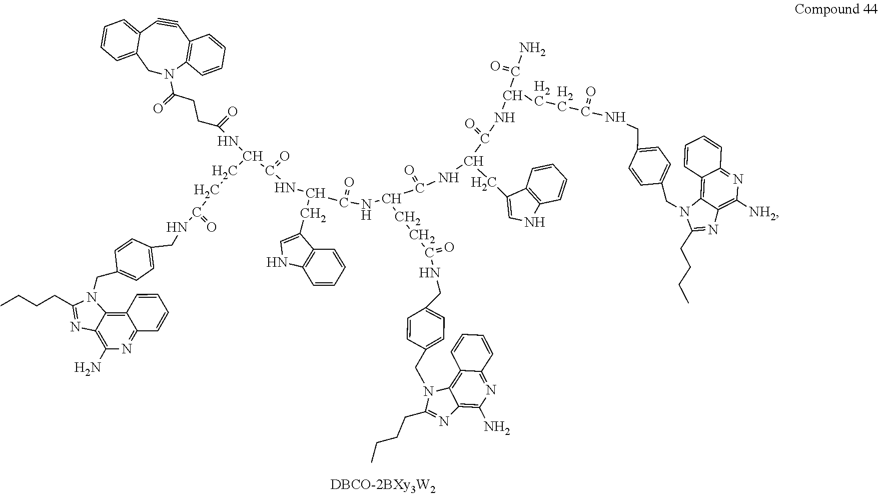 [SMILES: CCCCc1nc2c(N)nc3ccccc3c2n1Cc1ccc(CNC(=O)CCC(NC(=O)C(Cc2c[nH]c3ccccc23)NC(=O)C(CCC(=O)NCc2ccc(Cn3c(CCCC)nc4c(N)nc5ccccc5c43)cc2)NC(=O)C(Cc2c[nH]c3ccccc23)NC(=O)C(CCC(=O)NCc2ccc(Cn3c(CCCC)nc4c(N)nc5ccccc5c43)cc2)NC(=O)CCC(=O)N2Cc3ccccc3C#Cc3ccccc32)C(N)=O)cc1]

Compound 44, referred to as DBCO-2BXy₃W₂, 2BXy₃W₂ or DBCO-(Glu(2BXy)₃(Trp)₂), was prepared using Fmoc-Glu-Trp-Glu-Trp-Glu-NH₂ (SEQ ID NO:50) and Compound 4 as the starting materials. 500 mg of Fmoc-Glu-Trp-Glu-Trp-Glu-NH₂, (0.5 mmol, 1 eq), 595.6 mg of thiazoline-2-thiol (TT) (5 mmol, 10 eq), and 575.7 mg of 1-ethyl-3-(3-dimethylaminopropyl)carbodiimide (EDC) (3 mmol, 6 eq) were suspended in 26 mL of DCM. 18.3 mg of 4-(dimethylamino)pyridine (DMAP) (0.2 mmol, 0.3 eq) was added and the reaction mixture was stirred at room temperature. The reaction progress was monitored by analytical HPLC. After 4 hours, an additional four equivalents of TT and two equivalents of EDC were added. After stirring overnight, two equivalents of TT and a half equivalent of EDC were added. After 6 hours, the reaction was complete. The DCM was removed under vacuum and the solid was taken up in 6 mL of dry DMSO. 539.3 mg of Compound 4 (1.5 mmol, 3 eq) was added and the reaction mixture was stirred for 2 hours at room temperature. The conjugated intermediate was then precipitated from 300 mL of 1 M HCl and centrifuged at 3000 g at 4° C. for 10 minutes. The pellet was collected and washed once more with 1 M HCl and once with DI water. The final collected pellet was frozen and dried under vacuum. 809.06 mg of Fmoc-2BXy₃W₂—NH₂ (0.4 mmol, 1 eq)) was dissolved in 4 mL of 20% piperidine in DMF. The reaction mixture was stirred at room temperature for 1 hour. The deprotected intermediate was then precipitated from 100 mL of ether and centrifuged at 3000 g at 4° C. for 10 minutes. The product was collected as a solid pellet and then washed twice more with ether, followed by drying under vacuum to yield the intermediate. 729 mg NH₂-2BXy₃W₂—NH2 (0.4 mmol, 1 eq) was dissolved in 6 mL of dry DMSO. 488.8 mg of DBCO-NHS (1.2 mmol, 3 eq) was added and the reaction mixture was stirred at room temperature for 1 hour. The resulting product was purified on a preparatory HPLC system using a gradient of 36-46% acetonitrile/H2O (0.05% TFA) over 12 minutes on an Agilent Prep C-18 column, 50×100 mm, 5 μm. The resulting fractions were combined, frozen and lyophilized to give 239 mg (38.1% yield) of a spectroscopically pure of white powder. MS (ESI) Calculated for C₁₂₂H₁₂₆N₂₄O₁₀ m/z 2087.65 found 697 (m/3)⁺.

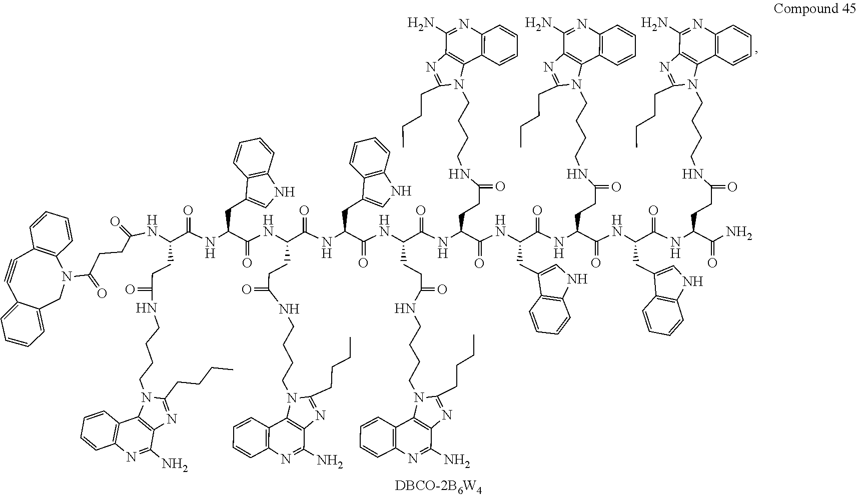 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)CCC(=O)N1Cc2ccccc2C#Cc2ccccc21)C(N)=O]

Compound 45, referred to as DBCO-2B₆W₄, 2B₆W₄ or DBCO-(Glu(2B)₆(Trp)₄), was synthesized using the same procedure as described for Compound 37, except Fmoc-(Glu-Trp-Glu-Trp-Glu)₂-NH₂(SEQ ID NO:51), was used as the starting material for conjugation of Compound 1. Compound 45 was purified on a preparatory HPLC system using a gradient of 24-45% acetonitrile/H₂O (0.05% TFA) over 10 minutes on an Agilent Prep-C18 column, 30×100 mm, 5 m. The fractions were collected, frozen and then lyophilized to obtain a spectroscopically pure (>95% AUC at 254 nm) white powder. MS (ESI) calculated for C₂₀₁H₂₃₆N₄₆O₁₈ m/z 3582.4, found 717.7 (M/5)⁺.

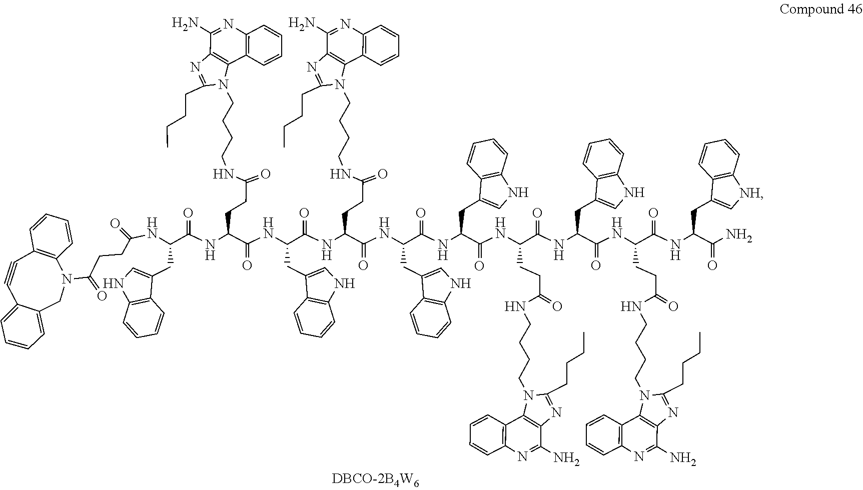 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)CCC(=O)N1Cc2ccccc2C#Cc2ccccc21)C(=O)N[C@@H](Cc1c[nH]c2ccccc12)C(N)=O]

Compound 46, referred to as DBCO-2B₄W₆, 2B₄W₆ or DBCO-(Glu(2B)₄(Trp)₆), was synthesized using the same procedure as described for Compound 37, except Fmoc-(Trp-Glu-Trp-Glu-Trp)₂-NH₂ (SEQ ID NO:52), was used as the starting material for conjugation of Compound 1. Compound 46 was purified on a preparatory HPLC system using a gradient of 24-45% acetonitrile/H₂O (0.05% TFA) over 10 minutes on an Agilent Prep-C18 column, 30×100 mm, 5 m. The fractions were collected, frozen and then lyophilized to obtain a spectroscopically pure (>95% AUC at 254 nm) white powder. MS (ESI) calculated for C₁₇₇H₁₉₆N₃₈O₁₆ m/z 3111.7, found 777.5 (M/4)⁺.

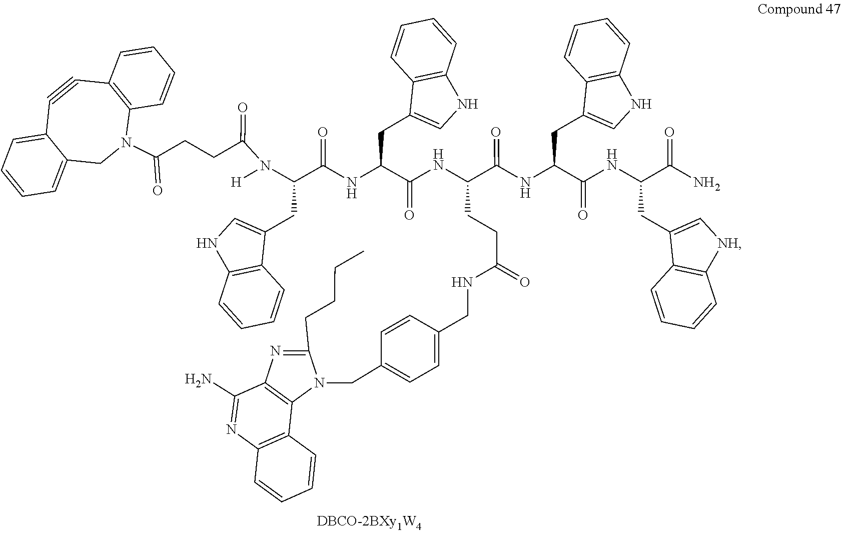 [SMILES: CCCCc1nc2c(N)nc3ccccc3c2n1Cc1ccc(CNC(=O)CC[C@H](NC(=O)[C@H](Cc2c[nH]c3ccccc23)NC(=O)[C@H](Cc2c[nH]c3ccccc23)NC(=O)CCC(=O)N2Cc3ccccc3C#Cc3ccccc32)C(=O)N[C@@H](Cc2c[nH]c3ccccc23)C(=O)N[C@@H](Cc2c[nH]c3ccccc23)C(N)=O)cc1]

Compound 47, referred to as DBCO-2BXy₁W₄, 2BXy₁W₄ or DBCO-(Glu(2BXy)₁(Trp)₄), was prepared using the same procedure as described for Compound 37, except Fmoc-Trp-Trp-Glu-Trp-Trp-NH₂ was used as the starting material. Compound 47 was purified on a preparatory HPLC system using a gradient of 40-70% acetonitrile/H₂O (0.05% TFA) over 16 minutes on an Agilent Prep-C18 column, 30×100 mm, 5 μm. The resulting fractions were collected, frozen and then lyophilized to obtain 3.4 mg (73.3% yield) of a spectroscopically pure (>95% AUC at 254 nm) white powder. MS (ESI) calculated for C₉₀H₈₅N₁₅O₉ m/z 1519.67, found 760.5 (M/2)⁺.

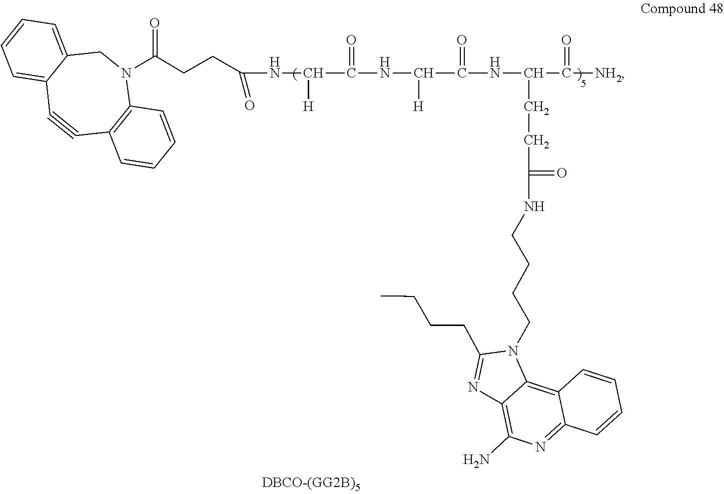

Compound 48, referred to as DBCO-(GG2B)₅, 2B₅G₁₀ or DBCO-(Glu(2B)₅(Gly)₁₀), was synthesized using the same procedure described for Compound 37, except Fmoc-(Gly-Gly-Glu)₅-NH₂ (SEQ ID NO:53) and Compound 1 were used as the starting materials. Compound 48 was purified on a preparatory HPLC system using a gradient of 22-42% acetonitrile/H₂O (0.05% TFA) over 12 minutes on an Agilent Prep-C18 column, 30×100 mm, 5 m. The product eluted at 7 minutes and the resulting fractions were collected, frozen and then lyophilized to obtain 22.8 mg (36.2% yield) of a spectroscopically pure (>95% AUC at 254 nm) white powder. MS (ESI) calculated C₁₅₄H₁₉₆N₄₂O₂₂ for m/z 2985.51, found 598.5 (M/5)⁺.

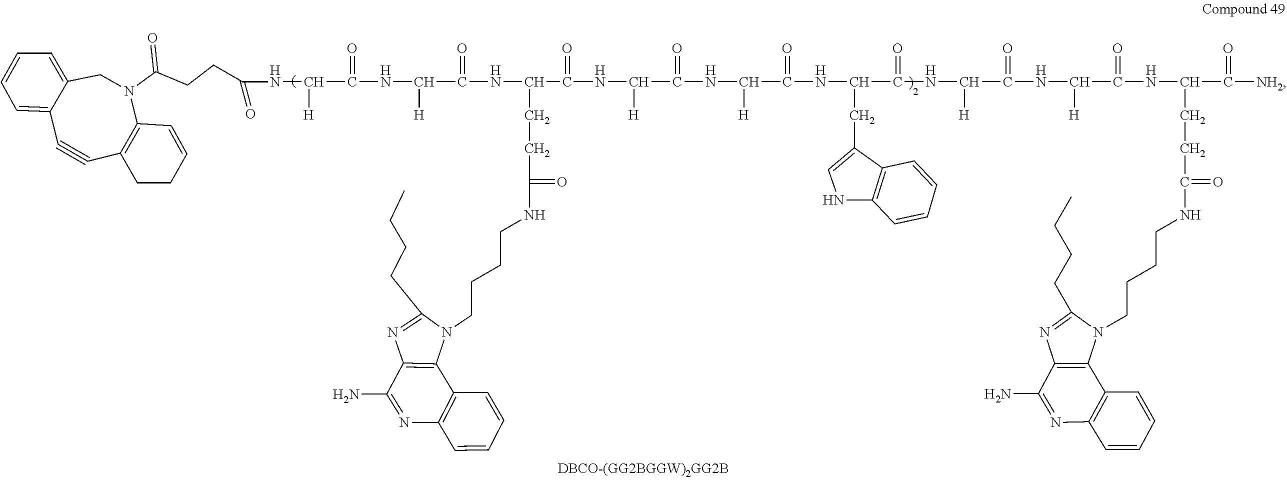

Compound 49, referred to as DBCO-(GG2BGGW)₂GG2B, 2B₃W₂G₁₀ or DBCO-(Glu(2B)₃(Trp)₂(Gly)₁₀), was synthesized from Fmoc-(Gly-Gly-Glu-Gly-Gly-Trp)₂-Gly₂-Glu-NH₂, precursor prepared by solid-phase peptide synthesis and Compound 1. 235.4 mg of Fmoc-(Gly-Gly-Glu-Gly-Gly-Trp)₂-Gly₂-Glu-NH₂ (SEQ ID NO:54), (0.15 mmol, 1 eq) was dissolved in 2 mL of 20% Piperidine in DMF. After 30 minutes the reaction was complete and the product was precipitated from 100 mL of ether and centrifuged at 3000 g at 4° C. for 10 minutes. The product was collected as a solid pellet and then washed twice more with ether, followed by drying under vacuum to yield ˜200 mg of the deprotected intermediate. 200 mg (0.15 mmol, 1 eq) of NH₂-(Gly-Gly-Glu-Gly-Gly-Trp)₂-Gly₂-Glu-NH₂ (SEQ ID NO:55), was dissolved in 2 mL of dry DMSO and 89.73 mg of DBCO-NHS (0.22 mmol, 1.5 eq) was added followed by TEA (0.22 mmol, 1.5 eq). The reaction mixture was stirred at room temperature for 1 hour. The resulting DBCO intermediate was purified on a preparative HPLC system using a gradient of 30-50% acetonitrile/H2O (0.05% TFA) over 12 minutes on an Agilent Prep C-18 column, 50×100 mm, 5 μm. The resulting fractions were combined, frozen and lyophilized to give the intermediate. 25 mg of DBCO-(Gly-Gly-Glu-Gly-Gly-Trp)₂-Gly₂-Glu-NH₂ (SEQ ID NO:56), (0.015 mmol, 1 eq) and 17.11 mg of Compound 1 (0.055 mmol, 3.6 eq) were dissolved in 1.2 mL of dry DMSO. TEA (0.183 mmol, 12 eq) was added and the reaction mixture was stirred at room temperature for 5 minutes. 19.17 mg of HATU (0.05 mmol, 3.3 eq) was added and the reaction mixture was stirred at room temperature. The progress of the reaction was monitored by LC-MS. 1.2 additional equivalents of Compound 1 and 1.1 equivalents HATU were added after 1 hour. After 2 hours, the reaction was complete. The resulting product was purified on a preparatory HPLC system using a gradient of 30-60% acetonitrile/H2O (0.05% TFA) over 12 minutes on an Agilent Prep C-18 column, 30×100 mm, 5 μm. The resulting fractions were combined, frozen and lyophilized to give a spectroscopically pure white powder. MS (ESI) Calculated for C₁₃₀H₁₅₆N₃₄O₂₀ m/z 2515.96 found 839 (m/3)⁺.

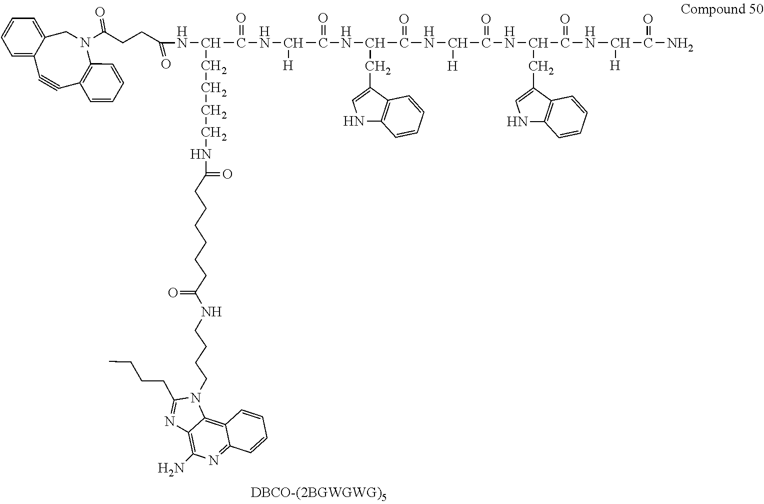 [SMILES: CCCCc1nc2c(N)nc3ccccc3c2n1CCCCNC(=O)CCCCCCC(=O)NCCCCC(NC(=O)CCC(=O)N1Cc2ccccc2C#Cc2ccccc21)C(=O)NCC(=O)NC(Cc1c[nH]c2ccccc12)C(=O)NCC(=O)NC(Cc1c[nH]c2ccccc12)C(=O)NCC(N)=O]

Compound 50, referred to as DBCO-(2BGWGWG)₅, 2B₅W₁₀G₁₅ or DBCO-(Glu(2B)₅(Trp)₁₀(Gly)₁₅), was synthesized from an Fmoc-(Lys-Gly-Trp-Gly-Trp-Gly)₅-NH₂ (SEQ ID NO:57), peptide precursor that was prepared by solid-phase peptide synthesis and Compound 6. 49.8 mg (0.01 mmol, 1 eq) of Fmoc-(Lys-Gly-Trp-Gly-Trp-Gly)₅-NH₂ (SEQ ID NO:58), was dissolved in 0.5 mL of dry DMSO. To this solution was added 0.492 mL of Compound 6 (0.03 mmol, 2.5 eq) as a 40 mg/mL stock solution in dry DMSO. TEA (0.01 mmol, 1 eq) was added and the reaction mixture was stirred at room temperature for 4 hours. Analytical HPLC using a gradient of 45-65% acetonitrile/H2O (0.05% TFA) over 10 minutes showed complete conversion to the penta-substituted intermediate. The reaction was quenched by addition of amino-2-propanol (0.03 mmol, 2.5 eq) and then 0.5 mL of 20% piperidine in DMF was added and the reaction mixture was stirred at room temperature for 30 minutes. The reaction mixture was added to 50 mL of ether and centrifuged at 3000 g at 4° C. for 10 minutes. The product was collected as a solid pellet and then washed twice more with ether, followed by drying under vacuum to yield the deprotected intermediate. 73.4 mg of the deprotected intermediate (0.0131 mmol, 1 eq) was dissolved in 0.5 mL of dry DMSO, followed by the addition of 0.066 mL (0.0196 mmol, 1.5 eq) of DBCO-NHS (40 mg/mL) and TEA (0.0131 mmol, 1 eq). The reaction was stirred for 1 hour at room temperature and then quenched by the addition of amino-2-propanol (0.0196 mmol, 1.5 eq). The product was then precipitated from 50 mL of 1 M HCl and centrifuged at 3000 g at 4° C. for 10 minutes. The product was collected as a solid pellet and then washed once more with 1 M HCl and once more with DI water. The final collected pellet was dried under vacuum to yield 15.1 mg (26% yield) of the final product. MS (ESI) calculated for C₃₁₉H₃₉₆N₇₂O₄₂ m/z 5909.1 found 1183 (m/5)⁺.

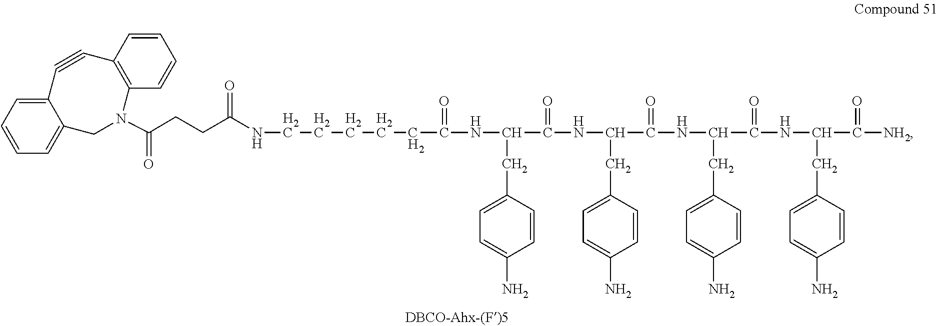 [SMILES: NC(=O)C(Cc1ccc(N)cc1)NC(=O)C(Cc1ccc(N)cc1)NC(=O)C(Cc1ccc(N)cc1)NC(=O)C(Cc1ccc(N)cc1)NC(=O)CCCCCNC(=O)CCC(=O)N1Cc2ccccc2C#Cc2ccccc21]

Compound 51, referred to as DBCO-Ahx-F′₅ or Ahx-F′₅ was synthesized by reacting 400 mg (0.4 mmol, 1 eq) of the precursor Ahx-(F′)₅-NH₂, which was prepared by solid phase peptide synthesis, with 171.05 mg of DBCO-NHS (0.4 mmol, 1.0 eq) and 258.1 mg of triethylamine (2.55 mmol, 6.0 eq) in 3.7 mL of DMSO. The DBCO-NHS was added in 4 increments of 0.25 eq. The reaction was run overnight at room temperature and HPLC indicated that the reaction was complete by 24 hours. Compound 51 was purified on a preparatory HPLC system using a gradient of 13-43% acetonitrile/H₂O (0.05% TFA) over 12 minutes on an Agilent Prep-C18 column, 50×100 mm, 5 μm. The product eluted at ˜5.7 minutes and the resulting fractions were collected, frozen and then lyophilized to obtain 217.0 mg (41.5% yield) of a spectroscopically pure (>95% AUC at 254 nm) white/yellow powder. MS (ESI) calculated for C₇₀H₇₆N₁₂O₉ m/z 1228.59, found 1228.7 (M+H)⁺.

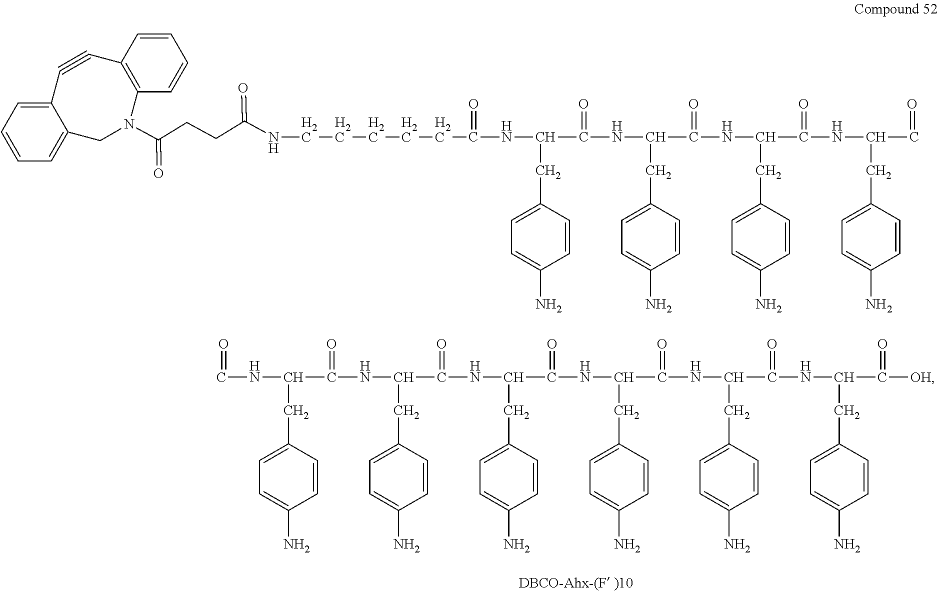

Compound 52, referred to as DBCO-Ahx-F′₁₀ or Ahx-F′₁₀ was synthesized by reacting 450 mg (0.26 mmol, 1 eq) of the precursor Ahx-(F′)₁₀-NH₂ (SEQ ID NO:59), which was prepared by solid phase peptide synthesis, with 103.4 mg of DBCO-NHS (0.26 mmol, 1.0 eq) and 286.1 mg of triethylamine (2.83 mmol, 11.0 eq) in 3.3 mL of DMSO. The DBCO-NHS was added in 4 increments of 0.25 eq. The reaction was run overnight at room temperature and HPLC indicated that the reaction was complete by 24 hours. Compound 52 was purified on a preparative HPLC system using a gradient of 15-45% acetonitrile/H₂O (0.05% TFA) over 12 minutes on an Agilent Prep-C18 column, 50×100 mm, 5 μm. The product eluted at ˜5.1 minutes and the resulting fractions were collected, frozen and then lyophilized to obtain 265.4 mg (50.6% yield) of a spectroscopically pure (>95% AUC at 254 nm) red/copper powder. MS (ESI) calculated for C₂₀₅H₂₂₆N₄₂O₂₄ m/z 3659.78, found 1221.3 (M/3+H)⁺.

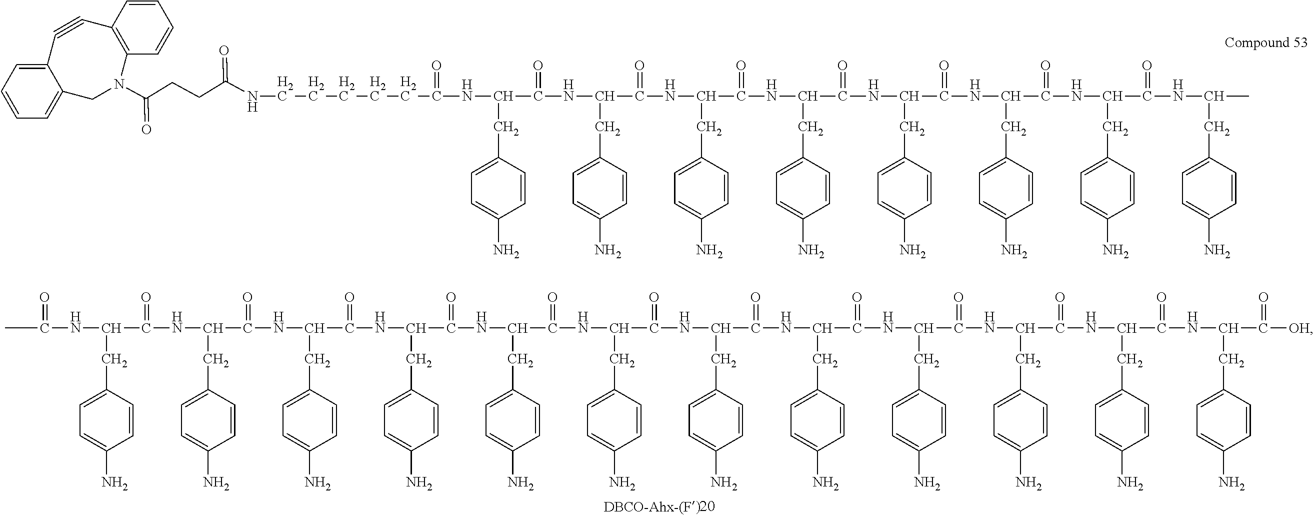

Compound 53, referred to as DBCO-Ahx-F′₂₀ or Ahx-F′₂₀ DBCO-Ahx-(F′)₂₀ (SEQ ID NO:60) was synthesized by reacting 480 mg (0.14 mmol, 1 eq) of the precursor Ahx-(F′)₂₀-NH₂ (SEQ ID NO:61), which was prepared by solid phase peptide synthesis, with 57.3 mg of DBCO-NHS (0.14 mmol, 1.0 eq) and 302.4 mg of triethylamine (2.99 mmol, 21.0 eq) in 3.0 mL of DMSO. The DBCO-NHS was added in 4 increments of 0.25 eq. The reaction was run overnight at room temperature and HPLC indicated that the reaction was complete by 24 hours. Compound 53 was purified on a preparative HPLC system using a gradient of 13-43% acetonitrile/H₂O (0.05% TFA) over 12 minutes on an Agilent Prep-C18 column, 50×100 mm, 5 m. The product eluted at ˜5.5 minutes and the resulting fractions were collected, frozen and then lyophilized to obtain 106.6 mg (20.5% yield) of a spectroscopically pure (94.4% AUC at 254 nm) brown/copper powder. MS (ESI) calculated for C₁₁₅H₁₂₆N₂₂O₁₄ m/z 2039.99, found 1020.5 (M/2+H)⁺.

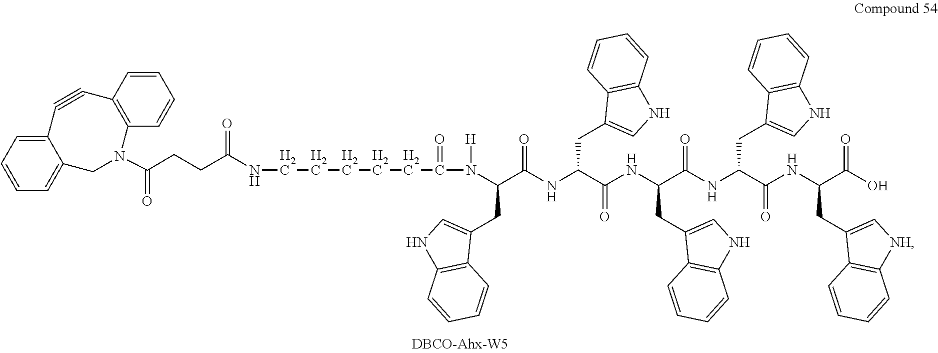 [SMILES: O=C(CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 54, referred to as DBCO-Ahx-W5 was synthesized by reacting 14.2 mg (0.035 mmol, 1 eq) of the precursor DBCO-NHS, with 37.5 mg of Ahx-(W)₅—NH₂ (SEQ ID NO:62) (0.035 mmol, 1 eq) that was prepared by solid phase peptide synthesis and 3.93 mg of triethylamine (0.039 mmol, 1.1 eq) in 0.5 mL of DMSO. The reaction was run overnight at room temperature and HPLC indicated that the reaction was complete by 24 hours. Compound 54 was precipitated out in twice with 1M HCL and once in H2O to obtain 34.3 mg (71.9% yield) of a spectroscopically pure (92.6% AUC at 254 nm) pink powder. MS (ESI) calculated for C₈₀H₇₆N₁₂O₉ m/z 1348.59, found 1348.4 (M+H)⁺.

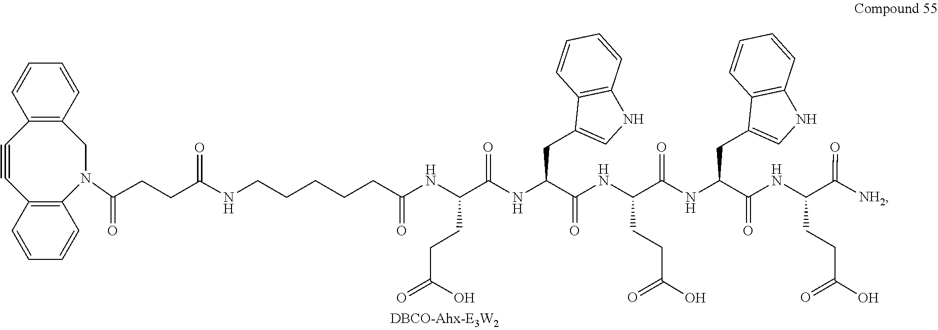 [SMILES: NC(=O)[C@H](CCC(=O)O)NC(=O)[C@H](Cc1c[nH]c2ccccc12)NC(=O)[C@H](CCC(=O)O)NC(=O)[C@H](Cc1c[nH]c2ccccc12)NC(=O)[C@H](CCC(=O)O)NC(=O)CCCCCNC(=O)CCC(=O)N1Cc2ccccc2C#Cc2ccccc21]

Compound 55, referred to as DBCO-Ahx-E₃W₂ or DBCO-Ahx-(Glu)₃(Trp)₂, was synthesized from a 6-aminohexanoic-Glu-Trp-Glu-Trp-Glu-NH₂ or Ahx-E₃W₂ precursor prepared by solid-phase peptide synthesis. 105 mg of Ahx-E₃W₂ (0.12 mmol, 1 eq) and 65.8 uL triethylamine (TEA) (0.47 mmol, 4 eq) were added to 525 uL anhydrous DMF and stirred at room temperature under ambient air for 5 minutes. 52.2 mg of DBCO-NHS ester (Scottsdale, Arizona, USA) (0.13 mmol, 1.1 eq) was then added while stirring vigorously and reacted for 1 hour. The reaction progress was monitored by HPLC (AUC 254 nm). After 1 hour the reaction was complete, and the reaction was quenched by adding amino-PEG₂₄-OH (San Diego, California, USA) (1 eq) and stirring for 1 hour. The reaction mixture was added dropwise to 5 mL of 0.2 M HCl to precipitate an off-white powder which was collected by centrifuging the solution at 4000 g at 4° C. for 5 minutes. The HCl solution was discarded and Compound 55 was collected as a solid off-white pellet. The off-white solid was re-suspended in 525 uL DMF and added dropwise to 5 mL of DI water and spun at 3000 g at 4° C. for 5 minutes; the DI water solution was discarded, and Compound 55 was collected as a solid pellet. This process was repeated and then the solid was collected and dried under vacuum to yield 117 mg of a spectroscopically pure (>95% AUC at 220 nm) white powder. MS (ESI) calculated for C₆₂H₆₈N₁₀O₁₄ m/z 1176.5, found 588.8 (M/2+H)⁺.

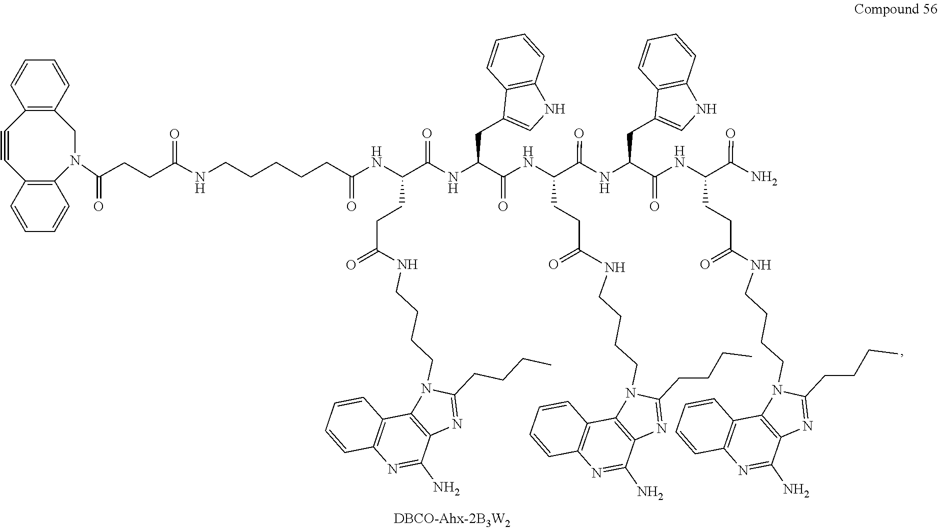 [SMILES: CCCCc1nc2c(N)nc3ccccc3c2n1CCCCNC(=O)CC[C@H](NC(=O)[C@H](Cc1c[nH]c2ccccc12)NC(=O)[C@H](CCC(=O)NCCCCn1c(CCCC)nc2c(N)nc3ccccc3c21)NC(=O)[C@H](Cc1c[nH]c2ccccc12)NC(=O)[C@H](CCC(=O)NCCCCn1c(CCCC)nc2c(N)nc3ccccc3c21)NC(=O)CCCCCNC(=O)CCC(=O)N1Cc2ccccc2C#Cc2ccccc21)C(N)=O]

Compound 56, referred to as DBCO-Ahx-2B₃W₂, DBCO-Ahx-E(2B)₃W₂ or DBCO-Ahx-Glu(2B)₃(Trp)₂ was synthesized by reacting Compound 55 and Compound 1 in the presence of HATU. 142.2 uL triethylamine (TEA) (1.02 mmol, 12 eq) was diluted in 1 mL anhydrous DMF, and 100 mg of Compound 55 (0.09 mmol, 1 eq) and 103 mg of Compound 1 (0.33 mmol, 3.9 eq) were added while stirring vigorously until fully dissolved. The reaction mixture was cooled to 4° C. by immersion in an ice bath for 5 minutes, then 106.6 mg of HATU (0.28 mmol, 3.3 eq) was added. The reaction mixture stirred vigorously for 1 hour at 4° C. and the reaction progress was monitored by HPLC (AUC 254 nm). The resulting product was purified on a preparative HPLC system using a gradient of 30-45% acetonitrile/H2O (0.05% TFA) over 12 minutes on an Agilent Prep C-18 column, 30×100 mm, 5 μm. The product eluted at ˜40% acetonitrile and the resulting fractions were combined, frozen and lyophilized to give 99.1 mg (60% yield) of a spectroscopically pure white powder (>95% AUC at 220 nm). MS (ESI) Calculated for C₁₁₆H₁₃₇N₂₅O₁₁ m/z 2056.1, found 685.4 (M/3+H)⁺.

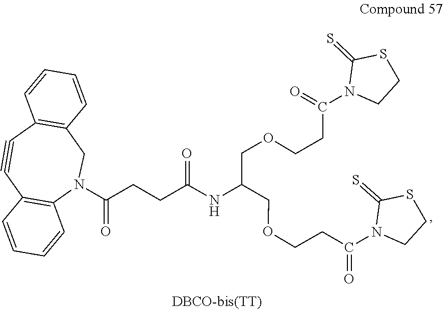 [SMILES: O=C(CCC(=O)N1Cc2ccccc2C#Cc2ccccc21)NC(COCCC(=O)N1CCSC1=S)COCCC(=O)N1CCSC1=S]

Compound 57, referred to as DBCO-2-amino-1,3-bis(carboxylethoxy)propane(TT)2 or DBCO-bis(TT) was synthesized by reacting 385.6 mg (0.74 mmol, 1 eq) of the precursor DBCO-2-Amino-1,3-bis(carboxylethoxy)propane, with 193.4 mg of 2-thiazoline-2-thiol (1.62 mmol, 2.2 eq) and 367.5 mg of 1-Ethyl-3-(3-dimethylaminopropyl) carbodiimide (EDC) (1.92 mmol, 2.6 eq) in and 4-dimethylaminopyridine in 4.0 mL of DCM. The reaction was run overnight at room temperature and HPLC indicated that the reaction was complete by 24 hours. The product eluted at 6.8 minutes on an Agilent analytical C18 column, 4.6×100 mm, 2.7 m. Compound 57 was extracted with ethyl acetate and 1M HCl and was dried on the rotovap to obtain 317.1 mg (59.3% yield) of an impure (27.0% AUC at 254 nm) yellow powder. MS (ESI) calculated for C₃₄H₃₆N₄O₆S₄ m/z 724.15, found 725.3 (M+H)⁺.

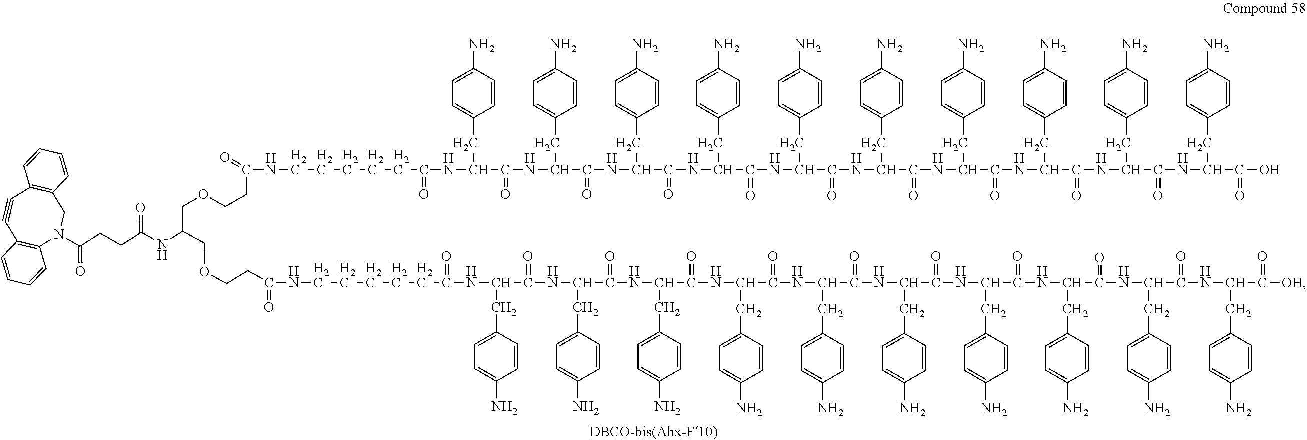

Compound 58, referred to as DBCO-2-amino-1,3-bis(carboxylethoxy)propane(Ahx-F′10)2 or DBCO-bis(Ahx-F′10) was synthesized by reacting 13.0 mg (0.018 mmol, 1 eq) of Compound 57, with 314.2 mg of Ahx-(F′)₁₀-NH₂ (SEQ ID NO:63) (0.18 mmol, 10 eq), which was prepared by solid phase peptide synthesis, and 199.5 mg of triethylamine (1.97 mmol, 11.0 eq) in 1.8 mL of DMSO. The reaction was run overnight at room temperature and HPLC indicated that the reaction was complete by 24 hours. Compound 58 was purified on a preparative HPLC system using a gradient of 5-25-35% acetonitrile/H₂O (0.05% TFA) over 14 minutes on an Agilent Prep-C18 column, 50×100 mm, 5 μm. The product eluted at ˜9.8 minutes and the resulting fractions were collected, frozen and then lyophilized to obtain 19.16 mg (26.8% yield) of a spectroscopically pure (83.4% AUC at 254 nm) orange powder. MS (ESI) calculated for C₂₂₀H₂₅₂N₄₄O₃₀ m/z 3989.95, found 1330.8 (M+3H)⁺.

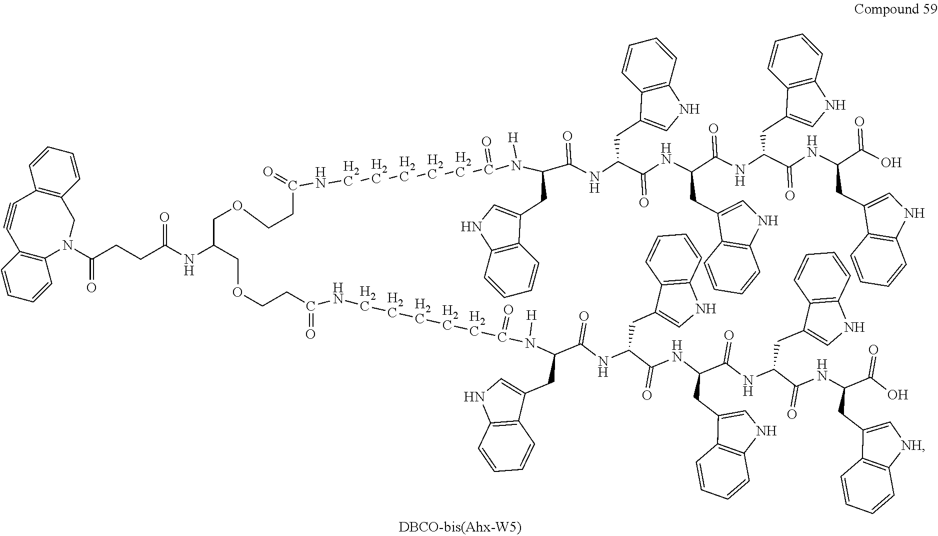 [SMILES: O=C(CCOCC(COCCC(=O)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O)NC(=O)CCC(=O)N1Cc2ccccc2C#Cc2ccccc21)NCCCCCC(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)N[C@H](Cc1c[nH]c2ccccc12)C(=O)O]

Compound 59, referred to as DBCO-2-Amino-1,3-bis(carboxylethoxy)propane(Ahx-W5)2 or DBCO-bis(Ahx-W5) was synthesized by reacting 13.0 mg (0.018 mmol, 1 eq) of Compound 57 with 41.3 mg of Ahx-(W)₅—NH₂ (0.039 mmol, 2.2 eq), which was prepared by solid phase peptide synthesis, and 9.1 mg of triethylamine (0.09 mmol, 2.3 eq) in 0.3 mL of DMSO. The reaction was run overnight at room temperature and HPLC indicated that the reaction was complete by 24 hours. Compound 59 was purified on a preparative HPLC system using a gradient of 15-60-90% acetonitrile/H₂O (0.05% TFA) over 16 minutes on an Agilent Prep-C18 column, 30×100 mm, 5 μm. The product eluted at ˜12.7 minutes and the resulting fractions were collected, frozen and then lyophilized to obtain 12.5 mg (30.8% yield) of a spectroscopically pure (>95% AUC at 254 nm) pink powder. MS (ESI) calculated for C₁₅₀H₁₅₂N₂₄O₂₀ m/z 2609.16, found 1305.0 (M+2H)⁺.

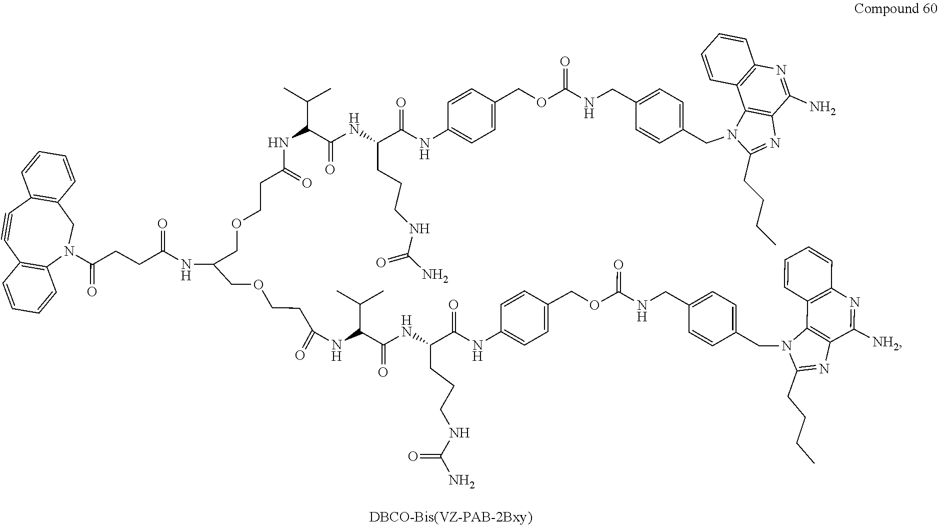

Compound 60, referred to as DBCO-(VZ-PAB-2Bxy)₂, or DBCO-Bis(VZ-PAB-2Bxy), was synthesized starting from an 2-Amino-1,3-bis(carboxylethoxy)propane precursor. 30 mg of 2-Amino-1,3-bis(carboxylethoxy)propane (0.11 mmol, 1 eq) and 76.96 μL of Triethylamine (TEA) (0.55 mmol, 5 eq) were combined in 0.5 mL of DMSO. 44.26 mg of DBCO-NHS (0.11 mmol, 1 eq) was added. The reaction progress was monitored by HPLC (AUC 254 nm). After 1 hour, HPLC showed the product and a small amount of excess DBCO-NHS. To one half of the reaction mixture was added 109.8 mg of Compound 26 (0.14 mmol, 2.6 eq). The reaction was cooled to 4° C. with an ice bath and then 46.18 mg of HATU (0.12 mmol, 2.2 eq) was added. The reaction progress was monitored by HPLC (AUC 254 nm). The resulting product, Compound 60, was purified on a preparatory HPLC system using a gradient of 30-60% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a spectroscopically pure (>95% AUC at 254 nm) off white powder. MS (ESI) calculated for C₁₁₀H₁₃₀N₂₂O₈₁₆ m/z 2015, found 1009 (M/2)⁺.

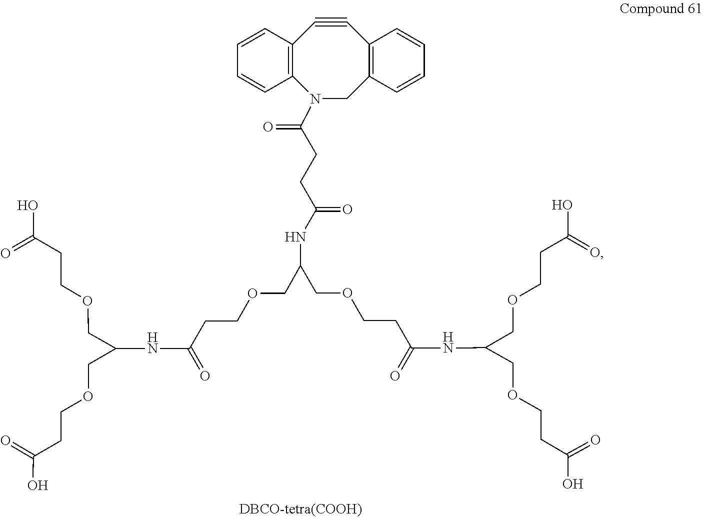 [SMILES: O=C(O)CCOCC(COCCC(=O)O)NC(=O)CCOCC(COCCC(=O)NC(COCCC(=O)O)COCCC(=O)O)NC(=O)CCC(=O)N1Cc2ccccc2C#Cc2ccccc21]

Compound 61, referred to as DBCO-2-amino-1,3-bis(carboxylethoxy)propane(COOH)₄ or DBCO-tetra(COOH), was synthesized by reacting 250 mg (0.34 mmol, 1.1 eq) of the precursor Compound 57 with 170 mg of DBCO-2-Amino-1,3-bis(carboxylethoxy)propane (0.6 mmol, 2 eq) and 190 mg of TEA (1.9 mmol, 6 eq) in 2.5 mL of DMF. The reaction was run for 1 hour at room temperature and HPLC indicated the reaction was complete. MS (ESI) calculated for C₄₆H₆₀N₄O₁₈ m/z 956.4, found 957.2 (M+H)⁺.

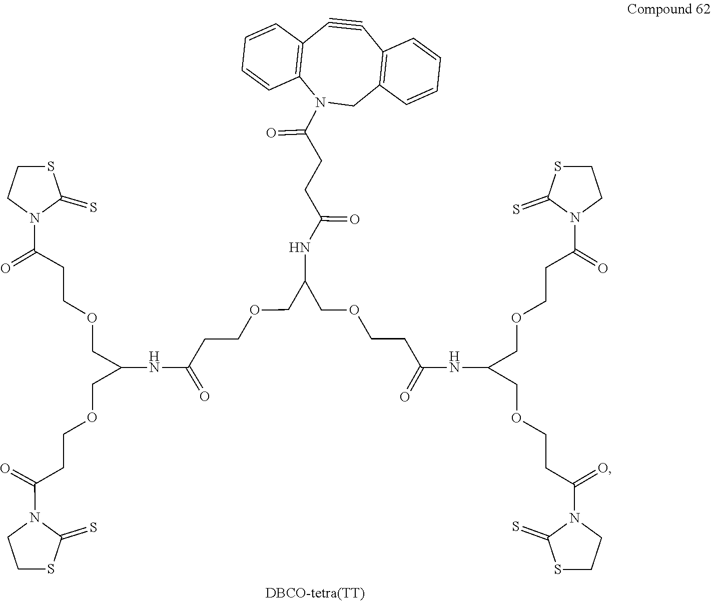 [SMILES: O=C(CCOCC(COCCC(=O)NC(COCCC(=O)N1CCSC1=S)COCCC(=O)N1CCSC1=S)NC(=O)CCC(=O)N1Cc2ccccc2C#Cc2ccccc21)NC(COCCC(=O)N1CCSC1=S)COCCC(=O)N1CCSC1=S]

Compound 62, referred to as DBCO-2-amino-1,3-bis(carboxylethoxy)propane(TT)₄ or DBCO-tetra(TT), was synthesized by reacting 178 mg (0.19 mmol, 1 eq) of the precursor Compound 61 with 115 mg of 2-thiazoline-2-thiol (0.96 mmol, 5.2 eq). TEA (2.98 mmol, 16 eq) was added and the reaction mixture was cooled in an ice bath for 5 minutes. 310 mg of HATU (0.8 mmol, 4.4 eq) was added and the reaction mixture was stirred in an ice bath. The progress of the reaction was monitored by LC-MS. After 2 hours, the reaction was complete. Compound 62 was precipitated in 1M HCl and once in H₂O. The resulting solid was dissolved in ACN and dried on rotovap to obtain 215 mg (85.0% yield) of an impure (53.0% AUC at 254 nm) yellow/brown oil. MS (ESI) calculated for C₅₈H₇₂N₈O₁₄S₈ m/z 1360.3, found 1361.0 (M+H)⁺.

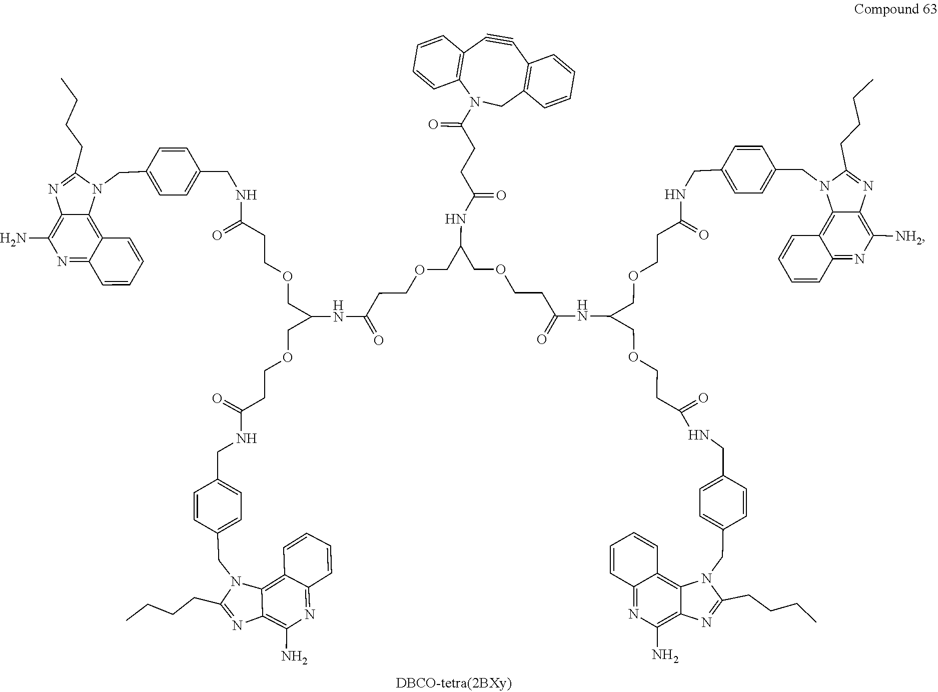 [SMILES: CCCCc1nc2c(N)nc3ccccc3c2n1Cc1ccc(CNC(=O)CCOCC(COCCC(=O)NCc2ccc(Cn3c(CCCC)nc4c(N)nc5ccccc5c43)cc2)NC(=O)CCOCC(COCCC(=O)NC(COCCC(=O)NCc2ccc(Cn3c(CCCC)nc4c(N)nc5ccccc5c43)cc2)COCCC(=O)NCc2ccc(Cn3c(CCCC)nc4c(N)nc5ccccc5c43)cc2)NC(=O)CCC(=O)N2Cc3ccccc3C#Cc3ccccc32)cc1]

Compound 63, referred to as DBCO-2-amino-1,3-bis(carboxylethoxy)propane(2BXy)₄ or DBCO-tetra(2BXy), was synthesized by reacting 16 mg (0.012 mmol, 1 eq) of the precursor Compound 62 with 17 mg of Compound 4 (0.047 mmol, 4 eq) and TEA (0.047 mmol, 4 eq) in 0.5 mL DMSO. The progress of the reaction was monitored by HPLC. After 1 hour, the reaction was complete. Compound 63 was purified on a preparative HPLC system using a gradient of 38-48% acetonitrile/H₂O (0.05% TFA) over 12 minutes on an Agilent Prep-C18 column, 30×100 mm, 5 μm. The product eluted at ˜4.0 minutes and the resulting fractions were collected, frozen and then lyophilized to obtain 13.6 mg (49.8% yield) of a spectroscopically pure (98.3% AUC at 254 nm) white powder. MS (ESI) calculated for C₁₃₄H₁₅₂N₂₄O₁₄ m/z 2321.2, found 775.0 (M/3+H)⁺.

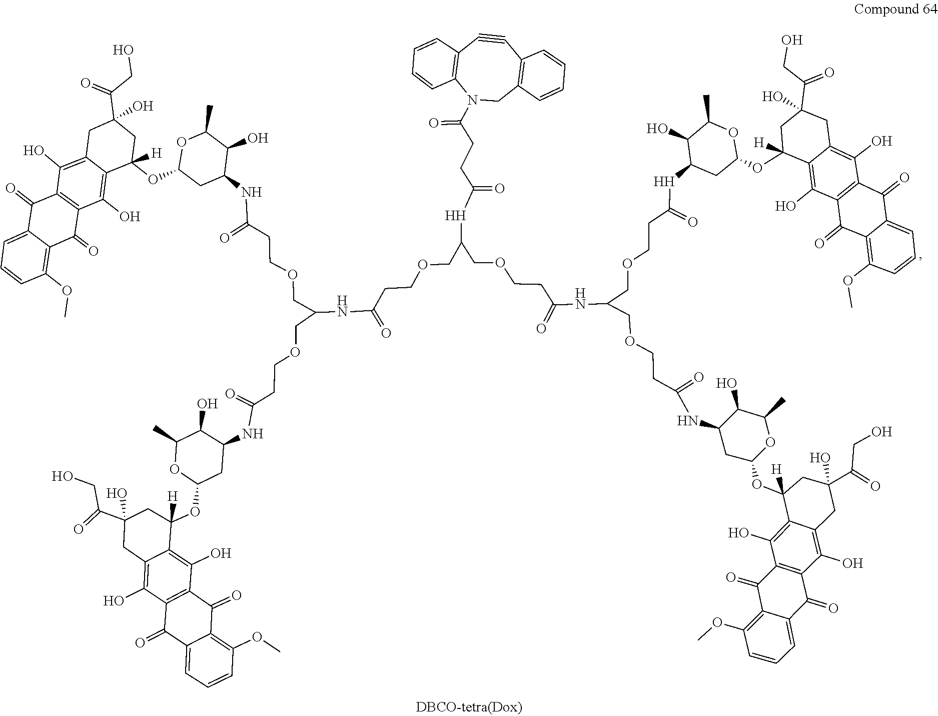

Compound 64 referred to as DBCO-2-amino-1,3-bis(carboxylethoxy)propane(Doxorubicin)₄ or DBCO-tetra(Dox), was synthesized by reacting 23 mg (0.017 mmol, 1 eq) of the precursor Compound 62 with 40 mg of Doxorubicin Hydrochloride (0.069 mmol, 4 eq) and TEA (0.138 mmol, 8 eq) in 1.5 mL DMSO. The progress of the reaction was monitored by HPLC. After 1 hour, the reaction was complete. Compound 64 was purified on a preparative HPLC system using a gradient of 38-48% acetonitrile/H₂O (0.05% TFA) over 12 minutes on an Agilent Prep-C18 column, 30×100 mm, 5 μm. The product eluted at ˜7.5 minutes and the resulting fractions were collected, frozen and then lyophilized to obtain 5.3 mg (20.1% yield) of a spectroscopically pure (95.9% AUC at 254 nm) red powder. MS (ESI) calculated for C₁₅₄H₁₆₈N₈O₅₈ m/z 3059.0.

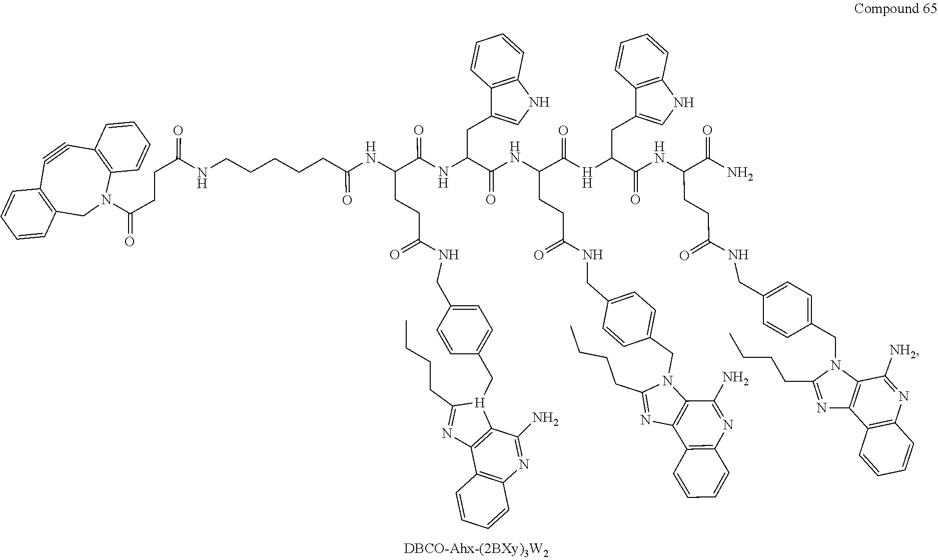

Compound 65, referred to as DBCO-Ahx-(2BXy)₃W₂, DBCO-Ahx-E(2BXy)₃W₂ or DBCO-Ahx-Glu(2BXy)₃(Trp)₂ was synthesized by first dissolving Compound 55 (234.8 mg, 0.199 mmol, 1 eq.) in DMF (4.7 mL) to make a 50 mg/mL solution. Compound 4 (280 mg, 0.780 mmol, 3.9 eq.) was added, along with TEA (333.6 uL, 2.393 mmol, 12 eq.). The reaction was chilled in an ice bath to 4 C for 10 minutes with stirring. HATU (250.3 mg, 0.658 mmol, 3.3 eq.) was then added to the reaction. The reaction was stirred for 1.5 hours at 4 C until HPLC indicated the reaction was complete. The reaction was precipitated with 2×100 mL of 1% KHSO4, followed by 1×100 mL H2O. The resulting product was dried, dissolved in DMSO, then purified by reverse phase flash chromatography on a 60 g Biotage Safar C18 Bio Duo column over a 3-step gradient: 25% acetonitrile/H2O (0.05% TFA v/v) over 3 column volumes (CVs), followed by 25-40% acetonitrile/H2O (0.05% TFA v/v) 40 CVs and 40% acetonitrile/H2O (0.05% TFA v/v) 2 CVs. The product eluted at ˜35% acetonitrile and the resulting fractions were collected and the solvent removed to obtain a 91.0% yield of a spectroscopically pure (99.6% AUC at 220 nm) solid. MS (ESI) Calculated for C₁₂₈H₁₃₇N₂₅O₁₁ m/z 2200.09, found 1101.4 (M/2+H)⁺.

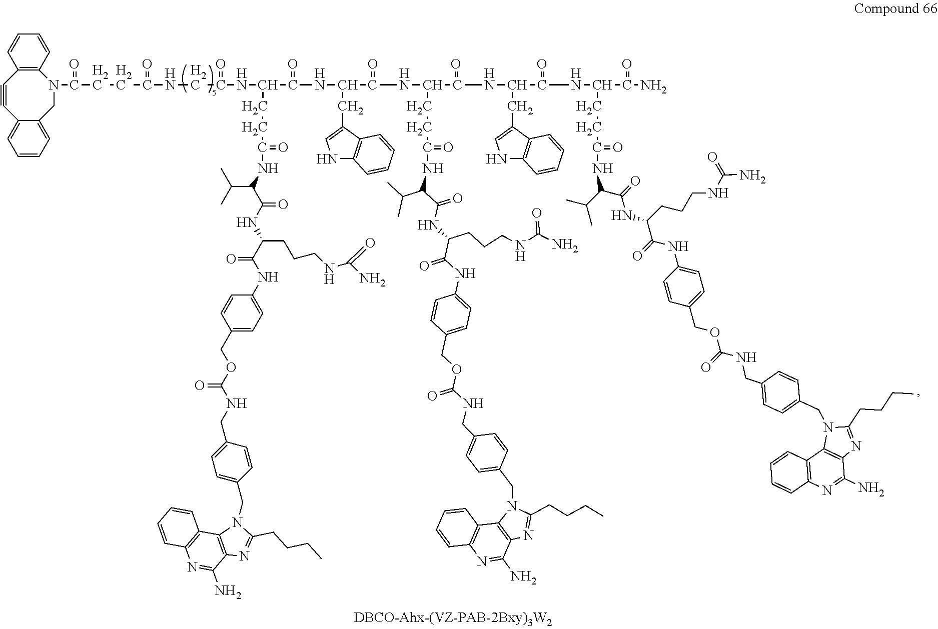

Compound 66, referred to as DBCO-Ahx-(VZ-PAB-2Bxy)₃W₂ or DBCO-Ahx-E(VZ-PAB-2Bxy)₃W₂ was synthesized using the same procedure as described for Compound 65, except Compound 26 (6 eq) was used instead of Compound 4. The reaction was precipitated with 2× in 1% KHSO4 and the resulting product was dried, dissolved in DMSO and was purified on a preparatory HPLC system using a gradient of 40-60% acetonitrile/H₂O (0.05% TFA) over 10 minutes on an Agilent Prep-C18 column, 30×100 mm, 5 μm. The product eluted at ˜6.7 minutes and the resulting fractions were collected, frozen and then lyophilized to obtain a 48% yield of a spectroscopically pure (98.7% AUC at 220 nm) solid. MS (ESI) Calculated for C₁₈₅H₂₁₈N₄₀O₂₆ m/z 3415.7, found 1139.8 (M/3)⁺.

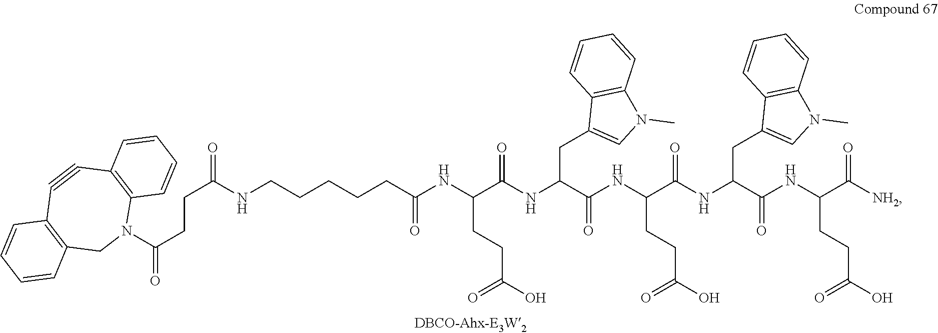 [SMILES: Cn1cc(CC(NC(=O)C(CCC(=O)O)NC(=O)C(Cc2cn(C)c3ccccc23)NC(=O)C(CCC(=O)O)NC(=O)CCCCCNC(=O)CCC(=O)N2Cc3ccccc3C#Cc3ccccc32)C(=O)NC(CCC(=O)O)C(N)=O)c2ccccc21]

Compound 67, referred to as DBCO-Ahx-E₃W′₂ was synthesized by first dissolving the precursor H2N-Ahx-EW′EW′E (100.2 mg, 0.109 mmol, 1 eq.) (SEQ ID NO:64), which was prepared by solid phase peptide synthesis, in 0.5 mL DMSO. Triethylamine (TEA) (76.1 uL, 0.546 mmol, 5 eq.) was added and the reaction was stirred for 5 minutes. DBCO-NHS (48.3 mg, 0.120 mmol, 1.1 eq.) was added and the reaction was stirred for 1 hour at room temperature until HPLC indicated the reaction was complete. The reaction was quenched by the addition of amino-2-propanol (4.6 uL, 0.060 mmol) and TEA (16.7 uL, 0.120 mmol) and the reaction mixture was stirred at room temperature for 30 minutes. The reaction was precipitated with 2× of 1% KHSO4, followed by 1×H2O. The resulting product was dried and resulted in a 94% yield of a spectroscopically pure (93% AUC at 220 nm) solid. MS (ESI) Calculated for C₆₄H₇₂N₁₀O₁₄ m/z 1204.52, found 1205.3 (M+H)⁺.

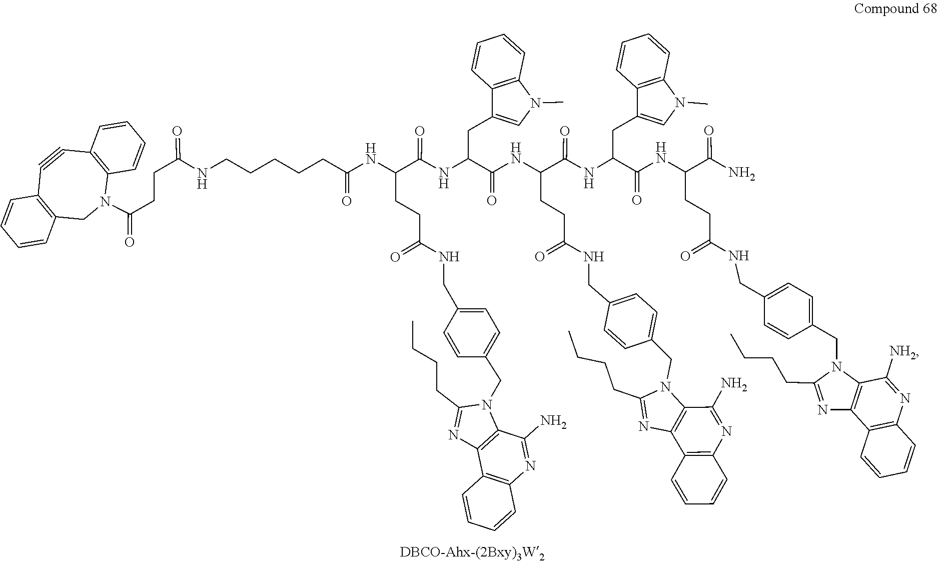 [SMILES: CCCCc1nc2c3ccccc3nc(N)c2n1Cc1ccc(CNC(=O)CCC(NC(=O)C(Cc2cn(C)c3ccccc23)NC(=O)C(CCC(=O)NCc2ccc(Cn3c(CCCC)nc4c5ccccc5nc(N)c43)cc2)NC(=O)C(Cc2cn(C)c3ccccc23)NC(=O)C(CCC(=O)NCc2ccc(Cn3c(CCCC)nc4c5ccccc5nc(N)c43)cc2)NC(=O)CCCCCNC(=O)CCC(=O)N2Cc3ccccc3C#Cc3ccccc32)C(N)=O)cc1]

Compound 68, referred to as DBCO-Ahx-(2Bxy)₃W′₂, or DBCO-Ahx-E(2BXy)₃W′₂ was synthesized by reacting Compound 67 (40.3 mg, 0.033 mmol, 1 eq.) and Compound 4 (53 mg, 0.163 mmol, 4.9 eq.) in 0.8 mL DMF with triethylamine (TEA) (74.5 uL, 0.535 mmol, 16.2 eq.). The reaction was chilled in an ice bath to 4 C for 10 minutes and then HATU (56 mg, 0.147 mmol, 4.5 eq.) was added. The reaction was stirred vigorously for 2 hours until HPLC indicated the reaction was complete. The reaction was precipitated with 1× of 1% KHSO4. The resulting product was dissolved in DMSO, then purified by reverse phase flash chromatography on a 25 g Biotage Safar C18 Bio Duo column over a 4-step gradient: 30% acetonitrile/H2O (0.05% TFA v/v) over 3 column volumes (CVs), followed by 30-40% acetonitrile/H2O (0.05% TFA v/v) 40 CVs, 40-45% acetonitrile/H2O (0.05% TFA v/v) 10 CVs and 45-95% acetonitrile/H2O (0.05% TFA v/v) 10 CVs. The product eluted at ˜35% acetonitrile and the resulting fractions were collected and the solvent removed to obtain a 22.0% yield of a spectroscopically pure (95.8% AUC at 220 nm) solid. MS (ESI) Calculated for C₁₃₀H₁₄₁N₂₅O₁₁ m/z 2228.12, found 1115.6 (M/2+H)⁺.

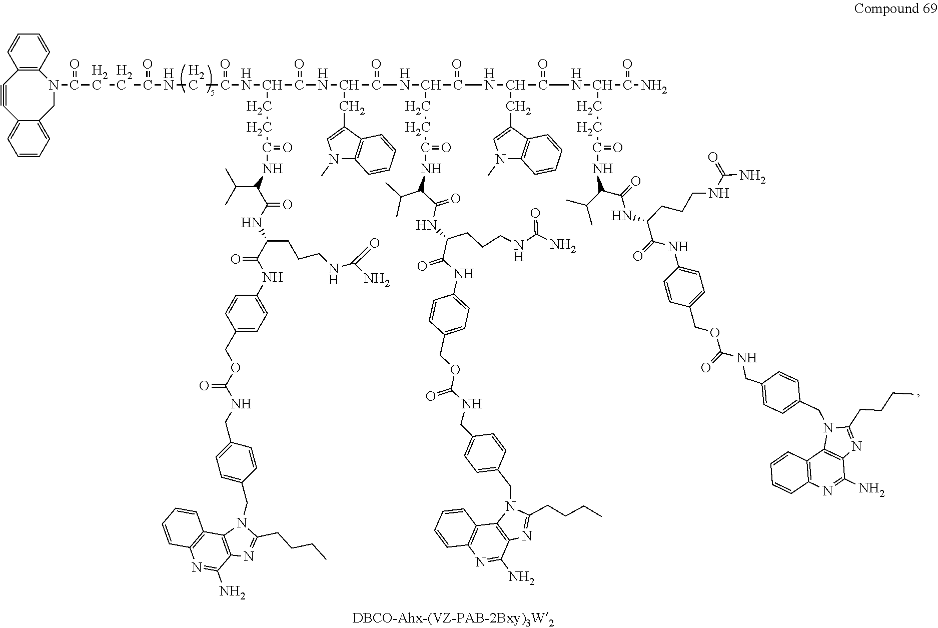

Compound 69, referred to as DBCO-Ahx-(VZ-PAB-2Bxy)₃W′₂ or DBCO-Ahx-E(VZ-PAB-2Bxy)₃W′₂ was synthesized using the same procedure as described for Compound 68, except Compound 26 (6 eq) was used instead of Compound 4. The reaction was precipitated with 2× in 1% KHSO4 and the resulting product was dried. MS (ESI) Calculated for C₁₈₇H₂₂₂N₄₀O₂₆ m/z 3443.73, found 1149.3 (M/3+H)⁺.

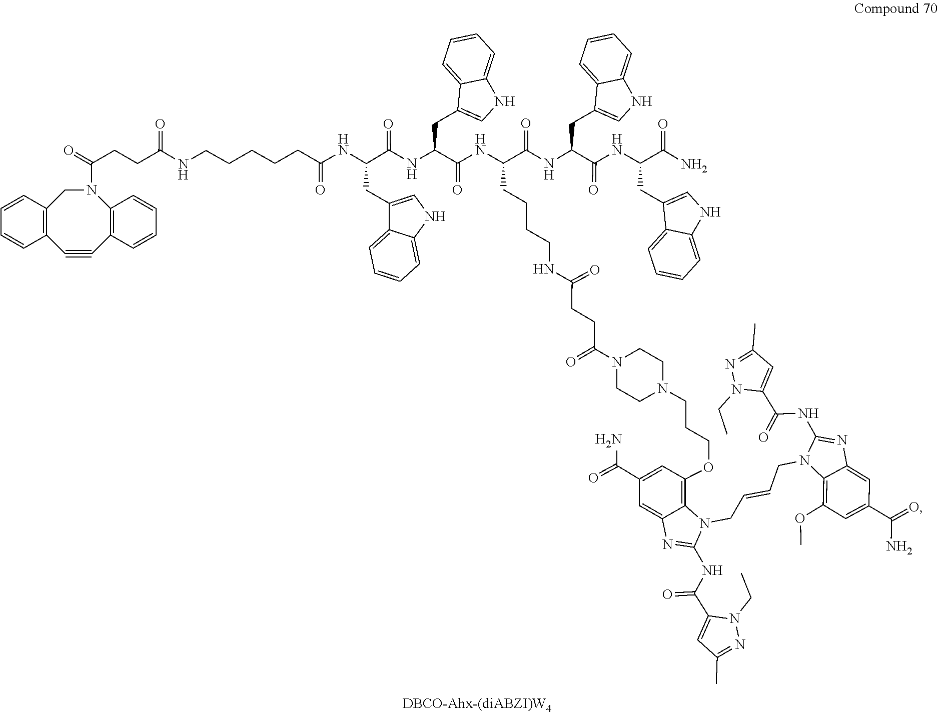 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CCC(=O)NCCCC[C@H](NC(=O)[C@H](Cc4c[nH]c5ccccc45)NC(=O)[C@H](Cc4c[nH]c5ccccc45)NC(=O)CCCCCNC(=O)CCC(=O)N4Cc5ccccc5C#Cc5ccccc54)C(=O)N[C@@H](Cc4c[nH]c5ccccc45)C(=O)N[C@@H](Cc4c[nH]c5ccccc45)C(N)=O)CC3)c21]

Compound 70, referred to as DBCO-Ahx-(diABZI)W₄ or DBCO-Ahx-E(diABZI)W₄, was synthesized by first synthesizing intermediate 70-a, referred to as Fmoc-Ahx-WWK(COOH)WW. The precursor Fmoc-Ahx-WWKWW (200 mg, 0.163 mmol, 1 eq) (SEQ ID NO:), which was synthesized by solid phase peptide synthesis, was dissolved in 2 mL DMF and DIEA (42.6 uL, 0.245 mmol, 1.5 eq) was added. Succinic anhydride as a 100 mg/mL solution in DMSO (302.1 uL, 1.85 eq) was added and the reaction was stirred for 1 hour at room temperature until HPLC indicated the reaction was complete. 70-a was purified by reverse phase flash chromatography on a 60 g Biotage Safar C18 Bio Duo column over a 3-step gradient: 25% acetonitrile/H2O (0.05% TFA v/v) over 1 column volume (CVs), followed by 25-95% acetonitrile/H2O (0.05% TFA v/v) 15 CVs, and 95% acetonitrile/H2O (0.05% TFA v/v) 2 CVs. The product eluted at ˜65% acetonitrile and the resulting fractions were collected and the solvent removed to obtain a 70% yield of a spectroscopically pure (87% AUC at 220 nm) solid.

70-b. Intermediate 70-a was deprotected by dissolving in 20% piperidine/DMF and stirring for 20 minutes until HPLC indicated the reaction was complete. The deprotected 70-a was precipitated into diethyl ether and pelleted by centrifuging at 3000 rpm for 5 minutes. The diethyl ether supernatant was decanted and fresh diethyl ether was added. The pellet was broken up with a spatula and the product was again pelleted by centrifugation. The diethyl ether was decanted to provide an oil which was resuspended in H2O and then dried by lyophilization. The deprotected intermediate was dissolved in DMSO and TEA (27.6 uL, 0.198 mmol, 2.3 eq) was added. The mixture was stirred at room temperature for 5 minutes followed by the addition of DBCO-NHS (55.4 mg, 0.138 mmol, 1.6 eq) The reaction was stirred for 3 hours at room temperature until HPLC indicated that the reaction was complete. Amino-2-propanol (7.3 uL, 0.095 mmol, 0.7 eq) was added to quench the remaining DBCO-NHS. After stirring for 30 minutes at room temperature, HPLC indicated that the quenching was complete. The product was precipitated into 1% KHSO4 and pelleted by centrifuging at 3000 rpm for 5 minutes. The supernatant was decanted and fresh 1% KHSO4 was added. The process of pelleting and decanting was repeated followed by a final wash with DI water. After decanting the water, the product was dried by lyophilization resulting in a spectroscopically pure (94% AUC at 220 nm) solid.

Compound 70. Intermediate 70-b (17.6 mg, 0.013 mmol, 1 eq) and Compound 7 (12.4 mg 0.014 mmol, 1.1 eq) were dissolved in 176 μL of DMF. TEA (5.3 uL, 0.038 mmol, 3 eq) was added and the reaction was chilled to 4 C in an ice bath. HATU (5.8 mg, 0.015 mmol, 1.2 eq) was added and the reaction was stirred at 4 C for 1.5 hours until HPLC indicated that the reaction was complete. The product was precipitated into 1% KHSO4 and pelleted by centrifuging at 3000 rpm for 5 minutes. The supernatant was decanted and fresh 1% KHSO4 was added. The process of pelleting and decanting was repeated followed by a final wash with DI water. After decanting the water, the product was dried by lyophilization. Compound 70 was purified by reverse phase flash chromatography on a 10 g Biotage Safar C18 Bio Duo column over a 3-step gradient: 39% acetonitrile/H2O (0.05% TFA v/v) over 1 column volume (CVs), followed by 39-59% acetonitrile/H2O (0.05% TFA v/v) 25 CVs, and 95% acetonitrile/H2O (0.05% TFA v/v) 2 CVs. The product eluted at ˜46% acetonitrile and the resulting fractions were collected and dried to obtain a 70% yield of a spectroscopically pure (87% AUC at 220 nm) solid. MS (ESI) Calculated for C₁₂₁H₁₃₃N₂₇O₁₆ m/z 2220.04, found 1111.8 (M/2+H)⁺.

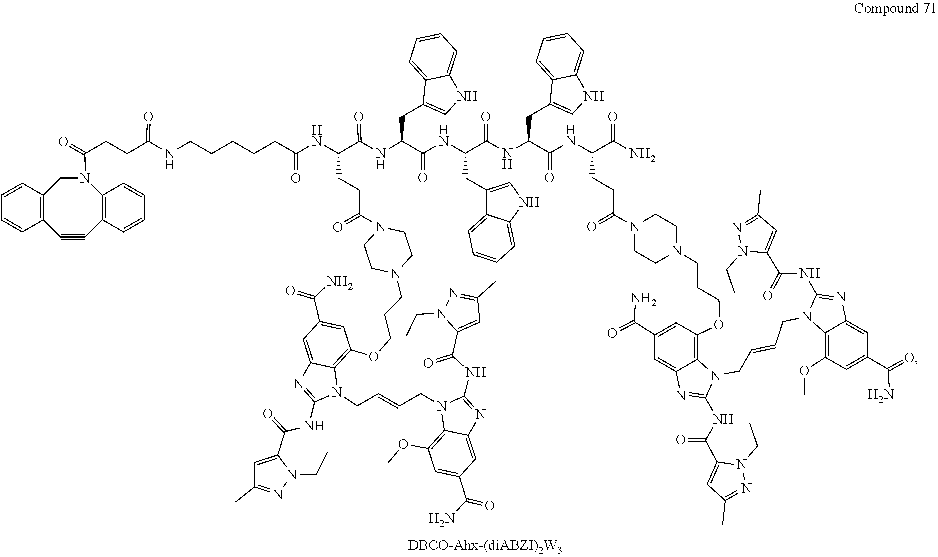 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)CC[C@H](NC(=O)[C@H](Cc4c[nH]c5ccccc45)NC(=O)[C@H](Cc4c[nH]c5ccccc45)NC(=O)[C@H](Cc4c[nH]c5ccccc45)NC(=O)[C@H](CCC(=O)N4CCN(CCCOc5cc(C(N)=O)cc6nc(NC(=O)c7cc(C)nn7CC)n(C/C=C/Cn7c(NC(=O)c8cc(C)nn8CC)nc8cc(C(N)=O)cc(OC)c87)c56)CC4)NC(=O)CCCCCNC(=O)CCC(=O)N4Cc5ccccc5C#Cc5ccccc54)C(N)=O)CC3)c21]

Compound 71, referred to as DBCO-Ahx-(diABZI)₂W₃ or DBCO-Ahx-E(diABZI)₂W₃ was synthesized by first synthesizing intermediate 71-a, referred to as Fmoc-Ahx-TT. Fmoc-6-aminohexanoic acid (2.0 g, 5.56 mmol), 2-mercaptothiazoline (1.4 g, 12.0 mmol), EDC (2.8 g, 14.0 mmol) and DMAP (68 mg, 0.57 mmol) were combined in DCM (120 ml). The reaction was stirred at room temperature for 16 hours until HPLC indicated the reaction was complete. The reaction mixture was washed with 2×200 mL 1M HCL and 1×100 mL brine and the organic layer was dried with MgSO₄ and then removed under vacuum. 71-a was then purified by flash chromatography on a 100 g Biotage Safar SilicaD column over a 1-step gradient: 0-5% methanol in DCM over 3 column volumes (CVs), The product eluted at ˜3% methanol and the resulting fractions were collected and the solvent removed to obtain a 1.8 g (71% yield) of a spectroscopically pure (87% AUC at 220 nm) yellow solid. MS (ESI) Calculated for C₂₄H₂₆N₂O₃S₂ m/z 454.14, found 477.9 (M+Na)⁺.

Compound 71-b. To intermediate 71-a (1380.5 uL, 0.303 mmol, 1.3 eq) as a 100 mg/mL solution in DMSO, were added H2N-EWWWE (SEQ ID NO:65) (194.8 mg, 0.234 mmol, 1 eq.) and DIEA (69.1 uL, 0.397 mmol, 1.7 eq.). The reaction was stirred at room temperature for 4 hours until HPLC indicated that the reaction was complete. 71-b, Fmoc-Ahx-EWWWE, was purified by reverse phase flash chromatography on a 60 g Biotage Safar C18 Bio Duo column over a 3-step gradient: 35% acetonitrile/H2O (0.05% TFA v/v) over 1 column volume (CVs), followed by 35-65% acetonitrile/H2O (0.05% TFA v/v) for 25 CVs, and 95% acetonitrile/H2O (0.05% TFA v/v) for 2 CVs. The product eluted at ˜52% acetonitrile and the resulting fractions were collected and dried to obtain a 70% yield of a spectroscopically impure (87% AUC at 220 nm) solid.

Compound 71. Fmoc-Ahx-EWWWE would be deprotected in a 20% piperidine/DMF solution for 20 minutes at RT. After the reaction is complete, the product is precipitated twice in diethyl ether. After the final decanting of the diethyl ether, the pellet is resuspended in H2O and lyophilized to dryness. The resulting product, H2N-Ahx-EWWWE, is then dissolved in DMSO to make a 100 mg/mL solution and TEA was added. The reaction is stirred for 5 minutes at RT and then DBCO-NHS is added. Upon completion as indicated by HPLC, the remaining DBCO-NHS is quenched with amino-2-propanol. The resulting reaction mixture is flash purified. The resulting product, DBCO-Ahx-EWWWE, is then dissolved in DMF to make a 100 mg/mL solution, TEA and Compound 7 are added and the reaction is chilled in an ice bath to 4 C. Once chilled, HATU is added and the reaction proceeds for 2 hours at 4 C. Once complete as indicated by HPLC, the resulting mixture is purified by prep HPLC, clean fractions are combined and solvent is removed.

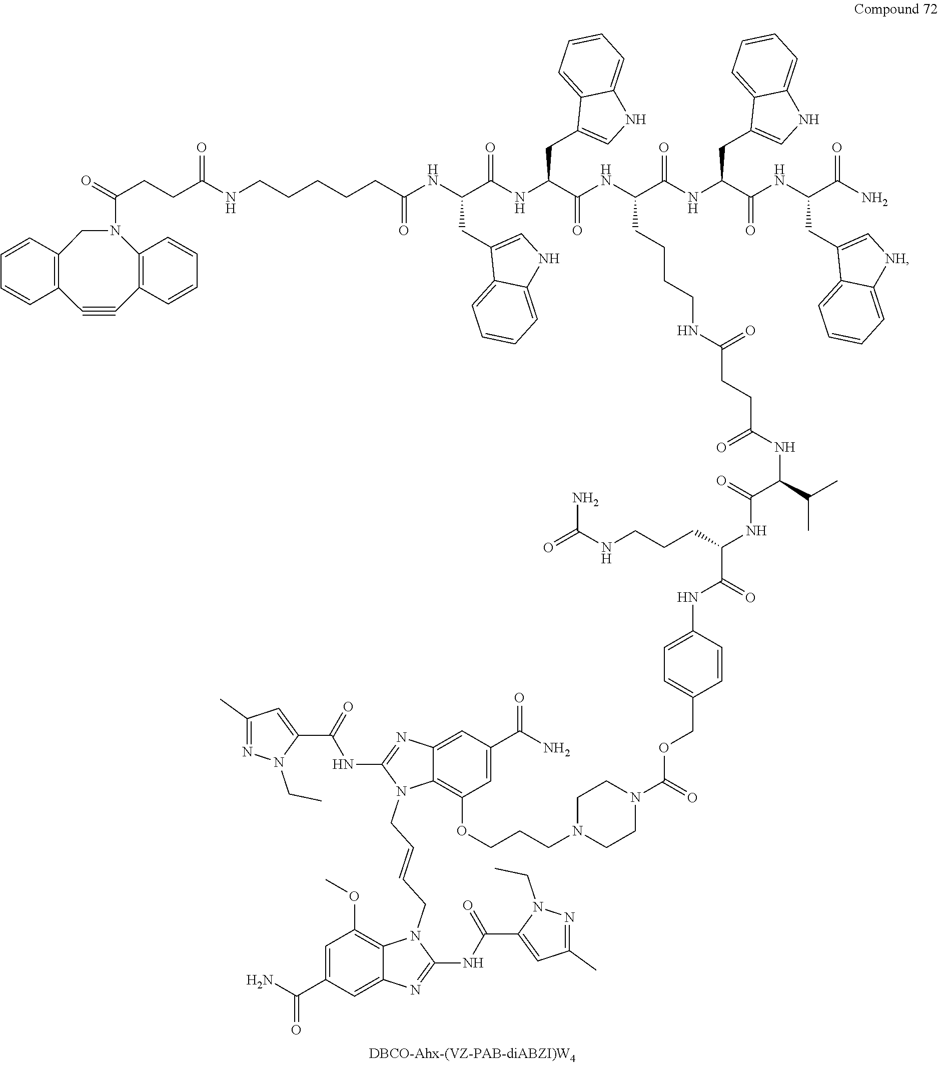 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](CCCNC(N)=O)NC(=O)[C@@H](NC(=O)CCC(=O)NCCCC[C@H](NC(=O)[C@H](Cc5c[nH]c6ccccc56)NC(=O)[C@H](Cc5c[nH]c6ccccc56)NC(=O)CCCCCNC(=O)CCC(=O)N5Cc6ccccc6C#Cc6ccccc65)C(=O)N[C@@H](Cc5c[nH]c6ccccc56)C(=O)N[C@@H](Cc5c[nH]c6ccccc56)C(N)=O)C(C)C)cc4)CC3)c21]

Compound 72, referred to as DBCO-Ahx-(VZ-PAB-diABZI)W₄ or DBCO-Ahx-E(VZ-PAB-diABZI)W₄ was synthesized using the same procedure as described for Compound 70, except Compound 27 was used instead of Compound 7. The reaction was precipitated with 2× of 1% KHSO4, followed by 1×H2O. The resulting solid was dried and then purified by reverse phase flash chromatography on a 10 g Biotage Safar C18 Bio Duo column over a 3-step gradient: 5% acetonitrile/H2O (0.05% TFA v/v) over 1 column volume (CVs), followed by 5-95% acetonitrile/H2O (0.05% TFA v/v) 20 CVs, and 95% acetonitrile/H2O (0.05% TFA v/v) 2 CVs. The product eluted at ˜60% acetonitrile and the resulting fractions were collected and the solvent removed a spectroscopically impure (87% AUC at 220 nm) solid. MS (ESI) Calculated for C₁₄₀H₁₆₀N₃₂O₂ m/z 2625.24 found 1314.8 (M/2+H)⁺.

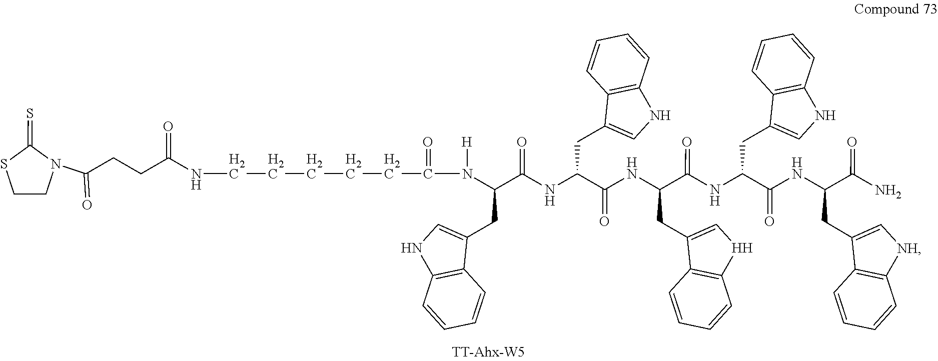

Compound 73, referred to as TT-Ahx-W5 was synthesized starting from an NH2-Ahx-W5 (SEQ ID NO:66) prepared by solid phase peptide synthesis. 500 mg of NH2-Ahx-W5 was dissolved in 5 mL of DMF. 263 μL of TEA was added and the solution was stirred at rt for 5 minutes followed by the addition of 47.15 mg of succinic anhydride. The reaction progress was monitored by HPLC (AUC 254 nm). After 1 hour, HPLC showed clean conversion to the succinic acid product. 116.8 mg of Thiazoline-2-thiol (0.98 mmol, 1.6 eq) was added. The solution was cooled to 4° C. with an ice bath and 232.9 mg of HATU (0.61 mmol, 1.3 eq) was added. The reaction progress was monitored by HPLC (AUC 254 nm). After 1 hour, HPLC showed clean conversion product. The reaction mixture was added to 100 mL of 0.2 M HCl and pelleted by centrifuging at 3000 RPM for 5 minutes. The supernatant was decanted and 100 mL of fresh 0.2 M HCl was added. The pellet was broken up with a spatula and the product was again pelleted by centrifuging at 3000 RPM for 5 minutes. The supernatant was decanted and 100 mL of DI water was added. The pellet was broken up with a spatula and the product was pelleted by centrifuging at 3000 RPM for 5 minutes. The supernatant was decanted and the sample was frozen and dried by lyophilization. Compound 73 was used without further purification. MS (ESI) calculated for C₆₈H₇₁N₁₃O₈S₂ m/z 1261.5, found 1262.5 (M/2)⁺.

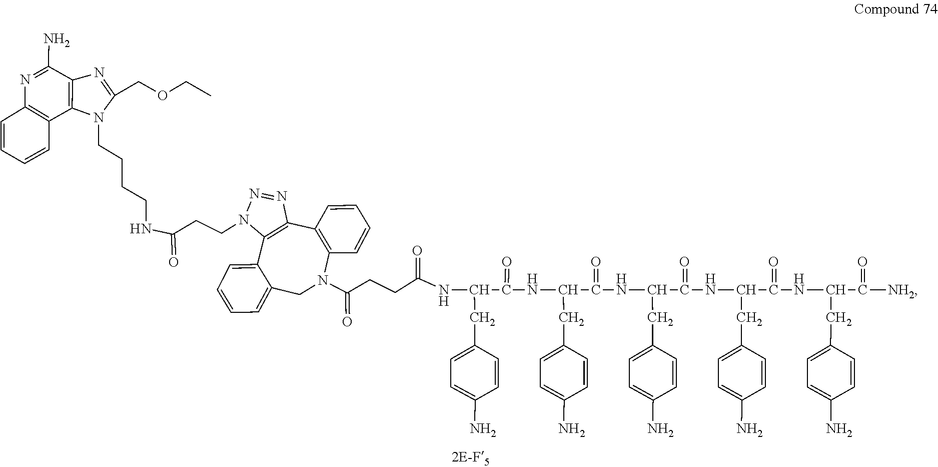 [SMILES: CCOCc1nc2c(N)nc3ccccc3c2n1CCCCNC(=O)CCn1nnc2c1-c1ccccc1CN(C(=O)CCC(=O)NC(Cc1ccc(N)cc1)C(=O)NC(Cc1ccc(N)cc1)C(=O)NC(Cc1ccc(N)cc1)C(=O)NC(Cc1ccc(N)cc1)C(=O)NC(Cc1ccc(N)cc1)C(N)=O)c1ccccc1-2]

Compound 74, referred to as 2E-F′₅ was prepared by reacting 0.5 mg of Compound 32 in DMSO at 20 mg/mL with 1.0 mole equivalents of Compound 3, which resulted in the complete conversion of starting material to Compound 74. MS (ESI) calculated for C₁₈₄H₉₂N₂₀O₉ m/z 1524.7.

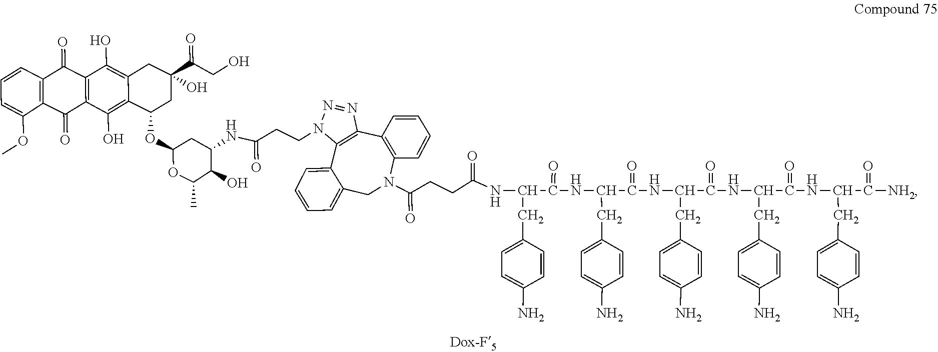 [SMILES: COc1cccc2c1C(=O)c1c(O)c3c(c(O)c1C2=O)C[C@@](O)(C(=O)CO)C[C@@H]3O[C@H]1C[C@H](NC(=O)CCn2nnc3c2-c2ccccc2CN(C(=O)CCC(=O)NC(Cc2ccc(N)cc2)C(=O)NC(Cc2ccc(N)cc2)C(=O)NC(Cc2ccc(N)cc2)C(=O)NC(Cc2ccc(N)cc2)C(=O)NC(Cc2ccc(N)cc2)C(N)=O)c2ccccc2-3)[C@@H](O)[C@H](C)O1]

Compound 75, referred to as Dox-F′₅ was produced by reacting 0.5 mg of Compound 32 in DMSO at 20 mg/mL with 1.0 mole equivalents of azide functionalized doxorubicin, which resulted in the complete conversion of starting material to Compound 75. MS (ESI) calculated for C₉₄H₉₈N₁₆O₁₉ m/z 1754.7.

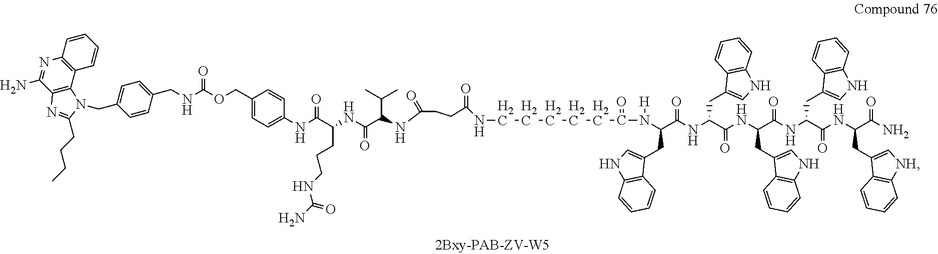

Compound 76, also referred to as 2Bxy-PAB-ZV-W5 was synthesized using Compound 73 and Compound 26 as starting materials. 25 mg of Compound 26 (0.033 mmol, 1 eq) was dissolved in 900 μL of DMSO. 9.11 uL of TEA (0.065 mmol, 2 eq) was added and the solution was stirred at rt for five minutes. 41.3 mg of Compound 73 (0.033 mmol, 1 eq) was added. The reaction was monitored by HPLC at 254 nm. After 1 hour at rt the reaction was complete. The resulting product, Compound 76, was purified on a preparatory HPLC system using a gradient of 35-65% acetonitrile/H₂O (0.05% TFA) over 12 minutes on an Agilent Prep-C18 column, 30×100 mm, 5 m. The product eluted at −7 minutes and the resulting fractions were collected, frozen and then lyophilized to obtain a spectroscopically pure (>90% AUC at 254 nm) off white powder. MS (ESI) calculated for C₁₀₆H₁₁₈N₂₂O₁₃ m/z 1908.25, found 958.92 (M/2)⁺.

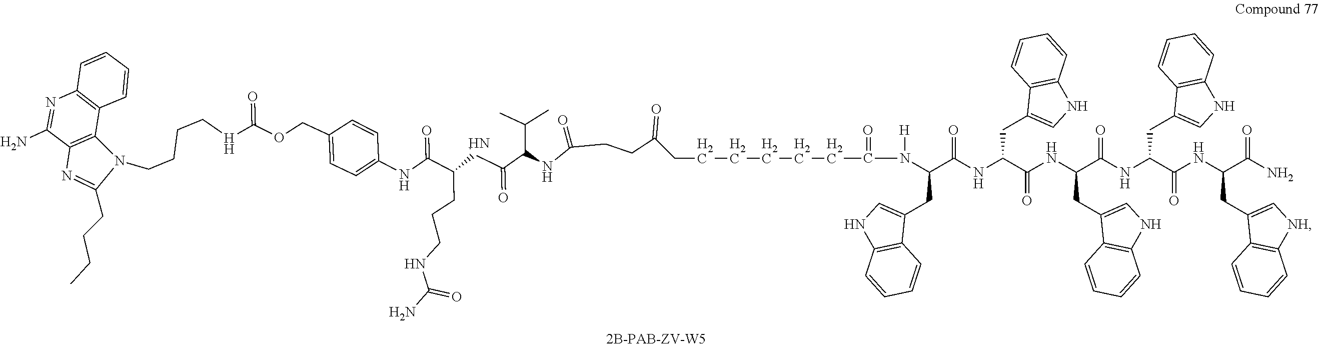

Compound 77 also referred to as 2B-PAB-ZV-W5 was prepared using the same procedure as Compound 76 except Compound 162 was used instead of Compound 26 and was purified using the same Prep HPLC method. MS (ESI) calculated for C₁₀₂H₁₁₈N₂₂O₁₃ m/z 1858.92, found 931.1 (M/2)⁺.

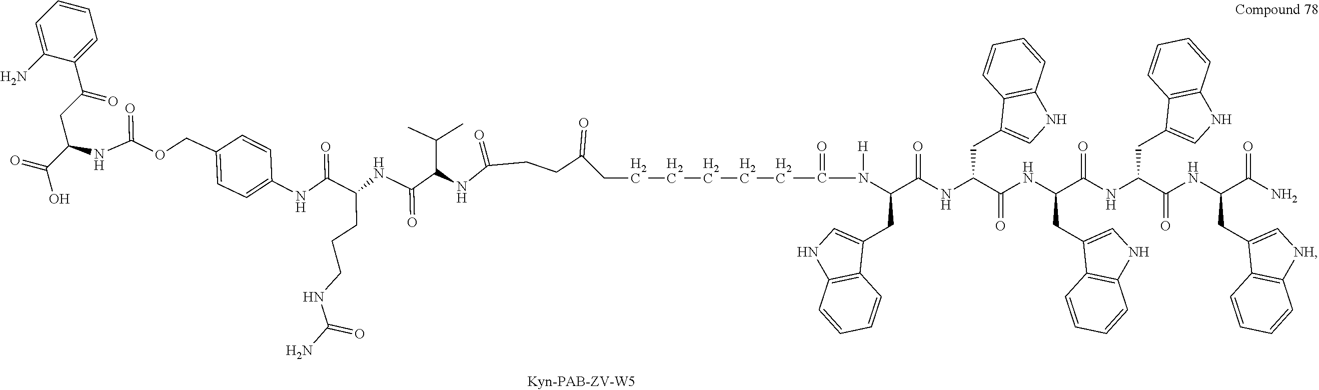

Compound 78 also referred to as Kyn-PAB-ZV-W5 was prepared using the same procedure as Compound 76 except Compound 163 was used instead of Compound 26. Compound 78 was purified on a preparatory HPLC system using a gradient of 40-50% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a spectroscopically pure (>95% AUC at 254 nm) off white powder. MS (ESI) calculated for C₉₄H₁₀₅N₁₉O₁₆ m/z 1755.8, found 1756.99 (M+H)⁺.

Amphiphiles with Dendron-Based S Blocks Having Cone Architecture

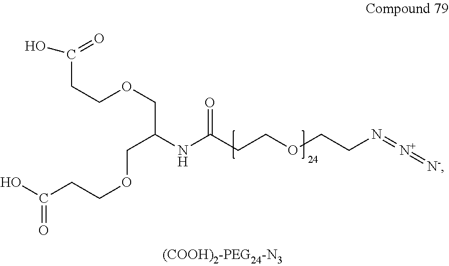

Compound 79, referred to as (COOH)₂—PEG₂₄-N₃ or bis(COOH)-PEG₂₄-N₃ was synthesized by reacting 2.8 g of N₃-P₂₄-NHS ester (2.2 mmol, 1 eq) and 0.57 g of 2-amino-1,3-bis(carboxylethoxy)propane HCl salt (2.1 mmol, 0.95 eq) dissolved in 30 mL anhydrous DCM. Triethylamine (3 mL, 22.1 mmol, 10 eq) was added to the reaction mixture. The reaction was stirred at room temperature for 3 hours until HPLC indicated the reaction was complete. The reaction solvent was removed under vacuum and the reaction mixture was redissolved in 1:1 DMSO/H2O w/0.05% TFA. The product was purified by flash C18 chromatography on a 12 g Biotage SNAP C18 column using a 2-step gradient: 0% acetonitrile in H2O (0.05% TFA) over 3 column volumes (CVs), followed by 0-60% acetonitrile in H2O (0.05% TFA) over 20 CVs. The product eluted at ˜25% acetonitrile and the resulting fractions were collected and the solvent removed under vacuum to yield 2.0 g (65.2% yield) of a spectroscopically pure (>97% AUC at 220 nm) white oil. MS (ESI) calculated for C₆₀H₁₁₆N₄O₃₁ m/z 1388.8, found 1412.6 (M+Na+H)⁺.

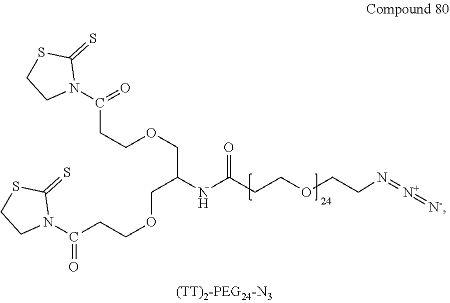

Compound 80, referred to as (TT)₂-PEG₂₄-N₃ or bis(TT)-PEG₂₄-N₃ was synthesized by reacting 2.0 g of Compound 79 (1.5 mmol, 1 eq) and 1.2 g of HATU (3.2 mmol, 2.2 eq) in 24 mL of DCM. The mixture was cooled on ice for 5 min and 1.6 mL of triethylamine (11.7 mmol, 8 eq) was added. The mixture was stirred on ice for 5 min and 0.45 g of thizoline-2-thiol (TT) (3.8 mmol, 2.6 eq) was added. The reaction mixture was stirred at room temperature for 2 hours until HPLC indicated the reaction was complete. The product was purified by flash chromatography on a 100 g Biotage Safar SilicaD column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 2.0 g (85.3% yield) of a spectroscopically pure (96.1% AUC at 220 nm) yellow oil. MS (ESI) Calculated for C₆₆H₁₂₂N₉O₂₉S₄ m/z 1590.7, found 782.3 ((M-N₃)/2)⁺.

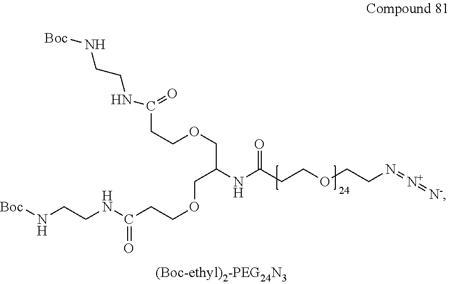

Compound 81, referred to as (Boc-ethyl)₂-PEG₂₄-N₃ was synthesized by reacting 347 mg of Compound 80 (0.2 mmol, 1 eq) and 83 mg of N-boc-ethylenediamine (0.5 mmol, 2.4 eq) in 3.5 mL of DCM. Triethylamine (73 uL, 0.5 mmol, 2.4 eq) was added and the reaction mixture was stirred at room temperature for 1 hour until HPLC indicated the reaction was complete. The solvent was removed under vacuum and the product was dissolved in DMSO and purified on a preparative HPLC system using a gradient of 27-57% acetonitrile/H₂O (0.05% TFA) over 12 minutes on an Agilent Prep-C18 column, 30×100 mm, 5 μm. The product eluted at ˜7.2 minutes and the resulting fractions were collected, frozen and then lyophilized to obtain 189 mg (51.8% yield) of a spectroscopically pure (94.7% AUC at 220 nm) white solid. MS (ESI) Calculated for C₇₄H₁₄₄N₈O₃₃ m/z 1672.98, found 1574.6 (M-Boc+H)⁺.

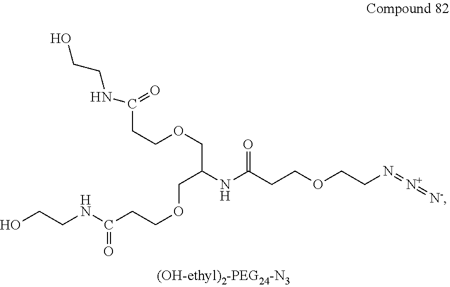

Compound 82, referred to as (OH-ethyl)₂-PEG₂₄-N₃ was synthesized following the same procedure as Compound 81, except ethanolamine was used instead of N-boc-ethylenediamine. Compound 82 was purified on a preparative HPLC system using a gradient of 15-45% acetonitrile/H₂O (0.05% TFA) over 12 minutes on an Agilent Prep-C18 column, 30×100 mm, 5 m. The product eluted at ˜7.1 minutes and the resulting fractions were collected, frozen and then lyophilized to obtain an 81.2% yield of a spectroscopically pure (98.3% AUC at 220 nm) white solid. MS (ESI) Calculated for C₆₄H₁₂₆N₆O₃₁ m/z 1474.9, found 1476.6 (M+H)⁺.

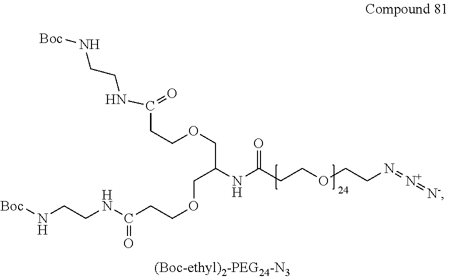

Compound 83, referred to as (COOH-ethyl)₂-PEG₂₄-N₃ was synthesized following the same procedure as Compound 81, except beta-alanine was used instead of N-boc-ethylenediamine and MeOH was used as the solvent. Compound 83 was purified on a preparative HPLC system using a gradient of 13-43% acetonitrile/H₂O (0.05% TFA) over 2 minutes on an Agilent Prep-C18 column, 30×100 mm, 5 μm. The product eluted at ˜9.2 minutes and the resulting fractions were collected, frozen and then lyophilized to obtain a 37.2% yield of as spectroscopically pure (90.0% AUC at 220 nm) white solid. MS (ESI) Calculated for C₆₆H₁₂₆N₈O₃₃ m/z 1530.8, found 1533.6 (M+H)⁺.

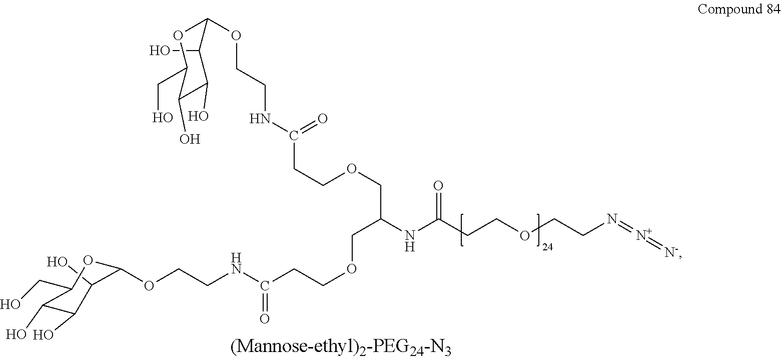

Compound 84, referred to as (Mannose-ethyl)₂-PEG₂₄-N₃ was synthesized following the same procedure as Compound 81, except 2-aminoethyl-a-mannopyranoside (Broadpharm (San Diego, CA)) was used instead of N-boc-ethylenediamine and DMSO was used as the solvent. Compound 84 was purified on a preparatory HPLC system using a gradient of 15-45% acetonitrile/H₂O (0.05% TFA) over 12 minutes on an Agilent Prep-C18 column, 30×100 mm, 5 m. The product eluted at ˜7.0 minutes and the resulting fractions were collected, frozen and then lyophilized to obtain a 70.0% yield of a spectroscopically pure (98.2% AUC at 220 nm) white solid. MS (ESI) Calculated for C₇₆H₁₄₆N₆O₄₁ m/z 1799.0.

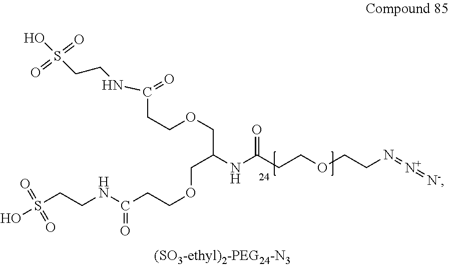

Compound 85, referred to as (SO₃-ethyl)₂-PEG₂₄-N₃ was synthesized following the same procedure as Compound 81, except taurine was used instead of N-boc-ethylenediamine and 2:1 DMSO/PBS was used as the solvent. Compound 85 was purified on a preparatory HPLC system using a gradient of 15-40% acetonitrile/H₂O (0.05% TFA) over 12 minutes on an Agilent Prep-C18 column, 30×100 mm, 5 μm. The product eluted at ˜6.7 minutes and the resulting fractions were collected, frozen and then lyophilized to obtain a 47.1% yield of a spectroscopically pure (>99% AUC at 220 nm) white solid. MS (ESI) Calculated for C₆₄H₁₂₆N₆O₃₅S₂ m/z 1602.8, found 802.4 (M/2+H)⁺.

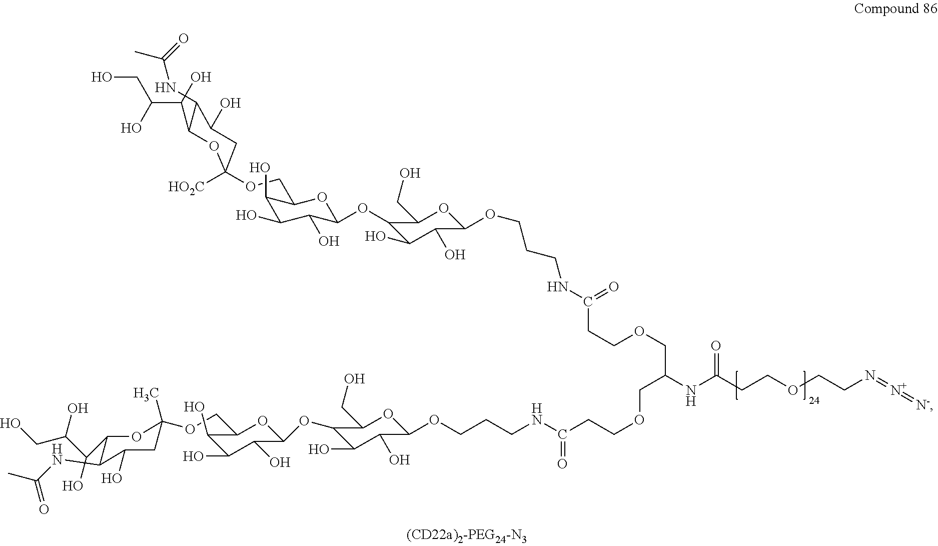

Compound 86, referred to as (CD22a)₂-PEG₂₄-N₃ was synthesized following the same procedure as Compound 81, except CD22a amine (WuXi Biologics, China) was used instead of N-boc-ethylenediamine and DMSO was used as the solvent. Compound 86 was purified on a preparatory HPLC system using a gradient of 15-35% acetonitrile/H₂O (0.05% TFA) over 12 minutes on an Agilent Prep-C18 column, 30×100 mm, 5 μm. The product eluted at ˜8.1 minutes and the resulting fractions were collected, frozen and then lyophilized to obtain a 68.8% yield of a spectroscopically pure (98.3% AUC at 220 nm) white solid. MS (ESI) Calculated for C₁₁₂H₂₀₄N₈O₆₇ m/z 2733.3, found 1367.8 (M/2+H)⁺.

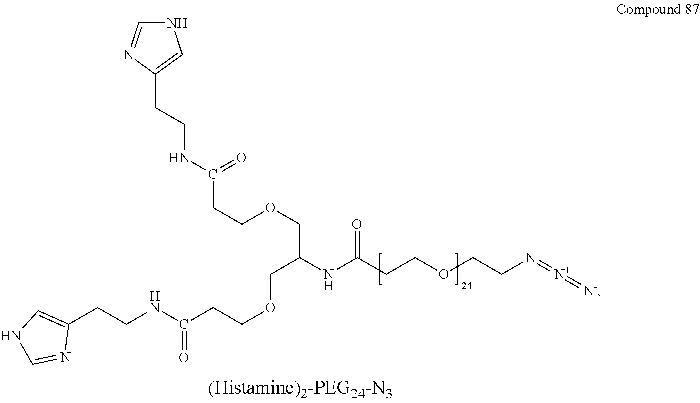

Compound 87, referred to as (Histamine)₂-PEG₂₄-N₃ was synthesized following the same procedure as Compound 81, except histamine was used instead of N-boc-ethylenediamine and DMSO was used as the solvent. Compound 87 was purified on a preparatory HPLC system using a gradient of 18-38% acetonitrile/H₂O (0.05% TFA) over 10 minutes on an Agilent Prep-C18 column, 30×100 mm, 5 μm. The product eluted at ˜5.3 minutes and the resulting fractions were collected, frozen and then lyophilized to obtain a 56.4% yield of a spectroscopically pure (>99% AUC at 220 nm) white solid. MS (ESI) Calculated for C₇₀H₁₃₀N₁₀O₂₉ m/z 1574.9 found 788.6 (M/2+H)⁺.

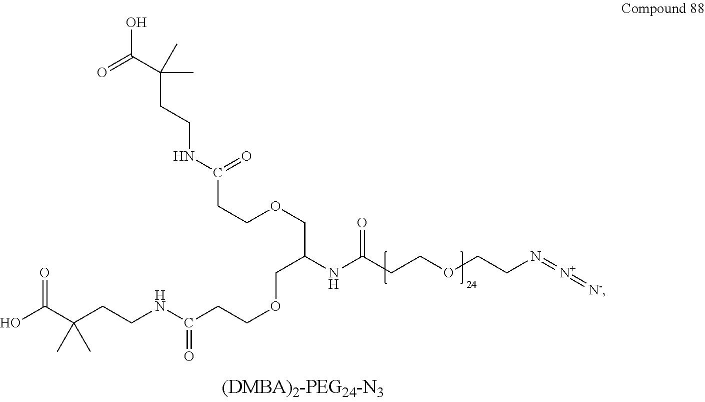

Compound 88, referred to as (DMBA)₂-PEG₂₄-N₃ was synthesized following the same procedure as Compound 81, except 4-amino-2,2,-dimethylbutanoic acid was used instead of N-boc-ethylenediamine and DMSO was used as the solvent. Compound 88 was purified on a preparatory HPLC system using a gradient of 25-45% acetonitrile/H₂O (0.05% TFA) over 10 minutes on an Agilent Prep-C18 column, 30×100 mm, 5 μm. The product eluted at ˜5.8 minutes and the resulting fractions were collected, frozen and then lyophilized to obtain a 49.3% yield of a spectroscopically pure (93.3% AUC at 220 nm) white solid. MS (ESI) Calculated for C₇₂H₁₃₈N₆O₃₃ m/z 1614.9 found 808.7 (M/2+H)⁺.

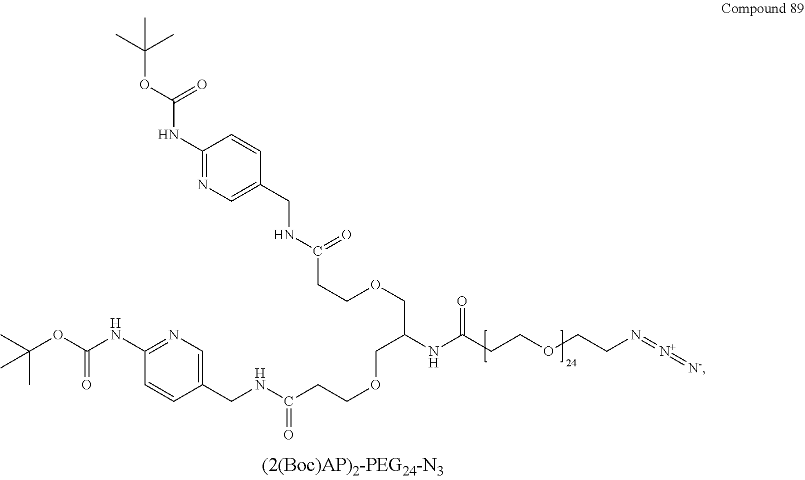

Compound 89, referred to as (2(Boc)AP)₂—PEG₂₄-N₃ was synthesized following the same procedure as Compound 81, except 5-aminomethyl-2-(Boc-amino)-pyridine was used instead of N-boc-ethylenediamine and DMSO was used as the solvent. Compound 89 was purified on a preparatory HPLC system using a gradient of 28-48% acetonitrile/H₂O (0.05% TFA) over 12 minutes on an Agilent Prep-C18 column, 30×100 mm, 5 μm. The product eluted at ˜4.9 minutes and the resulting fractions were collected, frozen and then lyophilized to obtain a 58.7% yield of a spectroscopically pure (86.1% AUC at 220 nm) white solid. MS (ESI) Calculated for C₈₂H₁₄₆N₁₀O₃₃ m/z 1799.0 found 900.8 (M/2+H)⁺.

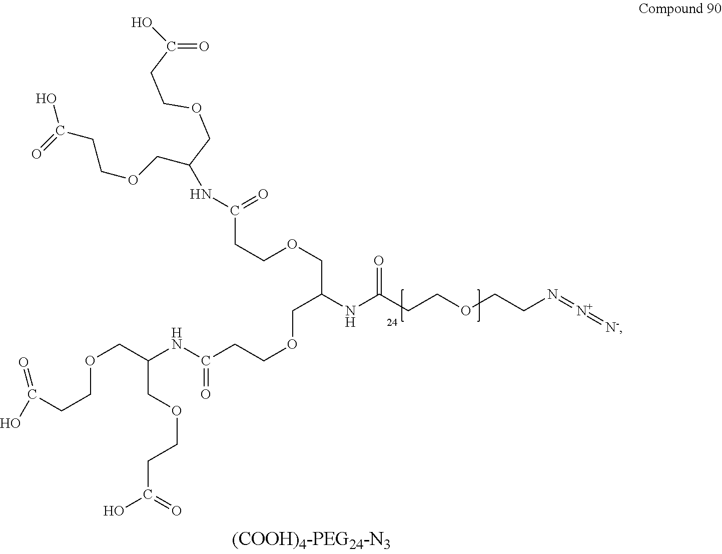

Compound 90, referred to as (COOH)₄—PEG₂₄-N₃ or tetra(COOH)-PEG₂₄-N₃ was synthesized by reacting 1.5 g of Compound 80 (0.9 mmol, 1 eq) and 0.4 g of 2-amino-1,3-bis(carboxylethoxy)propane HCl salt (1.4 mmol, 1.6 eq) dissolved in 35 mL of anhydrous DCM. Triethylamine (2.5 mL, 18.3 mmol, 19 eq) was added to the reaction mixture. The reaction was stirred at room temperature for 4 hours until HPLC indicated the reaction was complete. Compound 90 was not purified. MS (ESI) Calculated for C₇₈H₁₄₆N₆O₄₁ m/z 1823.0, found 912.4 (M/2+H)⁺.

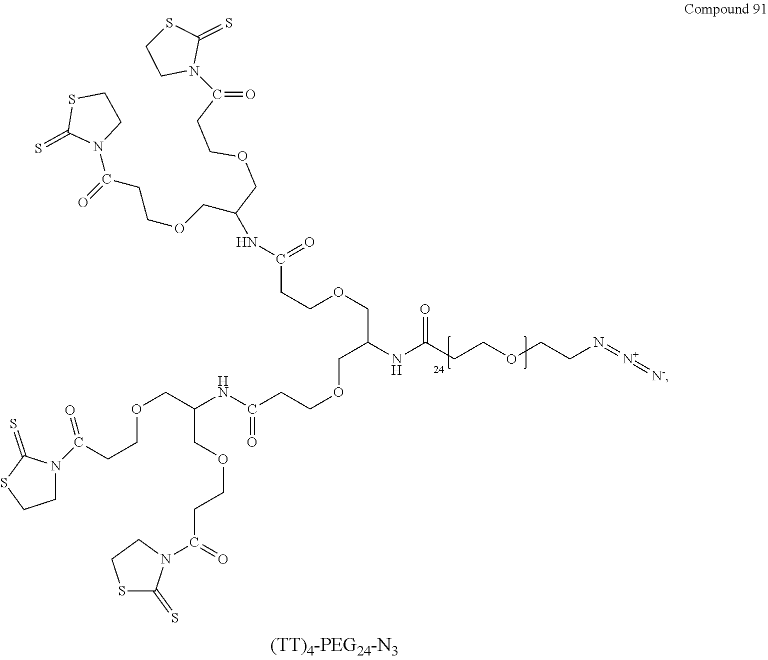

Compound 91, referred to as (TT)₄-PEG₂₄-N₃ was synthesized by reacting 1.5 g of Compound 90 (0.8 mmol, 1 eq) and 1.4 g of HATU (3.7 mmol, 4.4 eq) in 3 mL of DCM. Triethylamine (1.9 mL, 13.5 mmol, 16 eq) was added and the reaction mixture was stirred for 5 min. Thiazoline-2-thio (TT) (0.5 g, 4.4 mmol, 5 eq) was added and the reaction mixture was stirred at room temperature for 3 hours until HPLC indicated the reaction was complete. The product was purified by flash chromatography on a 100 g Biotage Safar SilicaD column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 0.8 g (42% yield) of a yellow oil (70% AUC at 220 nm). MS (ESI) Calculated for C₉₀H₁₅₈N₁₀O₃₇S₈ m/z 2228.8, found 962.8 (M/2)⁺.

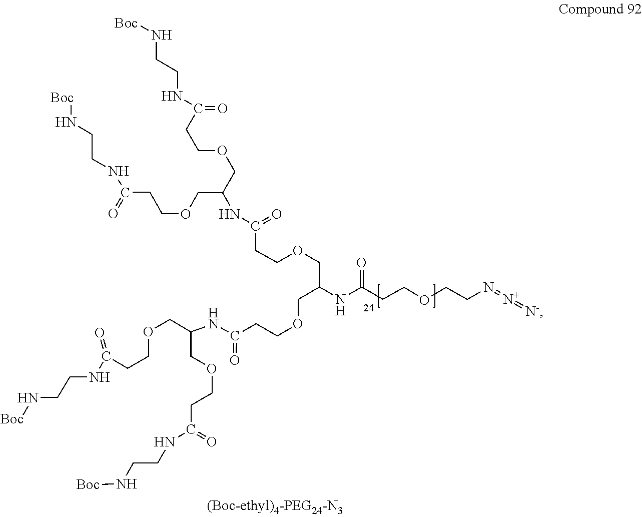

Compound 92, referred to as (Boc-ethyl)₄-PEG₂₄-N₃ was synthesized by reacting 246 mg of Compound 91 (0.1 mmol, 1 eq) and 84 mg of N-boc-ethylenediamine (0.5 mmol, 4.8 eq) in 2.6 mL of DCM. Triethylamine (74 uL, 0.5 mmol, 4.8 eq) was added and the reaction mixture was stirred at room temperature for 1 hour until HPLC indicated the reaction was complete. The solvent was removed under vacuum and the product was dissolved in DMSO and purified on a preparative HPLC system using a gradient of 32-60% acetonitrile/H₂O (0.05% TFA) over 12 minutes on an Agilent Prep-C18 column, 50×100 mm, 5 μm. The product eluted at ˜7.1 minutes and the resulting fractions were collected, frozen and then lyophilized to obtain 100.4 mg (40.0% yield) of a spectroscopically pure (94.9% AUC at 220 nm) white solid. MS (ESI) Calculated for C₁₀₆H₂₀₂N₁₄O₄₅ m/z 2392.8, found 1197.0 (M/2+H)⁺.

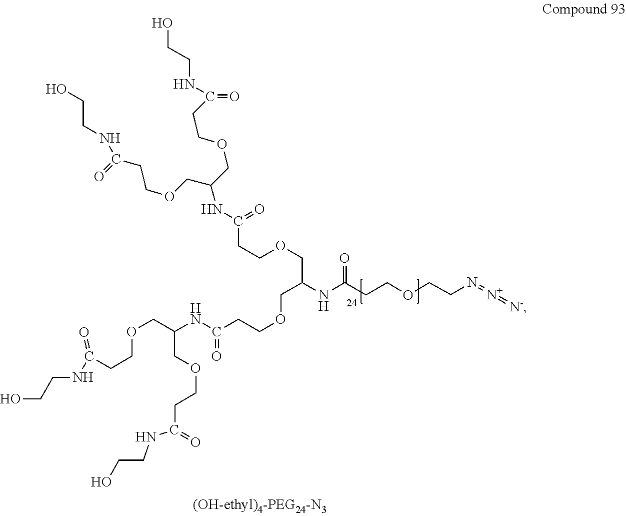

Compound 93, referred to as (OH-ethyl)₄-PEG₂₄-N₃ was synthesized following the same procedure as Compound 92, except ethanolamine was used instead of N-boc-ethylenediamine. Compound 93 was purified on a preparative HPLC system using a gradient of 17-37% acetonitrile/H₂O (0.05% TFA) over 12 minutes on an Agilent Prep-C18 column, 30×100 mm, 5 m. The product eluted at ˜7.0 minutes and the resulting fractions were collected, frozen and then lyophilized to obtain a 43.6% yield of a spectroscopically pure (97.2% AUC at 220 nm) white solid. MS (ESI) Calculated for C₈₆H₁₆₆N₁₀O₄₁ m/z 1995. 1, found 998.6 (M/2+H)⁺.

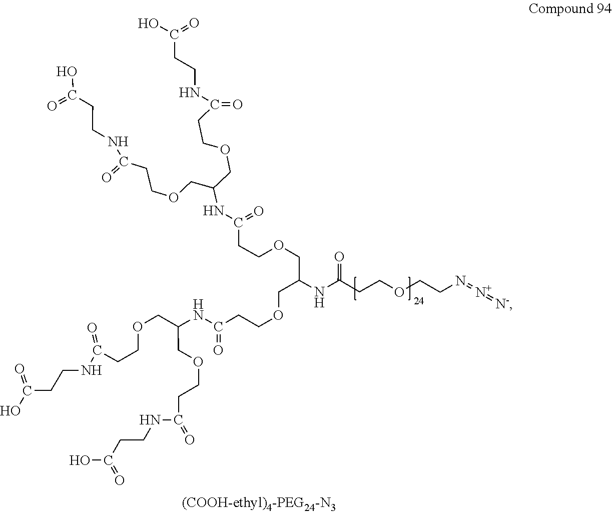

Compound 94, referred to as (COOH-ethyl)₄-PEG₂₄-N₃ was synthesized following the same procedure as Compound 92, except beta-alanine was used instead of N-boc-ethylenediamine and MeOH was used as the solvent. Compound 94 was purified on a preparative HPLC system using a gradient of 19-39% acetonitrile/H₂O (0.05% TFA) over 12 minutes on an Agilent Prep-C18 column, 30×100 mm, 5 μm. The product eluted at ˜6.8 minutes and the resulting fractions were collected, frozen and then lyophilized to obtain a 34.3% yield of as spectroscopically pure (86.0% AUC at 220 nm) white solid. MS (ESI) Calculated for C₉₀H₁₆₆N₁₀O₄₅ m/z 2107.1, found 1054.7 (M/2+H)⁺.

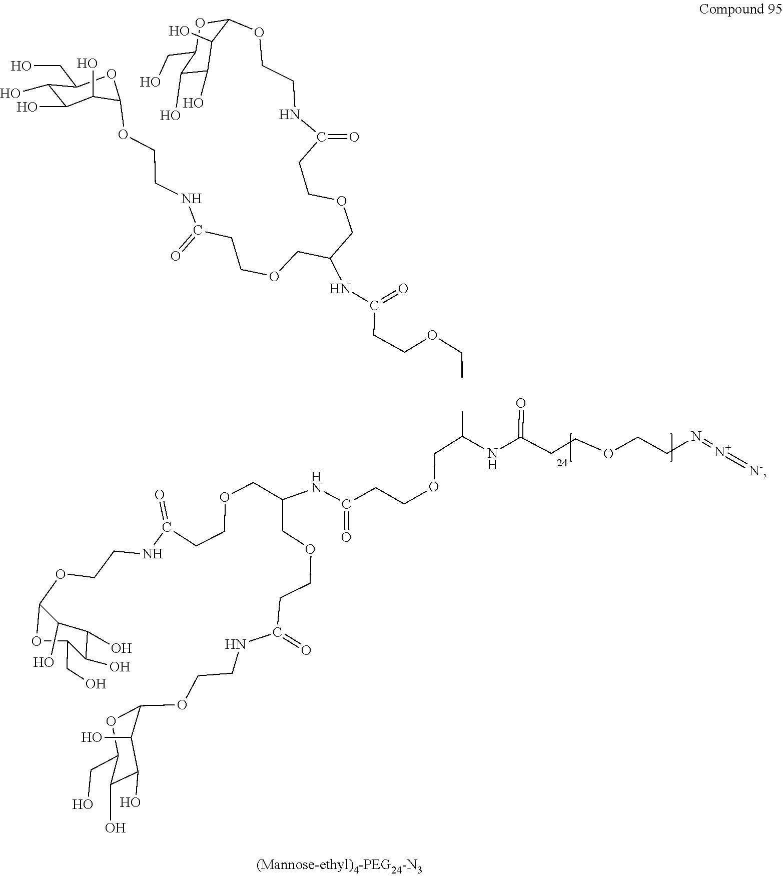

Compound 95, referred to as (Mannose-ethyl)₄-PEG₂₄-N₃ was synthesized following the same procedure as Compound 92, except 2-aminoethyl-a-mannopyranoside (Broadpharm (San Diego, CA)) was used instead of N-boc-ethylenediamine and DMSO was used as the solvent. Compound 95 was purified on a preparative HPLC system using a gradient of 15-35% acetonitrile/H₂O (0.05% TFA) over 12 minutes on an Agilent Prep-C18 column, 30×100 mm, 5 m. The product eluted at ˜7.3 minutes and the resulting fractions were collected, frozen and then lyophilized to obtain a 35.6% yield of a spectroscopically pure (98.5% AUC at 220 nm) white solid. MS (ESI) Calculated for C₁₃₃H₂₅₂N₁₀O₆₁ m/z 2965.7, found 1322.9 (M/2)⁺.

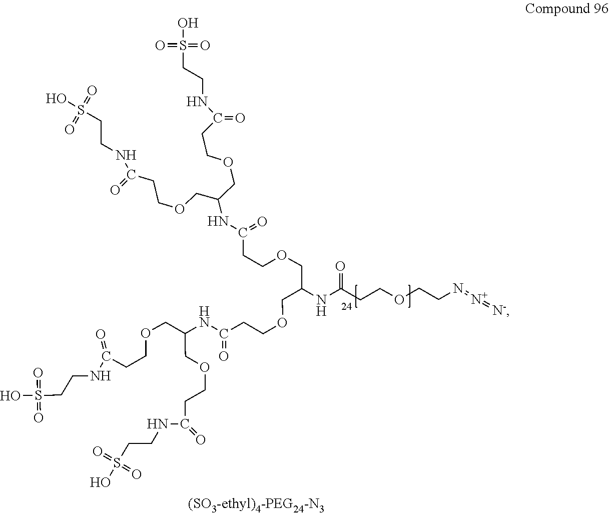

Compound 96, referred to as (SO₃-ethyl)₄-PEG₂₄-N₃ was synthesized following the same procedure as Compound 92, except taurine was used instead of N-boc-ethylenediamine and 2:1 DMSO/PBS was used as the solvent. Compound 96 was purified on a preparative HPLC system using a gradient of 5-45% acetonitrile/H₂O (0.05% TFA) over 12 minutes on an Agilent Prep-C18 column, 30×100 mm, 5 μm. The product eluted at ˜7.4 minutes and the resulting fractions were collected, frozen and then lyophilized to obtain a 30.2% yield of a spectroscopically pure (96.9% AUC at 220 nm) white solid. MS (ESI) Calculated for C₈₆H₁₆₆N₁₀O₄₉S₄ m/z 2251.0, found 1126.7 (M/2)⁺.

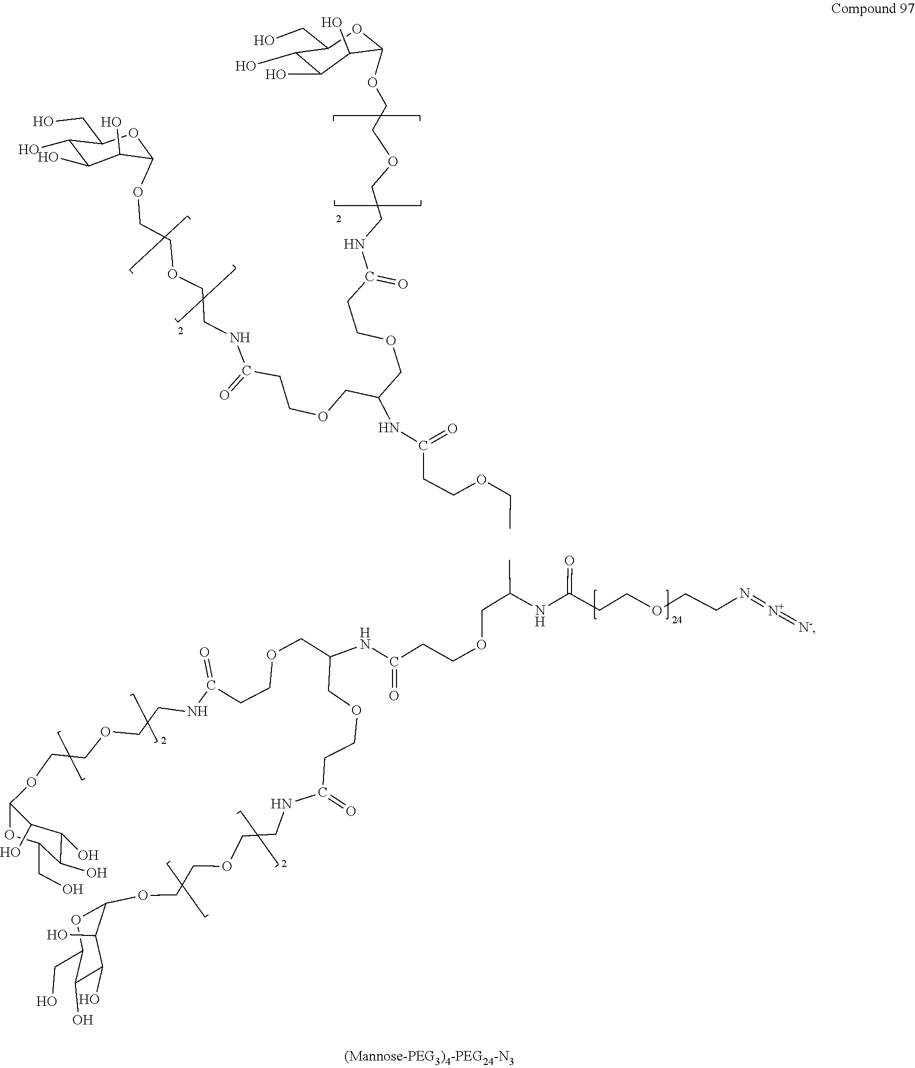

Compound 97, referred to as (Mannose-PEG₃)₄-PEG₂₄-N₃ or Tetra(Mannose-PEG₃)-PEG₂₄-N₃ was synthesized following the same procedure as Compound 92, except a-Mannose-PEG₃-amine (CarboSynthUSA (San Diego, CA)) was used instead of N-boc-ethylenediamine and DMSO was used as the solvent. Compound 97 was purified on a preparatory HPLC system using a gradient of 15-35% acetonitrile/H₂O (0.05% TFA) over 12 minutes on an Agilent Prep-C18 column, 50×100 mm, 5 μm. The product eluted at ˜7.3 minutes and the resulting fractions were collected, frozen and then lyophilized to obtain a 49.5% yield of a spectroscopically pure (97.2% AUC at 220 nm) white solid. MS (ESI) Calculated for C₁₂₆H₂₃₈N₁₀O₆₉ m/z 2995.5, found 999.9 (M/3)⁺.

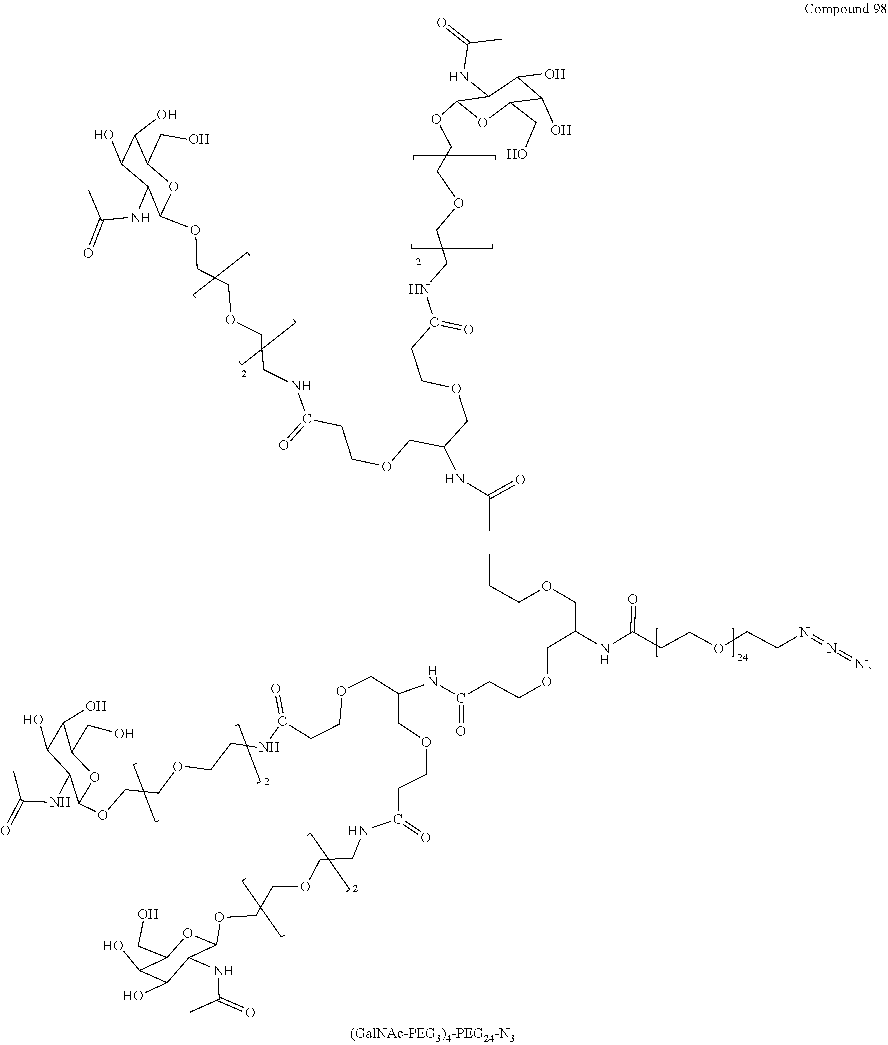

Compound 98, referred to as (GalNAc-PEG₃)₄-PEG₂₄-N₃ or Tetra(GalNAc-PEG₃)-PEG₂₄-N₃ was synthesized following the same procedure as Compound 92, except b-n-acetylgalactose-PEG3-amine (CarboSynthUSA (San Diego, CA)) was used instead of N-boc-ethylenediamine and DMSO was used as the solvent. Compound 98 was purified on a preparatory HPLC system using a gradient of 15-35% acetonitrile/H₂O (0.05% TFA) over 12 minutes on an Agilent Prep-C18 column, 30×100 mm, 5 μm. The product eluted at ˜6.7 minutes and the resulting fractions were collected, frozen and then lyophilized to obtain a 51.6% yield of a spectroscopically pure (97.0% AUC at 220 nm) white solid. MS (ESI) Calculated for C₁₃₄H₂₅₀N₁₄O₆₉ m/z 3159.7, found 1054.8 (M/3)⁺.

### Amphiphilic Block Copolymers of Formula S-B-U-H-[D] Having Cone Architecture

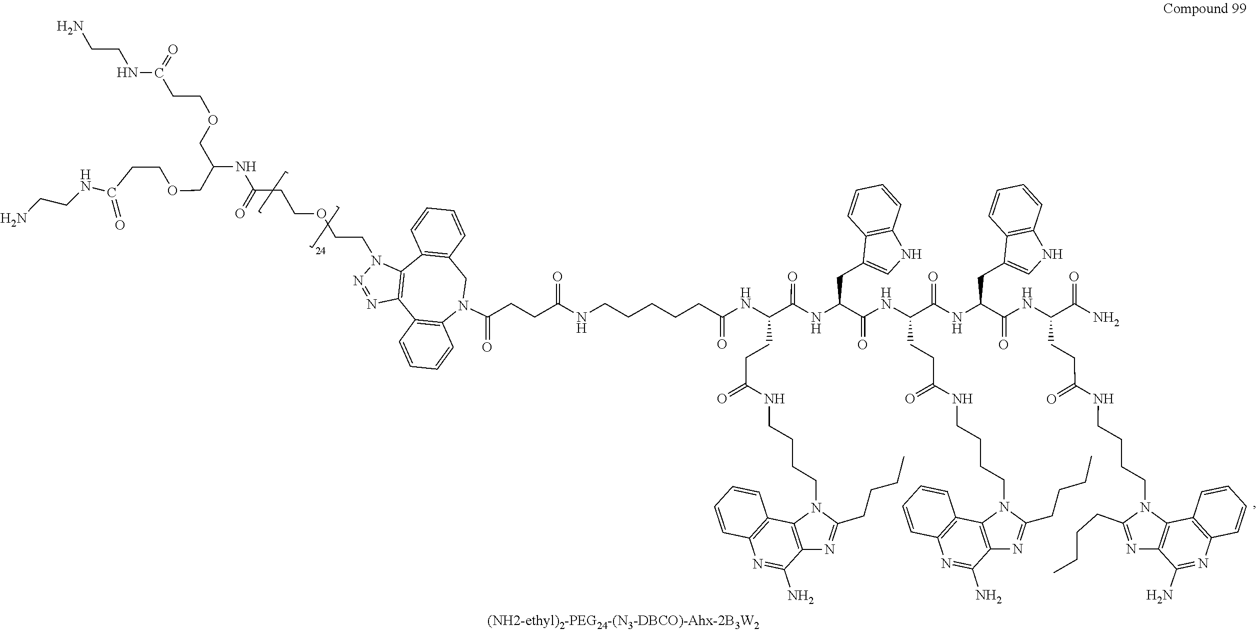

Compound 99, referred to (NH2-ethyl)₂-PEG₂₄-(N₃-DBCO)-Ahx-2B₃W₂ was synthesized by reacting Compound 81 (0.001 mmol, 1.0 eq) dissolved in anhydrous DMSO and Compound 56 (0.001 mmol, 1.05 eq) as a 50 mM solution in anhydrous DMSO. The reaction mixture was stirred overnight at room temperature. The DMSO solvent was then removed. The boc protected intermediate was deprotected by resuspending in 100% trifluoroacetic acid (TFA) (200 uL) for 1 minute, after which the TFA was removed with a stream of air. The remaining solution was washed twice with diethyl ether (200 uL). HPLC indicated the deprotection was complete and resulted in 2.1 mg (38.4% yield) of a spectroscopically pure (89.6% AUC at 220 nm) off-white solid. MS (ESI) calculated for C₁₈₀H₂₅₆N₃₃O₄₀ m/z 3531.3, found 1177.8 (M/3)⁺.

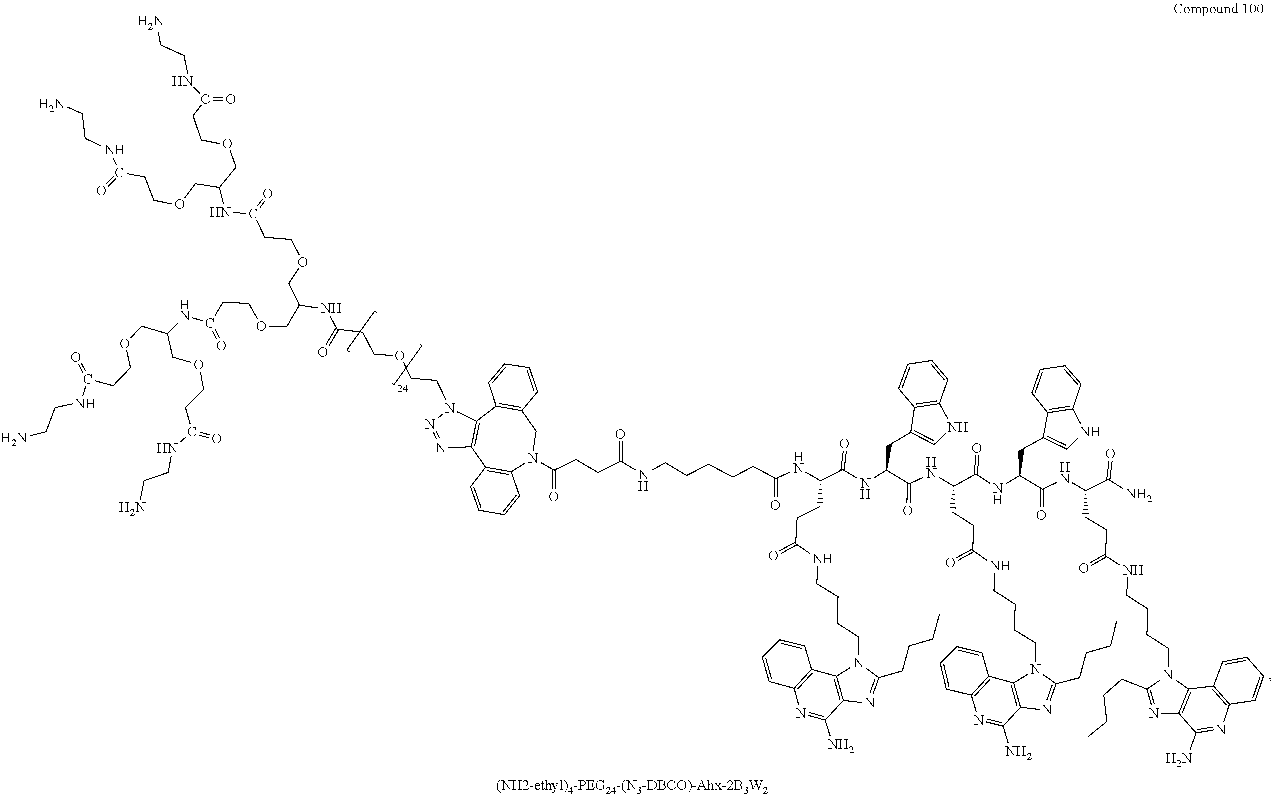

Compound 100, referred to (NH2-ethyl)₄-PEG₂₄-(N₃-DBCO)-Ahx-2B₃W₂ was synthesized using the same procedure as Compound 99, except Compound 92 was used in place of Compound 81 and resulted in a spectroscopically pure (92.7% AUC at 220 nm) off-white solid. MS (ESI) calculated for C₂₀₂H₃₀₇N₃₉O₄₈ m/z 4047.3, found 1013.3 (M/4+H)⁺.

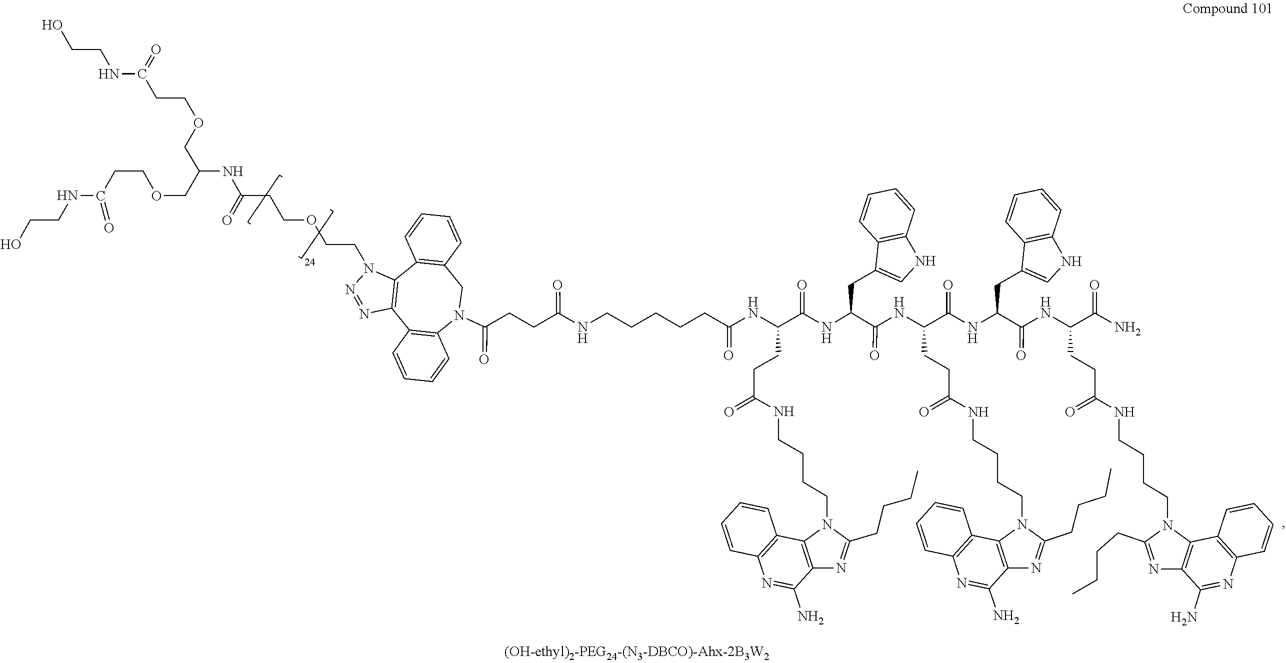

Compound 101, referred to (OH-ethyl)₂-PEG₂₄-(N₃-DBCO)-Ahx-2B₃W₂ was synthesized by reacting Compound 82 (0.001 mmol, 1.0 eq) with Compound 56 (0.001 mmol, 1.05 eq) in anhydrous DMSO for 16 hours at room temperature. HPLC was monitored to evaluate reaction progress and indicated complete conversion of Compound 82 to Compound 101, resulting in a spectroscopically pure (89.2% AUC at 220 nm) colorless oil. MS (ESI) calculated for C₁₈₀H₂₆₃N₃₁O₄₂ m/z 3530.9, found 1178.3 (M/3+H)⁺.

Compounds 102-115 were produced in a similar manner as that described for Compound 101. Table 3 provides a summary of the synthesis and characterization of compounds 102-115.

TABLE 3
Amphiphiles of formula S-B-U-H-[D] having dendron architecture, i.e., having
a solubilizing block (S) comprising a dendron amplifier.

| | (S-B-U1) | U2-H[D] | | | |
|---|---|---|---|---|---|
| Cmpd | Compound # | Compound # | | m/z | MS (ESI) |
| # | Product S-B-U-H-[D] | | Charge | Theo. | m/n Found |
| 102 | Compound 83 | Compound 56 | −2 | 3586.9 | 1197.0 |
| | (COOH-ethyl)₂-PEG₂₄-(N3-DBCO)-Ahx-2B₃W₂ | | | | (M/3 + H)⁺ |
| 103 | Compound 84 | Compound 56 | 0 | 1799.0 | 1286.6 |
| | (Mannose-ethyl)₂-PEG₂₄-(N3-DBCO)-Ahx-2B₃W₂ | | | | (M/3 + H)⁺ |
| 104 | Compound 85 | Compound 56 | −2 | 2658.9 | 1221.0 |
| | (SO3-ethyl)₂-PEG₂₄-(N3-DBCO)-Ahx-2B₃W₂ | | | | (M/3 + H)⁺ |
| 105 | Compound 86 | Compound 56 | −2 | 4789.4 | 1198.7 |
| | (CD22a)₂-PEG₂₄-(N3-DBCO)-Ahx-2B₃W₂ | | | | (M/4 + H)⁺ |
| 106 | Compound 93 | Compound 56 | 0 | 4051.2 | 1351.9 |
| | (OH-ethyl)₄-PEG₂₄-(N3-DBCO)-Ahx-2B₃W₂ | | | | (M/3 + H)⁺ |
| 107 | Compound 94 | Compound 56 | −4 | 4163.2 | 1389.2 |
| | (COOH-ethyl)₄-PEG₂₄-(N3-DBCO)-Ahx-2B₃W₂ | | | | (M/3 + H)⁺ |
| 108 | Compound 94 | Compound 54 | −4 | 3455.7 | 1153.1 |
| | (COOH-ethyl)₄-PEG₂₄-(N3-DBCO)-Ahx-W₅ | | | | (M/3 + H)⁺ |
| 109 | Compound 95 | Compound 56 | 0 | 4698.4 | 1176.3 |
| | (Mannose-ethyl)₄-PEG₂₄-(N3-DBCO)-Ahx-2B₃W₂ | | | | (M/4 + H)⁺ |
| 110 | Compound 95 | Compound 54 | 0 | 3990.9 | 1331.9 |
| | (Mannose-ethyl)₄-PEG₂₄-(N3-DBCO)-Ahx- | | | | (M/3 + H)⁺ |
| | WWWWW | | | | |
| 111 | Compound 96 | Compound 56 | −4 | 4307.1 | 1437.2 |
| | (SO3-ethyl)₄-PEG₂₄-(N3-DBCO)-Ahx-2B₃W₂ | | | | (M/3 + H)⁺ |
| 112 | Compound 95 | Compound 52 | 0 | 4681.3 | 1172.1 |
| | (Mannose-ethyl)₄-PEG₂₄-(N3-DBCO)-Ahx-(F′)10 | | | | (M/4 + H)⁺ |
| 113 | Compound 97 | Compound 54 | 0 | 4343.21 | 1449.0 |
| | (Mannose-PEG₃)₄-PEG₂₄-(N₃-DBCO)-Ahx-W₅ | | | | (M/3 + H)⁺. |
| 114 | Compound 97 | Compound 56 | 0 | 5051.6 | 1264.6 |
| | (Mannose-PEG₃)₄-PEG₂₄-(N₃-DBCO)-Ahx-2B₃W₂ | | | | (M/4 + H)⁺. |
| 115 | Compound 98 | Compound 54 | 0 | 4510.7 | 1503.8 |
| | (GalNAc-PEG₃)₄-PEG₂₄-(N₃-DBCO)-Ahx-W₅ | | | | (M/3)⁺ |
| 116 | Compound 95 | Compound 60 | 0 | 5012.3 | 1054 |
| | Tetra(Mannose-ethyl)-PEG₂₄-(N₃-DBCO)-bis(VZ- | | | | (M/4 + H)⁺. |
| | PAB-2Bxy)₂, | | | | |
| 117 | Compound 90 | Compound 60 | −4 | 3835 | 1281 |

| | |
|---|---|
| TetraCOOH)-PEG₂₄-(N₃-DBCO)-bis(VZ-PAB-2Bxy)2, | (M/3 + H)⁺. |

Net charge is the predicted net charge of the amphiphile in aqueous buffer at pH 7.4

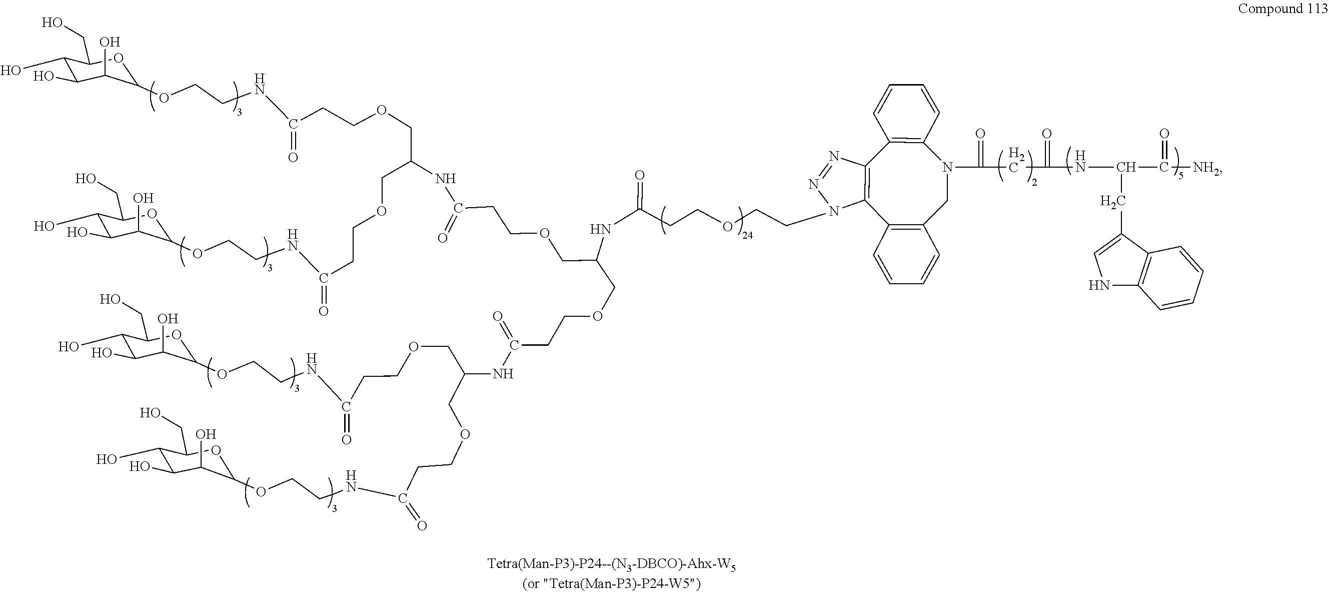

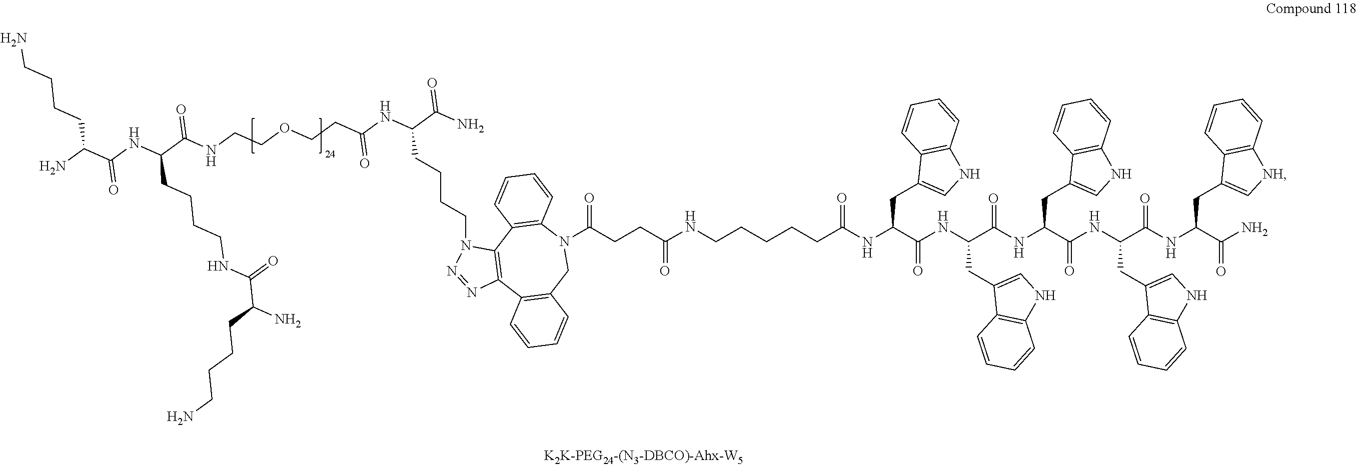

Compound 118, K₂K-PEG₂₄-(N₃-DBCO)-Ahx-W₅ was synthesized by reacting the peptide-based dendron comprising a C-terminal Lys(N₃), K₂K-PEG₂₄-{Lys(N₃)}, with Compound 54 in a similar manner as was described for Compound 101. Upon completion, the reaction was purified on a preparatory HPLC system using a gradient of 31-51% acetonitrile/H₂O (0.05% TFA) over 12 minutes on an Agilent Prep-C18 column, 30×100 mm, 5 μm. The product eluted at ˜6.7 minutes and the resulting fractions were collected, frozen and then lyophilized to obtain a spectroscopically pure (>99% AUC at 220 nm) solid. MS (ESI) Calculated for C₁₅₅H₂₂₇N₂₅O₃₇ m/z 3030.7, found 1516.6 (M/2+H)⁺.

Compounds 118-120 were produced in a similar manner as that described for Compound 101. Table 4 provides a summary of the synthesis and characterization of compounds 118-120.

TABLE 4
Non-limiting examples of amphiphiles with solubilizing blocks comprising a
dendron amplifier and either amine or carboxylic acid solubilizing groups.

| | | U2-H-[D] | | | |
|---|---|---|---|---|---|
| Cmpd | (S-B-U1) Compound # | Compound # | | m/z | MS (ESI) |
| # | Product S-B-U-H-[D] | | Charge | Theo. | m/n Found |
| 118 | K₂K-PEG₄-{Lys(N₃)} | Compound 54 | +4 | 2150.14 | 1076.3 |
| | K₂K-PEG₄-((Lys(N₃)-DBCO)-Ahx-W₅ | | | | (M/2 + H)⁺ |
| 119 | K₂K-{Lys(N₃)} | Compound 54 | +4 | 3030.66 | 952.8 |
| | K₂K-((Lys(N₃)-DBCO)-Ahx-W₅ | | | | (M/3 + H)⁺ |
| 120 | {Glutaric acid}₄K₂K-PEG₄-{Lys(N₃)} | Compound 54 | −4 | 2606.27 | 1304.2 |

| | |
|---|---|
| {Glutaric acid}₄K2K-PEG₄-((Lys(N₃)-DBCO)-Ahx-W₅ | (M/2 + H)⁺ |

Single letter abbreviations are used for amino acid sequences in the above table; Peptide-based starting materials were manufactured by solid-phase peptide synthesis by Genscript (Piscataway, NJ). Net charge is the predicted net charge of the amphiphile in aqueous buffer at pH 7.4.

Impact of Amphiphile Composition on pH-Responsiveness and Cell Uptake

Our initial studies investigated how the composition and architecture of the solubilizing block impact the capacity of amphiphiles of formula S-[B]-[U]-H-[D] to assemble into nanoparticle micelles. While linear amphiphiles typically required net charge greater than +4 or less than −4 to ensure stable nanoparticle micellization, dendron amphiphiles formed stable nanoparticle micelles with neutral charge. In contrast to the linear and dendron structures, brush-based amphiphiles exhibited greater particle size variability that was independent of net charge. Altogether, these data indicate that the amphiphile architecture has a major impact on hydrodynamic behavior as well as the requirements (e.g., net charge required) for micellization and that amphiphiles with dendron architecture may be more tolerant of low net charge than other amphiphile architectures.

Based on these findings, our studies focused on how the composition of the solubilizing group impacts micellization and particle size stability of amphiphiles with solubilizing groups comprising a dendron amplifier.

For use as solubilizing groups of amphiphiles, carboxylic acids should be deprotonated at pH near physiologic pH, e.g., pH 7.4, to ensure that the amphiphile has net negative charge. However, the pKa of carboxylic acids can be influenced by their chemical environment as well as substituent groups. Therefore, several amphiphiles comprising solubilizing groups further comprising carboxylic acids were synthesized and their solubility over a range of pH from pH 7.4 to pH 6.5 was evaluated.

Compounds 121, 122 and 107 comprise a PEG-based dendron amplifier with a terminal functional group (FGt) consisting of carboxylic acid that is the solubilizing group (SG) or is linked to SG via the linker X5, wherein X5-SG is —NH—(CH₂)—COOH and —NH—(CH₂)₂-COOH, respectively. Structures and synthesis of Compounds 121, 122 and 107 are provided below.

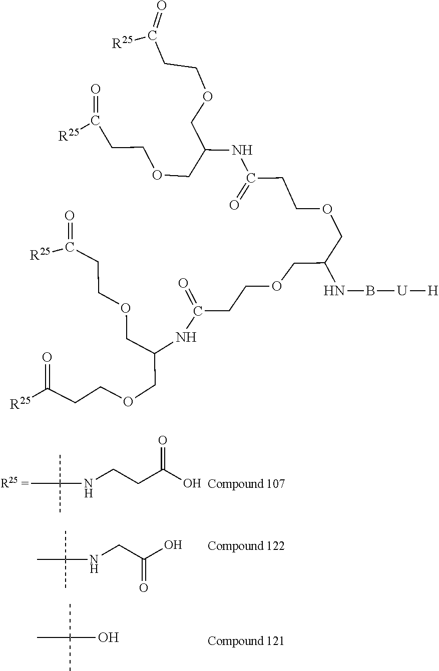

Compound 121, referred to (COOH)₄—PEG₂₄-(N₃-DBCO)-Ahx-2B₃W₂ or Tetra(COOH)-PEG₂₄-(N₃-DBCO)-Ahx-2B₃W₂ was synthesized by reacting Compound 90 with Compound 56 in a similar manner as described for Compound 101. MS (ESI) calculated for C₁₉₄H₂₈₃N₃₁O₅₂ m/z 3879.1, found 971.2 (M/4+H)⁺.

Compound 122, referred to (COOH-methyl)₄-PEG₂₄-(N₃-DBCO)-Ahx-2B₃W₂, Tetra(COOH-methyl)-PEG₂₄-(N₃-DBCO)-Ahx-2B₃W₂ or Tetra(Gly)-PEG₂₄-(N₃-DBCO)-Ahx-2B₃W₂ was synthesized in two steps. First, (COOH-methyl)₄-PEG₂₄-N₃ was synthesized by reacting Compound 91 with glycine in a similar manner as described for Compound 81. (COOH-methyl)₄-PEG₂₄-N₃ was then reacted with Compound 56 in a similar manner as described for Compound 101 to yield Compound 122. C₂₀₂H₂₉₅N₃₅O₅₆ m/z 4107.1, found 1370.4 (M/3+H)⁺.

Compounds 124, 125 and 126 comprise a peptide-based, i.e., lysine-based, dendron amplifier with a terminal functional group (FGt) consisting of an amine that is linked to SG via the linker X5, wherein X5-SG is —C(O)—(CH₂)₃-COOH for Compound 124 and X5-SG is —C(O)—(CH₂)₂-COOH for Compounds 125 and 126. Structures and synthesis of Compounds 124, 125 and 126 are provided below.

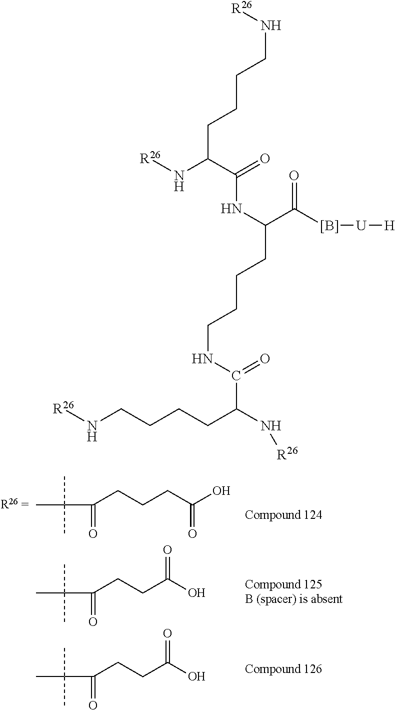

Compound 124, referred to as {Glutaric acid}4K2K{PEG24}{Lys(N3-DBCO)}-Ahx-2B₃W₂ was synthesized by reacting the peptide-based dendron comprising a C-terminal Lys(N3), {Glutaric acid}4K2K{PEG24}{Lys(N3)}, with Compound 56 in a similar manner as was described for Compound 101. MS (ESI) calculated for C₂₁₁H₃₁₁N₃₇O₅₂ m/z 4195.3, found 1050.2 (M/4+H)⁺.

Compound 125, referred to as {Succinic acid}4K2K{Lys(N3-DBCO)}-Ahx-2B₃W2 was synthesized by reacting the peptide-based dendron comprising a C-terminal Lys(N3), {Succinic acid}4K2K {Lys(N3)}, with Compound 56 in a similar manner as was described for Compound 101. MS (ESI) calculated for C₁₅₆H₂₀₂N₃₆O₂₇ m/z 3011.6, found 1005.3 (M/3+H)⁺.

Compound 126, referred to as {Succinic acid}4K2KK{PEG24-N3-DBCO)}-Ahx-2B₃W₂ was synthesized in two steps. First, the epsilon amine of the C-terminal lysine of the peptide-based dendron {Succinic acid}4K2KK was reacted with NHS-PEG24-N₃ to yield {Succinic acid}4K2KK{PEG24-N3}, which was then reacted with Compound 56 in a similar manner as was described for Compound 101 to yield Compound 125. MS (ESI) calculated for C₂₀₇H₃₀₃N₃₇O₅₂ m/z 4139.2, found 1036.3 (M/4+H)⁺.

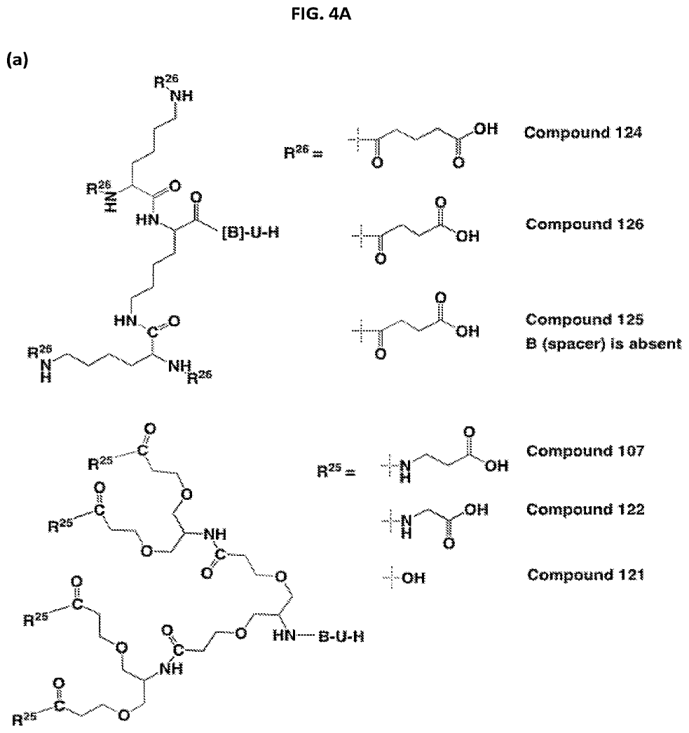

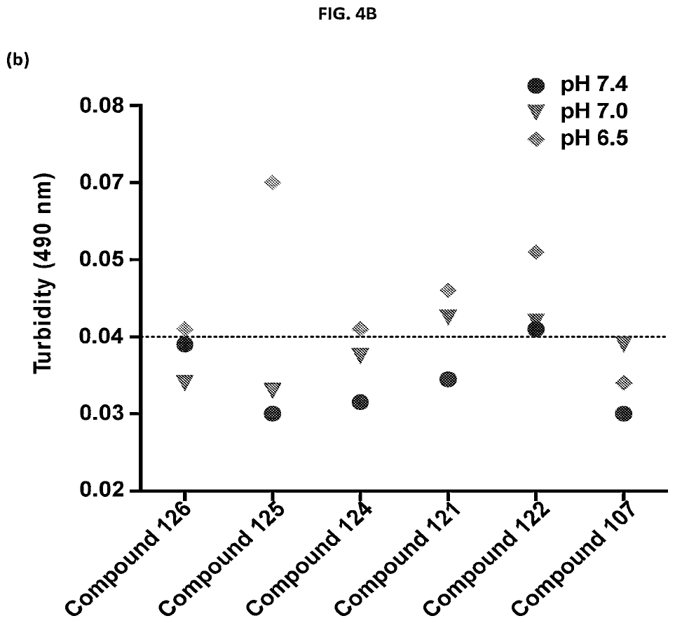

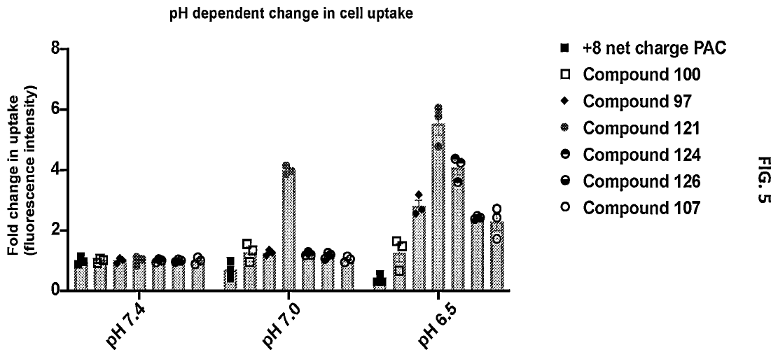

The pH-responsive properties of the 6 different compositions (Compounds 107, 121, 122, 124, 125 and 126) of amphiphiles further comprising carboxylic acid groups was evaluated using turbidity measurements. In brief, each amphiphile was suspended in 1×PBS buffer at either pH 7.4, 7.0 or pH 6.5 at a final concentration of 0.1 mM and turbidity (OD at 490 nm) was assessed using a UV-Vis spectrophotometer (FIG. 4).

Except for Compound 122, which exhibited some aggregation at pH 7.4, all the other amphiphiles comprising carboxylic acids formed nanoparticle micelles with stable particle size at pH 7.4 (FIG. 4B). Notably, Compound 121 formed aggregates at pH 7.0 and pH 6.5, indicating pH-responsiveness near physiologic pH, between about pH 7.4 and 7.0, whereas Compounds 107, 124, 125 and 126 exhibited stable particle sizes down to at least pH 7.0, with all except Compound 107 undergoing aggregation at pH 6.5.

A notable finding was that pH-dependent changes in particle size stability were also associated with pH-dependent changes in cell uptake. Accordingly, Compound 121, which aggregated at pH 7.0 or less (FIG. 4B) had a greater than 4-fold increase in splenocyte uptake at pH 7.0 or less as compared with pH 7.4, and Compounds 124, 126 and 107, which showed moderate to extensive aggregation at pH less than pH 7.0 showed minimal to no change in cell uptake at pH 7.0 but greater than 2-fold increase in cell uptake at pH 6.5 as compared with cell uptake at pH 7.4 (FIG. 5). A non-limiting explanation is that with decreasing pH a greater portion of the solubilizing groups comprising carboxylic acids become protonated thereby resulting in decreased solubilizing and the propensity for aggregation, causing an increase in turbidity as well as more efficient uptake by antigen presenting cells. Based on these unexpected findings, preferred compositions comprise an amphiphile with dendron architecture comprising carboxylic acid solubilizing groups.

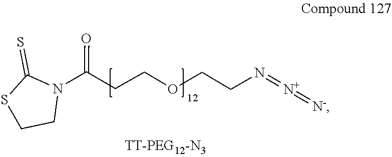

Compound 127, referred to as TT-PEG₁₂-N₃ was synthesized by reacting 50 mg of N₃-PEG₁₂-COOH (0.08 mmol, 1 eq) with 33.0 mg of HATU (0.09 mmol, 1.4 eq) in 330 μL of DCM. To the mixture, 43.3 uL of triethylamine (0.31 mmol, 4 eq) was added. The mixture was stirred for 5 minutes and 12.6 mg of thizoline-2-thiol (TT) (0.11 mmol, 1.4 eq) was added. The reaction mixture stirred at room temperature for 2 hours until HPLC indicated the reaction was complete. The product was purified by flash chromatography on a 10 g Biotage Safar Silica HC column over a 2-step gradient: 0% methanol in DCM over 3 column volumes (CVs), followed by 0-7% methanol in DCM over 20 CVs. The product eluted at ˜5% methanol and the resulting fractions were collected and the solvent removed to obtain 24 mg (41.0% yield) of a spectroscopically pure (92.0% AUC at 220 nm) yellow oil. MS (ESI) Calculated for C₃₀H₅₆N₄O₁₃S₂ m/z 744.3, found 745.3 (M+H)⁺.

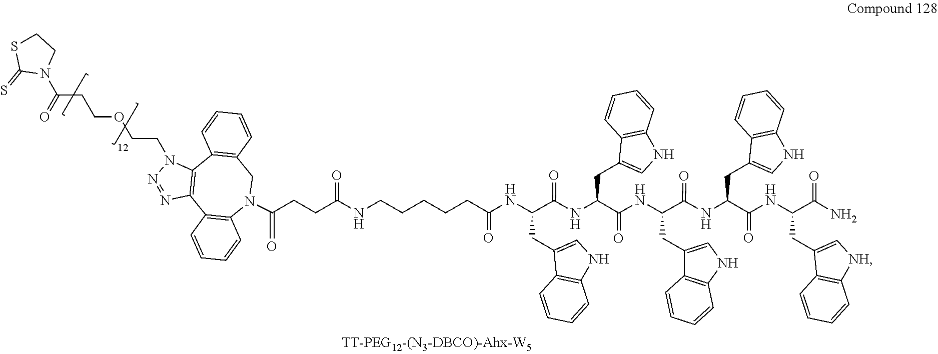

Compound 128, referred to as TT-PEG₁₂-(N₃-DBCO)-Ahx-W₅ was synthesized by reacting 14.2 mg of Compound 127 (0.02 mmol, 1 eq) with 28.4 mg of Compound 54 (0.02 mmol, 1.1 eq) in 400 μL of anhydrous DMSO. The reaction was mixed at room temperature for 16 hours, until HPLC indicated the reaction was complete. The reaction was not purified and resulted in an 83% pure (AUC at 220 nm) product. MS (ESI) Calculated for C₁₁₀H₁₃₃17O₂₁S₂ m/z 2091.9, found 1048.4 (M/2+H)⁺.

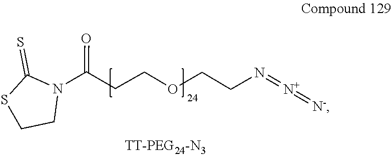

Compound 129, referred to as TT-PEG₂₄-N₃ was synthesized by reacting 2.55 g of N₃-PEG₂₄-COOH (2.2 mmol, 1 eq) with 0.92 g of HATU (2.4 mmol, 1.1 eq) in 24 mL of DCM. To the mixture, 1.2 mL of triethylamine (8.7 mmol, 4 eq) was added. The mixture was stirred for 5 minutes and 0.29 g of thizoline-2-thiol (TT) (2.5 mmol, 1.1 eq) was added. The reaction mixture was stirred at room temperature for 2 hours until HPLC indicated the reaction was complete. The reaction mixture was diluted with 200 mL of dichloromethane (DCM) and then washed with 2×200 mL 0.1 M HCl and the 1×200 mL DI H₂O. The organic layer was dried with Na₂SO₄ and then removed under vacuum, resulting in a yellow oil. The product was then purified by flash chromatography on a 100 g Biotage Safar Silica HC column over a 2-step gradient: 0% methanol in DCM over 3 column volumes (CVs), followed by 0-8% methanol in DCM over 20 CVs. The product eluted at ˜5% methanol and the resulting fractions were collected and the solvent removed to obtain 1.8 g (62.5% yield) of an 84% pure (AUC at 220 nm) yellow oil. MS (ESI) Calculated for C₅₄H₁₀₄N₄O₂₈S₂ m/z 1272.6, found 1273.6 (M+H)⁺.

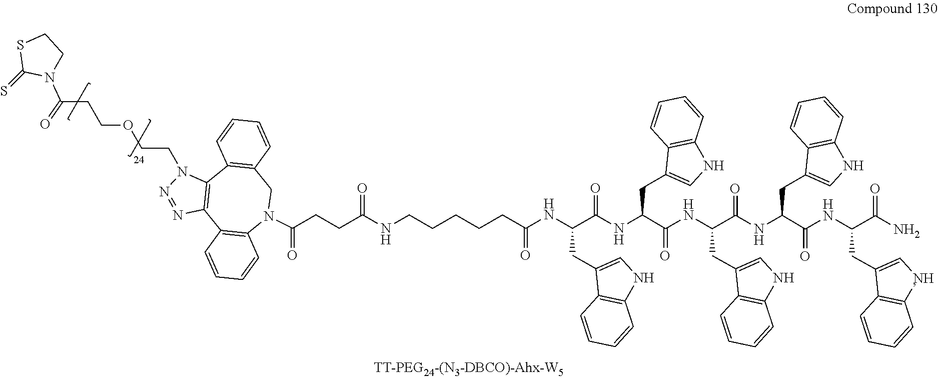

Compound 130, referred to as TT-PEG₂₄-(N₃-DBCO)-Ahx-W₅ was synthesized by reacting 12.7 mg of Compound 129 (0.01 mmol, 1 eq) with 14.8 mg of Compound 54 (0.01 mmol, 1.1 eq) in 500 μL of DMSO. The reaction was mixed at room temperature for 16 hours, until HPLC indicated the reaction was complete. The reaction was not purified and resulted in an 84% pure (AUC at 220 nm) product. MS (ESI) Calculated for C₁₃₄H₁₈₁N₁₇O₃₃S₂ m/z 2622.1, found 1311.8 (M/2+H)⁺.

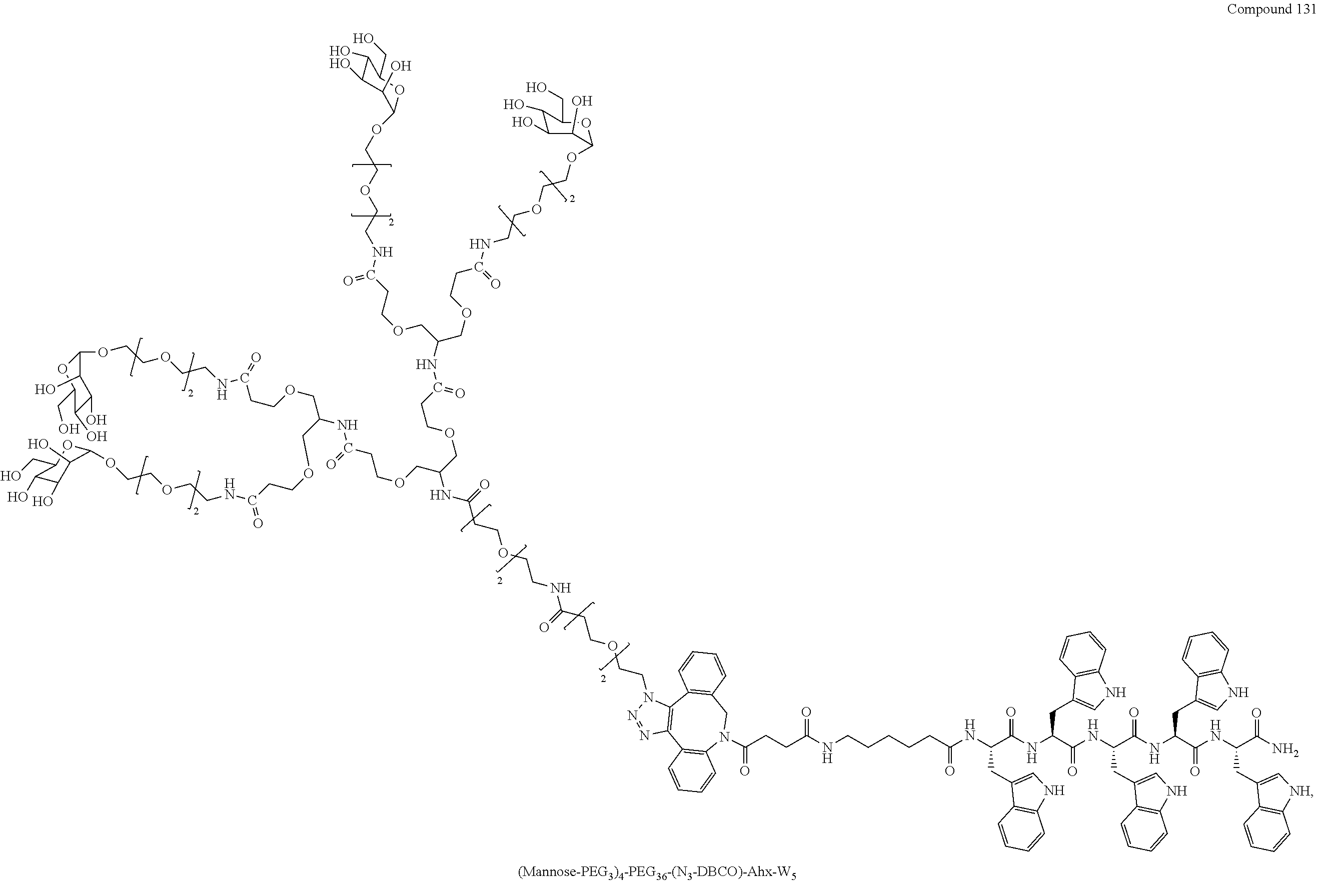

Compound 131, referred to as (Mannose-PEG₃)₄-PEG₂₄-PEG₁₂-(N₃-DBCO)-Ahx-W₅, (Mannose-PEG₃)₄-PEG₃₆-Ahx-W₅ or Tetra(Mannose-PEG₃)-PEG₃₆-Ahx-W₅, was synthesized by first synthesizing (Mannose-PEG₃)₄-PEG₂₄-NH₂ by reacting 45 mg Compound 97 (15 umol, 1 eq) with 43 mg tris(2-carboxyethyl)phosphine hydrochloride (TCEP) (150 umol, 10 eq) in 1 mL anhydrous DMSO. The reaction was mixed for 16 hours at room temperature, when HPLC indicated that all of Compound 97 was converted to (Mannose-PEG₃)-PEG₂₄-NH₂. To the reaction mixture 28.9 mg of Compound 128 (13.8 umol, 1 eq) and 40 uL triethylamine (TEA) (287 umol, 20 eq) was added. The reaction was stirred for 1 hour at room temperature, when HPLC indicated the reaction was complete. Compound 131 was purified on a preparative HPLC system using a gradient of 32-52% acetonitrile/H₂O (0.05% TFA) over 12 minutes on an Agilent Prep-C18 column, 50×100 mm, 5 μm. The product eluted at ˜6 minutes and the resulting fractions were collected, frozen and then lyophilized to obtain a 47.9% yield of a spectroscopically pure (96.2% AUC at 220 nm) colorless oil. MS (ESI) Calculated for C₂₃₃H₃₆₈N₂₄O₉₀ m/z 4942.5, found 1237.2 (M/4+H)⁺.

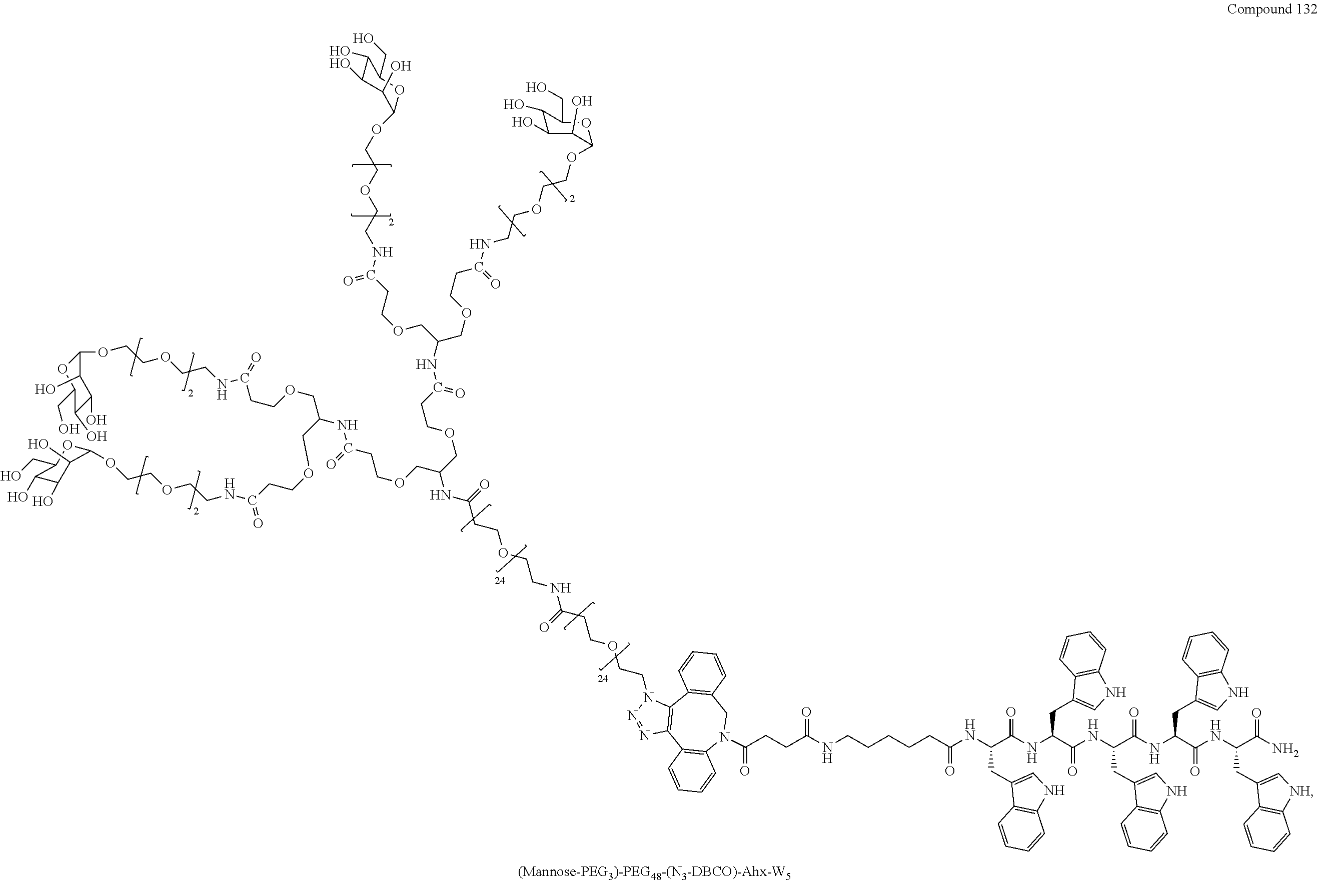

Compound 132, referred to as (Mannose-PEG₃)₄-PEG₂₄-PEG₂₄-Ahx-W₅, (Mannose-PEG₃)₄-PEG₄₈-Ahx-W₅, Tetra(Mannose-PEG₃)-PEG₄₈-Ahx-W₅ was synthesized following the same procedure at Compound 131, except that once Compound 97 was fully converted to (Mannose-PEG₃)-PEG₂₄-NH2, Compound 130 was added instead of Compound 128. Compound 132 was purified on a preparative HPLC system using a gradient of 32-52% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a 40.9% yield of a spectroscopically pure (98.2% AUC at 220 nm) colorless oil. MS (ESI) Calculated for C₂₅₇H₄₁₆N₂₄O₁₀₂ m/z 5470.8, found 1095.9 (M/5+H)⁺.

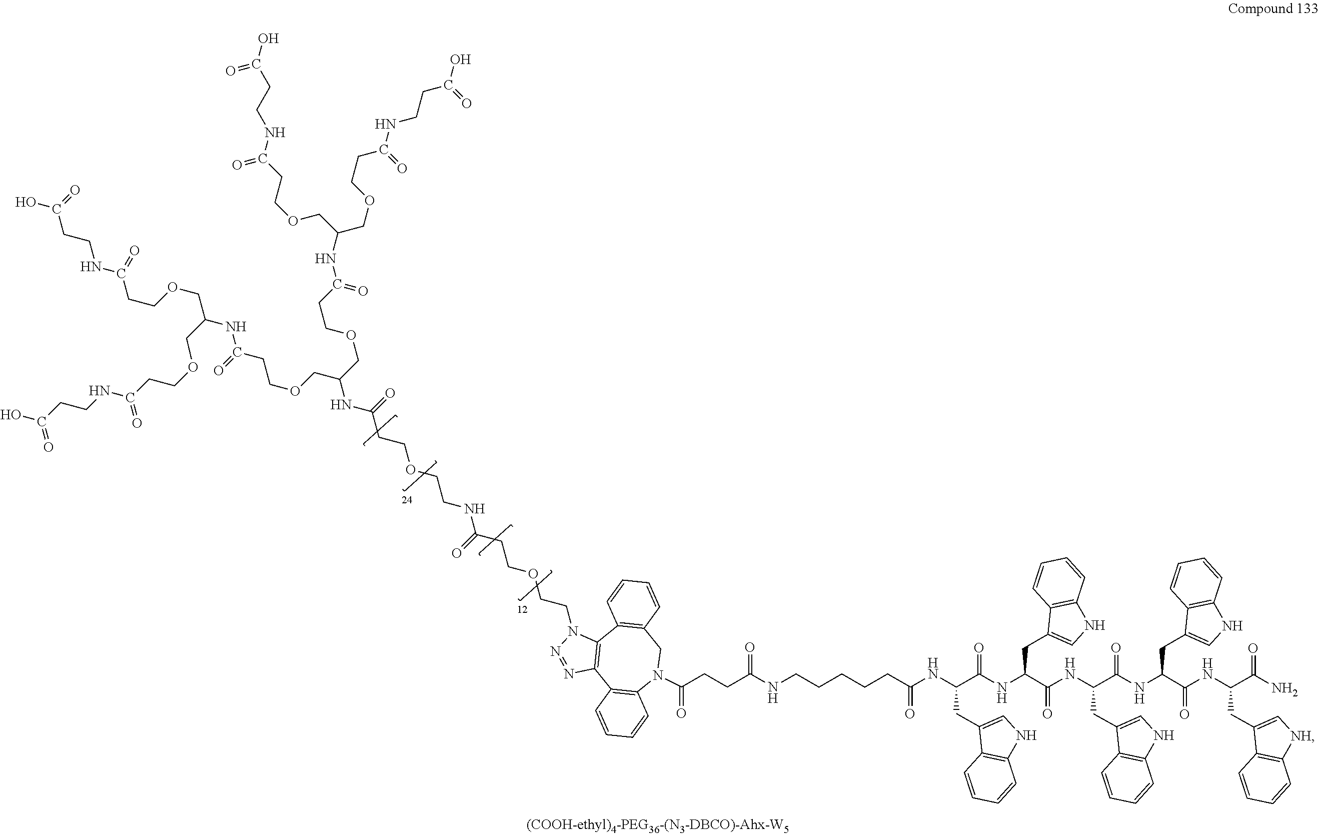

Compound 133, referred to as referred to as (COOH-ethyl)₄-PEG₂₄-PEG₁₂-(N₃-DBCO)-Ahx-W₅, (COOH-ethyl)₄-PEG₃₆-(N₃-DBCO)-Ahx-W₅ or Tetra(COOH-ethyl)-PEG₃₆-Ahx-W₅, was synthesized following the same procedure as Compound 131, except Compound 94 was used in place of Compound 97. Compound 133 was purified on a preparative HPLC system using a gradient of 34-54% acetonitrile/H₂O (0.05% TFA) over 12 minutes on an Agilent Prep-C18 column, 30×100 mm, 5 μm. The product eluted at ˜7 minutes and the resulting fractions were collected, frozen and then lyophilized to obtain a 62.3% yield of a spectroscopically pure (94.6% AUC at 220 nm) colorless oil. MS (ESI) Calculated for C₁₉₇H₂₉₆N₂₄O₆₆ m/z 4054.1, found 1352.6 (M/3+H)⁺.

### Amphiphiles of Formula S-[B]-[U]-H-[D] Having Linear Architecture

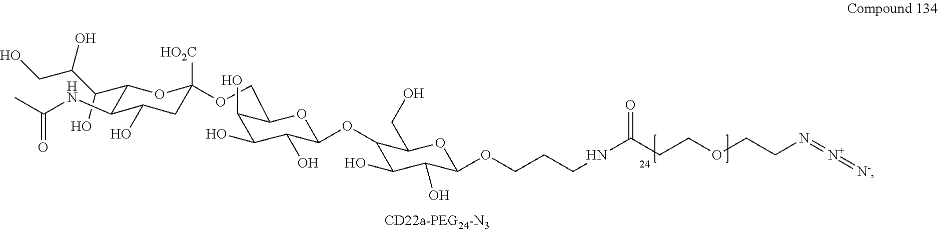

Compound 134, referred to CD22a-PEG₂₄-N₃ was synthesized by dissolving 74.8 mg of CD22a Amine (0.11 mmol, 1 eq) in 3.75 mL anhydrous DMSO. 49.9 uL TEA (0.36 mmol, 3.3 eq) was added and the solution was stirred at room temperature for five minutes. 165.1 mg of NHS-PEG₂₄-N₃ (0.13 mmol, 1.2 eq) was added to the reaction mixture and the reaction was stirred at room temperature for 1 hour when LC-MS indicated reaction was complete. The product was purified on a preparative HPLC system using a gradient of 15-45% acetonitrile/H2O (0.05% TFA) over 12 minutes on an Agilent Prep-C18 column, 30×100 mm, 5 m. The product eluted at ˜6 minutes and the resulting fractions were collected, frozen and then lyophilized to obtain 167 mg (73.7% yield) of a spectroscopically pure (94.9% AUC at 220 nm) white solid. MS (ESI) Calculated for C₇₇H₁₄₅N₅O₄₄ m/z 1843.9, found 923.1 (M/2+H)⁺.

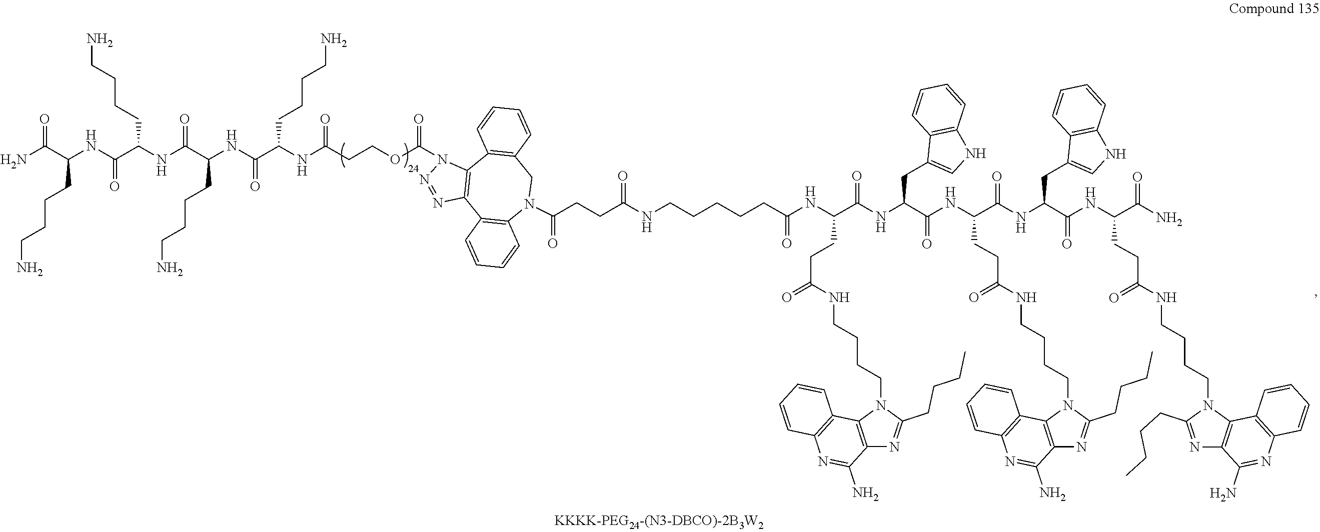

Compound 135, referred to KKKK-PEG₂₄-(N3-DBCO)-Ahx-2B3W2 was synthesized by reacting 1 equivalent of Compound 56 with 1 equivalent of azide-PEG₂₄-KKKK in DMSO at room temperature. The reaction progress was monitored by HPLC, which confirmed complete conversion of starting materials to Compound 135 after 16 hours. MS (ESI) Calculated for C₁₉₀H₂₈₃N₃₇O₄₁ m/z 3739.1, found 923.1 (M/4+H)⁺.

Similar reaction conditions were used to produce Compounds 164, and 136-152 summarized in Table 5 as was used for Compound 135.

TABLE 5
Amphiphiles of formula S-[B]-[U]-H-[D] having linear architecture.

| | | Starting U2-H-[D] | | | |
|---|---|---|---|---|---|
| Cmpd | Starting (S-B-U1) | Compound # | Net | Theo. | |
| # | Product (S-B-U-H-[D]) | | charge | MW | Found MW |
| 164 | KKKKKKK-PEG₂₄-X | Compound 60 | +8 | 4211.3 | Conforms |
| | KKKKKKK-PEG₂₄-(Lys(N₃)-DBCO)-bis(VZ-PAB-2Bxy)₂ | | | | |
| 136 | N₃-PEG₄-KKKKKKKK-NH₂ | Compound 56 | +8 | 3371.8 | Conforms |
| | KKKKKKKK-PEG4-(N3-DBCO)-Ahx-2B₃W₂ | | | | |
| 137 | N₃-PEG₁₂-KKKKKKKK-NH₂ | Compound 56 | +8 | 3724.2 | Conforms |
| | KKKKKKKK-PEG₁₂-(N₃-DBCO)-Ahx-2B₃W₂ | | | | |
| 138 | N₃-PEG₂₄-KKKKKKKK-NH₂ | Compound 56 | +8 | 4252.9 | Conforms |
| | KKKKKKKK-PEG₂₄-(N₃-DBCO)-Ahx-2B₃W₂ | | | | |
| 139 | KKKKKKKKK-PEG₃₆-X | Compound 56 | +9 | 5037.8 | Conforms |
| | KKKKKKKKK-PEG₃₆-(N₃-DBCO)-Ahx-2B₃W₂ | | | | |
| 140 | NH₂-PEG₍₅₀₀₀₎-N₃ | Compound 56 | +1 | 7056.1 | Conforms |
| | NH₂-PEG₍₅₀₀₀₎-(N₃-DBCO)-Ahx-2B₃W₂ | | | | |
| 141 | N₃-PEG₂₄-KKKKKKKK-NH₂ | Compound 54 | +8 | 3545.2 | Conforms |
| | KKKKKKKK-PEG₂₄-(N₃-DBCO)-Ahx-WWWWW | | | | |
| 142 | KKKKKKK- | Compound 56 | +8 | 4854.1 | Conforms |
| | SGSGSGSGSGSGSGSGSGSGSGSGX- | | | | |
| | NH₂ | | | | |
| | KKKKKKK-SGSGSGSGSGSGSGSGSGSGSGSGX-(N₃- | | | | |
| | DBCO)-Ahx-2B₃W₂ | | | | |
| 143 | N₃-PEG₂₄-DDDDDDD-COOH | Compound 56 | −8 | 4133.2 | Conforms |
| | DDDDDDD-PEG₂₄-(N₃-DBCO)-Ahx-2B₃W₂ | | | | |
| 144 | N₃-PEG₂₄-DDDDDDD-COOH | Compound 54 | −8 | 3425.7 | Conforms |
| | DDDDDDD-PEG₂₄-(N₃-DBCO)-Ahx-WWWWW | | | | |
| 145 | CD22a-PEG₂₄-N3 | Compound 56 | −1 | 3901.1 | Conforms |
| | CD22a-PEG₂₄-(N3-DBCO)-Ahx-2B₃W₂ | | | | |
| 146 | CD22a-PEG₂₄-N3 | Compound 54 | −1 | 3193.4 | Conforms |
| | CD22a-PEG₂₄-(N3-DBCO)-Ahx-WWWWW | | | | |
| 147 | N₃-PEG₂₄-KKKKKKKK-NH₂ | Compound 52 | +8 | 4235.8 | 1059.8 |
| | KKKKKKKK-PEG₂₄-(N₃-DBCO)-Ahx-F′10 | | | | (M/4 + H)⁺ |
| 148 | N₃-PEG₂₄-DDDDDDD-COOH | Compound 52 | −8 | 4116.1 | 1372.7 |
| | DDDDDDD-PEG₂₄-(N₃-DBCO)-Ahx-F′10 | | | | (M/3)⁺ |
| 149 | KKKKKKKKK-PEG₂₄-X | Compound 56 | +10 | 4509.2 | 1128.4 |
| | KKKKKKKKK-PEG₂₄-(N3-DBCO)-Ahx-2B₃W₂ | | | | (M/4 + H)⁺ |
| 150 | N₃-PEG₂₄-DDDDDDDDD-NH₂ | Compound 56 | −10 | 4264.3 | 1067.4 |
| | DDDDDDDDD-PEG₂₄-N₃-(N3-DBCO)-Ahx-2B₃W₂ | | | | (M/4 + H)⁺ |
| 151 | OOOOOOOOO-PEG₂₄-X | Compound 56 | +10 | 4383.0 | 1096.9 |
| | OOOOOOOOO-PEG₂₄-X-(N3-DBCO)-Ahx-2B₃W₂ | | | | (M/4)⁺ |
| 152 | kkkkkkkkk-PEG₂₄-X | Compound 56 | +10 | 4509.4 | 1128.5 |

| | |
|---|---|
| kkkkkkkkk-PEG₂₄-(N3-DBCO)-Ahx-2B₃W₂ | (M/4 + H)⁺ |

Single letter abbreviations are used for amino acid sequences in the above table; X = azidolysine; O = Ornithine; and k = d-lysine. Peptide-based starting materials were manufactured by solid-phase peptide synthesis by Genscript (Piscataway, NJ) and NH2-PEG₍₅₀₀₀₎-N₃ was obtained from Polysciences (Warrington, PA). Peptide-based starting material sequences are written from N- to C-terminus, and C-terminal —NH₂ and —COOH indicate that the peptides are terminated with either amide or carboxylic acid groups, respectively, or alternatively the sequences may be terminated with a functionalized PEG. Unless otherwise specified, any C-terminal X, azidolysine, is terminated with an amide and is implicit in the sequences (i.e., not shown). Net charge is the predicted net charge of the amphiphile in aqueous buffer at pH 7.4.

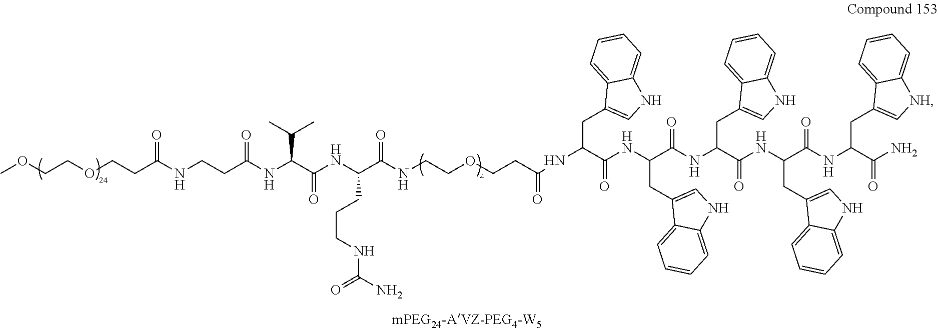

Compound 153, referred to as mPEG₂₄-A′VZ-PEG₄-W₅ was synthesized by reacting mPEG₂₄-NHS ester (0.0025 mmol, 1.0 eq) with H2N-A′VZ-PEG₄-W₅ (0.0025 mmol, 1.05 eq) (SEQ ID NO:67), which was made by solid phase synthesis, in anhydrous DMSO. The reaction was stirred for 16 hours at room temperature until HPLC indicated the reaction was complete. Upon completion, the reaction was purified on a preparatory HPLC system using a gradient of 35-55% acetonitrile/H₂O (0.05% TFA) over 12 minutes on an Agilent Prep-C18 column, 30×100 mm, 5 μm. The product eluted at ˜6.4 minutes and the resulting fractions were collected, frozen and then lyophilized to obtain a spectroscopically pure (>99% AUC at 220 nm) solid. MS (ESI) Calculated for C₁₃₂H₂₀₁N₁₇O₄₀ m/z 2664.42, found 1311.6 (M/2)⁺.

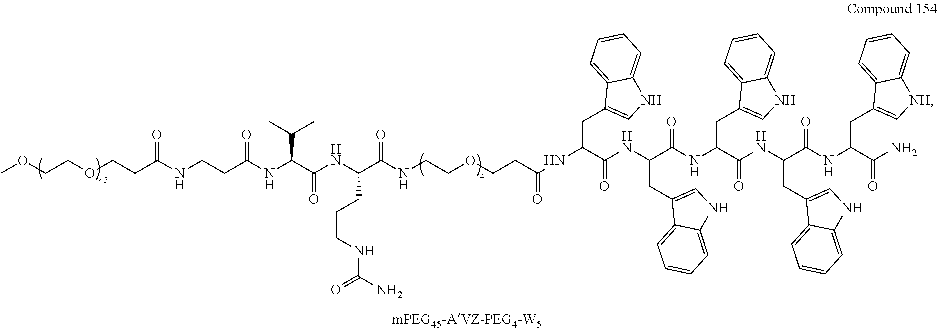

Compound 154, referred to as mPEG₄₅-A′VZ-PEG₄-W₅ or mP2000_{mw}-A′VZ-P4-W5 was synthesized and purified using the same procedure as described for Compound 153, except mPEG2000_{mw}-NHS ester was used instead of mPEG₂₄-NHS ester. The resulting fractions were collected, frozen and then lyophilized to obtain a spectroscopically pure (>99% AUC at 220 nm) solid. The starting mPEG2000_{mw}-NHS exists as a polydisperse product with an average mw of 2000. An exact formula or found mass, therefore, cannot be determined for this product

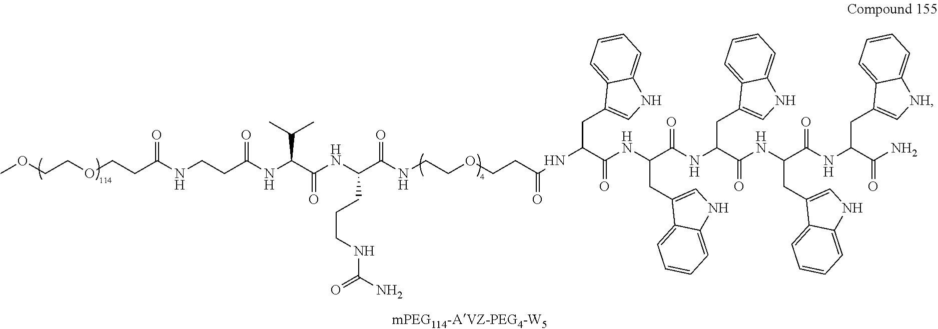

Compound 155, referred to as mPEG₁₁₄-A′VZ-PEG₄-W₅ was mP5000_{mw}-A′VZ-P4-W5 synthesized and purified using the same procedure as described for Compound 153, except mPEG5000_{mw}-NHS ester was used instead of mPEG₂₄-NHS ester. The resulting fractions were collected, frozen and then lyophilized to obtain a spectroscopically pure (95.8% AUC at 220 nm) solid. The starting mPEG5000_{mw}-NHS exists as a polydisperse product with an average mw of 5000. An exact formula or found mass, therefore, cannot be determined for this product

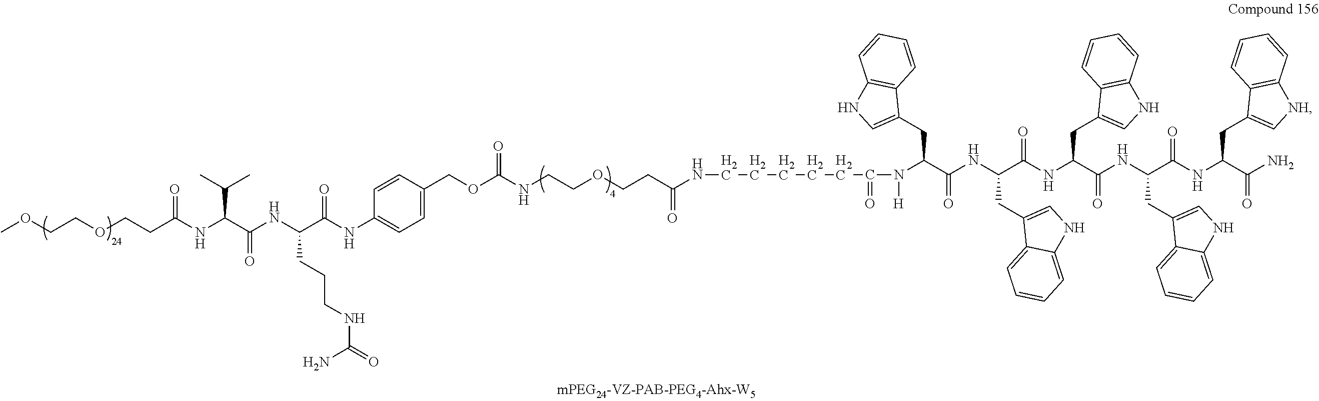

Compound 156, referred to as mPEG₂₄-VZ-PAB-PEG₄-Ahx-W₅ was synthesized by first synthesizing intermediate 156-a, referred to as Fmoc-PEG₄-AHx-W₅. The precursor NH₂-Ahx-W₅ (0.282 mmol, 1 eq) (SEQ ID NO:68), which was synthesized by solid phase synthesis, was dissolved in 32 mL DMSO and triethylamine (55 uL, 0.395 mmol, 1.4 eq) was added. Fmoc-PEG₄-NHS as a 100 mg/mL solution in DMSO (1.810 mL, 0.310 mmol, 1.1 eq.) was added and the reaction was stirred for 2 hours at room temperature until HPLC indicated the reaction was complete. 156-a was purified by reverse phase flash chromatography on a 60 g Biotage Safar C18 Bio Duo column over a 3-step gradient: 45% acetonitrile/H2O (0.05% TFA v/v) over 1 column volume (CVs), followed by 45-85% acetonitrile/H2O (0.05% TFA v/v) 15 CVs, and 95% acetonitrile/H2O (0.05% TFA v/v) 2 CVs. The product eluted at ˜65% acetonitrile and the resulting fractions were collected and the solvent removed to obtain a 64% yield. MS (ESI) Calculated for C₈₇H₉₅N₁₃O₁₃ m/z 1529.72, found 765.8 (M/2)⁺.

156-b. Intermediate 156-a was deprotected by dissolving in 20% piperidine/DMF and stirring for 20 minutes until HPLC indicated the reaction was complete. The deprotected 156-a was precipitated into diethyl ether and pelleted by centrifuging at 3000 rpm for 5 minutes. The diethyl ether supernatant was decanted and fresh diethyl ether was added. The pellet was broken up with a spatula and the product was again pelleted by centrifugation. The diethyl ether was decanted to provide an oil which was resuspended in H2O and then dried by lyophilization. The deprotected intermediate 156-a (27.9 mg, 0.021 mmol, 1 eq) was dissolved in DMF and Fmoc-VZ-PAB-PNP (18.0 mg, 0.023 mmol, 1.1 eq) was added. The reaction was stirred for 16 hours at room temperature until HPLC indicated that the reaction was complete. The product was precipitated in 10 mL of diethyl ether and centrifuged. The supernatant was decanted and fresh diethyl ether was added. The process of centrifuging and decanting was repeated. After decanting the diethyl ether, the resulting oily pellet was resuspended in DI water and lyophilized to yield the intermediate 156-c in 98% yield and 77% purity.

Compound 156. Intermediate 156-b was deprotected by dissolving in 20% piperidine/DMF and stirring for 20 minutes until HPLC indicated the reaction was complete. The product was precipitated in 10 mL of diethyl ether and centrifuged. The supernatant was decanted and fresh diethyl ether was added. The process of centrifuging and decanting was repeated. After decanting the diethyl ether, the resulting oily pellet was resuspended in DI water and lyophilized. To the deprotected intermediate 156-b in DMSO, mPEG24-NHS (1.1 eq.) and triethylamine (5 eq.) will be added.

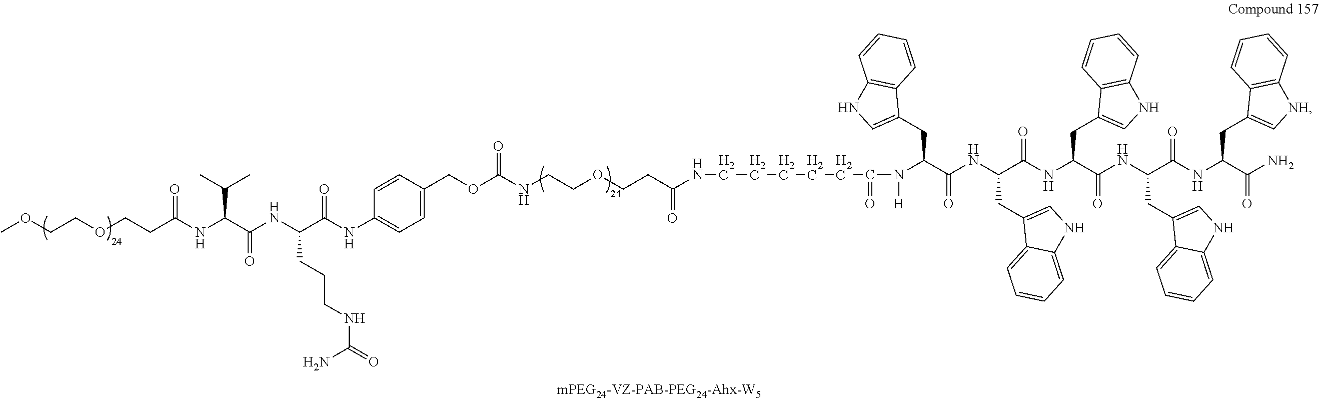

Compound 157, referred to as mPEG₂₄-VZ-PAB-PEG₂₄-Ahx-W₅ was synthesized using the same procedure as described for Compound 156, except mPEG₂₄-NHS ester was used instead of mPEG₄-NHS ester when synthesizing intermediate 157-a.

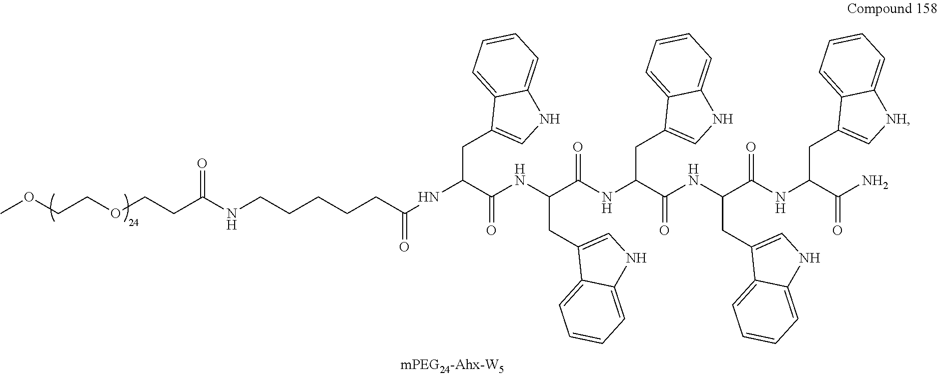

Compound 158, referred to as mPEG₂₄-Ahx-W₅, was synthesized by reacting mPEG₂₄-NHS ester (0.011 mmol, 1.5 eq) with NH₂-Ahx-W5 (0.0075 mmol, 1 eq) (SEQ ID NO:69), which was made by solid phase synthesis, in anhydrous DMSO with triethylamine (0.064 mmol 8 eq). The reaction was stirred for 4.5 hours at room temperature until HPLC indicated the reaction was complete. The reaction was purified by reverse phase flash chromatography on a 10 g Biotage Safar C18 Bio Duo column over a 3-step gradient: 35% acetonitrile/H2O (0.05% TFA v/v) over 1 column volume (CVs), followed by 35-65% acetonitrile/H2O (0.05% TFA v/v) 20 CVs, and 65% acetonitrile/H2O (0.05% TFA v/v) 2 CVs. The product eluted at ˜43% acetonitrile and the resulting fractions were collected and the solvent removed to obtain spectroscopically pure (94.2% AUC at 220 nm) solid. MS (ESI) Calculated for C₁₁₃H₁₆₆N₁₂O₃₂ m/z 2203.17, found 1102.8 (M/2)⁺.

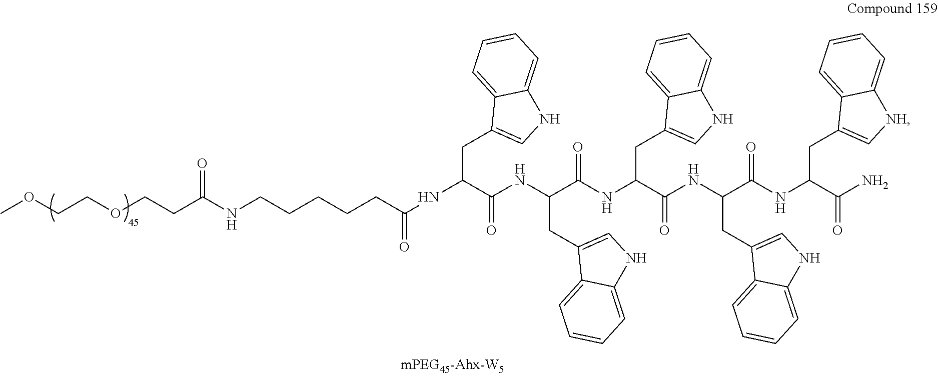

Compound 159, referred to as mPEG₄₅-Ahx-W₅ or mPEG2000_{mw}-Ahx-W₅ was synthesized using the same procedure as described for Compound 158, except mPEG2000_{mw}-NHS ester was used instead of mPEG₂₄-NHS ester. The reaction was purified by reverse phase flash chromatography on a 10 g Biotage Safar C18 Bio Duo column over a 3-step gradient: 30% acetonitrile/H2O (0.05% TFA v/v) over 1 column volume (CVs), followed by 30-60% acetonitrile/H2O (0.05% TFA v/v) 20 CVs, and 60% acetonitrile/H2O (0.05% TFA v/v) 2 CVs. The resulting fractions were collected and the solvent removed to obtain spectroscopically pure (>99% AUC at 220 nm) solid.

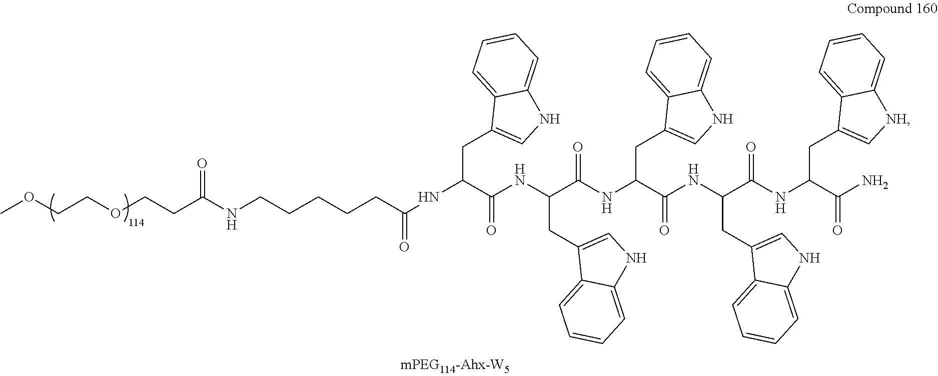

Compound 160, referred to as mPEG₁₁₄-Ahx-W₅ or mPEG5000_{mw}-Ahx-W₅ was synthesized using the same procedure as described for Compound 158, except mPEG5000_{mw}-NHS ester was used instead of mPEG₂₄-NHS ester, with HPLC indicating the reaction was complete after 30 hours. The reaction was purified by reverse phase flash chromatography on a 10 g Biotage Safar C18 Bio Duo column over a 3-step gradient: 30% acetonitrile/H2O (0.05% TFA v/v) over 1 column volume (CVs), followed by 30-60% acetonitrile/H2O (0.05% TFA v/v) 20 CVs, and 60% acetonitrile/H2O (0.05% TFA v/v) 2 CVs. The resulting fractions were collected and the solvent removed to obtain spectroscopically pure (91.5% AUC at 220 nm) solid.

Compound 161, mPEG₁₁₄-Ahx-2B₃W2

Compound 161, referred to as mPEG₁₁₄-Ahx-2B₃W₂ or mPEG5000_{mw}-Ahx-2B₃W₂ was synthesized using the same procedure as described for Compound 135, except mPEG5000_{mw}-azide was used instead of azide-PEG₂₄-KKKK. The reaction progress was monitored by HPLC, which confirmed complete conversion of starting materials to Compound 161 after 30 hours.

### Kinetics and Distribution of Particles Comprising a First Amphiphile and Optionally a Second Amphiphile

The data shown in FIG. 5 suggest the pH responsiveness of amphiphiles can impact cell uptake by splenocytes, with relatively lower cell uptake at higher pH, e.g., pH 7.4, as compared with lower pH. The tumor microenvironment is often described as having a lower pH than pH 7.4 due to the Warburg effect. Based on these considerations, it was hypothesized that amphiphiles exhibiting pH responsiveness may selectively accumulate in tumors due to the relatively lower pH of the tumor microenvironment as compared with blood pH 7.4.

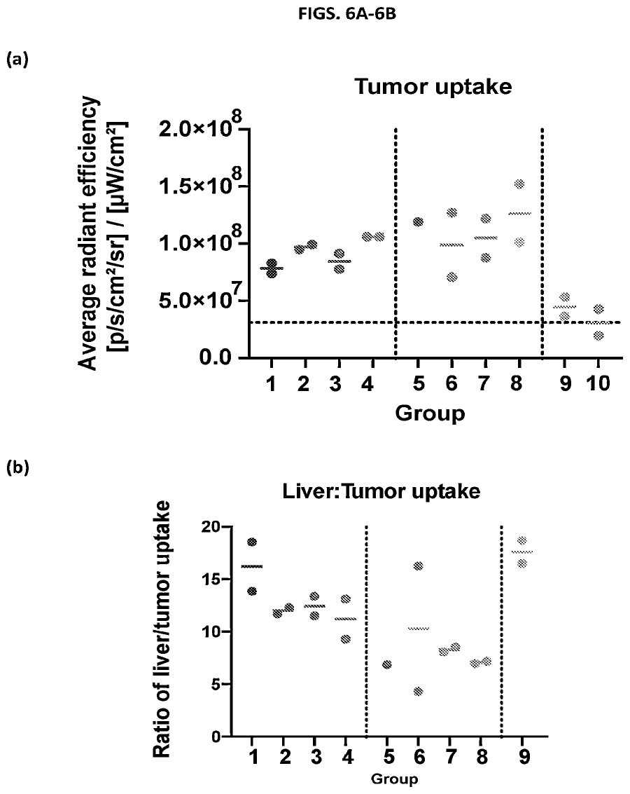

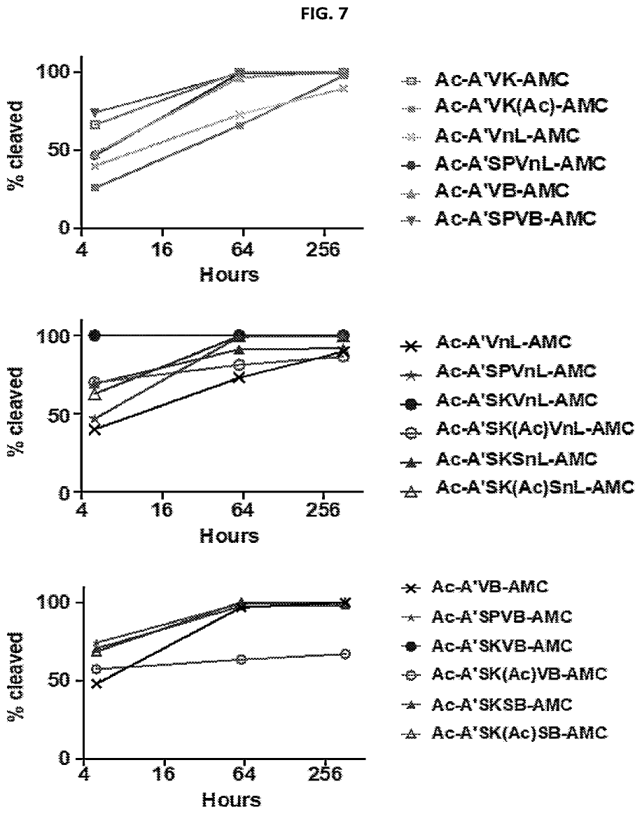

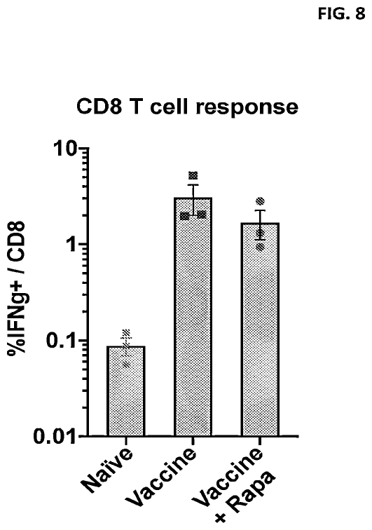

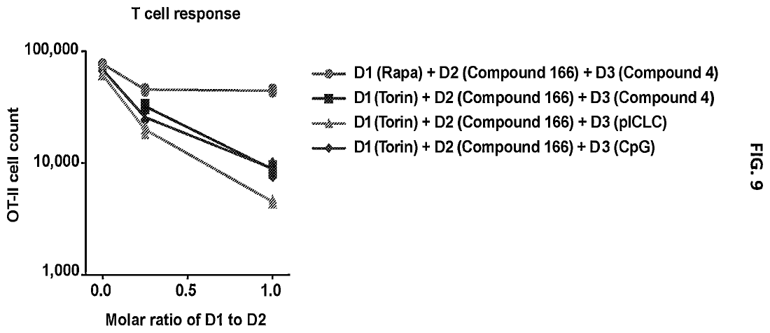

To evaluate the capacity of pH-responsive amphiphiles comprising solubilizing blocks with dendron architecture and carboxylic acid solubilizing groups to promote drug molecule accumulation in tumors, several compositions of particles comprising a first amphiphiphile and optionally a second amphiphile (Table 6) were evaluated for kinetics and distribution following IV administration in tumor (MC38)-bearing animals (FIG. 6). Among the particles with only a first amphiphile, tumor uptake and selectivity (Tumor: Liver uptake ratio) was higher for the amphiphiles that are more sensitive to pH changes, with Compounds 121 (Group 4), 124 and 126, which exhibit particle size and stability changes at pH 7.0, 6.5 and 6.4, respectively showing greater tumor uptake and selectivity than Compound 107 (Group 1), which is less sensitive to pH change.

TABLE 6
Composition of particles evaluate for kinetics and distribution.

| | | Second | Molar | (Dh, |
|---|---|---|---|---|
| Group | First amphiphile | amphiphile | ratio* | nm) |
| 1 | Compound 107 | None | N.A. | 13.6 |
| 2 | Compound 126 | None | N.A. | 13.4 |
| 3 | Compound 124 | None | N.A. | 20.2 |
| 4 | Compound 121 | None | N.A. | 138 |
| 5 | Compound 107 | Compound 161 | 9:1 | 13.8 |
| 6 | Compound 126 | Compound 161 | 9:1 | 15.8 |
| 7 | Compound 124 | Compound 161 | 9:1 | 13.0 |
| 8 | Compound 121 | Compound 161 | 9:1 | 12.2 |
| 9 | Kₙ-SIINFEKL-Ax647-2B₃W₂ | None | N.A. | 34 |

| | |
|---|---|
| 10 | Naïve |

*Molar ratio is the ratio of the first amphiphile to the second amphiphile. The particle size (hydrodynamic diameter, Dh) was determined by dynamic light scattering.

While all the compositions comprising amphiphiles with carboxylic acid solubilizing groups had greater tumor uptake and selectivity than an amphiphile comprising amine solubilizing groups and having net positive charge (Group 9), the compositions comprising the first amphiphile and a second amphiphile comprising a linear hydrophilic polymer had the highest tumor uptake and selectivity (FIG. 6). A non-limiting explanation is that the second amphiphile decreases clearance by liver, thereby providing longer circulation time and increased uptake by tumor, leading to overall greater tumor selectivity.

### Therapies for Preventing or Inhibiting an Unwanted Immune Response

For certain therapies used to induce a T cell response, activation of the immune system may also result in induction of antibodies against the therapy, such as antibodies against specific components of the carrier (if present), e.g., amphiphiles, or drug molecules. A current challenge is that the anti-carrier or -drug molecule antibodies can lead to reduced efficacy of the therapy upon subsequent administrations via increased clearance rates of the therapeutic.

Herein, we evaluated the use of inhibitors of mTOR to suppress antibody responses or antibody and T cell responses against different therapeutic modalities.

We first evaluated whether an inhibitor of mTORC1, rapamycin, could be used to suppress T cell responses against an antigen in the presence of a TLR-7/8a immunostimulant. Mice were treated at days 0 and 7 with particles comprising Compound 165, a charged modified peptide antigen conjugate of formula C-A-U-H-D, either alone or with 0.1 molar equivalents of rapamycin (Table 7). T cell responses were assessed by flow cytometry at day 13 and showed that both treatments induced CD8 T cell responses of comparable magnitude directed against the encoded antigen, GQAEPDRAHYNIVTFCCKCD (FIG. 8).

TABLE 7

| C-A-H composition | Ratio | Particle size, |
|---|---|---|
| (Compound number) | C-A-H:D1 | d. nm. |
| — | — | N.A. |
| KKKKKKKVRGQAEPDRAHYNIVTFCCKCDSPVZX(DBCO-Ahx-2B3W2) | — | 13.0 |
| (Compound 165) | | |
| KKKKKKKVRGQAEPDRAHYNIVTFCCKCDSPVZX(DBCO-Ahx-2B3W2) | Rapa, 0.1 | 13.8 |
| (Compound 165) | | |

These data show that combined mTORC1 inhibitors do not fully suppress proinflammatory T cell responses, particularly Th1-type CD4 T cells driven by TBET expression. While it may be beneficial to suppress antibodies while allowing for T cell responses to be induced against antigens encoded by the therapy or antigens present within the subject receiving treatment, for some treatments it may be beneficial to suppress both antibody and (pro-inflammatory) T cell responses.

It was unknown a priori if combined inhibitors of mTORC1 and mTORC2 could improve suppression of antibody and CD4 and/or CD8 T cell responses against antigens present within or encoded by the therapy or antigens present within the subject receiving treatment as compared with inhibitors of mTORC1 alone. Additionally, the class of inhibitors of mTORC1 and mTORC2 and their preferred ratio with other component of the therapy were unknown, e.g., molar ratio of an amphiphilic carrier (S-B-[U]-H-[D]) to a first drug molecule (D1) selected from an inhibitor of mTORC1 and mTORC2, or ratio of a first drug molecule (D1) selected from an inhibitor of mTORC1 and mTORC2 to that of a second drug molecule (D2) or expression system (D2e).

As a model system for assessing the capacity of inhibitors of mTORC1 and mTORC2 to suppress T cell responses, we evaluated whether an inhibitor of mTORC1 (Rapamycin) or an inhibitor of mTORC1 and mTORC2 (Torin) could suppress T cell responses generated against a second drug molecule (D2) of formula A-U-H comprising a peptide antigen (A) in the presence of a third drug molecule (D3) selected from either a TLR-7/8a (Compound 4), a TLR-3a (pICLC) or a TLR-9a (CpG). Accordingly, particles comprising Compound 113, an amphiphilic carrier of formula S-B-U-H, further comprising a first drug molecule (D1), a second drug molecule (D2) and a third drug molecule (D3) as summarized in Table 8, were incubated with a coculture of antigen presenting cells and CD4 T cell clones (“OT-II cells”) that recognize the antigen (A) sequence ESLKISQAVHAAHAEINEAGREVVG encoded by D2, Compound 166.

TABLE 8

| Group | S-B-H composition | | | | Ratio |
|---|---|---|---|---|---|
| number | (Compound number) | D1 | D2 | D3 | SBH:D2:D3 |
| 11 | Tetra(Man-P3)-P24-W5 (Compound 113) | Rapa | Compound 166 | Compound 4 | 1:1:0.5 |
| 13 | Tetra(Man-P3)-P24-W5 (Compound 113) | Torin | Compound 166 | Compound 4 | 1:1:0.5 |
| 12 | Tetra(Man-P3)-P24-W5 (Compound 113) | Torin | Compound 166 | plCLC | 1:1:80* |
| 10 | Tetra(Man-P3)-P24-W5 (Compound 113) | Torin | Compound 166 | CpG | 1:1:2 |

*Molar amount to determine the molar ratio for pICLC was calculated assuming an average MW of a nucleotide base of ~500 g/mol.

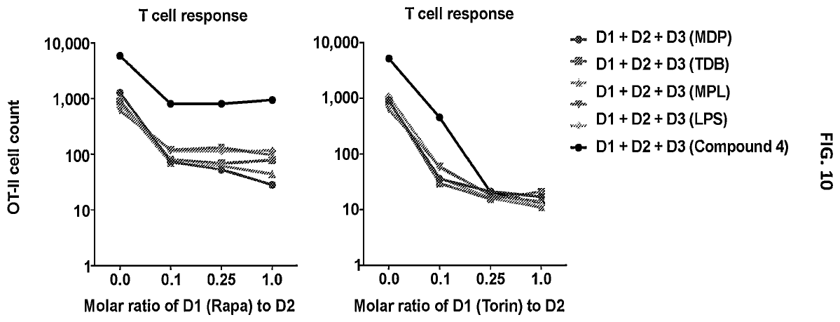

The data show that rapamycin (“Rapa”) marginally suppresses T cell proliferation, whereas the combined mTORC1 and mTORC2 inhibitor results in dose-dependent inhibition of T cells and nearly complete suppression of proinflammatory T cells, including Tbet expressing Th1-type CD4 T cells, even in the presence of potent immunostimulants (i.e., the TLR-3, TLR-7/8 and TLR-9 agonists pICLC, CpG and Compound 4) (FIG. 9).

Additional studies were undertaken to further assess the impact that the molar ratio of SBH to D1 and the molar ratio of D1 to D2 had on the capacity of D1 to suppress an immune response, e.g., a T cell response induced against D2. Particles comprising Compound 113, an amphiphilic carrier of formula S-B-U-H, further comprising a first drug molecule (D1), a second drug molecule (D2) and a third drug molecule (D3) as summarized in Table 9, were incubated with a coculture of antigen presenting cells and CD4 T cell clones (“OT-II cells”) that recognize the antigen (A) sequence ESLKISQAVHAAHAEINEAGREVVG encoded by D2, Compound 166.

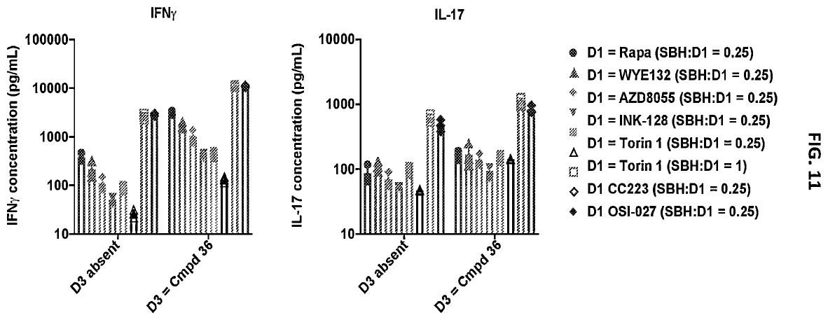

Notably, T cell suppression was maximized when molar ratios of SBH to D1 (and D1 to D2) were greater than or equal to 0.25 with responses plateauing at ratios above 0.25 (FIG. 10).

TABLE 9

| S-B-H composition | | | | Ratio |
|---|---|---|---|---|
| (Compound number) | D1 | D2 | D3 | SBH:D2:D3 |
| Tetra(Man-P3)-P24-W5 | Rapa | Compound 166 | MDP | 1:1:1 |
| (Compound 113) | | | | |
| Tetra(Man-P3)-P24-W5 | Rapa | Compound 166 | TDB | 1:1:0.1 |
| (Compound 113) | | | | |
| Tetra(Man-P3)-P24-W5 | Rapa | Compound 166 | MPL | 1:1:0.25 |
| (Compound 113) | | | | |
| Tetra(Man-P3)-P24-W5 | Rapa | Compound 166 | LPS | 1:1:0.25 |
| (Compound 113) | | | | |
| Tetra(Man-P3)-P24-W5 | Rapa | Compound 166 | Compound 4 | 1:1:0.5 |
| (Compound 113) | | | | |
| Tetra(Man-P3)-P24-W5 | Torin | Compound 166 | MDP | 1:1:1 |
| (Compound 113) | | | | |
| Tetra(Man-P3)-P24-W5 | Torin | Compound 166 | TDB | 1:1:0.1 |
| (Compound 113) | | | | |
| Tetra(Man-P3)-P24-W5 | Torin | Compound 166 | MPL | 1:1:0.25 |
| (Compound 113) | | | | |
| Tetra(Man-P3)-P24-W5 | Torin | Compound 166 | LPS | 1:1:0.25 |
| (Compound 113) | | | | |
| Tetra(Man-P3)-P24-W5 | Torin | Compound 166 | Compound 4 | 1:1:0.5 |
| (Compound 113) | | | | |

Moreover, while the combined mTORC1/mTORC2 inhibitor Torin resulted in greater T cell suppression as compared with Rapamycin, the extent of T cell suppression against D2 was also impacted by the composition of the innate immune stimulus provided by D3 (FIG. 10). Accordingly, rapamycin exhibited greater suppression of T cell responses when combined with D3 selected from a NOD-like receptor agonist (muramyl dipeptide (MDP)), a mincle receptor agonist (trehalose dibehenate (TDB)) and TLR4 agonists (LPS and MPL) as compared with D3 selected from a TLR-7/8a (Compound 4).

These data suggest that mTORC1 inhibition may be sufficient for suppressing immune responses, e.g., antibody and/or T cell responses, against antigens present within or encoded by the therapy or antigens present within the subject receiving treatment in the presence of an inflammatory context with low or absent IL-12 and interferons, but that inhibiting both mTORC1 and mTORC2 is critical to suppressing immune responses against antigens present within or encoded by the therapy or antigens present within the subject receiving treatment in the presence of IL-12 and interferons.

As the inflammatory context of the host, i.e., the subject receiving treatment is often unknown, the above results (FIG. 10) suggest that use of combined mTORC1 and mTORC2 inhibitors, such as Torin, may be helpful for ensuring adequate suppression of unwanted immune responses generated against antigens present within or encoded by a therapy or the host. For instance, peptide/protein-based therapeutics, cell-based therapies, and expression systems are often limited by immune responses generated against the peptide/protein-based therapeutic, cell-based therapy and expression system. As disclosed herein, such unwanted immune responses could be mitigated through using combined mTORC1 and mTORC2 inhibitors.

The above data show that use of mTOR inhibitors, particularly, combined mTORC1/mTORC2 inhibitors can be effective for suppressing unwanted immune responses. However, such molecules can be limited low solubility in aqueous solutions and potential off-target toxicity. To address these challenges, we developed amphiphiles of formula S-B-[U]-H as carriers to improve solubility and promote improved uptake by antigen-presenting cells to reduce off-target toxicity.

While the amphiphiles of formula S-B-[U]-H were generally found to be effective carriers for inhibitors of mTOR providing high drug encapsulation efficiencies, certain ratios of the amphiphile to drug molecule were found to be preferred for maximizing efficacy while ensuring stable formulations of nanoparticle micelles. Accordingly, it was found that macrolide based mTOR inhibitors, such as rapamycin, generally formed stable nanoparticle micelles up to a ratio of S-B-[U]-H to macrolide of about 1:1, though, more preferably about 1:0.5, though often no more than 1:0.25. In contrast, mTOR inhibitors comprising small molecule drugs with planar aromatic structure, such as Torin 1, Torin 2, INK128, AZD8055, OSI-027, KU63794, WYE-354, etc., were found to load more efficiently into nanoparticle micelles comprising amphiphiles of formula S-B-[U]-H, wherein H comprises aromatic groups, with loading up to 1:1 molar ratio of S-B-[U]-H to D being well tolerated and forming nanoparticles with stable hydrodynamic size.

Particles comprising amphiphiles of formula S-B-[U]-H and drug molecules selected from inhibitors of mTORC1 and mTORC2 were generally found to be effective for reducing or eliminating unwanted immune responses; however, certain classes of inhibitors of mTORC1 and mTORC2 were found to be more effective than other classes.

TABLE 10

| S-B-U-H composition | | | | Ratio |
|---|---|---|---|---|
| (Compound number) | D1 | D2 | D3 | SBH:D1:D2:D3 |
| Tetra(Man-P3)-P24-W5 (Compound 113) | Rapa | Compound 166 | — | 1:0.25:1 |
| Tetra(Man-P3)-P24-W5 (Compound 113) | WYE132 | Compound 166 | — | 1:0.25:1 |
| Tetra(Man-P3)-P24-W5 (Compound 113) | AZD8055 | Compound 166 | — | 1:0.25:1 |
| Tetra(Man-P3)-P24-W5 (Compound 113) | INK128 | Compound 166 | — | 1:0.25:1 |
| Tetra(Man-P3)-P24-W5 (Compound 113) | Torin 1 | Compound 166 | — | 1:0.25:1 |
| Tetra(Man-P3)-P24-W5 (Compound 113) | Torin 1 | Compound 166 | — | 1:1:1 |
| Tetra(Man-P3)-P24-W5 (Compound 113) | CC223 | Compound 166 | — | 1:0.25:1 |
| Tetra(Man-P3)-P24-W5 (Compound 113) | OSI-027 | Compound 166 | — | 1:0.25:1 |
| Tetra(Man-P3)-P24-W5 (Compound 113) | Rapa | Compound 166 | Compound 4 | 1:0.25:1:0.5 |
| Tetra(Man-P3)-P24-W5 (Compound 113) | WYE132 | Compound 166 | Compound 4 | 1:0.25:1:0.5 |
| Tetra(Man-P3)-P24-W5 (Compound 113) | AZD8055 | Compound 166 | Compound 4 | 1:0.25:1:0.5 |
| Tetra(Man-P3)-P24-W5 (Compound 113) | INK128 | Compound 166 | Compound 4 | 1:0.25:1:0.5 |
| Tetra(Man-P3)-P24-W5 (Compound 113) | Torin 1 | Compound 166 | Compound 4 | 1:0.25:1:0.5 |
| Tetra(Man-P3)-P24-W5 (Compound 113) | Torin 1 | Compound 166 | Compound 4 | 1:1:1:0.5 |
| Tetra(Man-P3)-P24-W5 (Compound 113) | CC223 | Compound 166 | Compound 4 | 1:0.25:1:0.5 |
| Tetra(Man-P3)-P24-W5 (Compound 113) | OSI-027 | Compound 166 | Compound 4 | 1:0.25:1:0.5 |

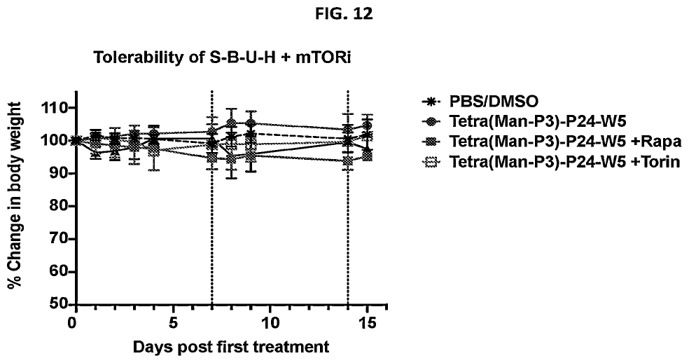

Particles comprising Compound 113, an amphiphilic carrier of formula S-B-U-H, further comprising a first drug molecule (D1), a second drug molecule (D2) and optionally a third drug molecule (D3) as summarized in Table 10, were incubated with a coculture of antigen presenting cells and CD4 T cell clones (“OT-II cells”) that recognize the antigen (A) sequence ESLKISQAVHAAHAEINEAGREVVG encoded by D2, Compound 166. The cell culture supernatant was assessed for the cytokines IFNγ and IL-17 to assess for the proliferation of Th1- and Th17-type CD4 T cells, respectively (FIG. 11). Notable, while all of the mTOR inhibitors (Table 10) exhibited comparable capacity to suppress Th17-type CD4 T cell responses, even in the presence of a potent immunostimulant (Compound 4), the composition of the mTOR inhibitor was found to have significant impact on the magnitude of Th1-type CD4 T cell response induced, with Torin 1, INK-128, AZD8055 and WYE132 leading to substantially improved T cell response suppression as compared with Rapa, CC223 and OSI-027 (FIG. 11).These results led to the identification of Torin 1, INK-128 and AZD8055, and any derivatives or salt forms thereof, as preferred drug molecules for suppressing unwanted immune responses.

As there is generally limited preclinical and clinical toxicity data available for formulations of combined mTORC1 and mTORC2 inhibitors, we next assessed formulations of particles amphiphiles and inhibitors of mTOR for safety and tolerability in vivo in mice. Particles comprising Compound 113 and either no drug molecule or an mTOR inhibitor (Table 11) were administered to C57BL/6 mice (n=3/group) at 50 nmol, 100 nmol and 200 nmol of S-B-U-H on days 0, 7 and 14 and body weight was assessed prior to and following each treatment (FIG. 12).

TABLE 11

| S-B-U-H composition | D1, ratio | Particle size, |
|---|---|---|
| (compound number) | S-B-H:D | d. nm. |
| Tetra(Man-P3)-P24-W5 | — | N.A. |
| (Compound 113) | | |
| Tetra(Man-P3)-P24-W5 | — | 15.9 |
| (Compound 113) | | |
| Tetra(Man-P3)-P24-W5 | Rapa, 0.1 | 67.7 |
| (Compound 113) | | |
| Tetra(Man-P3)-P24-W5 | Torin 1, 0.25 | 12.4 |
| (Compound 113) | | |

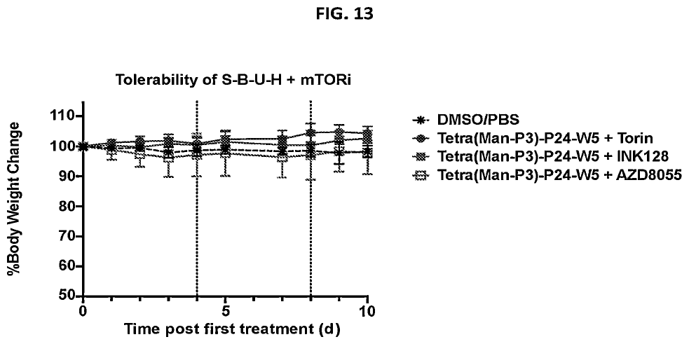

The tolerability of particles comprising Compound 113 and an mTOR inhibitor (Table 12) were administered to C57BL/6 mice (n=3/group) at 50 nmol, 100 nmol and 200 nmol of S-B-U-H on days 0, 4 and 8 and body weight was assessed prior to and following each treatment (FIG. 13).

TABLE 12

| S-B-U-H composition | D1, ratio | Particle size, |
|---|---|---|
| (compound number) | S-B-H:D:1 | d. nm. |
| Tetra(Man-P3)-P24-W5 (Compound 113) | — | N.A. |
| Tetra(Man-P3)-P24-W5 (Compound 113) | Torin, 0.25 | 11.9 |
| Tetra(Man-P3)-P24-W5 (Compound 113) | INK128, 0.25 | 47.5 |
| Tetra(Man-P3)-P24-W5 (Compound 113) | AZD8055, 0.25 | 9.2 |

The data show that particles comprising amphiphiles of formula S-B-[U]-H and drug molecules selected from inhibitors of mTORC1 and/or mTORC2 are generally well tolerated and do not cause overt toxicity in an animal species predictive of safety and tolerability in other mammals, including humans.

### Impact of the Ratio of Amphiphile to Drug Molecule

To assess the impact that the ratio of amphiphile to drug molecule (D) has on particle size and stability, the mTORC1/2 inhibitor Torin 1 was admixed with an amphiphile of Formula V, Compound 113, at varying molar ratios ranging from 1:1 to 1:16 moles of amphiphile to moles of drug. Notably, the amphiphile markedly improved the solubility of Torin 1 in both DMSO and aqueous buffer allowing for stable nanoparticle micelles with up to 1:4 molar ratio of amphiphile to drug molecule in aqueous buffer (Table 13).

TABLE 13

| | | | | | Concentration | Concentration | |
|---|---|---|---|---|---|---|---|
| S-B-U-H composition | | Molar ratio | Particle size, | Turbidity | S-B-U-H | S-B-U-H | |
| (Compound number) | D1 | S-B-H to D1 | d · nm. | (OD 490 nm) | 10% DMSO v/v PBS | 10% DMSO v/v PBS | |
| Tetra(Man-P3)-P24-W5 | Torin-1 | 1 | 12.96 | 0.041 | .1 mM | .1 | mM |
| (Compound 113) | | 2 | 13.71 | 0.04 | .1 mM | .2 | mM |
| | | 4 | 19.45 | 0.043 | .1 mM | .4 | mM |
| | | 8 | 69.54 | 0.051 | .1 mM | .8 | mM |
| | | 16 | 32.26 | 0.076 | .1 mM | 1.6 | mM |
| | | 1 | 12.96 | 0.054 | .8 mM | .8 | mM |

### Lysine-Based Dendrons Via SPPS

Amphiphiles described herein were produced in solution phase, on-resin by solid-phase peptide synthesis (SPPS) or using a combination of on-resin and solution phase. For example, an amphiphile of Formula VI comprising mannose SG and a lysine-based dendron amplifier was prepared using both on-resin and solution phase synthesis.

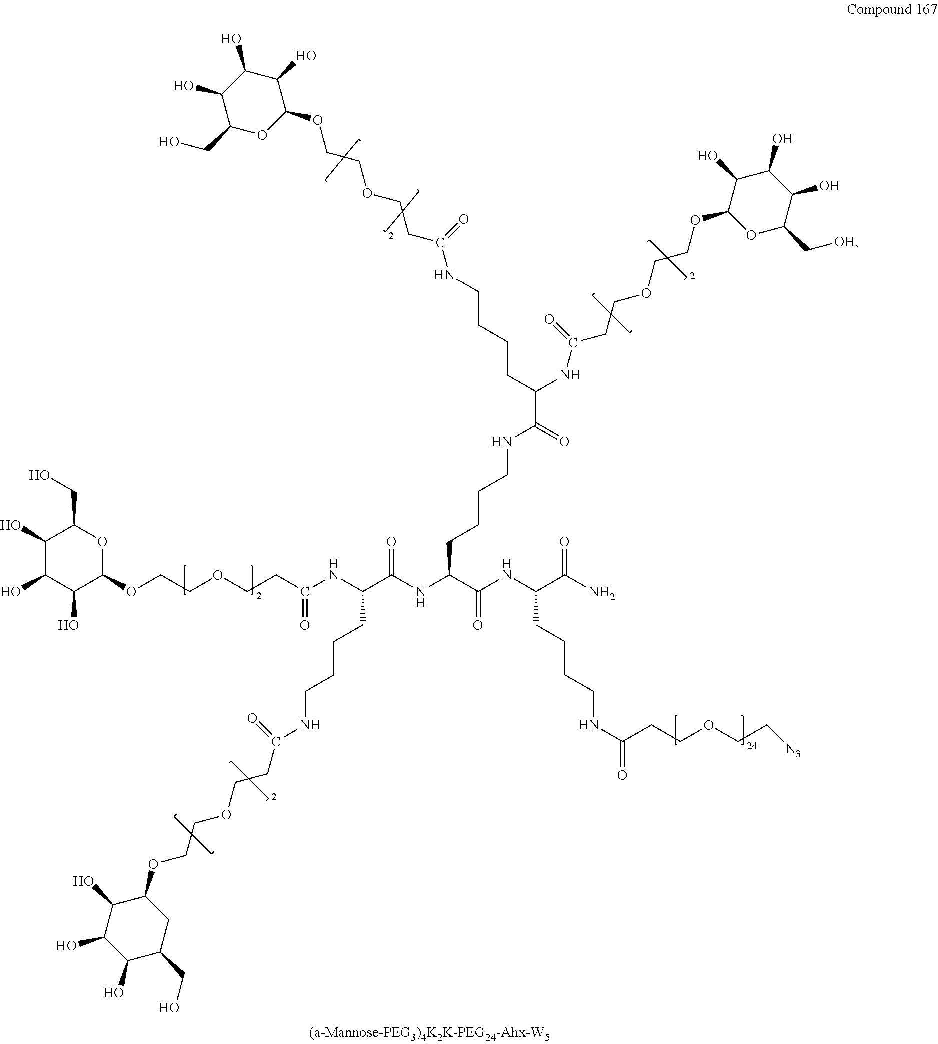

Compound 167, referred to as (a-Mannose-PEG₃)₄K₂K-PEG₂₄-X or Tetra(Man-P3)K₂K-P24-X, where X=azidolysine, was synthesized using Fmoc based solid phase peptide synthesis, resulting in a spectroscopically pure (97.0% AUC at 220 nm) colorless oil. MS (ESI) calculated for C₁₂₇H₂₃₈N₁₂O₆₅ m/z 2971.6, found 1486.9 (M/2+H)⁺.

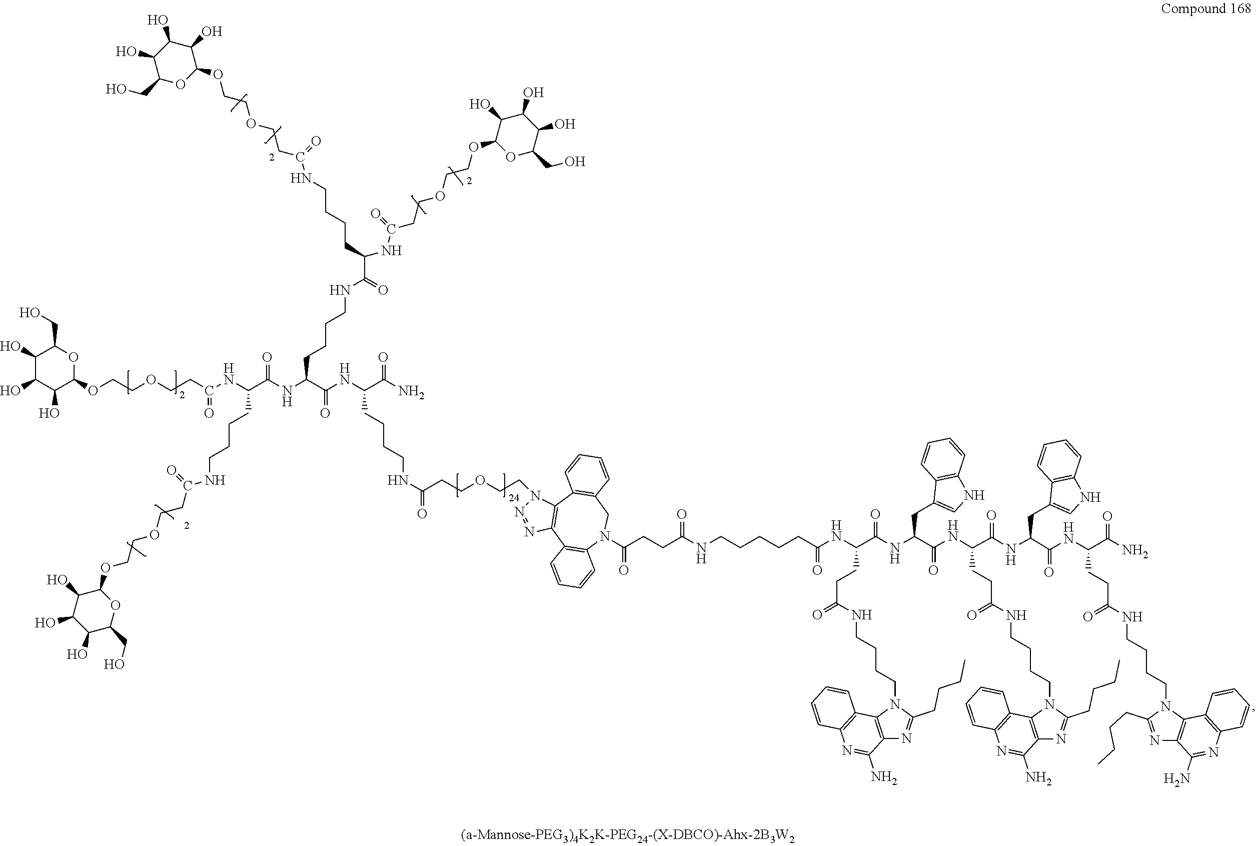

Compound 168, referred to as (a-Mannose-PEG₃)₄K₂K-PEG₂₄-(X-DBCO)-Ahx-2B₃W2 or Tetra(Man-P3)K₂K-P24-(X-DBCO)-Ahx-2B₃W2, was synthesized by reacting Compound 167 (0.001 mmol, 1.0 eq) with Compound 56 (0.001 mmol, 1.05 eq) in anhydrous DMSO for 16 hours at room temperature. HPLC was monitored to evaluate reaction progress and indicated complete conversion of Compound 167 to Compound 168, resulting in a spectroscopically pure (97.5% AUC at 220 nm) colorless solution. MS (ESI) calculated for C₂₄₃H₃₇₅N₃₇O₇₆ m/z 5027.7.4, found 1258.6 (M/4+H)⁺.

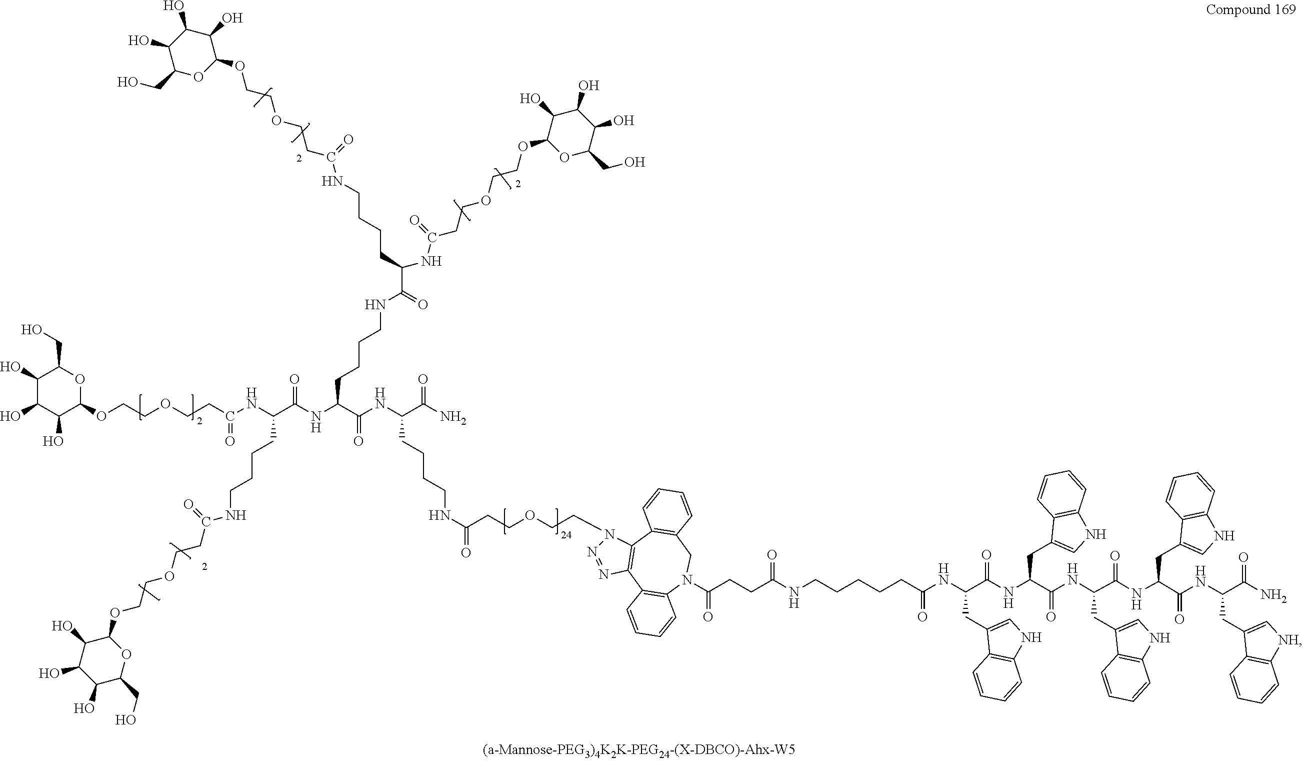

Compound 169, referred to as (a-Mannose-PEG₃)₄K₂K-PEG₂₄-(X-DBCO)-Ahx-W₅ or Tetra(Man-P3)K₂K-P24-(X-DBCO)-Ahx-W₅, was synthesized following the same procedure as Compound 168, except that Compound 54 was used in place of Compound 56, resulting in a spectroscopically pure (94.7% AUC at 220 nm) colorless solution. MS (ESI) calculated for C₂₀₇H₃₁₅N₂₅O₇₃ m/z 4319.2, found 865.0 (M/5+H)⁺.

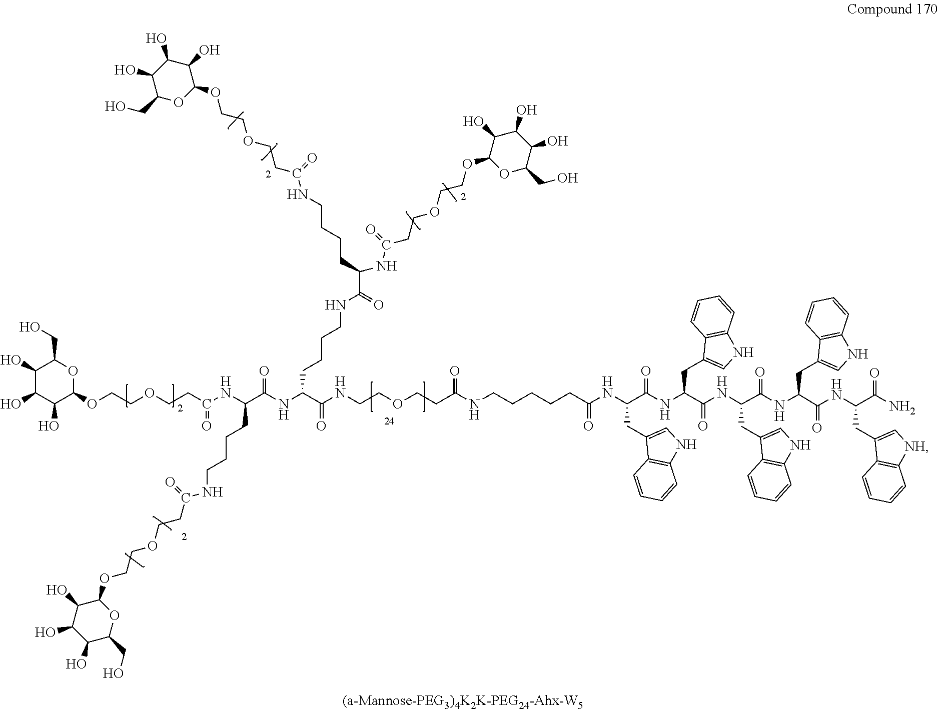

Compound 170, referred to as (a-Mannose-PEG₃)₄K₂K-PEG₂₄-Ahx-W₅ or Tetra(Man-P3)K₂K-P24-Ahx-W₅, was synthesized using Fmoc based solid phase peptide synthesis resulting in a spectroscopically pure (97.9% AUC at 220 nm) off-white solid. MS (ESI) calculated for ₁₈₂H₂₈₉N₁₉O₇₀ m/z 3861.0, found 1288.4 (M/3+H)⁺.

### Impact of Amphiphile and Drug Molecule Composition on Tolerability

Compounds 171-176 were produced in a similar manner as that described for Compound 168. Table 14 provides a summary of the synthesis and characterization of compounds 171-176.

TABLE 14

| | | U2-H-[D] | | | |
|---|---|---|---|---|---|
| Cmpd | (S-B-U1) Compound # | Compound # | | m/z | MS (ESI) |
| # | Product S-B-U-H-[D] | | Charge | Theo. | m/n Found |
| 171 | Compound 98 | Compound 56 | 0 | 5217.6 | 1305.4 |
| | Tetra(GalNAc-P3)-P24-(N3-DBCO)-Ahx-2B₃W₂ | | | | (M/4 + H)⁺ |
| 172 | mPEG5000-N3 | Compound 54 | 0 | 6349.2 | |
| | mPEG5000-(N₃-DBCO)-Ahx-W₅ | | | | |
| 173 | Compound 97 | Compound 65 | 0 | 5199.0 | 1300.4 |
| | Tetra(Man-P3)-P24-Ahx-(N3-DBCO)-2BXy₃W₂ | | | | (M/4 + H)⁺ |
| 174 | Compound 98 | Compound 65 | 0 | 5363.2 | 1341.4 |
| | Tetra(GalNAc-P3)-P24-(N3-DBCO)-Ahx-2BXy₃W₂ | | | | (M/4 + H)⁺ |
| 175 | Compound 94 | Compound 65 | −4 | 4310.0 | 1078.2 |
| | Tetra(COOH-ethyl)-P24-(N3-DBCO)-Ahx-2BXy₃W₂ | | | | (M/4 + H)⁺ |
| 176 | mPEG5000-N3 | Compound 65 | 0 | 7201.7 | |

mPEG5000-(N3-DBCO)-Ahx-2BXy3W2

Single letter abbreviations are used for amino acid sequences in the above table; Peptide-based starting materials were manufactured by solid-phase peptide synthesis by Genscript (Piscataway, NJ). mPEG5000-N3 was obtained from BroadPharm (San Diego, CA). Net charge is the predicted net charge of the amphiphile in aqueous buffer at pH 7.4.

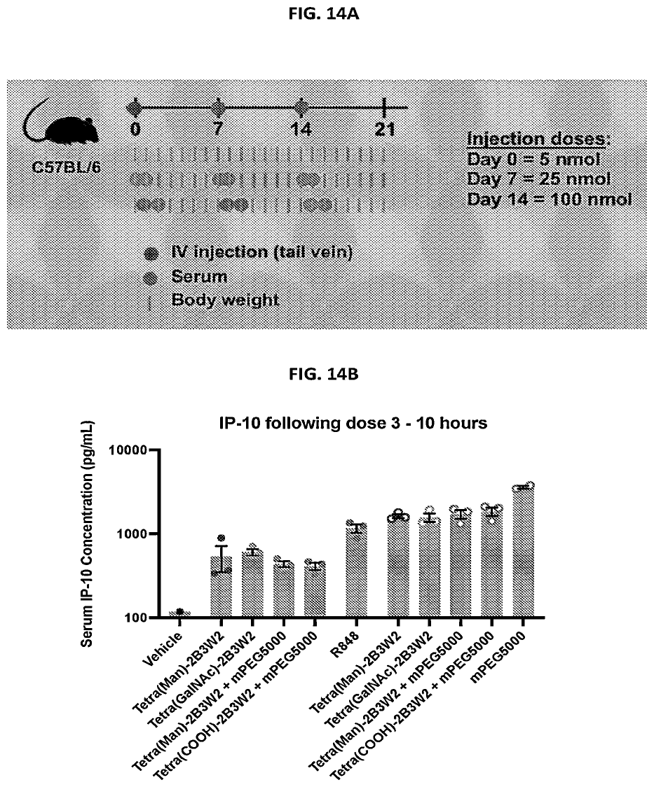

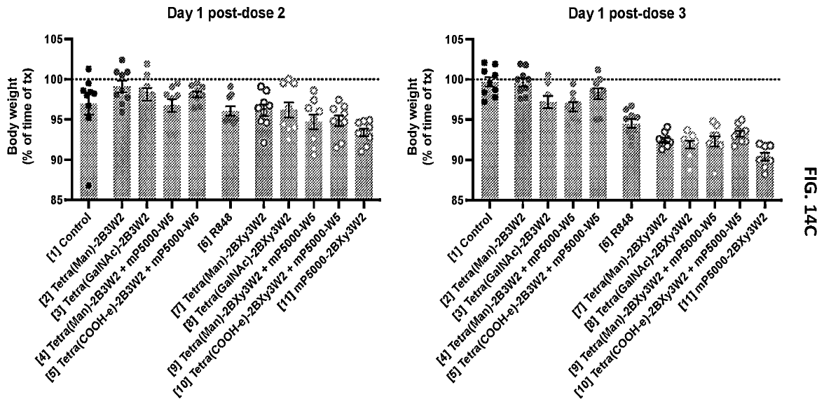

It was unknown how the surface group (SG) would impact the tolerability of amphiphiles of formula S-B-H-D carrying an immunostimulant. Therefore, to evaluate the impact of surface group composition on tolerability, amphiphiles of varying SG and drug molecule composition were prepared and evaluated in mice (Table 15 and FIG. 14).

TABLE 15

| | | | Ratio of 1^{st} | | |
|---|---|---|---|---|---|
| | | | amphiphile | | |
| | 1^{st} amphiphile | 2^{nd} amphiphile | to 2^{nd} | | Rh |
| Grp | (Compound #) | (Compound #) | amphiphile | Turbidity | (nm) |
| 1 | Vehicle control | — | — | — | — |
| 2 | Tetra(Man-P3)- | — | — | 0.039 | 12.52 |
| | 2B3W2 | | | | |
| | (Compound 114) | | | | |
| 3 | Tetra(GalNAc-P3)- | — | — | 0.04 | 11.25 |
| | 2B3W2 | | | | |
| | (Compound 171) | | | | |
| 4 | Tetra(Man-P3)- | mPEG5000- | 10:1 | 0.041 | 12.75 |
| | 2B3W2 | W5 | | | |
| | (Compound 114) | (Compound | | | |
| | | 172) | | | |
| 5 | Tetra(ethyl-COOH)- | mPEG5000- | 10:1 | 0.042 | 12.54 |
| | 2B3W2 | W5 | | | |
| | (Compound 107) | (Compound | | | |
| | | 172) | | | |
| 6 | R848 | — | — | 0.039 | — |
| 7 | Tetra(Man-P3)- | — | — | 0.039 | 12.35 |
| | 2BXy3W2 | | | | |
| | (Compound 173) | | | | |
| 8 | Tetra(GalNAc-P3)- | — | — | 0.041 | 13.08 |
| | 2BXy3W2 | | | | |
| | (Compound 174) | | | | |
| 9 | Tetra(Man-P3)- | mPEG5000- | 10:1 | 0.041 | 14.64 |
| | 2BXy3W2 | W5 | | | |
| | (Compound 173) | (Compound | | | |
| | | 172) | | | |
| 10 | Tetra(ethyl-COOH)- | mPEG5000- | 10:1 | 0.039 | 13.16 |
| | 2BXy3W2 | W5 | | | |
| | (Compound 175) | (Compound | | | |
| | | 172) | | | |
| 11 | mPEG5000- | — | — | 0.039 | 18.2 |
| | 2BXy3W2 | | | | |
| | (Compound 176) | | | | |

The results show that SG composition and drug molecule potency have a major impact on tolerability (FIG. 14). Notably, all amphiphiles carrying the small molecule, 2B, improved tolerability as compared with R848, with the compositions comprising mannose or carboxylic acids leading to the highest improvement in tolerability.

1. A composition comprising a first amphiphile and optionally a second amphiphile each having the formula S-[B]-[U]-H,
wherein S, independently for each occurrence, is a solubilizing block;
B, independently for each occurrence, is a spacer;
H, independently for each occurrence, is a hydrophobic block;
U, independently for each occurrence, is a linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof;
and at least one drug molecule (D) is noncovalently associated with or covalently bonded directly or via a suitable linker X1 to the first amphiphile and/or the second amphiphile.

2.-3. (canceled)

4. The composition according to claim 1, wherein the dendron amplifier comprises repeating monomer units of 1 to 10 generations having between 2 to 6 branches per generation.

5. (canceled)

6. The composition according to claim 4, wherein the repeating monomer units are selected from
(a) -FG1-(CH₂)_{y2}CH(R¹)₂, -FG1-(CH₂)_{y2}C(R¹)₃, -FG1-(CH₂CH₂O)_{y2}CH(R¹)₂, -FG1-(CH₂CH₂O)_{y2}C(R¹)₃, -FG1-CH(R¹)₂, and -FG1-C(R¹)₃,
wherein
R¹, independently for each occurrence, is selected from —(CH₂)_{y3}-FG2, —(OCH₂CH₂)_{y3}-FG2, and —CH₂(OCH₂CH₂)_{y3}-FG2;
y2 and y3, independently for each occurrence, are an integer of repeating units from 1 to 6;
FG1 is a first functional group; and
FG2 is a second functional group, or
(b) hydroxy acids, amino acids, polyols, polyamines, and amino alcohols.

7.-9. (canceled)

10. The composition according to claim 1, wherein the first amphiphile has the structure:

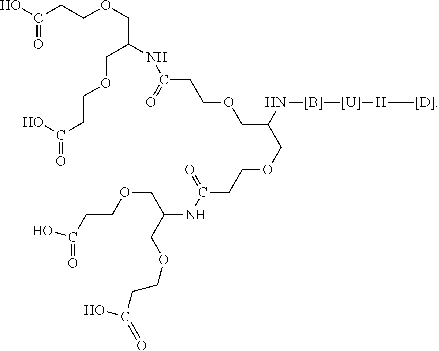

11. The composition according to claim 1, wherein the dendron amplifier comprises one or more polyethylene oxide (PEG) monomer units.

12. The composition according to claim 1, where the composition comprises a second amphiphile having the formula S-[B]-[U]-H.

13. The composition according to claim 12, wherein
the S of the second amphiphile is a hydrophilic polymer; and/or
the B of the second amphiphile is selected from peptides or hydrophilic polymers comprising from between about 1 to about 36 monomer units; and/or
the B of the first amphiphile is selected from hydrophilic polymers comprising from between about 1 to about 36 monomer units.

14. (canceled)

15. The composition according to claim 13, wherein the hydrophilic polymer comprises monomeric units selected from HEMA, HPMA, PEG, and zwitterionic betaine monomers.

16.-17. (canceled)

18. The composition according claim 12, wherein the second amphiphile has the formula S-X6-B-[U]-H,
wherein the linker X6 is selected from enzyme degradable peptides and pH sensitive linkers.

19.-24. (canceled)

25. The composition according to claim 1, wherein the H of the first amphiphile and/or second amphiphile comprises a higher alkane, an aromatic group, a fatty acid, a sterol, a polyunsaturated hydrocarbon, squalene, saponins, or a poly(amino acid), and/or a dendron amplifier.

26.-27. (canceled)

28. The composition according to claim 1, wherein each H independently comprises a poly(amino acid) comprising monomers selected from hydrophobic amino acids (M), reactive amino acids (N), spacer amino acids (O), charged amino acids (P), and combinations thereof, provided that at least one of M or N is present.

29. The composition according to claim 28, wherein each H independently comprises a poly(amino acid) having the formula:
-(M)ₘ-(N)ₙ-(O)ₒ-(P)ₚ-R³,
wherein
m, n, o and p each independently denote an integer of 0 to 100, wherein the sum of m, n, o, and p is less than or equal to 100, and at least one of m and n is not 0;
R³ is selected from hydrogen, NH₂, NH—CH₃, NH—(CH₂)_{y5}CH₃, OH, or a D either connected directly or through a suitable linker X1; and
y5 is an integer selected from 1 to 6.

30.-31. (canceled)

32. The composition according to claim 29, wherein
P, when present, is 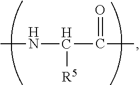
wherein each R⁵, independently, is a group that comprises 1 to 2 charged functional groups; and/or
O, when present, is 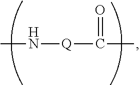
wherein each Q, independently, is selected from (CH₂)_{y6} and (CH₂CH₂O)_{y7}CH₂CH₂; each y6 is independently selected from an integer from 1 to 6; and each y7 is independently selected from an integer from 1 to 4; and/or
N, when present, is 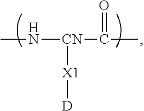
wherein each X1, independently, is a suitable linker; and each D, independently, is a drug molecule; and/or
M, when present, is 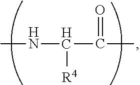
wherein each R⁴, independently, is 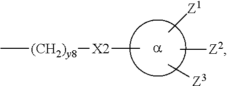
wherein α is aryl or heteroaryl; X2 is present or absent and when present is a suitable linker; y8 is selected from an integer from 0 and 6; and Z¹, Z², and Z³ are each independently selected from hydrogen, fluorine, hydroxy, amino, alkyl, and fluoroalkyl.

33.-46. (canceled)

47. The composition according to claim 32, wherein each R⁴ is independently selected from:

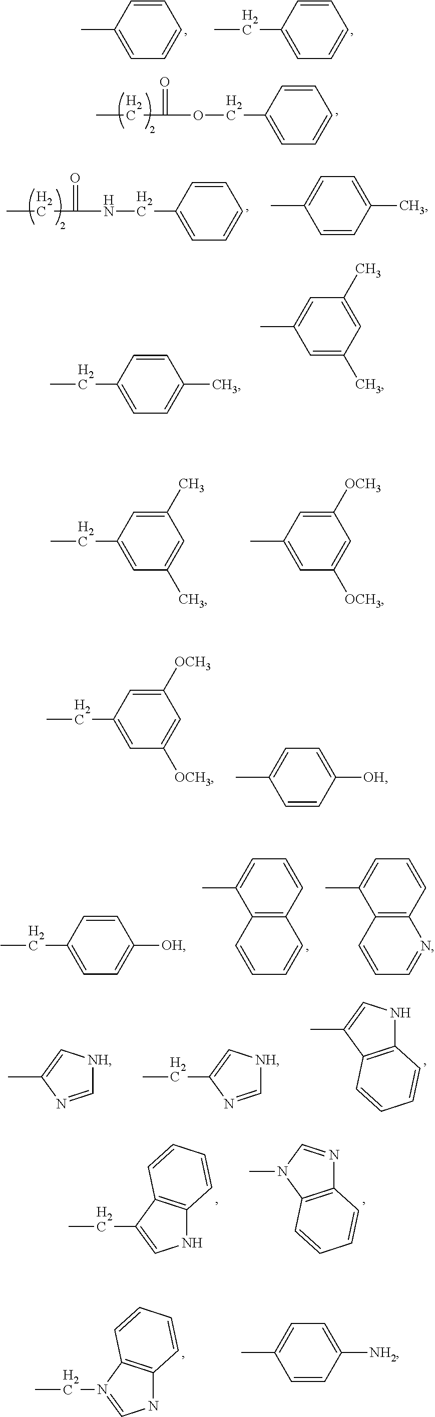

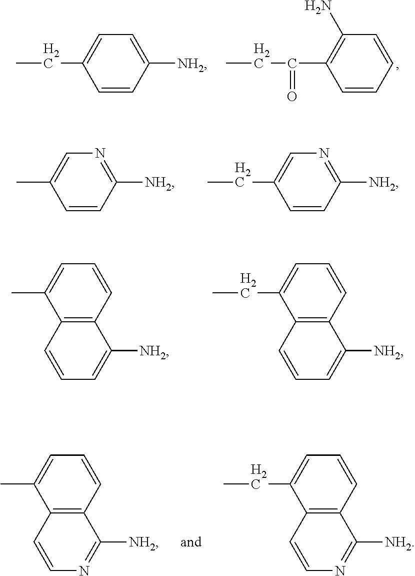

48.-61. (canceled)

62. The composition according to claim 1, wherein the at least one drug molecule D is an inhibitor of mTORC1 and/or mTORC2.

63.-76. (canceled)

77. The composition according to claim 1, wherein the first amphiphile and/or the second amphiphile have the structure: 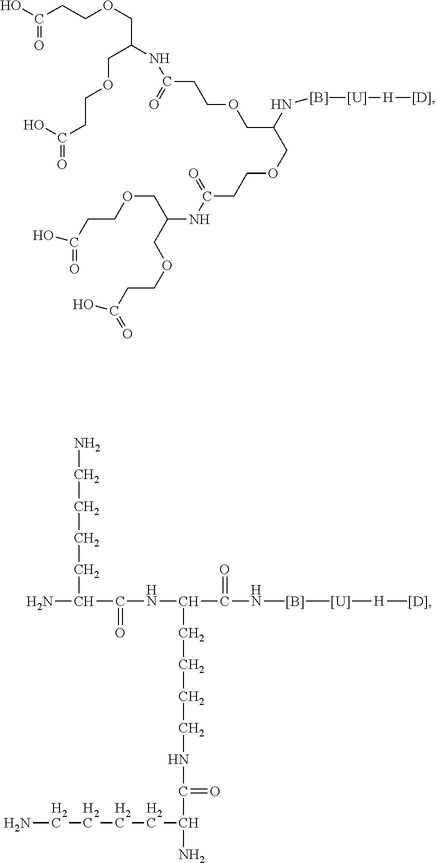 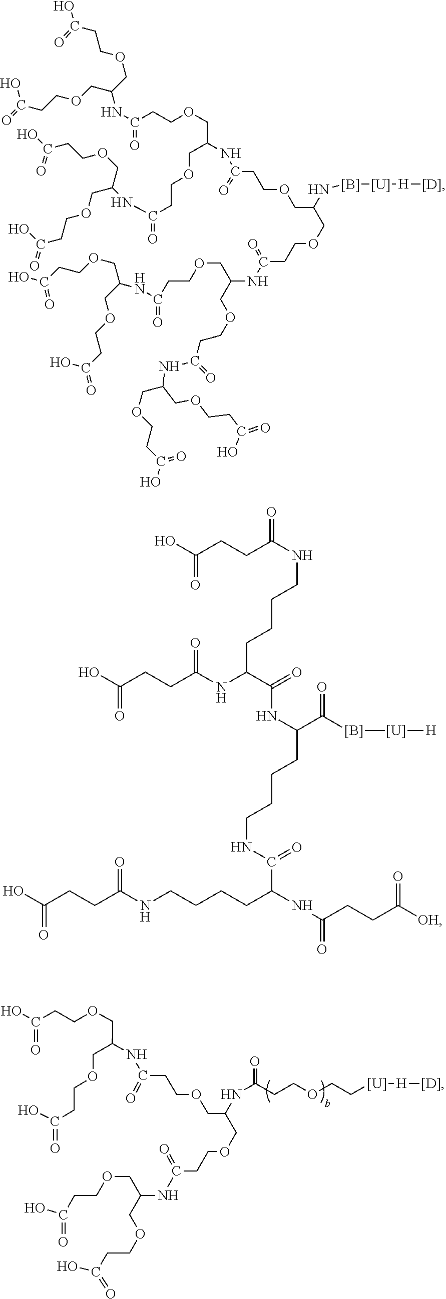
wherein b is an integer from 1 to 48, 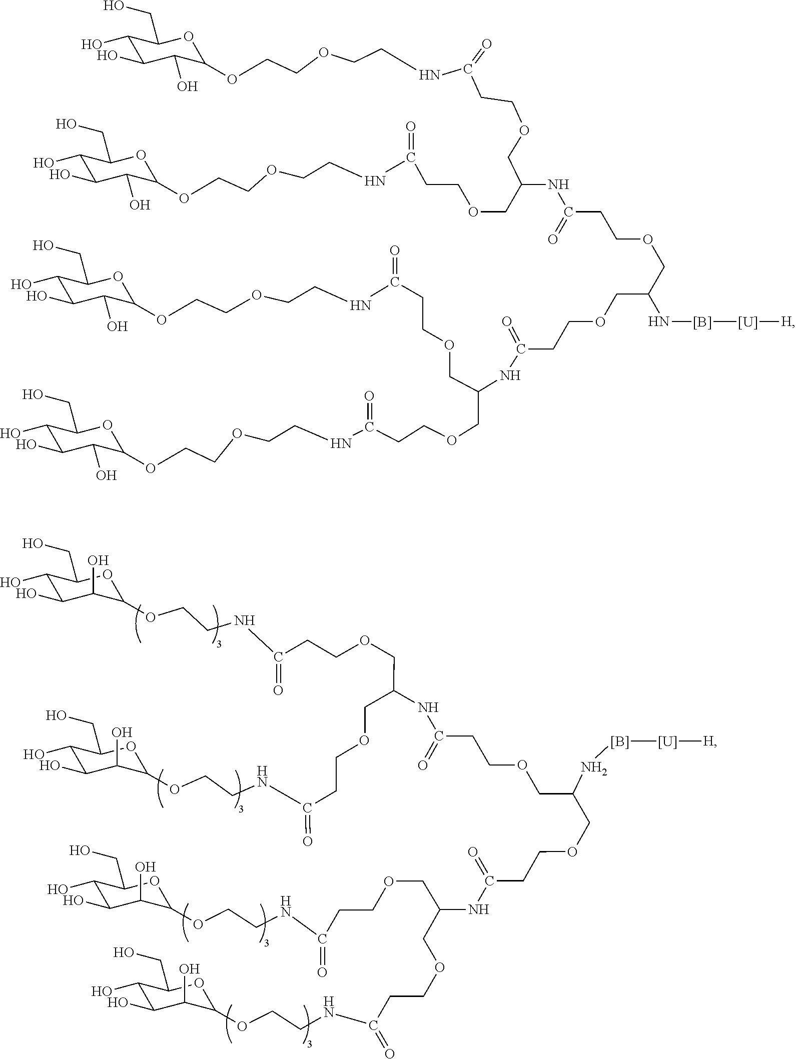 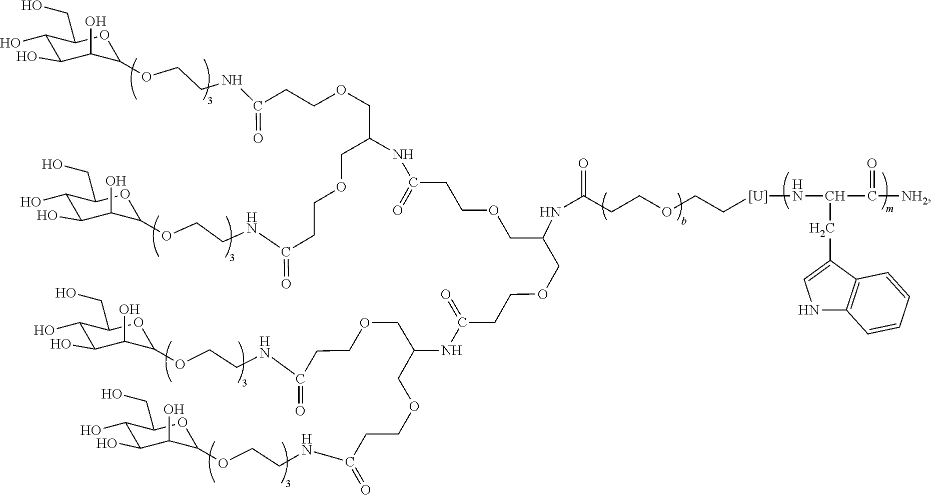
wherein b is an integer from 1 to 36, 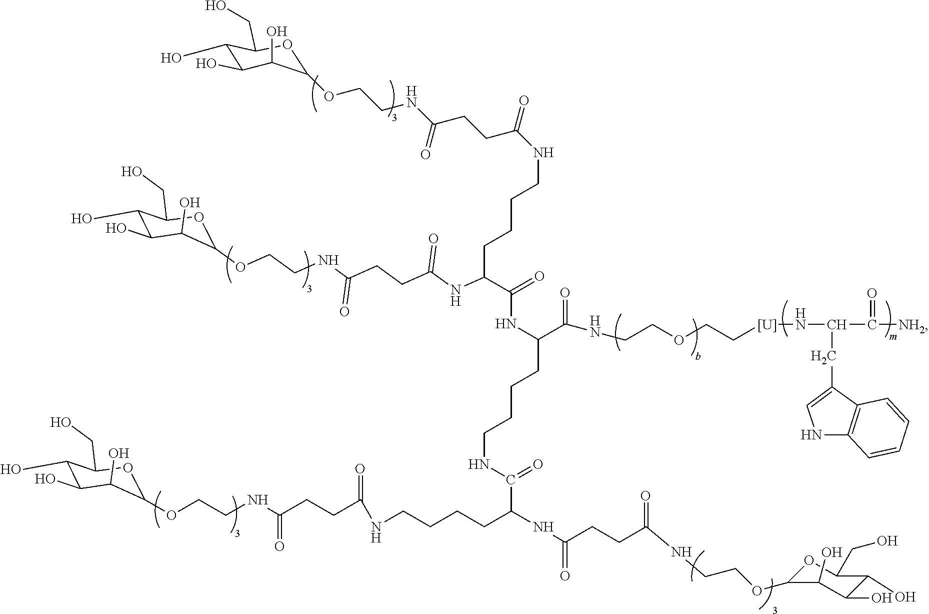
wherein b is an integer from 1 to 36, 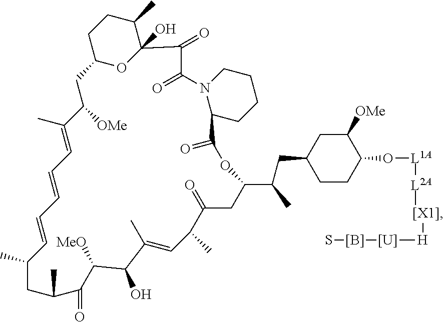
wherein L^{1A} is an alkylene linker; and L^{2A} is an amino, amido, ester, peptide, or nitrogen containing heterocyclyl linker, 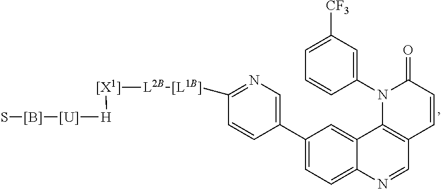
wherein L^{1B} is an alkylene-C(O)-NH— linker; and L^{2A} is an amino, amido, peptide, or nitrogen containing heterocyclyl linker, or 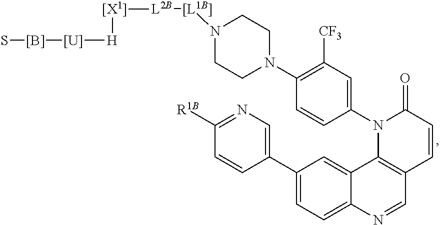
wherein L^{1B} is a 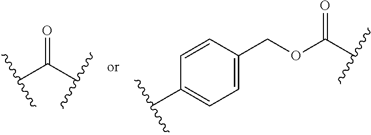
linker L^{2B} is an amino, amido, or peptide linker; and R^{1B} is NH₂ or

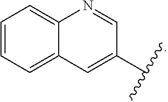 [SMILES: *c1cnc2ccccc2c1]

78.-79. (canceled)

80. The composition according to claim 1, wherein the first amphiphile and/or the optional second amphiphile have the structure:

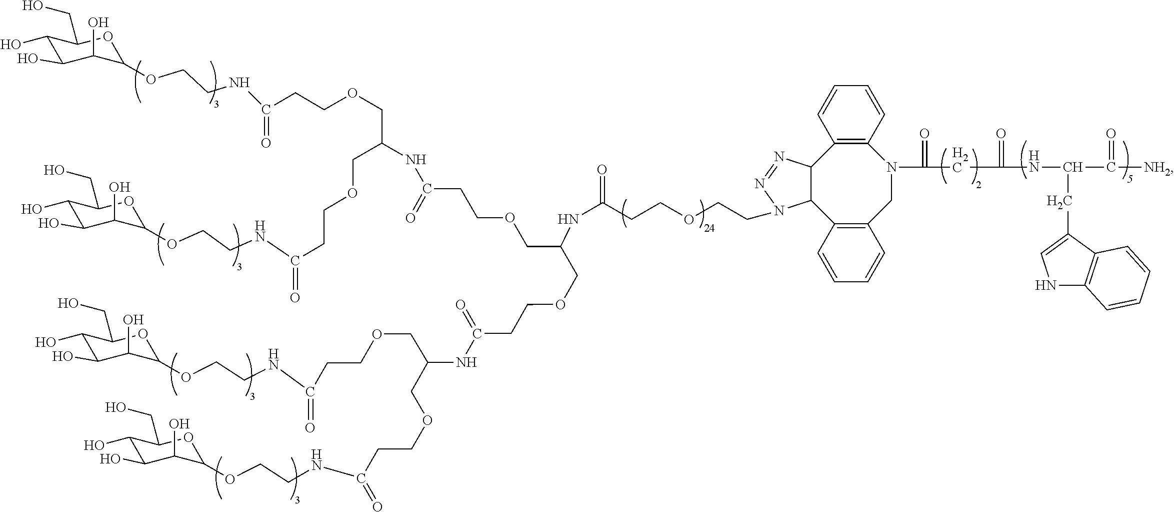

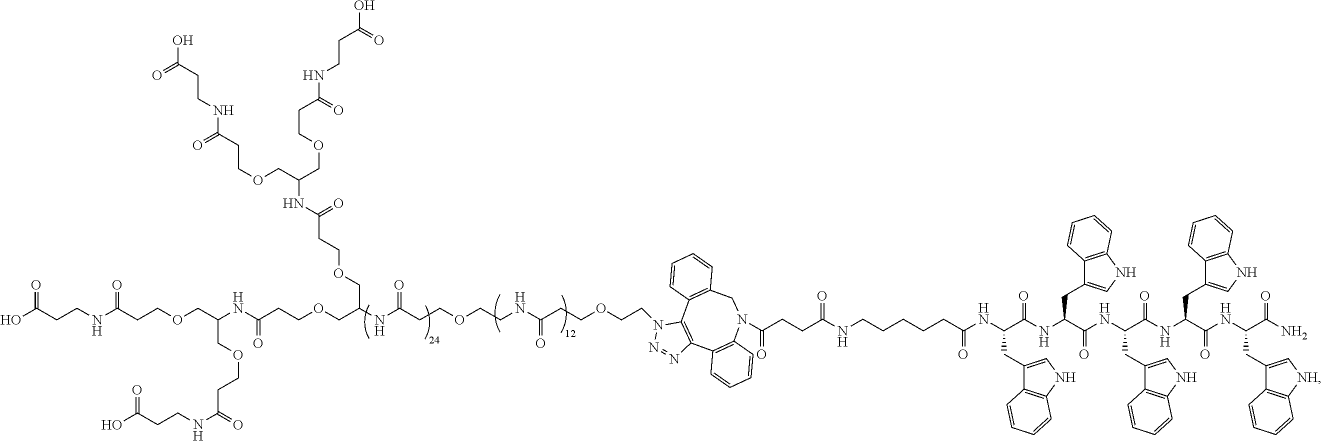

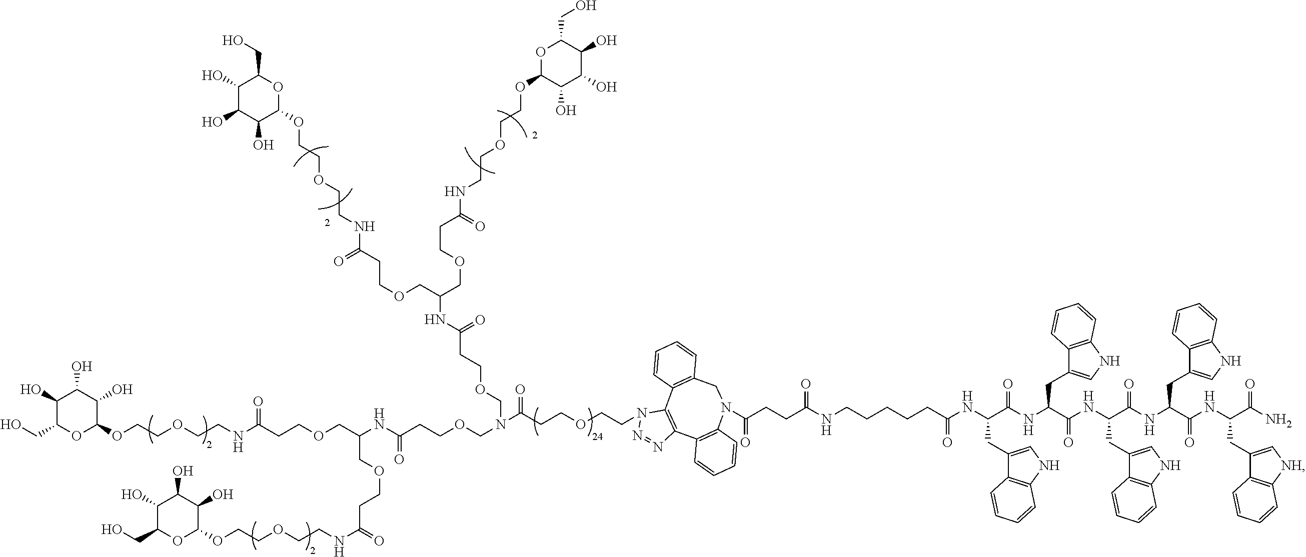

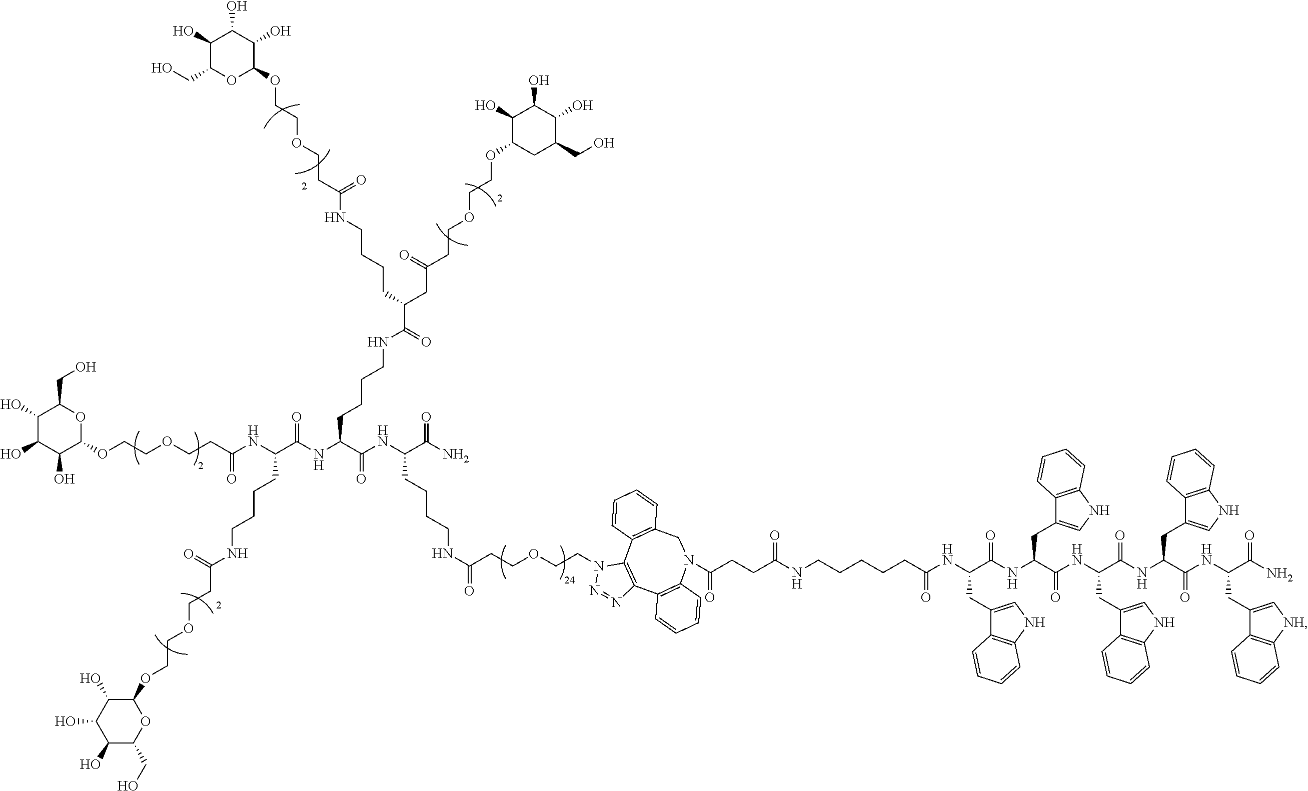

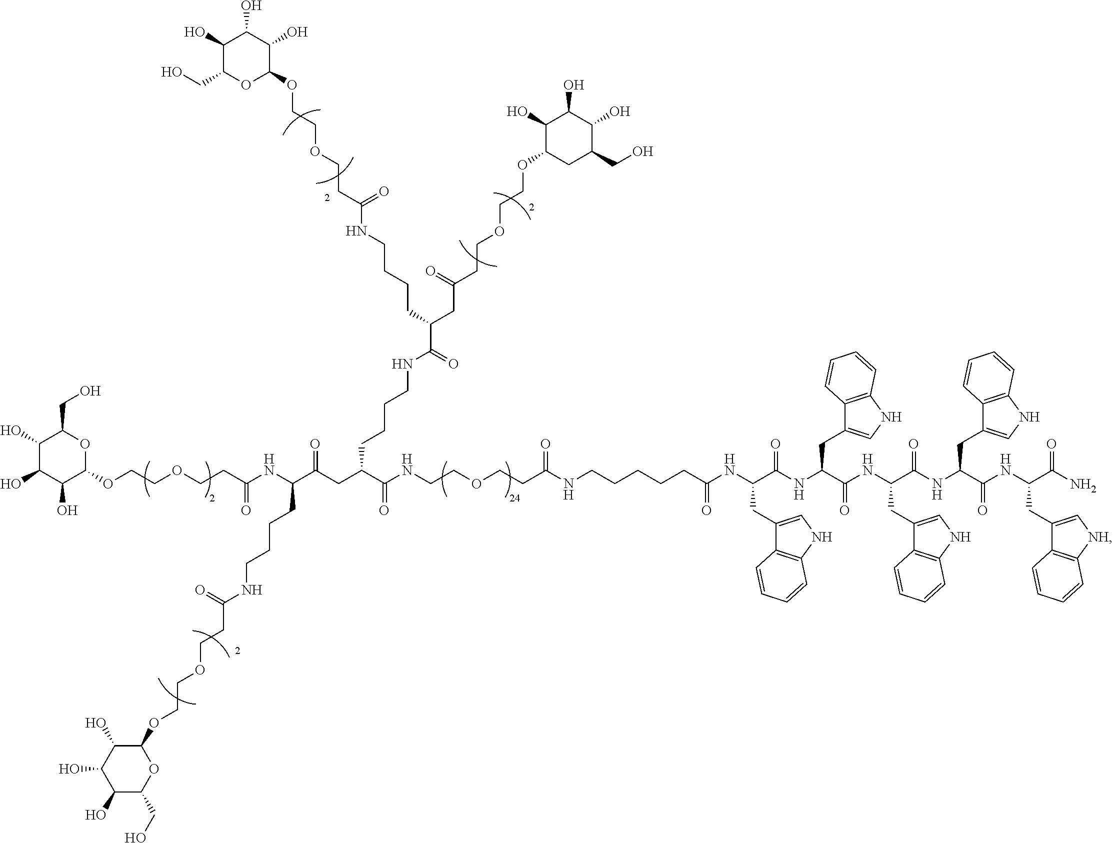

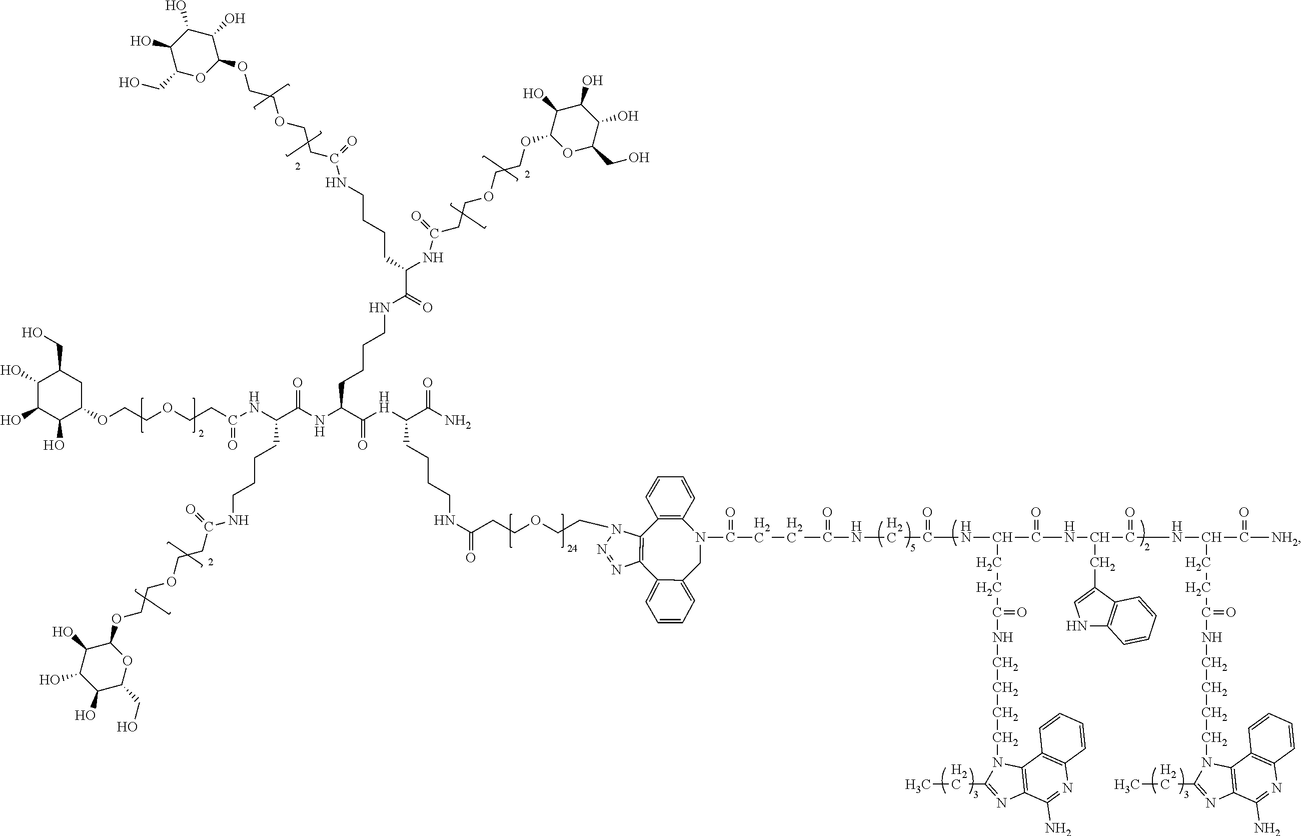

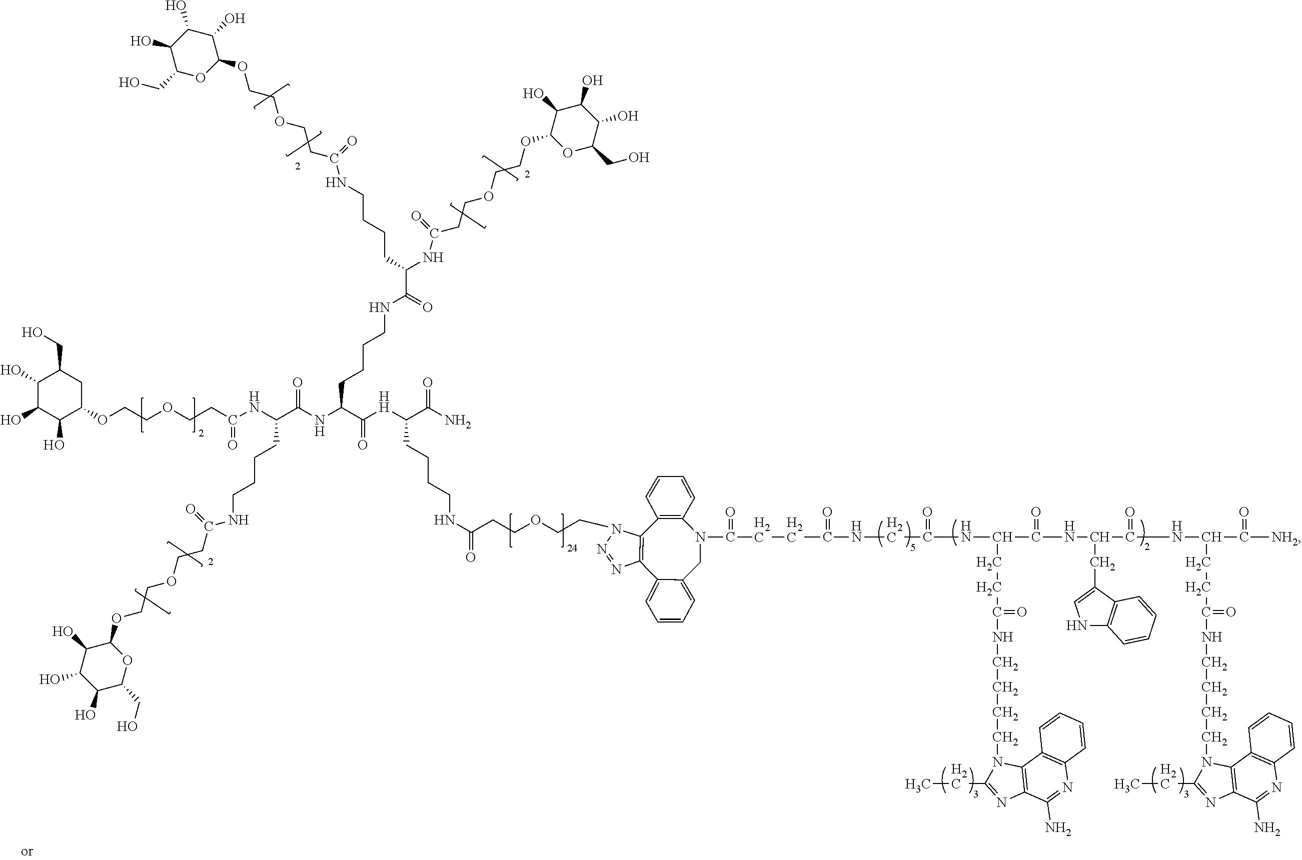

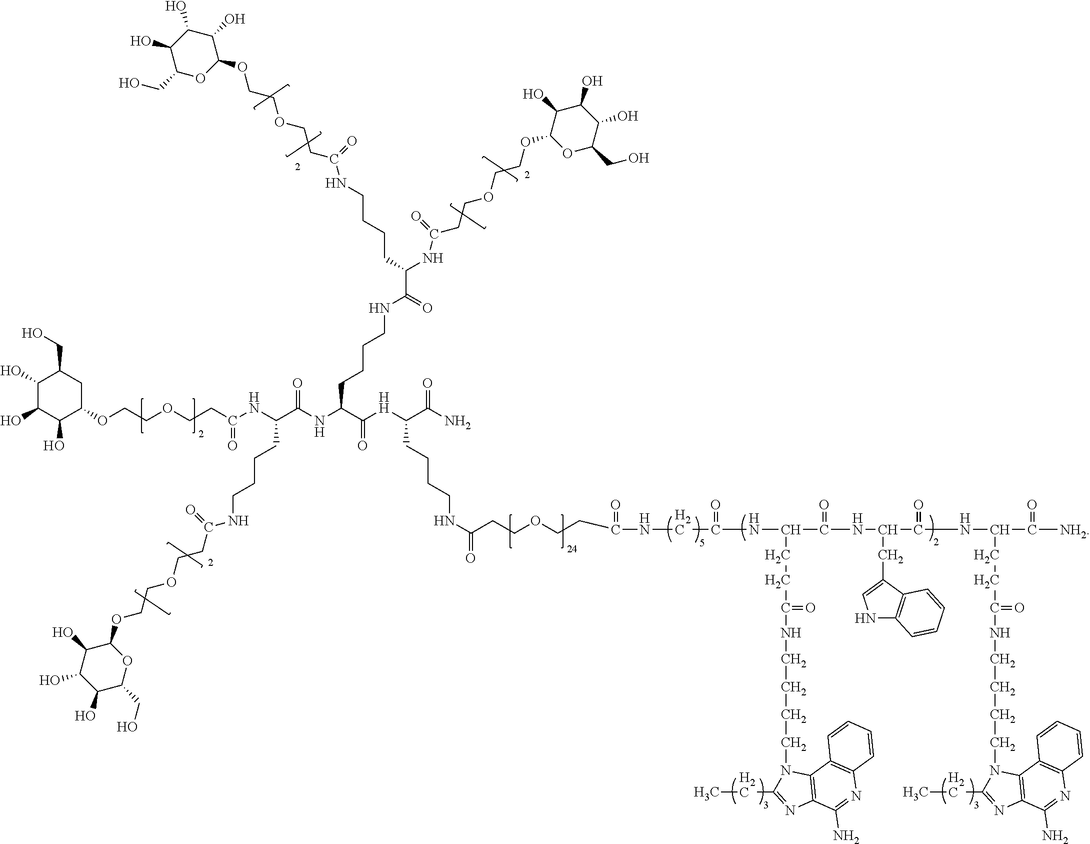

81. A method of selectively delivering the at least one drug molecule (D) to a target cell or tissue in a subject, comprising administering to the subject a composition according to claim 1.

82.-85. (canceled)

86. A method of treating a cancer, an infectious disease, a chronic viral infection, or an inflammatory disease in a subject in need thereof, comprising administering to the subject a composition according to claim 1.

87. (canceled)

88. A method of preventing or inhibiting an immune response in a subject against a second drug molecule (D2) or an expression system (D2e), wherein the method comprises administering to the subject a composition comprising:
at least one amphiphile; and
a first drug molecule (D1) selected from inhibitors of mTORC1 and/or mTORC2,
wherein the composition is administered to the subject at a time 1 (T1) and the second drug molecule (D2) or the expression system (D2e) is administered to the subject at a time (T2).

89.-120. (canceled)

121. The method according to claim 88, wherein the at least one amphiphile of the composition has the formula S-[B]-[U]-H,
wherein S, independently for each occurrence, is a solubilizing block;
B, independently for each occurrence, is a spacer;
H, independently for each occurrence, is a hydrophobic block;
U, independently for each occurrence, is a linker;
[ ] denotes that the group is optional; and
- denotes that the two adjacent groups are directly attached to one another by a covalent bond or indirectly to one another via a suitable linker X,
wherein the S of the first amphiphile comprises a dendron amplifier linked to two or more solubilizing groups (SGs) each independently selected from an amino, phosphate, hydroxyl, carboxylic acid, phosphoserinyl, an agonist of CD22a, and a sugar moiety, or any combinations thereof,
each sugar moiety is independently selected from mannose, glucosyl, glucosamino, N-acetyl glucosyl, galactosyl, galactosamino, sialyl lewix x, and N-acetyl galactosaminyl, or any combinations thereof; and
the first drug molecule (D1) is noncovalently associated with or covalently bonded directly or via a suitable linker X1 to the at least one amphiphile.

122.-134. (canceled)
